(19) United States
(12) Patent Application Publication
(10) Pub. No.: US 2007/0274946 A1
(43) Pub. Date: Nov. 29, 2007

(54) Tolerance to Graft Prior to Thymic Reactivation

(75) Inventors: Richard Lennox Boyd, Hampton (AU); Gabrielle Lianne Goldberg, Brighton (AU); Ann Patricia Chidgey, Black Rock (AU); Jayne Suzanne Sutherland, Port Melbourne (AU)

Correspondence Address: WILMER CUTLER PICKERING HALE AND DORR LLP, 60 STATE STREET, BOSTON, MA, 02109, US

(73) Assignee: Norwood Immunoloty, Ltd., Chelsea Heights (AU)

(21) Appl. No.: 10/553,608

(22) PCT Filed: Apr. 19, 2004
(86) PCT No.: PCT/US04/11919

§ 371 Date: Jul. 5, 2007

Related U.S. Application Data
(63) Continuation-in-part of application No. 10/419,039, filed on Apr. 18, 2003, now abandoned.
Application No. 10/419,039 is a continuation-in-part of application No. 09/976,596, filed on Oct. 12, 2001, now abandoned.
Application No. 09/976,596 is a continuation-in-part of application No. 09/965,462, filed on Sep. 26, 2001, now abandoned.
Application No. 09/965,462 is a continuation-in-part of application No. 09/755,646, filed on Jan. 5, 2001, now abandoned.
Application No. 09/755,646 is a continuation-in-part of application No. 09/795,286, filed on Oct. 13, 2000, now abandoned.
Application No. 09/755,646 is a continuation-in-part of application No. 09/795,302, filed on Oct. 13, 2000, now abandoned.

(30) Foreign Application Priority Data
Oct. 13, 2000 (AU) PR0745
Apr. 15, 1999 (AU) PP9778
Apr. 17, 2000 (AU) PCT/AU00/00329

(51) Int. Cl. A61K 48/00; A61K 35/12; A61P 37/00; A61P 41/00; A61K 38/20

(52) U.S. Cl. 424/85.2; 424/85.1; 424/93.21; 424/93.7

(57) ABSTRACT

The present disclosure provides methods for inducing tolerance in a recipient to a mismatched graft of an organ, tissue, and/or cells, by disrupting sex steroid signaling in the patient, wherein the bone marrow and other immune cell functionality is improved without, prior to, or concurrently with, thymic regeneration. In some embodiments, sex steroid signaling in the patient, is interrupted or ablated by the administration of LHRH agonists, LHRH antagonists, anti-LHRH receptor antibodies, anti-LHRH vaccines, anti-androgens, anti-estrogens, selective estrogen receptor modulators (SERMs), selective androgen receptor modulators (SARMs), selective progesterone response modulators (SPRMs), ERDs, aromatase inhibitors, or various combinations thereof.

### FIELD OF THE INVENTION

The present invention is in the fields of cellular immunology and graft transplantation. More specifically, the invention is directed to enhancing bone marrow (BM) hematopoiesis and functionality, enhancing BM engraftment following hematopoietic stem cell transplant (HSCT), and increasing the functionality of new and pre-existing T cells and other cells of the immune system. The invention also relates to the improvement of allogeneic graft acceptance by a recipient.

### BACKGROUND

The Immune System

The major function of the immune system is to distinguish “foreign” (i.e., derived from any source outside the body) antigens from “self” (i.e., derived from within the body) and respond accordingly to protect the body against infection. In more practical terms, the immune response has also been described as responding to danger signals. These danger signals may be any change in the property of a cell or tissue which alerts cells of the immune system that this cell/tissue in question is no longer “normal.” Such alerts may be very important in causing, for example, rejection of foreign agents such as viral, bacterial, parasitic and fungal infections; they may also be used to induce anti-tumor responses. However, such danger signals may also be the reason why some autoimmune diseases start, due to either inappropriate cell changes in the self cells which are then become targeted by the immune system (e.g., the pancreatic β-islet cells in diabetes mellitus) Alternatively, inappropriate stimulation of the immune cells themselves, can lead to the destruction of normal self cells, in addition to the foreign cell or microorganism which induced the initial response.

In normal immune responses, the sequence of events involves dedicated antigen presenting cells (APC) capturing foreign antigen and processing it into small peptide fragments which are then presented in clefts of major histocompatibility complex (MHC) molecules on the APC surface. The MHC molecules can either be of class I expressed on all nucleated cells (recognized by cytotoxic T cells (Tc or CTL)) or of class II expressed primarily by cells of the immune system (recognized by helper T cells (Th)). Th cells recognize the MHC II/peptide complexes on APC and respond. Factors released by these cells then promote the activation of either, or both of Tc cells or antibody producing B cells, which are specific for the particular antigen. The importance of Th cells in virtually all immune responses is best illustrated in HIV/AIDS where their absence through destruction by the virus causes severe immune deficiency, which eventually leads to death due to opportunistic infections. Inappropriate development of Th (and to a lesser extent Tc) can also lead to a variety of other conditions such as allergies, cancer, and autoimmunity.

The inappropriate development of such cells may be due to an abnormal thymus in which the structural organization is markedly altered e.g., in many autoimmune diseases, the medullary epithelial cells, which are required for development of mature thymocytes, are ectopically expressed in the cortex where immature T cells normally reside. This could mean that the developing immature T cells prematurely receive late stage maturation signals and in doing so become insensitive to the negative selection signals that would normally delete potentially autoreactive cells. Indeed this type of thymic abnormality has been found in NZB mice, which develop Lupus-like symptoms (Takeoka et al., (1999) Clin. Immunol. 90:388), and more recently in NOD mice, which develop type I diabetes (Thomas-Vaslin et al., (1997) P.N.A.S. USA 94:4598; Atlan-Gepner et al., (1999) Autoimmunity 3:249-260). It is not known how or when these forms of thymic abnormality develop, but it could be through the natural aging process or from destructive agents such as viral infections (changes in the thymus have been described in AIDS patients), stress, chemotherapy and radiation therapy (Mackall et al., (1995) N. Eng. J. Med. 332:143; Heitger et al., (1997) Blood 99:4053; Mackall and Gress, (1997) Immunol. Rev. 160:91). It is also possible that the defects are present at birth.

The ability to recognize antigen is encompassed in a plasma membrane receptor in T and B lymphocytes. These receptors are randomly generated by a complex series of rearrangements of many possible genes, such that each individual T or B cell has a unique antigen receptor. This enormous potential diversity means that for any single antigen the body might encounter, multiple lymphocytes will be able to recognize it with varying degrees of binding strength (affinity) and elicit varying degrees of responses. Since antigen receptor specificity arises by chance, the problem thus arises as to why the body does not self destruct through lymphocytes reacting against self antigens. Fortunately there are several mechanisms which prevent the T and B cells from doing so. Collectively, these mechanisms create a situation where the immune system is tolerant to self.

The most efficient form of self tolerance is to physically remove or kill any potentially reactive lymphocytes at the sites where they are produced. These sites include the thymus for T cells and the BM for B cells. This is called central tolerance. An important, additional method of tolerance is through regulatory Th cells which inhibit autoreactive cells either directly or via the production of cytokines. Given that virtually all immune responses require initiation and regulation by T helper cells, a major aim of any tolerance induction regime would be to target these T helper cells. Similarly, since Tc's are very important effector cells, their production is a major aim of strategies for, e.g., anti-cancer and anti-viral therapy. In addition, T regulatory cells (Tregs), such as CD4+CD25+ and NKT cells, provide a means whereby they can suppress potentially autoreactive cells.

The Thymus

The thymus essentially consists of developing thymocytes (T lymphocytes within the thymus) interspersed within the diverse stromal cells (predominantly epithelial cell subsets) which constitute the microenvironment and provide the growth factors (GF) and cellular interactions necessary for the optimal development of the T cells.

The thymus is an important organ in the immune system because it is the primary site of production of T lymphocytes. The role of the thymus is to attract appropriate BM-derived precursor cells from the blood, as described below, and induce their commitment to the T cell lineage, including the gene rearrangements necessary for the production of the T cell receptor (TCR) for antigen. Each T cell has a single TCR type and is unique in its specificity. Associated with this TCR production is cell division, which expands the number of T cells with that TCR type and hence increases the likelihood that every foreign antigen will be recognized and eliminated. However, a unique feature of T cell recognition of antigen is that, unlike B cells, the TCR only recognizes peptide fragments physically associated with MHC molecules. Normally, this is self MHC, and the ability or a TCR to recognize the self MHC/peptide complex is selected for in the thymus. This process is called positive selection and is an exclusive feature of cortical epithelial cells. If the TCR fails to bind to the self MHC/peptide complexes, the T cell dies by “neglect” because the T cells needs some degree of signalling through the TCR for its continued survival and maturation.

Since the outcome of the TCR gene rearrangements is a random event, some T cells will develop which, by chance, can recognize self MHC/peptide complexes with high affinity. Such T cells are thus potentially self-reactive and could be involved in autoimmune diseases, such as multiple sclerosis (MS), rheumatoid arthritis (RA), diabetes, thyroiditis and systemic lupus erythematosus (SLE). Fortunately, if the affinity of the TCR to self MHC/peptide complexes is too high, and the T cell encounters this specific complex in the thymus, the developing thymocyte is induced to undergo a suicidal activation and dies by apoptosis, a process called negative selection. This process is also called central tolerance. Such “high affinity for self” T cells die rather than respond because in the thymus they are still immature. The most potent inducers of this negative selection in the thymus are APC called dendritic cells (DC). DC deliver the strongest signal to the T cells, which causes deletion in the thymus. However, in the peripheral lymphoid organs where the T cells are more mature, the DC presenting the same MHC/peptide complex to the same TCR would cause activation of that T cell bearing the TCR.

Thymus Atrophy and Age

While the thymus is fundamental for a functional immune system, releasing about 1% of its T cell content into the bloodstream per day, one of the apparent anomalies of mammals and other animals is that this organ undergoes severe atrophy as a result of sex steroid production. This atrophy occurs gradually over a period of about 5-7 years, with the nadir level of T cell output being reached around 20 years of age (Douek et al., Nature (1998) 396:690-695) and is in contrast to the reversible atrophy induced during a stress response to corticosteroids. Structurally, the thymic atrophy involves a progressive loss of lymphocyte content, a collapse of the cortical epithelial network, an increase in extracellular matrix material, and an infiltration of the gland with fat cells (adipocytes) and lipid deposits (Haynes et al., (1999) J. Clin. Invest. 103: 453). This process may even begin in young children (e.g., around five years of age; Mackall et al., (1995) N. Eng. J. Med. 332:143), but it is profound from the time of puberty when sex steroid levels reach a maximum.

The impact of thymus atrophy is reflected in the periphery, with reduced thymic input to the T cell pool, which results in a less diverse TCR repertoire (as this can only be provided by the new naïve T cells). Altered cytokine profiles (Hobbs et al., (1993) J. Immunol. 150:3602; Kurashima et al., (1995) Int. Immunol. 7:97), changes in CD4⁺ and CD8⁺ subsets, biases towards memory as opposed to naïve T cells (Mackall et al., (1995) N. Engl. J. Med. 332:143), and a reduced ability to response to antigenic or mitogenic stimulation are also observed.

Since the thymus is the primary site for the production and maintenance of the peripheral T cell pool, this atrophy has been widely postulated as being the primary cause of the increased incidence of immune-based disorders in the elderly. In particular, conditions, such as general immunodeficiency, poor responsiveness to opportunistic infections and vaccines, and an increase in the frequency of autoimmune diseases, such as multiple sclerosis, rheumatoid arthritis, and lupus Doria et al., (1997) Mech. Age. Dev. 95: 131-142; Weyand et al., (1998) Mech. Age. Dev. 102: 131-147; Castle, (2000) Clin Infect Dis 31(2): 578-585; Murasko et al., (2002) Exp. Gerontol. 37:427-439), increase in incidence and severity with age. Such deficiencies of the immune system, are often illustrated by a decrease in T cell dependent immune functions (e.g., cytolytic T cell activity and mitogenic responses). While homeostatic mechanisms maintain T cell numbers in healthy individuals, when there is a major loss of T cells, e.g., in AIDS, and following chemotherapy or radiotherapy, adult patients are highly susceptible to opportunistic infections because all these conditions involve a loss of T cells and/or other blood cells (see below). Lymphocyte recovery is also severely retarded. The atrophic thymus is unable to reconstitute CD4+ T cells that are lost during HIV infection (Douek et al. Nature (1998) 396:690-695) and CD4+ T cells take three to four times longer to return to normal levels following chemotherapy in post-pubertal patients as compared to pre-pubertal patients (Mackall et al. (1995) N. Engl. J. Med. 332:143-149). As a consequence these patients lack the cells needed to respond to infections, and they become severely immune suppressed (Mackall et al. (1995) N. Eng. J. Med. 332:143; Heitger et al., (2002) Blood 99:4053). There is also an increase in cancers and tumor load in later life (Hirokawa, (1998) “Immunity and Ageing,” in PRINCIPLES AND PRACTICE OF GERIATIC MEDICINE, (M. Pathy, ed.) John Wiley and Sons Ltd; Doria et al., (1997) Mech. Age. Dev. 95: 131; Castle, (2000) Clin. Infect. Dis. 31:578).

However, recent work by Douek et al., ((1998) Nature 396:690) has shown thymic output occurs even if only very slight (about 5% of the young levels), in older humans (e.g., even sixty-five years old and above, and after anti-retroviral treatment in older HIV patients). This was exemplified by the presence of T cells with T Cell Receptor Excision Circles (TRECs); TRECs are formed as part of the generation of the TCR for antigen and are only found in newly produced T cells). Furthermore Timm and Thoman ((1999) J. Immunol. 162:711) have shown that although CD4⁺ T cells are regenerated in old mice post-bone marrow transplant (BMT), they appear to show a bias towards memory cells due to the aged peripheral microenvironment coupled to poor thymic production of naïve T cells. TREC levels has also been analysed following hematopoietic stem cell transplantation (Douek et al., (2000) Lancet 355:1875).

### Thymus and the Neuroendocrine Axis

The thymus is influenced to a great extent by its bidirectional communication with the neuroendocrine system (Kendall, (1988) “Anatomical and physiological factors influencing the thymic microenvironment,” in THYMUS UPDATE I, Vol. 1. (M. D. Kendall, and M. A. Ritter, eds.) Harwood Academic Publishers, p. 27). Of particular importance is the interplay between the pituitary, adrenals, and gonads on thymic function, including both trophic (thyroid stimulating hormone or TSH, and growth hormone or GH) and atrophic effects (luteinizing hormone or LH, follicle stimulating hormone or FSH, and adrenocorticotropic hormone or ACTH) (Kendall, (1988) “Anatomical and physiological factors influencing the thymic microenvironment,” in THYMUS UPDATE I, Vol. 1. (M. D. Kendall, and M. A. Ritter, eds.) Harwood Academic Publishers, p. 27; Homo-Delarche et al., (1993) Springer Sem. Immunopathol. 14:221). Indeed, one of the characteristic features of thymic physiology is the progressive decline in structure and function, which is commensurate with the increase in circulating sex steroid production around puberty, which in humans generally occurs from the age of 12-14 onwards (Hirokawa and Makinodan, (1975) J. Immunol. 114:1659; Tosi et al., (1982) Clin. Exp. Immunol. 47:497; and Hirokawa, et al., (1994) Immunol. Lett. 40:269).

The thymus essentially consists of developing thymocytes interspersed within the diverse stromal cells (predominantly epithelial cell subsets) which constitute the microenvironment and provide the growth factors and cellular interactions necessary for the optimal development of the T cells. The precise target of the hormones, as well as the mechanism by which they induce thymus atrophy and improved immune responses, has yet to be determined. Examination of testicular feminised mutant mice, however, indicates that functional sex steroid receptors must be expressed on the stromal cells of the thymus for atrophy to occur. The symbiotic developmental relationship between thymocytes and the epithelial subsets that controls their differentiation and maturation (Boyd et al., (1993) Immunol. Today 14:445) means that sex-steroid inhibition could occur at the level of either cell type, which would then influence the status of the other cell type. Bone marrow stem cells are reduced in number and are qualitatively different in aged patients. HSC are able to repopulate the thymus, although to a lesser degree than in the young. Thus, the major factor influencing thymic atrophy is appears to be intrathymic. Furthermore, thymocytes in older aged animals (e.g., those ≧18 months) retain their ability to differentiate to at least some degree (George and Ritter, (1996) Immunol. Today 17:267; Hirokawa et al., (1994) Immunology Letters 40:269; Mackall et al., (1998) Eur. J. Immunol. 28: 1886). However, recent work by Aspinall has shown that in aged mice there is a defect in thymocyte production, which is manifested as a block within the precursor triple negative population, namely the CD44+CD25+ (TN2) stage. (Aspinall et al., (1997) J. Immunol. 158:3037).

Hematopoiesis

Bone Marrow and Hematopoietic Stem Cells

The major cells of the immune system are the B and T lymphocytes (a major class of white blood cells), and the antigen presenting cells (APC). All immune cells are basically derived from hematopoietic stem cells (HSC) and their progeny, the Common Lymphoid Progenitor (CLP) and the Common Myeloid Progenitor (CMP), which are produced in the BM. Some of the precursor cells migrate to the thymus and are converted into T cells and thymic DC. DC play a role in inducing self-tolerance.

Only a small proportion (approximately 0.01% in young animals) of the HSC are released from the BM and find their way to the thymus via the blood supply, where they undergo division and maturation to form T cells, which are then returned into the general circulation. These new recent thymic emigrant (RTE) cells form a major part of the immune system, and are primarily Th and Tc and are important in maintaining a constant supply of new T cells with a diverse TCR repertoire for the initiation of almost all immune responses. Prior to leaving the thymus, Th and Tc cells can effectively distinguish foreign antigen because, as described above, T cells are “selected” in the thymus so that those T cells that leave recognize all of the cells in the body as self and, under normal circumstances, do not respond against them.

B cells are also ultimately derived from HSC and develop in the BM before exiting into the peripheral immune system. Following interactions with T cells, and other cells of the immune system, B cells develop into plasma cells that produce and release large amounts of antibodies, which help the body destroy infective organisms and abnormal cells.

Other HSC produced by the BM are also utilized for the production of all other blood cells, such as NK cells, regulatory cells, common myeloid progenitor derived cells, neutrophils, basophils and eosinophils, dendritic cells, monocytes, macrophages, platelets and red blood cells.

Hematopoietic Stem Cell Transplantation

Hematopoietic stem cell transplantation (HSCT)—also commonly known as bone marrow transplantation (BMT))—is a treatment used to enhance the recovery of the immune system in, e.g., certain critical cancer conditions. “HSCT” and “BMT” and “transplant” are used interchangeably and are herein defined as a transplant into a recipient, containing or enriched for HSC, BM cells, stem cells, and/or any other cells which gives rise to blood, thymus, BM and/or any other immune cells, including, but not limited to, HSC, epithelial cells, common lymphoid progenitors (CLP), common myelolymphoid progenitors (CMLP), multilineage progenitors (MLP), and/or mesenchymal stem cells in the BM. In some embodiments, the transplant may be a peripheral blood stem cell transplant (PBSCT). The HSC maybe be mobilized from the BM and then harvested from the blood, or contained within BM physically extracted from the donor. The HSC may be either purified, enriched, or simply part of the collected BM or blood, and are then injected into a recipient. Transplants may be allogeneic, autologous, syngeneic, or xenogenic, and may involve the transplant of any number of cells, including “mini-transplants,” which involve smaller numbers of cells. In some embodiments, HSCT is given prior to, concurrently with, or after sex steroid inhibition.

HSC is a nonlimiting exemplary type of cell, which may be transplanted and/or genetically modified, as used throughout this application. However, as will be readily understood by one skilled in the art, in practicing the inventions provided herein, HSC may be replaced with any one (or more) of a number of substitute cell types without undue experimentation, including but not limited to BM cells, stem cells, and/or any other cell which gives rise to blood, thymus, BM and/or any other immune cells, including, but not limited to, HSC, epithelial stem cells, CLP, CMLP, MLP, and/or mesenchymal stem cells in the BM. In some embodiments, HSC are derived from a fetal liver and/or spleen.

Both chemotherapy and radiation therapy can destroy cancer cells. However, because of the lack of specificity, these therapies also kill healthy cells, including virtually all white blood cells (WBC), as well as the HSC in the BM. It is this destruction of WBC and HSC that leads to the patient's need for HSCT. HSCT allows, for example, stem cells and their progeny cells that were damaged by, e.g., chemotherapy or radiation treatment to be replaced with healthy stem cells that can ultimately produce the blood cells that the patient needs.

HSCT is the basic treatment for a number of hematological cancers, such as leukemias and lymphomas (cancers of the blood and immune system cells), as well as non-malignant immune disorders such as severe combined immunodeficiency, Fanconi's anemia, myelodysplastic syndromes, amyloidosis, aplastic anemia, Diamond Blackfan anemia, hemophagocytic lymphohitiocytosis, Kostmann syndrome, Wiskott-Aldrich syndrome, thrombocytopenias, and hemoglobinopathologies, such as sickle cell disease and thalassemia. Leukemia and lymphoma, which are commonly treated by myeloablation or myelodepletion to rid the body of cancerous cells, are commonly followed by HSCT to recover immune function. The methods of the present invention either alone or in combination (concurrently or sequentially) with the administration of HSC mobilizing agents, such as cytokines (e.g., G-CSF or GM-CSF), or drugs (e.g., cyclophosphamide), allow faster and/or better engraftment and may also allow chemotherapy and radiation therapy to be given at higher doses and/or more frequently.

Modern clinical medical procedures often employ a transplantation of HSC derived from another donor's blood (an allogeneic HSCT), where advantage is taken of donor T cells reacting against the host cancer cells (graft versus leukaemia (GVL)) but this is counterbalanced by other T donor T cells reacting against the host in general (graft versus host (GVH) disease) which can be fatal. Since the success of HSCT, and hence patient survival, is directly related to the number of HSC injected and the speed of engraftment, using the methods of the present invention means that current HSCT programs will be more successful and that many more patients will be able to receive HSCT than is currently possible.

Mechanisms of enhancing HSC mobilization from the BM are important in ensuring that as many HSC as possible are available for collection from a donor. GM-CSF and G-CSF are presently used for this purpose, but other agents, such as chemotherapy and cytokines have also been shown to be effective. The ability to more effectively mobilize HSC has application beyond hematological repair. Recent studies have shown that HSC are multipotent and may be utilized for repair of damaged tissues, e.g., cardiac muscle, skeletal muscle, liver, bone, connective tissue, epithelial tissue, pancreas, vasculature.

One limitation of current HSCT strategies is associated with infection, particularly viral, fungal, and encapsulated bacteria, due to prolonged immunodeficiency and this remains a significant cause of post-transplantal morbidity and mortality in adults. The infections associated with HSCT are generally very difficult to control, even with modern antimicrobial reagents. Children generally recover immune capacity within months after HSCT (Parkman et al., (1997) Immunol. Rev. 157:73), in contrast to the delay in lymphoid recovery in adult recipients which may last years and even then be a very poor reflection of the young optimum. This delay in adults is dependent on a variety of factors, but the susceptibility to infection is primarily due to the well-recognized decline in T and B cell production with age (Parkman et al., (1997) Immunol. Rev. 157:73).

A second limitation of current HSCT strategies occurs when the grafted cells ‘reject’ the recipient of the cells. This is known clinically as “graft versus host disease” (GVHD). An autologous transplant may avoid GVHD. However, the overall anti-cancer success rates of autologous transplants are lower as compared to allogeneic transplants. In cancer patients, autologous transplants have the disadvantage that they do not produce a Graft Versus Tumor (GVT) effect (which is similar to the GVH effect) and there is the risk that cancerous cells may be returned to the patient with the transplant. It has been discovered that sex steroid inhibition in murine allogeneic HSCT models and castrated recipients of allogeneic HSCT improves post-transplant reconstitution of cells of both the myeloid and lymphoid lineages. Data presented herein shows a significant increase in T and B cell reconstitution without an exacerbation of GVHD or loss of GVT activity (see, e.g., Example 19).

A further limitation of HSCT treatments is the lack of donors to treat all the potential candidates. Although umbilical cord blood (UCB) has been utilized to a fairly limited extent, there are few cells from each donor and as a consequence this has been mainly used in children where the total number of HSC required is lower (HSC number required is linked to patient body weight). Other than UCB, donors are in limited supply and there must be an acceptable MHC match or the risk of GVH is high. If less cells were required, as a result of improved engraftment or a less rigorous match was required, thus reducing the risk of rejection or GVH, potentially HSCT could be used more widely, for example to treat autoimmune disease, and sources such as cord blood could be utilized (e.g., 1.5×10⁷ cells/kg for recipient engraftment).

T Cells

T cells are the major component of the immune system, and are produced in the thymus. The most important T cells are Th cells because these are the cells that initiate virtually all immune responses. The absence of these Th cells (e.g., caused by HIV infection, chemotherapy, radiation, etc.) directly results immunosuppression and the consequent susceptibility to infections and tumors, and death occurs quickly. An important role of a subset of Th cells is to regulate immune responses. The balance between enhancement and suppression of T and B cell function has a major effect on e.g., whether a vaccine is efficacious, whether a cancer or tumor is attacked, or whether a transplant is tolerated or rejected.

The thymus, while being very active in the young, progressively declines in both size and functional output with age. This is particularly evident at the onset of puberty. Since thymocyte export is directly related to the cellularity in the thymus (Scollay et al., (1980) Eur. J. Immunol. 10:210; Berzins et al., (1998) J. Exp. Med. 187:1839), age-related thymic atrophy results in a gradual decrease in recent thymic emigrants (RTEs) (Steffens et al., (2000) Clin. Immunol. 97:95; Sempowski et al., (2002) Mol. Immunol. 38:841-848; Sutherland et al., (submitted)) and a decrease in the naïve to memory T cell ratio (Ernst et al., (1990) J. Immunol. 145:1295); Kurashima et al., (1995) Int. Immunol. 7:97; Utsuyama et al., (1992) Mech. Ageing Dev. 63:57) resulting in a restricted TCR repertoire in both CD4⁺ and CD8⁺ T cells (Mosley et al., (1998) Cell. Immunol. 189: 10; LeMaoult et al., (2000) J. Immunol. 165:2367). Consequently, T cell proliferation in response to non-specific and receptor-mediated (CD3/TCR) stimulation is severely compromised with age (Hertogh-Huijbregts et al., (1990) Mech. Ageing Dev. 53:141-155; Flurkey et al., (1992) J. Gerontol. 47:B115; Kirschmann et al., (1992) Cell. Immunol. 139:426).

With increasing age there is a gradual decline in the immune function of humans; children respond very well, younger adults reasonably so, but from middle age and older, this response can be very poor. This decline indicates the presence of deficiencies or alterations in one of more of the three major cell types involved in virtually all response: (i) antigen presenting cells (which capture antigen and present it to, and thereby activate, T lymphocytes); (ii) T lymphocytes, and (iii) B lymphocytes. Deficiencies or alterations in any one of these three cell types may explain why the immune response to stimulation against antigens may be suboptimal. The deficiency or alterations may be at the level of the cell or may refer to quantity or functionality. Of these, the most likely defect is encompassed within the T cell compartment because of the dramatic decline in thymus function with age primarily due to the impact of sex steroids. This leads to a loss of new or “naïve” T cells exported into the bloodstream, which are needed for responses to new antigens. In addition to the numerical loss of potential responding T cells, the pre-existing T cells may be suppressed to some degree by the presence of sex steroids.

Cancer Therapy

As indicated above, chemotherapy and radiotherapy used to treat cancers are often deleterious to the patient's non-cancerous cells, particularly the blood cells. The major limitation to increasing frequency and dose of such treatments is the ability of the patient to survive the treatment and avoid susceptibility to opportunistic infection as a result of the compromised immune system. Thus it would greatly benefit the patient if the immune recovery was more rapid or the damage less severe.

### SUMMARY OF THE INVENTION

The present invention relates to methods for enhancing donor graft acceptance by a patient recipient by enhancing BM haemopoieses and functionality, enhancing BM engraftment following HSCT, and increasing the functionality of preexisting T cells and other immune cells by disrupting sex steroid and other hormonal signaling. Immune capacity will also be enhanced by increased levels of naïve T cells produced through renewed thymic function, and also B lymphocytes and other cells of the immune system produced via activated BM function.

It has been discovered that interruption of sex steroid and/or other hormonal signaling enhances the functionality of BM, HSC, T cells and other cells of the immune system, either by direct effects or indirect effects. This discovery has been exploited to produce the present invention which, in one aspect, provide methods of enhancing BM haemopoiesis and/or functionality. In some embodiments, haemopoiesis and/or functionality of pre-existing BM is improved. In another embodiment, a patient receives an HSCT, and the HSC haemopoiesis and/or engraftment is improved in the patient.

In one aspect, the invention provides methods of enhancing engraftment following HSCT. In one embodiment, engraftment is enhanced in the BM. In another embodiment, engraftment and/or reconstitution is enhanced in the thymus, whereby thymic recovery is ultimately induced. In yet another embodiment, engraftment and/or reconstitution is enhanced in the spleen and/or other lymphoid organs, tissues, and/or blood. In some embodiments, the HSC are allogeneic, and in other embodiments, the HSC are autologous. In one embodiment, the number of T cell precursors is increased as compared to the number that would have been present in a patient that had HSCT without undergoing interruption of sex steroid signaling. In other embodiments total white blood cells, donor-derived DC, BM precursors, HSC, CLP, MLP, lymphocytes, myeloid cells, granulocytes, neutrophils, macrophage, NK, NKT, platelets, naïve T cells, memory T cells, helper T cells, effector T cells, regulatory T cells, RBC, B cells, donor-, and/or host-derived peripheral T cells, APC, and/or donor derived peripheral B cells are increased as compared to the number that would have been present in a patient that had HSCT without undergoing interruption of sex steroid signaling. In yet other embodiments, a patient is also treated with a cytokine (e.g., IL-7, SCF, IL-11, G-CSF, or GM-CSF) or hormone (e.g., growth hormone, or its mediator insulin dependent growth factor (IGF-1) or any member of the fibroblast growth factor family e.g., FGF 7/Keratinocyte Growth Factor (KGF)) following HSCT to enhance immune recovery and/or engraftment following HSCT to enhance immune recovery and/or engraftment. In another embodiment, the present invention either alone, or in combination (concurrently or sequentially) with the administration of hemopoietic agents, such as cytokines (e.g., G-CSF or GM-CSF), allow faster and/or better engraftment and/or homing to the target tissue and/or enhance recovery of immune cells.

In a second aspect of the invention, methods of enhancing the functionality of immune cells in a patient following HSCT are provided. In one embodiment, the immune cells are T cells. In another embodiment, the T cell proliferative responsiveness to T cell receptor (TCR) stimulation is improved. In another embodiment, the T cell responsiveness to an antigen (e.g., tetanus toxoid (TT) or pokeweed mitogen (PWM)) stimulation is improved. In one embodiment, the T cell responsiveness is improved to a recall antigen (i.e., an improved T cell memory response). In yet another embodiment the T cell proliferative responsiveness in respect of co-stimulatory or secondary signaling is improved. In some embodiments, the kinetics of T cell responsiveness is improved. In other embodiments, the T cell response to antigen presented by APC is improved. In some embodiments of the invention, immune cell responsiveness is improved within about five-, four-, three- or two months post transplant. In certain embodiments of the invention, immune cell responsiveness is improved within about one month post-transplant. In other embodiments of the invention, immune cell responsiveness is improved within two weeks post-transplant. In another embodiment of the invention, immune cell responsiveness is improved within one week post-transplant. In yet other embodiments of the invention, immune cell responsiveness is improved within three days post transplant. In other embodiments the immune response is improved after 3 or more months post treatment involving at this time input from newly thymic derived T cells in addition to preexisting T cells.

In a third aspect, the invention provides methods of enhancing pre-existing immune cell functionality, including, but not limited to, immune cells in the periphery. In one embodiment, the cells are T cells. In another embodiment, the cells are DC or other APC. In yet another embodiment, the cells are NK cells or regulatory cells, such as CD4+CD25+ T cells and natural killer T (NKT) cells. In one embodiment of the invention, T cell proliferative responsiveness to TCR stimulation is improved in the patient. In another embodiment, the T cell responsiveness to antigen (e.g., TT, PWM, or KLH) stimulation is improved. In yet another embodiment the T cell proliferative responsiveness to secondary or co-stimulatory signaling is improved. In other embodiments, the T cell response to antigen presented by APC is improved. In one specific embodiment, a GnRH/LHRH analog has a direct or indirect effect on the responsiveness of preexisting immune cells. In some embodiments, sex steroid analogs (agonist and antagonists thereto), such as GnRH/LHRH analogs, are used in the methods of the invention to disrupt sex steroid-mediated signaling, immune cells or BM. In other embodiments, sex steroid analogs directly stimulate (i.e., directly increase the functional activity of) the thymus, BM, and/or preexisting cells of the immune system, such as T cells, B cells, and DC.

In a fourth aspect, the invention provides methods of enhancing HSC engraftment and mobilization in a patient, or in a blood, HSC, or BM donor. In one embodiment disrupting sex steroid signaling increases the number and/or functionality of peripheral immune progenitor cells such as HSC, CD34+ cells, CLP, or CMP. One embodiment provides a method for enhancing HSC mobilization comprising disrupting sex steroid signaling, either alone or in combination with administration of an HSC mobilizing agent, for example, cytokines, GM-CSF, G-CSF, CSF, chemotherapeutics, cyclophosphamide, flt-3 ligand, KGF/FGF 7 or other members of the FGF family or IL-7.

In one embodiment, the present invention provides methods to allow chemotherapy and radiation therapy to be given at higher doses and/or more frequently and/or allow faster recovery of or less damage to the immune system after chemotherapy and radiation therapy.

In a fifth aspect, the invention provides for disruption of sex steroid mediated signaling to, and subsequent reactivation of, the thymus. In one embodiment, castration is used to disrupt the sex steroid mediated signaling. In a particular embodiment, chemical castration is used. In another embodiment, surgical castration (e.g., by removal of the testes or by ovariectomy) is used. In some embodiments, complete inhibition of sex steroid signaling occurs. In another embodiment, partial disruption of sex steroid signaling occurs. In one embodiment, castration reverses the state of the thymus towards its pre-pubertal state, thereby reactivating it. In another embodiment, castration modifies the level of other molecules which enhances immune cell responsiveness and/or proliferation and/or activation state by having, e.g., a direct effect on preexisting immune cells.

In a sixth aspect, the invention provides for disruption of sex steroid mediated signaling to, and subsequent reactivation of, the thymus. In one embodiment, castration is used to disrupt the sex steroid mediated signaling. In a particular embodiment, chemical castration is used. In another embodiment, surgical castration (e.g., by removal of the testes or by ovariectomy) is used. In some embodiments, complete inhibition of sex steroid signaling occurs. In another embodiment, partial disruption of sex steroid signaling occurs. In one embodiment, castration reverses the state of the thymus towards its pre-pubertal state, thereby reactivating it. In another embodiment, castration modifies the level of other molecules, which enhance immune cell responsiveness and/or proliferation and/or activation state by having, e.g., a direct effect on pre-existing immune cells.

In certain embodiments, sex steroid mediated signaling may be directly or indirectly blocked (e.g., inhibited, inactivated or made ineffectual) by the administration of modifiers of sex hormone production, action, binding or signaling, including but not limited to agents which bind a sex hormone or its receptor, agonists or antagonists of sex hormones, including, but not limited to, LHRH/GnRH, anti-estrogenic and anti-androgenic agents, SERMs, SARMs, anti-estrogen antibodies, anti-androgen ligands, anti-estrogen ligands, LHRH ligands, passive (antibody) or active (antigen) anti-LHRH (or other sex steroid) vaccinations, or combinations thereof (“blockers”). In one embodiment, one or more blocker is used. In some embodiments, the one or more blocker is administered by a sustained peptide-release formulation. Examples of sustained peptide-release formulations are provided in WO 98/08533, the entire contents of which are incorporated herein by reference.

### DESCRIPTION OF THE FIGURES

FIGS. 1A-B: Castration rapidly regenerates thymus cellularity. FIGS. 1A-B are graphic representations showing that the changes in thymus weight and thymocyte number pre- and post-castration. Thymus atrophy results in a significant decrease in thymocyte numbers with age, as measured by thymus weight (FIG. 1A) or by the number of cells per thymus (FIG. 1B). For these studies, aged (i.e., 2-year old) male mice were surgically castrated. Thymus weight in relation to body weight (FIG. 1A) and thymus cellularity (FIG. 1B) were analyzed in aged (1 and 2 years) and at 2-4 weeks post-castration (post-cx) male mice. A significant decrease in thymus weight and cellularity was seen with age compared to young adult (2-month) mice. This decrease in thymus weight and cell number was restored by castration, although the decrease in cell number was still evident at 1 week post-castration (FIG. 1C). By 2 weeks post-castration, cell numbers were found to increase to approximately those levels seen in young adults (FIG. 1B). By 3 weeks post-castration, numbers have significantly increased from the young adult and these were stabilized by 4 weeks post-castration (FIG. 1B). Results are expressed as mean±1SD of 4-8 mice per group (FIG. 1A) or 8-12 mice per group (FIG. 1B). **=p≦0.01; ***=p≦0.001 compared to young adult (2 month) thymus and thymus of 2-6 wks post-castrate mice.

FIGS. 2A-D: Castration restores the CD4:CD8 T cell ratio in the periphery. For these studies, aged (2-year old) mice were surgically castrated and analyzed at 2-6 weeks post-castration for peripheral lymphocyte populations. FIGS. 2A and 2B show the total lymphocyte numbers in the spleen. Spleen numbers remain constant with age and post-castration because homeostasis maintains total cell numbers within the spleen (FIGS. 2A and 2B). However, cell numbers in the lymph nodes in aged (18-24 months) mice were depleted (FIG. 2B). This decrease in lymph node cellularity was restored by castration (FIG. 2B). FIG. 2C shows that the ratio of B cells to T cells did not change with age or post-castration in either the spleen or lymph node, as no change in this ratio was seen with age or post-castration. However, a significant decrease (p<0.001) in the CD4+:CD8+ T cell ratio was seen with age in both the (pooled) lymph node and the spleen (FIG. 2E). This decrease was restored to young adult (i.e., 2 month) levels by 4-6 weeks post-castration (FIG. 2D).

Results are expressed as mean±SD of 4-8 (FIGS. 2A, 2C, and 2E) or 8-10 (FIGS. 2B, 2D, and 2F) mice per group. *=p≦0.05; **=p≦0.01; ***=p≦0.001 compared to young adult (2-month) and post-castrate mice.

FIG. 3: Thymocyte subpopulations are retained in similar proportions despite thymus atrophy or regeneration by castration. For these studies, aged (2-year old) mice were castrated and the thymocyte subsets analyzed based on the markers CD4 and CD8. Representative Fluorescence Activated Cell Sorter (FACS) profiles of CD4 (X-axis) vs. CD8 (Y-axis) for CD4−CD8−DN, CD4+CD8+DP, CD4+CD8− and CD4−CD8+ SP thymocyte populations are shown for young adult (2 months), aged (2 years) and aged, post-castrate animals (2 years, 4 weeks post-cx). Percentages for each quadrant are given above each plot. No difference was seen in the proportions of any CD4/CD8 defined subset with age or post-castration. Thus, subpopulations of thymocytes remain constant with age and there was a synchronous expansion of thymocytes following castration.

FIGS. 4A-B: Regeneration of thymocyte proliferation by castration. Mice were injected with a pulse of BrdU and analyzed for proliferating (BrdU⁺) thymocytes. For these studies, aged (2-year old) mice were castrated and injected with a pulse of bromodeoxyuridine (BrdU) to determine levels of proliferation. Representative histogram profiles of the proportion of BrdU+ cells within the thymus with age and post-castration are shown (FIG. 4A). No difference was observed in the total proportion of proliferation within the thymus, as this proportion remains constant with age and following castration (FIG. 4A and left graph in FIG. 4B). However, a significant decrease in number of BrdU⁺ cells was seen with age (FIG. 4B, right graph). By 2 weeks post-castration, the number of BrdU⁺ cells increased to a number that similar to seen in young adults (i.e., 2 month) (FIG. 4B, right graph). Results are expressed as mean±1SD of 4-14 mice per group. ***=p≦0.001 compared to young adult (2-month) control mice and 2-6 weeks post-castration mice.

FIGS. 5A-H: Castration enhances proliferation within all thymocyte subsets. For these studies, aged (2-year old) mice were castrated and injected with a pulse of bromodeoxyuridine (BrdU) to determine levels of proliferation. Analysis of proliferation within the different subsets of thymocytes based on CD4 and CD8 expression within the thymus was performed. FIG. 5A shows that the proportion of each thymocyte subset within the BrdU+ population did not change with age or post-castration. However, as shown in FIG. 5B, a significant decrease in the proportion of DN (CD4−CD8−) thymocytes proliferating was seen with age. FIG. 5C shows that no change in the total proportion of BrdU+ cells (i.e., proliferating cells) within the TN subset was seen with age or post-castration. However, a significant decrease in proliferation within the TN1 (CD44+CD25−CD3−CD4−CD8−) subset (FIG. 5E) and significant increase in proliferation within TN2 (CD44+CD25+CD3−CD4−CD8−) subset (FIG. 5F) was seen with age. This was restored post-castration (FIGS. 5D-F). Results are expressed as mean±1SD of 4-17 mice per group. *=p<0.05; **=p≦0.01 (significant); ***=p≦0.001 (highly significant) compared to young adult (2-month) mice; ˆ=significantly different from 2-6 weeks post-castrate mice (FIGS. 5E-H).

FIGS. 6A-C: Castration increases T cell export from the aged thymus. For these studies, aged (2-year old) mice were castrated and were injected intrathymically with FITC to determine thymic export rates. The number of FITC+ cells in the periphery was calculated 24 hours later. As shown in FIG. 6A, a significant decrease in recent thymic emigrant (RTE) cell numbers detected in the periphery over a 24 hours period was observed with age. Following castration, these values had significantly increased by 2 weeks post-cx. As shown in FIG. 6B, the rate of emigration (export/total thymus cellularity) remained constant with age, but was significantly reduced at 2 weeks post-castration. With age, a significant increase in the ratio of CD4⁺ to CD8⁺ RTE was seen; this was normalized by 1-week post-cx (FIG. 6C).

Results are expressed as mean±1SD of 4-8 mice per group. *=p≦0.05; **=p≦0.01; ***=p≦0.001 compared to young adult (2-month) mice for (FIG. 6A) and compared to all other groups (FIGS. 6B and 6C). ˆ=p≦0.05 compared to aged (1- and 2-year old) non-cx mice and compared to 1-week post-cx, aged mice.

FIGS. 7A-B: Castration enhances thymocyte regeneration following T cell depletion. 3-month old mice were either treated with cyclophosphamide (intraperitoneal injection with 200 mg/kg body weight cyclophosphamide, twice over 2 days) (FIG. 7A) or exposed to sublethal irradiation (625 Rads) (FIG. 7B). For both models of T cell depletion studied, castrated (Cx) mice showed a significant increase in the rate of thymus regeneration compared to their sham-castrated (ShCx) counterparts. Analysis of total thymocyte numbers at 1 and 2-weeks post-T cell depletion (TCD) showed that castration significantly increases thymus regeneration rates after treatment with either cyclophosphamide or sublethal irradiation (FIGS. 7A and 7B, respectively). Data is presented as mean±1SD of 4-8 mice per group. For FIG. 7A, ***=p≦0.001 compared to control (age-matched, untreated) mice; ˆ=p≦0.001 compared to both groups of castrated mice. For FIG. 7B, ***=p≦0.001 compared to control mice; ˆp≦0.001 compared to mice castrated 1-week prior to treatment at 1-week post-irradiation and compared to both groups of castrated mice at 2-weeks post-irradiation.

FIGS. 8A-B: Total lymphocyte numbers within the spleen and lymph nodes post-cyclophosphamide treatment. For these studies, (3 month old) mice were depleted of lymphocytes using cyclophosphamide (intraperitoneal injection with 200 mg/kg body weight cyclophosphamide, twice over 2 days) and either surgically castrated or sham-castrated on the same day as the last cyclophosphamide injection. Thymus, spleen and lymph nodes (pooled) were isolated and total cellularity evaluated. Sham-castrated mice had significantly lower cell numbers in the spleen at 1 and 4-weeks post-treatment compared to control (age-matched, untreated) mice (FIG. 8A). A significant decrease in cell number was observed within the lymph nodes at 1 week post-treatment for both treatment groups (FIG. 8B). At 2-weeks post-treatment, Cx mice had significantly higher lymph node cell numbers compared to ShCx mice (FIG. 8B). Each bar represents the mean±1SD of 7-17 mice per group. *=p≦0.05; **=p≦0.01 compared to control (age-matched, untreated). ˆp≦0.05 compared to castrate mice.

FIG. 9: Changes in thymus (open bars), spleen (gray bars) and lymph node (black bars) cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=3-4 per treatment group and time point). Chemical castration is comparable to surgical castration in regeneration of the immune system post-cyclophosphamide treatment.

FIGS. 10A-C: Changes in thymus (FIG. 10A), spleen (FIG. 10B) and lymph node (FIG. 11C) cell numbers following irradiation (625 Rads) one week after surgical castration. For these studies, young (3-month old) mice were depleted of lymphocytes using sublethal (625 Rads) irradiation. Mice were either sham-castrated or castrated 1-week prior to irradiation. A significant increase in thymus regeneration (i.e., faster rate of thymus regeneration) was observed with castration (FIG. 10A). Note the rapid expansion of the thymus in castrated animals when compared to the non-castrate (irradiation alone) group at 1 and 2 weeks post-treatment. (n=3-4 per treatment group and time point). No difference in spleen (FIG. 10B) or lymph node (FIG. 10C) cell numbers was seen with castrated mice. Lymph node cell numbers were still chronically low at 2-weeks post-treatment compared to control mice (FIG. 10C). Results are expressed as mean±1SD of 4-8 mice per group. *=p≦0.05; **=p≦0.01 compared to control mice; ***=p≦0.001 compared to control and castrated mice.

FIGS. 11A-C: Changes in thymus (FIG. 11A), spleen (FIG. 11B) and lymph node (FIG. 11C) cell numbers following irradiation and castration on the same day. For these studies, young (3-month old) mice were depleted of lymphocytes using sublethal (625 Rads) irradiation. Mice were either sham-castrated or castrated on the same day as irradiation. Castrated mice showed a significantly faster rate of thymus regeneration compared to sham-castrated counterparts (FIG. 11A). Note the rapid expansion of the thymus in castrated animals when compared to the non-castrate group at 2 weeks post-treatment. No difference in spleen (FIG. 11B) or lymph node (FIG. 11C) cell numbers was seen with castrated mice. Lymph node cell numbers were still chronically low at 2-weeks post-treatment compared to control mice (FIG. 11C). Results are expressed as mean±1SD of 4-8 mice per group. *=p≦0.05; **=p≦0.01 compared to control mice; ***=p≦0.001 compared to control and castrated mice.

FIG. 12A-B: Total lymphocyte numbers within the spleen and lymph nodes post-irradiation treatment. 3-month old mice were either castrated or sham-castrated 1-week prior to sublethal irradiation (625 Rads). Severe lymphopenia was evident in both the spleen (FIG. 12A) and (pooled) lymph nodes (FIG. 12B) at 1-week post-treatment. Splenic lymphocyte numbers were returned to control levels by 2-weeks post-treatment (FIG. 12A), while lymph node cellularity was still significantly reduced compared to control (age-matched, untreated) mice (FIG. 12B). No differences were observed between the treatment groups. Each bar represents the mean±1SD of 6-8 mice per group. **=p≦0.01; ***=p≦0.001 compared to control mice.

FIGS. 13A-B: Castration restores responsiveness to HSV-1 immunization. Mice were immunized in the hind foot-hock with 4×10⁵ pfu of HSV. On Day 5 post-infection, the draining lymph nodes (popliteal) were analyzed for responding cells. FIG. 13A shows the lymph node cellularity following foot-pad immunization with Herpes Simplex Virus-1 (HSV-1). Note the increased cellularity in the aged post-castration as compared to the aged non-castrated group. FIG. 13B illustrates the overall activated cell number as gated on CD25 vs. CD8 cells by FACS (i.e., the activated cells are gated on CD8+CD25+ cells). Castration of the aged mice restored the immune response to HSV-1 with CTL numbers equivalent to young mice. Results are expressed as mean±1SD of 8-12 mice. **=p≦0.01 compared to both young (2-month) and castrated mice

FIGS. 14A-C: V□10 expression (HSV-specific) on CTL (cytotoxic T lymphocytes) in activated LN (lymph nodes) following HSV-1 inoculation. Despite the normal Vβ10 responsiveness in aged (i.e., 18 months) mice overall, in some mice a complete loss of Vβ10 expression was observed. Representative histogram profiles are shown. Note the diminution of a clonal response in aged mice and the reinstatement of the expected response post-castration.

FIGS. 15A-B: Castration enhances activation following HSV-1 infection. FIG. 15A shows representative FACS profiles of activated (CD8⁺CD25⁺) cells in the LN of HSV-1 infected mice. No difference was seen in proportions of activated CTL with age or post-castration. As shown in Castration of the aged mice restored the immune response to HSV-1 with CTL numbers equivalent to young mice. Results are expressed as mean±1SD of 8-12 mice. **=p≦0.01 compared to both young (2-month) and castrated mice.

FIG. 16: Specificity of the immune response to HSV-1. Popliteal lymph node cells were removed from mice immunized with HSV-1 (removed 5 days post-HSV-1 infection), cultured for 3-days, and then examined for their ability to lyse HSV peptide pulsed EL 4 target cells. CTL assays were performed with non-immunized mice as control for background levels of lysis (as determined by ⁵¹Cr-release). Aged mice showed a significant (p≦0.01, **) reduction in CTL activity at an E:T ratio of both 10:1 and 3:1 indicating a reduction in the percentage of specific CTL present within the lymph nodes. Castration of aged mice restored the CTL response to young adult levels since the castrated mice demonstrated a comparable response to HSV-1 as the young adult (2-month) mice. Results are expressed as mean of 8 mice, in triplicate±1 SD. **=p≦0.01 compared to young adult mice; ˆ=significantly different to aged control mice (p≦0.05 for E:T of 3:1; p≦0.01 for E:T of 0.3:1).

FIGS. 17A-B: Analysis of V□ TCR expression and CD4⁺ T cells in the immune response to HSV-1. Popliteal lymph nodes were removed 5 days post-HSV-1 infection and analyzed ex-vivo for the expression of CD25, CD8 and specific TCR V□ markers (FIG. 17A) and CD4/CD8 T cells (FIG. 17B). The percentage of activated (CD25⁺) CD8⁺ T cells expressing either V□10 or V□8.1 is shown as mean±1SD for 8 mice per group in FIG. 17A. No difference was observed with age or post-castration. However, a decrease in CD4/CD8 ratio in the resting LN population was seen with age (FIG. 17B). This decrease was restored post-castration. Results are expressed as mean±1SD of 8 mice per group. ***=p≦0.001 compared to young and post-castrate mice.

FIGS. 18A-D: Castration enhances regeneration of the thymus (FIG. 18A), spleen (FIG. 18B) and BM (FIG. 18D), but not lymph node (FIG. 18C) following BM transplantation (BMT) of Ly5 congenic mice. 3 month old, young adults, C57/BL6 Ly5.1+ (CD45.1+) mice were irradiated (at 6.25 Gy), castrated, or sham-castrated 1 day prior to transplantation with C57/BL6 Ly5.2+ (CD45.2+) adult BM cells (10⁶ cells). Mice were killed 2 and 4 weeks later and the), thymus (FIG. 18A), spleen (FIG. 18B), lymph node (FIG. 18C) and BM (FIG. 18D) were analyzed for immune reconstitution. Donor/Host origin was determined with anti-CD45.2 (Ly5.2), which only reacts with leukocytes of donor origin. There were significantly more donor cells in the thymus of castrated mice 2 and 4 weeks after BMT compared to sham-castrated mice (FIG. 18A). Note the rapid expansion of the thymus in castrated animals when compared to the non-castrate group at all time points post-treatment. There were significantly more cells in these spleen and BM of castrated mice 2 and 4 weeks after BMT compared to sham-castrated mice (FIGS. 18B and 18D). There was no significant difference in lymph node cellularity 2, 4, and 6 weeks after BMT (FIG. 18C). Castrated mice had significantly increased congenic (Ly5.2) cells compared to non-castrated animals. Data is expressed as mean±1SD of 4-5 mice per group. *=p≦0.05; **=p≦0.01.

FIGS. 19A-C: Castration increases BM and thymic cellularity following congenic BMT. As shown in FIG. 19A, there are significantly more cells in the BM of castrated mice 2 and 4 weeks after BMT. BM cellularity reached untreated control levels (1.5×10⁷±1.5×10⁶) in the sham-castrates by 2 weeks. BM cellularity is above control levels in castrated mice 2 and 4 weeks after congenic BMT. FIG. 19b shows that there are significantly more cells in the thymus of castrated mice 2 and 4 weeks after BMT. Thymus cellularity in the sham-castrated mice is below untreated control levels (7.6×10⁷5.2×10⁶) 2 and 4 weeks after congenics BMT. 4 weeks after congenic BMT and castration thymic cellularity is increased above control levels. FIG. 19C shows that there is no significant difference in splenic cellularity 2 and 4 weeks after BMT. Spleen cellularity has reached control levels (8.5×10⁷±1.1×10⁷) in sham-castrated and castrated mice by 2 weeks. Each group contains 4 to 5 animals. Open bars indicate sham-castration; closed bars indicate castration.

FIG. 20: Castration increases the proportion of HSC following congenic BMT. Representative FACS dot plots illustrating c-kit (y-axis) versus sca-1 (x-axis) expression. HSC are c-kit^{hi}sca-1^{hi}. There is a significant increase in the proportion of donor-derived HSCs following castration, 2 and 4 weeks after BMT.

FIGS. 21A-B: Castration increases the proportion and number of HSC following congenic BMT. As shown in FIG. 21A, there was a significant increase in the proportion of HSCs following castration, 2 and 4 weeks after BMT (*p<0.05). FIG. 21B shows that the number of HSCs is significantly increased in castrated mice compared to sham-castrated controls, 2 and 4 weeks after BMT (*p<0.05 **p<0.01). Each group contains 4 to 5 animals. Open bars indicate sham-castration; closed bars indicate castration.

FIGS. 22A-B: There are significantly more donor-derived B cell precursors and B cells in the BM of castrated mice following BMT. As shown in FIG. 22A, there were significantly more donor-derived CD45.1⁺B220⁺IgM⁻ B cell precursors in the BM of castrated mice compared to the sham-castrated controls (*p<0.05). FIG. 22B shows that there were significantly more donor-derived B220⁺IgM⁺ B cells in the BM of castrated mice compared to the sham-castrated controls (*p<0.05). Each group contains 4 to 5 animals. Open bars indicate sham-castration; closed bars indicate castration.

FIG. 23: Castration does not effect the donor-derived thymocyte proportions following congenic BMT. 2 weeks after sham-castration and castration there is an increase in the proportion of donor-derived double negative (CD45.1⁺CD4⁻CD8⁻) early thymocytes. There are very few donor-derived (CD45.1⁺) CD4 and CD8 single positive cells at this early time point. 4 weeks after BMT, donor-derived thymocyte profiles of sham-castrated and castrated mice are similar to the untreated control.

FIG. 24: Castration does not increase peripheral B cell proportions following congenic BMT. There is no difference in splenic B220 expression comparing castrated and sham-castrated mice, 2 and 4 weeks after congenic BMT.

FIG. 25: Castration does not increase peripheral B cell numbers following congenics BMT. There is no significant difference in B cell numbers 2 and 4 weeks after BMT. 2 weeks after congenic BMT B cell numbers in the spleen of sham-castrated and castrated mice are approaching untreated control levels (5.0×10⁷±4.5×10⁶). Each group contains 4 to 5 animals. Open bars indicate sham-castration; closed bars indicate castration.

FIG. 26: Donor-derived triple negative, double positive and CD4 and CD8 single positive thymocyte numbers are increased in castrated mice following BMT. FIG. 26A shows that there were significantly more donor-derived triple negative (CD45.1⁺CD3⁻CD4⁻CD8⁻) thymocytes in the castrated mice compared to the sham-castrated controls 2 and 4 weeks after BMT (*p<0.05 **p<0.01). FIG. 26B shows there were significantly more double positive (CD45.1⁺CD4⁺CD8⁺) thymocytes in the castrated mice compared to the sham-castrated controls 2 and 4 weeks after BMT (*p<0.05 **p<0.01). As shown in FIG. 26C, there were significantly more CD4 single positive (CD45.1⁺CD3⁺CD4⁺CD8⁻) thymocytes in the castrated mice compared to the sham-castrated controls 2 and 4 weeks after BMT (*p<0.05 **p<0.01). FIG. 26D shows there were significantly more CD8 single positive (CD45.1⁺CD3⁺CD4⁻CD8⁺) thymocytes in the castrated mice compared to the sham-castrated controls 4 weeks after BMT (*p<0.05 **p<0.01). Each group contains 4 to 5 animals. Open bars indicate sham-castration; closed bars indicate castration.

FIG. 27: There are very few donor-derived, peripheral T cells 2 and 4 weeks after congenic BMT. As shown in FIG. 27A, there was a very small proportion of donor-derived CD4⁺ and CD8⁺ T cells in the spleens of sham-castrated and castrated mice 2 and 4 weeks after congenic BMT. FIG. 27B shows that there was no significant difference in donor-derived T cell numbers 2 and 4 weeks after BMT. 4 weeks after congenics BMT there are significantly less CD4⁺ and CD8⁺ T cells in both sham-castrated and castrated mice compared to untreated age-matched controls (CD4⁺−1.1×10⁷±1.4×10⁶, CD8⁺−6.0×10^{6±1.0×10}⁵) Each group contains 4 to 5 animals. Open bars indicate sham-castration; closed bars indicate castration.

FIG. 28: Castration increases the number of donor-derived DC in the thymus 4 weeks after congenics BMT. As shown in FIG. 28A, donor-derived DC were CD45.1⁺CD11c⁺MHCII⁺. FIG. 28B shows there were significantly more donor-derived thymic DC in the castrated mice 4 weeks after congenic BMT (*p<0.05). Dendritic cell numbers are at untreated control levels 2 weeks after congenic BMT (1.4×10⁵±2.8×10⁴). 4 weeks after congenic BMT dendritic cell numbers are above control levels in castrated mice. Each group contains 4 to 5 animals. Open bars indicate sham-castration; closed bars indicate castration.

FIGS. 29A-C: Castration enhances immune cell reconstitution in allogeneic HSCT recipients. Lethally irradiated (1300 cGy) CBA/J recipients (3 month old) received transplants with B10.BR TCD BM (5×10⁶). Recipients were either castrated or sham-castrated one day before transplant. Animals were humanely killed on days 14, 28 and 42 and BM (FIG. 29A), thymus (FIG. 29B), and spleen (FIG. 29C) organ cellularity was assessed. *(p<0.05). Each group contained 4 to 5 animals.

FIGS. 30A-C: Castration enhances donor-derived HSC and B cells in allogeneic HSCT recipients. Castrated and sham-castrated recipients were transplanted as in described in FIG. 29. As shown in FIG. 30A, 14 days after HSCT there were very few donor-derived HSCs (Ly9.1⁻Lin⁻Sca-1⁺c-kit⁺) in both sham-castrated and castrated mice; however, by day 28, donor HSC numbers were 4-fold higher in the castrated group. Additionally, as shown in FIGS. 30B-C, there are significantly more donor-derived B cells in the BM (FIG. 30B) and spleen (FIG. 30C) of castrated mice. Central and peripheral B cell populations were analyzed using total BM or splenic cell counts and multicolor flow cytometry. B cells were separated into developmental stages based on CD45R, IgM and CD43 expression. Total B cells (CD45R⁺), pro-B cells (CD43⁺CD45R⁺IgM⁻), pre-B cells (CD43⁻CD45R⁺IgM⁻), immature B cells (CD43⁻CD45R⁺IgM⁺). Donor/host origin was determined with anti-Ly9.1, which only reacts with leukocytes of host origin. Each group contained 4 to 5 animals. Open bars indicate sham-castrated animals, and closed bars represent castrated animals. *(p<0.05) represents a significant increase in cell number in the castrated group compared to the sham-castrated control.

FIGS. 31A-E: Castration enhances thymocyte and peripheral T cell reconstitution as well as the number of host and donor-derived DC in allogeneic HSCT recipients. Castrated and sham-castrated recipients were transplanted as described in FIG. 29. Animals were humanely killed on days 14, 28 and 42, and thymocyte and T cell populations were analyzed using total thymic (FIG. 31A-F) or splenic (FIG. 31G) cell counts and multicolor flow cytometry. DC were defined as CD11c^{hi} Ia-k^{hi}. FIG. 31A depicts numbers of TN (CD3⁻CD4⁻CD8⁻) thymocytes. FIG. 31B depicts numbers of DP (CD4⁺CD8⁺) thymocytes. FIG. 31C depicts numbers of CD4+ SP (CD3⁺CD4⁺CD8⁻) thymocytes. FIG. 31D depicts numbers of CD8+ SP (CD3⁺CD4⁻CD8⁺) thymocytes. As shown in FIG. 331E, there are significantly more host-derived DC in castrated recipients at both 14 and 28 days after allogeneic HSCT as compared to sham-castrated control recipients. Additionally, as shown in FIG. 31F, there are significantly more donor-derived DC in castrated recipients 28 days following allogeneic HSCT, as compared to sham-castrated controls. FIG. 31G depicts numbers of peripheral T cells, which were identified using anti-CD3, anti-CD4 and anti-CD8. Donor/host origin was determined with anti-Ly9.1, which only reacts with leukocytes of host origin. Donor CD4 T cells were Ly9.1⁻CD3⁺CD4⁺CD8⁻ and donor CD8 T cells were Ly9.1⁻CD3⁺CD4⁻CD8⁺. Each group contained 4 to 5 animals. Open bars indicate sham-castrated animals, and closed bars represent castrated animals. *(p<0.05) and **(p<0.01) represent a significant increase in cell number in the castrated group compared to the sham-castrated control.

FIGS. 32A-G: Castration does not alter the function of donor-derived T cells following allogeneic HSCT. Castrated and sham-castrated recipients were transplanted as in FIGS. 29. T cell functionality was assessed 42 days after transplantation. FIG. 32A shows the number of donor-derived T cells (CD3⁺CD4⁺ and CD3⁺CD8⁺) six weeks after allo-HSCT. FIG. 32B shows that castration has no effect on the proliferative capability of T cells after allogeneic HSCT. FIG. 32C shows no difference in alloreactive T-cell proliferation in an MLR. Splenic T cells (4×10⁵ cells/well) from each group (n=5) were incubated with irradiated (20 Gy) BALB/C splenic stimulator cells (2×10⁵ cells/well) in 96-well plates for 5 days and [3H]-thymidine was added during the final 20 hours of culture. FIG. 32D shows no difference in cytolytic activity of donor-derived T cells. FIG. 32E shows intracellular IFNγ expression of alloreactive T cells. Splenic B6 T cells were harvested on day 42 from sham-castrated or castrated recipients as described above and incubated with irradiated (20 Gy) (BALB/C—third party) splenic stimulator cells in 24-well plates for 5 days. Cells were harvested, and restimulated with TCD, irradiated (20 Gy) (BALB/C or B10.BR internal biological control) splenic stimulator cells for 16 hours. Brefeldin A (10 mg/mL) was added after the first hour of incubation. Intracellular IFNγ expression in donor-derived CD3⁺CD8⁺ cells was measured by flow cytometric analysis. Representative plots are shown in FIG. 32E and graphically represented as the percentage of donor-derived CD8⁺ T cells that express IFN-γ in FIG. 32F. FIG. 32G shows that T cell functionality was significantly enhanced 48 hrs. after challenge when mice were castrated at the time of allo-HSCT. The DTH assay was performed at week 6 following allogeneic HSCT in sham-castrated and castrated mice, and the swelling was measured by subtracting left hind footpad swell from right hind one. Open bars indicate sham-castrated animals, and closed bars represent castrated animals.

FIGS. 33A-B: Castration does not aggravate GVHD or decrease GVL activity in allogeneic HSCT recipients. For the experiments depicted in FIG. 33A, lethally irradiated (1300 cGy) (B6×C3H) F1 recipients (3 months old) received transplants with B6 TCD BM cells (5×10⁶)+splenic T cells (0.5×10⁶). Survival is depicted as a Kaplan-Meier curve. Open circles represent a TCD-BM only (no T cells) control group (n=4). Closed circles represent sham-castrated recipients; and open squares represent castrated recipients. Each group contained 8 animals. For the experiments depicted in FIG. 33B, lethally irradiated, 3 month old B6D2F1/J recipients received P815 (H-2d) cells (1×10³), C57/BL6 TCD BM cells (5×10⁶) and C57/BL6 T cells (5×10⁵). Survival is depicted as a Kaplan-Meier curve. Open circles indicate a TCD-BM only (no T cells) control group (n=4). Closed circles represent sham-castrated recipients, and open squares represent castrated recipients. Each test group contained 8-9 animals.

FIGS. 34A-I: Castration and IL-7 treatment have an additive effect in the thymus following allogeneic HSCT. Castrated and sham-castrated recipients were transplanted described in FIG. 1. Recipients killed on day 14 (FIG. 34A) received, in addition, 10 g/day IL-7 or PBS (control) by intraperitoneal injection from day 0 to day 13. Recipients killed on day 28 (FIG. 34B) received 10 g/day IL-7 or PBS (control) from day 21 to day 28. Thymic cellularity was calculated from total cell counts. *(p<0.05) represents a significant increase in cell number in the castrated group compared to the sham-castrated control. Control: sham-castrated, PBS injected recipients; CX: castrated, PBS injected recipients; IL-7: sham-castrated, IL-7 injected recipients; and IL-7 & CX: castrated, IL-7 injected recipients. Semiquantitative RT-PCR was performed on whole BM 2 weeks after allo-HSCT and castration. After HPRT equilibration templates from castrated and sham-castrated mice were compared for the expression of TGFβ₁ and KGF (FIG. 34C). KGF^{-/-} and IL7^{-/-} mice were castrated and 2 weeks later thymic, BM and splenic cellularity were analysed. FIG. 34D-F shows the results from the thymus (FIG. 34D), BM (FIG. 34E), spleen (FIG. 34F) of KGF^{-/-} mice. FIG. 34G-I shows the results from the thymus (FIG. 34G), BM (FIG. 34H), and spleen (FIG. 341) of IL7−/− mice.

FIG. 35: Castration enhances engraftment in the BM, thymus, and spleen following HSCT. Mice were castrated 1 day before congenic HSCT. 5×10⁶ Ly5.1⁺ BM cells were injected intravenously into irradiated (800 rads) C57/BL6 mice. The BM, spleen and thymus were analyzed by flow cytometry at various time points (2-6 weeks) post-transplant. As shown in FIG. 35B, two weeks after castration and HSCT, there are significantly more cells in the BM of castrated mice as compared to sham-castrated controls. Similarly, as shown in FIG. 35C, there is a significant increase in thymic cell number 2, 4 and 6 weeks post-transplant as compared to sham castrated controls. As shown in FIG. 35C, in the periphery, splenic cell numbers are also significantly higher than controls 4 and 6 weeks post-transplant in the castrated recipients. Gray bars represent castrated recipients. Black bars represent sham-castrated controls.

FIG. 36A-B: Castration enhances engraftment of HSC in the BM following congenic HSCT. Mice were castrated 1 day before congenic HSCT. 5×10⁶ Ly5.1⁺ BM cells were injected intravenously into irradiated (800 rads) C57/BL6 mice. The BM was analyzed for lin-c-kit+sca-1+ HSC by flow cytometry at two weeks post-transplant (FIG. 36A). Two weeks after BMT transplantation and castration there are significantly more donor-derived HSCs in the BM of castrated mice compared to sham castrated controls (FIG. 36B).

FIGS. 37A-B: Castration enhances engraftment of HSC in the BM following congenic HSCT (2.5×10⁶ cells and 5×10⁶ cells). Mice were castrated 1 day before congenic HSCT. 2.5×10⁶ (FIG. 37A-B) or 5×10⁶ (FIG. 37C-D) Ly5.1⁺ BM cells were injected intravenously into irradiated (800 rads) C57/BL6 mice. The BM was analyzed for lin-c-kit+sca-1+ HSC by flow cytometry at two weeks post-transplant. FIGS. 37A-D depict percent of common lymphoid precursors in the BM. Two weeks after BMT transplantation and castration there is a significantly increased proportion of donor-derived HSCs in the BM of castrated mice compared to sham castrated controls.

FIGS. 38A-B: Castration enhances the rate of engraftment of donor-derived DC in the thymus following congenic HSCT (2.5×10⁶ cells and 5×10⁶ cells). 5×10⁶ Ly5.1⁺ BM cells were injected intravenously into irradiated (800 rads) C57/BL6 mice. Thymocytes were analyzed by flow cytometry at two weeks post-transplant (FIG. 38A). Donor-derived DC were defined as CD45.1⁺CD11c⁺MHC class II⁺ CD11b^{+ or -}. Donor-derived CD11b⁺ and CD11b⁻ DC are significantly increased in the thymii of castrated mice compared to sham-castrated controls 2 weeks after BMT (FIG. 38B).

FIGS. 39A-D: Castration enhances the rate of engraftment of donor-derived B cells in the spleen following congenic HSCT. 5×10⁶ Ly5.1⁺ BM cells were injected intravenously into irradiated (800 rads) C57/BL6 mice. Splenocytes were analyzed by flow cytometry at two weeks post-transplant (FIG. 39A-C). There are significantly more B220⁺ B cells in the spleens of castrated mice, as compared the sham-castrated controls, 2 weeks after congenics BMT (FIG. 39D).

FIG. 40: The phenotypic composition of peripheral blood lymphocytes was analyzed in human patients (all >60 years) undergoing LHRH agonist treatment for prostate cancer. Patient samples were analyzed before treatment and 4 months after beginning LHRH agonist treatment. Total lymphocyte cell numbers per ml of blood were at the lower end of control values before treatment in all patients. Following treatment, six out of nine patients showed substantial increases in total lymphocyte counts (in some cases a doubling of total cells was observed). Correlating with this was an increase in total T cell numbers in six out of nine patients. Within the CD4⁺ subset, this increase was even more pronounced with eight out of nine patients demonstrating increased levels of CD4 T cells. A less distinctive trend was seen within the CD8⁺ subset with four out of nine patients showing increased levels, albeit generally to a smaller extent than CD4⁺ T cells.

FIG. 41: Analysis of human patient blood before and after LHRH-agonist treatment demonstrated no substantial changes in the overall proportion of T cells, CD4 or CD8 T cells, and a variable change in the CD4:CD8 ratio following treatment. This indicates the minimal effect of treatment on the homeostatic maintenance of T cell subsets despite the substantial increase in overall T cell numbers following treatment. All values were comparative to control values.

FIG. 42: Analysis of the proportions of B cells and myeloid cells (NK, NKT and macrophages) within the peripheral blood of human patients undergoing LHRH agonist treatment demonstrated a varying degree of change within subsets. While NK, NKT and macrophage proportions remained relatively constant following treatment, the proportion of B cells was decreased in four out of nine patients.

FIG. 43: Analysis of the total cell numbers of B and myeloid cells within the peripheral blood of human patients post-treatment showed clearly increased levels of NK (five out of nine patients), NKT (four out of nine patients) and macrophage (three out of nine patients) cell numbers post-treatment. B cell numbers showed no distinct trend with two out of nine patients showing increased levels; four out of nine patients showing no change and three out of nine patients showing decreased levels.

FIGS. 44A-B: Chemical castration in humans enhances naïve and memory T cells. As shown in FIG. 44A, a significant increase in naïve (CD62L⁺CD45RA⁺CD45RO⁻) CD4⁺ T cells was observed following LHRH-A treatment. As shown in FIG. 44B, both naïve and memory (CD62L⁻CD45RA⁻CD45RO⁺) CD8⁺ T cells numbers were enhanced following the LHRH agonist treatment. Each bar represents the mean±1SD of 16 patients. *=p≦0.05; **=p≦0.01 compared to pre-treatment values.

FIG. 45A-B: Chemical castration in humans enhances peripheral blood lymphocyte numbers. The phenotypic composition of peripheral blood was analysed in human patients (all >60 years of age) undergoing chemical castration with a LHRH-A as part of their routine treatment for prostate cancer. Patients were analysed prior to treatment and at 4-months of treatment. As shown in FIG. 45A, total lymphocyte number per μl peripheral blood was significantly increased following LHRH-A treatment. T his was reflected by a significant increase in total T cells, CD4⁺ and CD8⁺ T cells (FIG. 45B).

FIG. 46A-B: LHRH-A treatment effectively depletes serum testosterone, and increases thymic function and T cell export. In the FIG. 46A experiment, prostate cancer patients were treated with LHRH-A for 4 months. Blood was analyzed by FACS and serum was analyzed by RIA both prior to treatment and following 4-months of LHRH-A treatment. As shown in FIG. 83A, no testosterone was detected in patient sera at 4-months of LHRH-A treatment. The bar represents the mean of 13 patients analyzed. In the FIG. 46B experiment, direct evidence for an increase in thymic function and T cell export was found following analysis of TREC levels in 10 patients. Within both the CD4⁺ and CD8⁺ T cell population, five out of ten patients showed an increase (>25% above initial presentation values) in absolute TREC levels (per ml of blood) by 4 months of LHRH-A treatment. This was also reflected in a proportional increase (per 1×10⁵ cells; data not shown). This correlated with six out of ten patients showing an overall increase in total TREC levels. Only 1 patient showed a decrease in total TRECs (about 30% decrease).

FIG. 47: Chemical castration in humans enhances NK numbers. Analysis was performed prior to LHRH-A treatment and at 4-months of treatment. A significant increase in NK cells, but not B cells, was observed with LHRH-A treatment. Results are presented as the mean±1SD of 13 patients. **=p≦0.01 compared to pre-treatment values.

FIGS. 48A-B: Chemical castration in humans does not increase proliferation of T cells. FIGS. 48A-B depict analyses of cellular proliferation performed using Ki-67 antigen detection. In all patients, levels of proliferation within naïve, activated and memory cell subsets for both CD4⁺ (FIG. 48A) and CD8⁺ T cells (FIG. 48B), was not altered with LHRH-A treatment.

FIGS. 49A-C: Analysis of natural killer (NK) cell recovery at various time points (2-8 weeks) following HSCT in control patients and LHRH-A treated patients. As shown in FIGS. 49A-B, respectively, a similar trend was observed for both control allogeneic and autologous transplant recipients. In contrast, allogeneic patients who were given LHRH-A treatment 3 weeks prior to HSCT showed a significantly higher number of NKT cells from D14-5M post-transplant (FIG. 49C; data is expressed as mean±1 SEM of 6-20 patients; *=p≦0.05).

FIG. 50: FACS analysis of NKT cell reconstitution at various time points (day 14, 21, 28 and 35) following HSCT in control patients. An early recovery was observed in allogeneic patients, and was seen predominantly within the CD8+ population early post-transplant, which indicated extrathymic routes of regeneration. Also, CD4+NKT cells were evident from 1 month post-transplant.

FIGS. 51A-B: B cell reconstitution following HSCT at various time points (2-12 months) following HSCT in control patients. As shown in FIG. 51B, B cell regeneration occurs occurring relatively faster in autologous transplant patients as compared to that of allogeneic patients (FIG. 51A). However, a return to control values (shaded) was not evident until at least 6 months post-transplant in both groups.

FIGS. 52A-B: CD4+ reconstitution following HSCT at various time points (2-12 months) following HSCT in control patients. While B cell numbers were returning to control values by 6 months post-transplant (see FIGS. 48A-B), CD4+ T cell numbers were severely reduced, even at 12 months post-transplant, in both autologous (FIG. 52B) and allogeneic (FIG. 52A) recipients.

FIGS. 53A-C: CD8+ regeneration following HSCT at various time points (2-12 months) following HSCT in control patients. As shown in FIG. 53A-B, CD8+ T cell numbers regenerated quite rapidly post-transplant in both allogeneic and autologous recipients, respectively. However, as shown in FIG. 53C, the CD8+ T cells are mainly of extrathymic origin as indicated by the increase in TCRγδ+ T CD8+ T cells, CD8αα T cells, and CD28⁻CD8⁺ T cells.

FIGS. 54A-B: FACS analysis of proliferation in various populations of CD4+ and CD8+ T cells before (FIG. 54A) and 28 days after (FIG. 54B) HSCT in control patients using the marker Ki-67. Cells were analyzed on the basis of naïve, memory and activated phenotypes using the markers CD45RO and CD27. The majority of proliferation occurred in CD8+ T cell subset, which further indicated that these cells were extrathymically derived and that the predominance of proliferation occurred within peripheral T cell subsets.

FIGS. 55A-D: Naïve CD4+ T cell regeneration at various time points (2-12 months) following HSCT in control patients and LHRH-A treated patients. FIG. 55A depicts FACS analysis of naïve CD4+ T cells (CD45RA+CD45RO−CD62L+) in control (no LHRH-A treatment) patients, and shows a severe loss of these cells throughout the study. As shown in FIGS. 55B-C, naïve CD4+ T cell began to regenerate in the control patients by 12 months post-HSCT in autologous transplant patients (FIG. 55C) but were still considerably lower than the control values in allogeneic control patients (FIG. 55B). These results indicated that the thymus was unable to restore adequate numbers of naïve T cells in control patients post-transplant due to the age of the patients. In contrast, in patients that were given LHRH-A 3-weeks prior to allogeneic HSCT showed a significantly higher number of naïve CD4+ T cells at both 9 & 12 months post-transplant compared to controls (FIG. 55D). This indicates enhanced regeneration of the thymic-dependent T cell pathway with sex steroid ablation therapy. Data is expressed as mean±1SEM of 6-20 patients. *=p≦0.05.

FIGS. 56A-D: TREC levels at various time points (1-12 months) following HSCT in control patients and LHRH-A treated patients. Analysis of TREC levels, which are only seen in recent thymic emigrants (RTE), emphasized the inability of the thymus to restore levels following transplant in both allogeneic (FIG. 52A) and autologous (FIG. 52B) patients. Again, this was due to the age of the patients, as well as the lack of thymic function due to thymic atrophy, which has considerable implications in the morbidity and mortality of these patients. In contrast, patients undergoing allogeneic peripheral blood stem cell transplantation demonstrated a significant increase in CD4+TREC+ cells/ml blood when treated with an LHRH-A prior to allogeneic transplantation (p≦0.01 at 9 months post-transplant compared to control (non-LHRH-A treated). Allogeneic patients who were given LHRH-A treatment showed a significantly higher number of CD4⁺TREC⁺ cells/ml blood at 9 months post-transplant (FIG. 56C) compared to controls. Autologous LHRH-A treated patients also showed significantly higher levels at 12 months post-transplant (FIG. 56D). This indicates enhanced regeneration of the thymus with sex steroid ablation therapy. Data is expressed as mean±1 SEM of 5-18 patients. *=p≦0.01.

FIG. 57A-C: LHRH-A administration enhances responsiveness to TCR specific stimulation following allogeneic (FIG. 57A-B) and autologous (FIG. 57C) HSCT. Three weeks prior to HSCT, patients were given LHRH-A. Patients who did not receive the agonist were used as control patients. Analysis of TCR specific stimulation was performed using 5 μg anti-CD3 and 10 μg anti-CD28 cross-linking at various time points (1-12 months) post-transplant. As shown in FIGS. 57A-B, allogeneic LHRH-A treated patients showed enhanced proliferative responses (assessed by ³H-Thymidine incorporation) compared to control patients at all time-points except 6 and 9 months (due to low patient numbers analyzed at this time). At 6 and 9 months post-transplant control patients had similar responsiveness to pre-treatment values. However at all other time-points, they were considerably lower. In contrast, LHRH-A treated patients had equivalent responsiveness at all time-points except 6 months compared to pre-treatment. LHRH-A treated patients showed enhanced proliferative responses (assessed by ³H-Thymidine incorporation) compared to control patients at 1, 3 and 4 months post-transplant. This indicates a contribution of direct peripheral T cell effects, as new CD4+ T cells are not evident until at least 1-2 months post-transplant (FIG. 57B; Data is expressed as mean±1SEM of 5-12 patients. *=p≦0.05; **=p≦0.01). As shown in FIG. 57C, autologous LHRH-A treated patients showed enhanced proliferative responses (assessed by ³H-Thymidine incorporation) compared to control patients at all time-points except 5 months. Restoration to pre-treatment values was observed by 12 months post-transplant in both control and LHRH-A treated patients.

FIG. 58A-B: LHRH-A administration enhances responsiveness to PWM and TT mitogenic stimulation following allogeneic HSCT. Three weeks prior to HSCT, patients were treated with LHRH-A. Patients who did not receive the agonist were used as control patients. Analysis of mitogenic responsiveness was performed using pokeweed mitogen (PWM) or tetanus toxoid (TT) at various time points (1-12 months) post-transplant. Patients treated with LHRH-A prior to HSCT showed an enhanced responsiveness to PWM (FIG. 58A) and TT (FIG. 58B) stimulation at all time-points studied compared to control patients.

FIG. 59A-B: LHRH-A administration enhances responsiveness to PWM and TT mitogenic stimulation following autologous HSCT. Three weeks prior to HSCT, patients were treated with LHRH-A. Patients who did not receive the agonist were used as control patients. Analysis of mitogenic responsiveness was performed using PWM or TT at various time point (1-12 months) post-transplant. Patients treated with LHRH-A prior to HSCT showed an enhanced responsiveness to PWM (FIG. 59A) and TT (FIG. 59B) stimulation at the majority of time-points studied compared to control patients (p≦0.001 at 3 months). By 12-months post-transplant, LHRH-A treated patients had restored responsiveness.

FIGS. 60A-D. LHRH-A treatment enhances the rate of engraftment in autologous HSCT patients. Three weeks prior to HSCT, patients were treated with LHRH-A (FIGS. 60A, C and D). Patients who did not receive the agonist were used as control patients (FIG. 60B). Total white blood cell (WBC) counts and granulocyte (G) counts per μl of blood were determined at days 14, 28, and 35 post transplant. As shown in FIG. 60A, autologous patients who were given LHRH-A treatment showed a significantly higher number of WBC at D14 post-transplant compared to controls (FIG. 60B) (p≦0.05), with 87% showing granulocyte engraftment (≧500 cells/μl blood) compared to 45% of controls (p≦0.05) at this time point. Autologous patients who were given LHRH-A treatment also showed a significantly higher number of neutrophils at D10-12 post-transplant compared to controls (FIG. 60C; data is expressed as mean±1SEM of 8-20 patients. *=p≦0.05). In addition, although not significant, autologous patients had higher lymphocyte counts throughout the time-points analyzed in LHRH-A treated compared to control group (FIG. 60D).

FIGS. 61A-D: LHRH-A treatment enhances the rate of engraftment in allogeneic HSCT patients. Three weeks prior to HSCT, patients were treated with LHRH-A (FIGS. 61A, C and D). Patients who did not receive the agonist were used as control patients (FIG. 61B). Total white blood cell (WBC) counts and granulocyte (G) counts per μl of blood were determined at day 14, 28, and 35 post transplant. As shown in FIG. 61A, allogeneic patients who were given LHRH-A treatment showed a significantly higher number of WBC at D14 post-transplant compared to controls (FIG. 61B) (p≦0.05) with 64% showing granulocyte engraftment (≧500 cells/μl blood) compared to 44% of controls at this time point. In addition, allogeneic patients who were given LHRH-A treatment showed a significantly higher number of neutrophils at D9, 12 & 19 post-transplant compared to controls (FIG. 61C; data is expressed as mean±1SEM of 8-20 patients. *=p≦0.05). Additionally, analysis of patients undergoing peripheral blood stem cell transplantation demonstrated a significant increase in lymphocyte counts when treated with an LHRH-A prior to allogeneic transplantation (p≦0.05 at days 10, 12, 13 & 17-21 post-transplant) (FIG. 61D).

FIGS. 62A-F. TCR specific peripheral T cell proliferative responses are enhanced within one week of castration. Eight week-old mice were castrated and analyzed for anti-CD3/anti-CD28 stimulated T cell proliferative response 3 days (FIGS. 62A, C, and E) and 7 days (FIGS. 62B, D, and F) after surgery. Peripheral (cervical, axillary, brachial and inguinal) lymph node (FIGS. 62A and B), mesenteric lymph node (FIGS. 62C and D), and spleen cells (FIGS. 62E and F) were stimulated with varying concentrations of anti-CD3 and co-stimulated with anti-CD28 at a constant concentration of 10 μg/ml for 48 hours. Cells were then pulsed with tritiated thymidine for 18 hours and proliferation was measured as ³H-T incorporation. Diamonds indicate castrated animals. Squares indicate sham-castrated control mice. n=4, p≦0.05 (non-parametric, unpaired, Mann-Whitney statistical test).

FIG. 63: LHRH-A administration enhances responsiveness to TCR specific stimulation following treatment for chronic cancer sufferers. Patients with chronic malignancies were treated with LHRH-A. Analysis of TCR specific stimulation was performed using anti-CD3 and anti-CD28 cross-linking from at various time points (day 7-12 months) following LHRH-A administration. LHRH-A treated patients showed enhanced proliferative responses (assessed by ³H-Thymidine incorporation) compared to pre-treatment levels in a cyclical fashion. This reflected the administration of the agonist with monthly depot injections. These results indicate a direct influence on peripheral T cells. However, the enhanced response seen at 12-months post-treatment reflect changes in thymic-derived T cells as well, since agonist administration was ceased from 4-months for all patients.

FIGS. 64A-E are graphs showing that thymectomy does not impact the effect of sex steroid inhibition/BMT on common lymphoid progenitors in the BM (FIG. 72A), total BM B cells (FIG. 72B), immature B cells in the BM (FIG. 64C), total cell numbers in the spleen (FIG. 72D) or on total B cells in the spleen (FIG. 72E).

### DETAILED DESCRIPTION OF THE INVENTION

The patent and scientific literature referred to herein establishes knowledge that is available to those with skill in the art. The issued U.S. patents, applications, published foreign applications, and references, including GenBank database sequences, that are cited herein are hereby incorporated by reference in its entirety to the same extent as if each was specifically and individually indicated to be incorporated by reference.

The present invention comprises methods for increasing the BM functionality following sex steroid ablation and/or interruption of sex steroid signalling, either without, prior to, or in combination with, thymus regeneration. “Increasing the function of BM” and “enhancing BM functionality” is herein defined as an improvement in the production and/or output of immune cells, including precursors, for example HSC (and consequent increases in blood cells) from the BM, including improvement in haemopoiesis and/or enhancement of engraftment following HSCT. “An improvement in output” includes, but is not limited to, an improved ability to mobilize immune cells, including HSC into the periphery or to a target tissue, in particular, immune or damaged tissue. In one embodiment, HSC haemopoiesis is improved. In another embodiment, HSC output is improved. In certain embodiments, blood and/or immune cell numbers are increased. In yet another embodiment, HSC engraftment is improved following HSCT. In another embodiment HSC mobilization into the periphery or homing to target tissue is improved. In yet another embodiment, proliferative ability, and/or the ability to differentiate into haematopoietic or non-haemopoitetic progeny is improved.

The present invention also comprises methods for increasing the function of T cells and other immune cells following sex steroid ablation and/or interruption of sex steroid, signaling, either without, prior to, or in combination with, thymus regeneration. The terms “immune cells” and “cells of the immune system” are used interchangeably and are herein defined as HSC, T cells, B cells, DC, and/or other blood cells, including, but not limited to HSC progeny, CLP, MLP, lymphocytes, myeloid cells, neutrophils, granulocytes, basophils, eosinophils, NK, NKT, platelets, red blood cells, monocytes, macrophage, naïve T cells, and precursors of the aforementioned. The cells may or may not be peripheral, and the cells may be found in any one or more of the BM, blood, spleen, lymph nodes, thymus. mucosal membranes, skin, or other tissues.

“Increased” or “enhanced functionality” of immune cells means that the immune cells are more able to provide an adequate required immune response, when compared to the immune response normally expected without sex steroid ablation. In one case, the immune cells are T cells. In other examples, the immune cells are B cells, DC, and/or HSC. “Increased functionality” includes, but is not limited to, improved killing of target cells; increased lymphocyte proliferative response; improved signaling ability; improved homing ability; improved APC activation, increased levels or activity of receptors, cell adhesion molecules, or co-stimulatory molecules; decreased apoptosis; increased release of cytokines, interleukins, and other growth factors; increased levels of antibody (Ab) in the plasma; and increased levels of innate immunity (e.g., natural killer (NK) cells, DC, neutrophils, macrophages, etc.) in the blood and throughout the body.

To generate new T lymphocytes, the thymus requires precursor cells; these can be derived from within the organ itself for a short time, but after 3-4 weeks, such cells are depleted and new hematopoietic stem cells (HSC) must be taken in, typically from the bone marrow via the blood. However, even in a normal functional young thymus, the uptake of such cells is very low, i.e., sufficient to maintain T cell production at homeostatically regulated levels and effectively restricted to HSC or at least prothymocytes which already have a preferential development along the T cell lineage. During sex steroid inhibition, the thymus becomes very receptive to new precursor cells circulating in the blood. By increasing the level of the blood precursor cells, the T cells derived from them will progressively enter the T cell pool. This means that a gene introduced into the precursors (e.g., the HSC) may be passed onto all progeny T cells and eventually be present in much of the T cell pool. The level of dominance of these cells over those derived from endogenous host HSC may be increased to by increasing the number of transferred exogenous HSC.

The present invention stems from the discovery that disrupting sex steroid signaling in a patient who requires a donor allograft transplantation, prior to, concurrently with, or after administration of donor cells (e.g., HSC), facilitates the acceptance by the patient of the donor allograft. In some embodiments, the patient also receives a transfer of cells, such as HSC, from the donor. As the thymus is reactivating, a new or modified immune system is created, one that no longer recognizes and/or responds to antigens on the allograft. In other words, the allograft is seen as “self,” and not as foreign.

In one embodiment, thymic grafts can be used in the methods of the invention to improve engraftment of the donor cells or tolerance to the donor graft. In some embodiments, thymic grafts are when the patient is athymic, when the patient's thymus is resistant to regeneration, or to hasten regeneration. In certain embodiments, a thymic xenograft to induce tolerance is used (see .e.g., U.S. Pat. No. 5,658,564). In other embodiments, an allogenic thymic graft is used.

The transplanted cells may be HSC, lymphoid progenitor cells, myeloid progenitor cells, epithelial stem cells or combinations thereof. The present invention also provides a new method for delivery of these cells which promotes uptake and/or differentiation of the cells into T cells. The transplanted cells may or may not be genetically modified. The cells are injected into a patient whose thymus is in the process of being reactivated by the methods of this invention. The optionally modified stem and progenitor cells are taken up by the thymus and converted into T cells, dendritic cells, NK cells, and other cells produced in the thymus. When genetic modifications are present in the transplanted cell, each of these new cells contains the genetic modification of the parent stem/progenitor cell.

In some embodiments, the cells are administered to the patient when the thymus begins to reactivate. In other embodiments, the cells are administered without, prior to, or concurrently with thymus regeneration. In yet other embodiments, the cells are administered when disruption of sex steroid mediated signaling is begun. In one embodiment, stem cells are transplanted into the recipient. The stem cells may be hematopoietic stem cells, epithelial stem cells, or combinations thereof. In another embodiment, progenitor cells are transplanted to the recipient. The progenitor cells may be lymphoid progenitor cells, myeloid progenitor cells, or combinations thereof. In yet another embodiment, the cells are CD34+ or CD34lo HSC. In some embodiments the transplanted cells are autologous. In other embodiments the transplanted cells are nonautologous.

In the event that nonautologous (donor) cells are used, tolerance to these cells is created during the process of thymus reactivation. During or after the initiation of blockage of sex steroid mediated signaling, the relevant (genetically modified (GM) or non-genetically modified) donor cells are transplanted into the recipient.

The donor cells are accepted by the thymus as belonging to the recipient and become part of the production of new T cells and DC by the thymus. The resulting population of T cells recognize both the recipient and donor as self, thereby creating tolerance for a graft from the donor. The graft may be cells, tissues or organs of the donor, or combinations thereof.

As herein defined, the phrase “creating tolerance” or “inducing tolerance” in a patient, and other similar phrases, refers to complete, as well as partial tolerance induction (e.g., a patient may become either more tolerant, or completely tolerant, to the graft, as compared to a patient that has not been treated according to the methods of the invention). Tolerance induction can be tested, e.g., by an MLR reaction, using methods known in the art.

In one embodiment, the methods of the invention use genetically modified HSC, lymphoid progenitor cells, myeloid progenitor cells, epithelial stem cells or combinations thereof (collectively referred to as GM cells) to produce an immune system resistant to attack by particular antigens.

The recipient's thymus may eventually be reactivated by the methods of disrupting sex steroid mediated signaling, as described in more detail below. This disruption reverses the hormonal status of the recipient. In certain embodiments, the recipient is post-pubertal. According to the methods of the invention, the hormonal status of the recipient is reversed such that the hormones of the recipient approach pre-pubertal levels. By lowering the level of sex steroid hormones in the recipient, the signalling of these hormones to the thymus is lowered, thereby allowing the thymus to be reactivated.

As described above, the aged (post-pubertal) thymus causes the body's immune system to function at less than peak levels (such as that found in the young, pre-pubertal thymus). “Post-pubertal” is herein defined as the period in which the thymus has reached substantial atrophy. In humans, this occurs by about 20-25 years of age, but may occur earlier or later in a given individual. “Pubertal” is herein defined as the time during which the thymus begins to atrophy, but may be before it is fully atrophied. In humans this occurs from about 10-20 years of age, but may occur earlier or later in a given individual. “Pre-pubertal” is herein defined as the time prior to the increase in sex steroids in an individual. In humans, this occurs at about 0-10 years of age, but may occur earlier or later in a given individual.

“Recipient,” “patient” and “host” are used interchangeably and are herein defined as a subject receiving sex steroid ablation therapy and/or therapy to interrupt sex steroid mediated signaling and/or, when appropriate, the subject receiving the HSC transplant. “Donor” is herein defined as the source of the transplant, which may be syngeneic, allogeneic or xenogeneic. In some instances, the patient may provide, e.g., his or her own autologous cells for transplant into the patient at a later time point Allogeneic HSC grafts may be used, and such allogeneic grafts are those that occur between unmatched members of the same species, while in xenogeneic HSC grafts the donor and recipient are of different species. Syngeneic HSC grafts, between matched animals, may also be used. The terms “matched,” “unmatched,” “mismatched,” and “non-identical” with reference to HSC grafts are herein defined as the MHC and/or minor histocompatibility markers of the donor and the recipient are (matched) or are not (unmatched, mismatched and non-identical) the same.

The terms “improving,” “enhancing,” or “increasing” tolerance in a patient to a graft or other exogenous antigen is herein defined as meaning that a patient's tolerance to the graft or other exogenous antigen is improved as compared to the tolerance which would have otherwise occurred in a patient without disruption of sex steroid signalling.

The terms thymus “regeneration,” “reactivation” and “reconstitution” and their derivatives are used interchangeably herein, and are herein defined as the recovery of an atrophied or damaged (e.g., by chemicals, radiation, graft versus host disease, infections, genetic predisposition) thymus to its active state. “Active state” is herein defined as meaning a thymus in a patient whose sex steroid hormone mediated signaling has been disrupted, achieves an output of T cells that is at least 10%, or at least 20%, or at least 40%, or at least 60%, or at least 80%, or at least 90% of the output of a pre-pubertal thymus (i.e., a thymus in a patient who has not reached puberty).

This invention may be used with any animal species (including humans) having sex steroid driven maturation and an immune system, such as mammals and marsupials. In some examples, the invention is used with large mammals, such as humans.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Disruption of Sex Steroid Mediated Signalling

The present invention further provides methods of disruption of sex steroid-mediated signaling in a patient, wherein the patient's thymus may or may not be subsequently reactivated. Additionally, the present invention provides methods of improving the functional status of immune cells (e.g., T cells) of the patient. With respect to T cells, the thymus begins to increase the rate of proliferation of the early precursor cells (CD3⁻CD4⁻CD8⁻ cells) and converts them into CD4⁺CD8⁺, and subsequently new mature CD3^{hi}CD₄⁺CD₈⁻ (T helper (Th) lymphocytes) or CD3^{hi}CD₄⁻CD₈⁺ (cytotoxic T lymphocytes (CTL)). The rejuvenating thymus increases its uptake of HSC, or other stem cells or progenitor cells capable of forming into T cells, from the blood stream and converts them into new T cells and intrathymic DC. The increased activity in the thymus resembles in many ways that found in a normal younger thymus (e.g., a prepubertal patient). The result of this renewed thymic output is increased levels of naïve T cells (those T cells which have not yet encountered antigen) in the blood. There is also an increase in the ability of the peripheral T cells to respond to stimulation, e.g., by cross-linking with anti-CD28 Abs, or by TCR stimulation with, e.g., anti-CD3 antibodies, or stimulation with mitogens, such as pokeweed mitogen (PWM) and this increased T cell responsiveness can occur before thymic regeneration, such as within 2, 3, 4, 5 6, 7, 14, or 21 days.

Additionally, in the event that nonautologous (donor) cells are transplanted into a recipient patient, tolerance to these cells is created during the process of thymus reactivation. During or after the initiation of blockage of sex steroid mediated signaling, the relevant (genetically modified (GM) or non-genetically modified) donor cells are transplanted into the recipient. These cells are accepted by the thymus as belonging to the recipient and become part of the production of new T cells and DC by the thymus. The resulting population of T cells recognize both the recipient and donor as self, thereby creating tolerance for a graft from the donor.

As used herein, “sex steroid ablation,” “inhibition of sex steroid-mediated signaling,” “sex steroid disruption” “interruption of sex steroid signaling” and other similar terms are herein defined as at least partial disruption of sex steroid (and/or other hormonal) production and/or sex steroid (and/or other hormonal) signaling, whether by direct or indirect action. In one embodiment, sex steroid signaling to the thymus is interrupted. As will be readily understood, sex steroid-mediated signaling can be disrupted in a range of ways well known to those of skill in the art, some of which are described herein. For example, inhibition of sex hormone production or blocking of one or more sex hormone receptors will accomplish the desired disruption, as will administration of sex steroid agonists and/or antagonists, or active (antigen) or passive (antibody) anti-sex steroid vaccinations.

A non-limiting method for creating disruption of sex steroid-mediated signalling is through castration. Methods for castration include, but are not limited to, chemical castration and surgical castration.

“Castration” is herein defined as the reduction or elimination of sex steroid production, action and/or distribution in the body. This effectively eventually returns the patient to a pre-pubertal status when the thymus is more fully functioning than immediately prior to castration. Surgical castration removes the patient's gonads. Methods for surgical castration are well known to routinely trained veterinarians and physicians. One non-limiting method for castrating a male animal is described in the examples below. Other non-limiting methods for castrating human patients include a hysterectomy or ovariectomy procedure (to castrate women) and surgical castration to remove the testes (to castrate men). In some clinical cases, permanent removal of the gonads via physical castration may be appropriate.

Chemical castration is a less permanent version of castration. As herein defined, “chemical castration” is the administration of a chemical for a period of time, which results in the reduction or elimination of sex steroid production, action and/or distribution in the body. A variety of chemicals are capable of functioning in this manner. Non-limiting examples of such chemicals are the sex steroid inhibitors and/or analogs described below. During the chemical delivery, and for a period of time afterwards, the patient's hormone production may be turned off or reduced. The castration may be reversed upon termination of chemical delivery or by delivery of the relevant sex hormones.

The terms “sex steroid analog,” “sex steroid ablating agent,” “sex steroid inhibitor,” “inhibitor of sex steroid signalling,” “modifier of sex steroid signalling,” and other similar terms are herein defined as any one or more pharmaceutical agent that will decrease, disrupt, prevent, or abolish sex steroid (and/or other hormone) mediated signalling. GnRH (also called LHRH or GnRH/LHRH herein), and analogs thereof, are nonlimiting exemplary inhibitors of sex steroid signalling used throughout this application. However, as will be readily understood by one skilled in the art, in practicing the inventions provided herein, GnRH/LHRH, or analogs thereof, may be replaced with any one (or more) of a number of substitute sex steroid inhibitors or analogs (or other blocker(s) or physical castration) which are described herein, without undue experimentation.

Any pharmaceutical drug, or other method of castration, that ablates sex steroids or interrupts sex steroid-mediated signaling may be used in the methods of the invention. For example, one nonlimiting method of, inhibiting sex steroid signaling, reactivating the thymus and/or enhancing the functionality of BM and immune cells is by modifying the normal action of GnRH on the pituitary (i.e., the release of gonadotrophins, FSH and LH) and consequently reducing normal sex steroid production or release from the gonads. Thus, in one case, sex steroid ablation is accomplished by administering one or more sex hormone analogs, such as a GnRH analog. GnRH is a hypothalamic decapeptide that stimulates the secretion of the pituitary gonadotropins, leutinizing hormone (LH) and follicle-stimulating hormone (FSH). Thus, GnRH agonists (e.g., in the form of Synarel® or Lupron®) initially result in over stimulation of the receptor and through feedback mechanisms will suppress the pituitary production of FSH and LH by desensitization of LHRH Receptors. These gonadotrophins normally act on the gonads to release sex steroids, in particular estrogens in females and testosterone in males; the release of which is significantly reduced by the absence of FSH and LH. The direct consequences of this are a drop in the plasma levels of sex steroids, and as a result, progressive release of the inhibitory signals on the thymus. A more rapid drop in circulating sex steroid levels can be achieved for example by the use of a GnRH antagonist.

In some embodiments, the sex steroid mediated signaling is disrupted by administration of a sex steroid analog, such as an analog of leutinizing hormone-releasing hormone (LHRH). Sex steroid analogs and their use in therapies and chemical castration are well known. Sex steroid analogs are commercially and their use in therapies and chemical castration are well known. Such analogs include, but are not limited to, the following agonists of the LHRH receptor (LHRH-R): buserelin (e.g., buserelin acetate, trade names Suprefact® (e.g., 0.5-02 mg s.c./day), Suprefact Depot®, and Suprefact® Nasal Spray (e.g., 2 μg per nostril, every 8 hrs.), Hoechst, also described in U.S. Pat. Nos. 4,003,884, 4,118,483, and 4,275,001); Cystorelin® (e.g., gonadorelin diacetate tetrahydrate, Hoechst); deslorelin (e.g., desorelin acetate, Deslorell®, Balance Pharmaceuticals); gonadorelin (e.g., gonadorelin hydrocholoride, trade name Factrel® (100 μg i.v. or s.c.), Ayerst Laboratories); goserelin (goserelin acetate, trade name Zoladex®, AstraZeneca, Aukland, NZ, also described in U.S. Pat. Nos. 4,100,274 and 4,128,638; and in GB 9112859 and GB 9112825); histrelin (e.g., histerelin acetate, Supprelin®, (s.c., 10 μg/kg.day), Ortho, also described in EP 217659); leuprolide (leuprolide acetate, trade name Lupron® or Lupron Depot®; Abbott/TAP, Lake Forest, Ill., also described in U.S. Pat. Nos. 4,490,291 3,972,859, 4,008,209, 4,992,421, and 4,005,063; and in DE 2509783); leuprorelin (e.g., leuproelin acetate, trade name Prostap SR® (e.g., single 3.75 mg dose s.c. or i.m./month), Prostap3® (e.g., single 11.25 mg dose s.c. every 3 months), Wyeth, USA, also described in Plosker et al., (1994) Drugs 48:930); lutrelin (Wyeth, USA, also described in U.S. Pat. No. 4,089,946); Meterelin® (e.g., Avorelina (e.g., 10-15 mg slow-release formulation), also described in EP 23904 and WO 91/18016); nafarelin (e.g., trade name Synarel® (i.n. 200-1800 μg/day), Syntex, also described in U.S. Pat. No. 4,234,571; WO 93/15722; and EP 52510); and triptorelin (e.g., triptorelin pamoate; trade names Trelstar LA® (11.25 mg over 3 months), Trelstar LA Debioclip® (pre-filled, single dose delivery), LA Trelstar Depot® (3.75 mg over one month), and Decapeptyl®, Debiopharm S.A., Switzerland, also described in U.S. Pat. Nos. 4,010,125, 4,018,726, 4,024,121, and 5,258,492; and in EP 364819). LHRH analogs also include, but are not limited to, the following antagonists of the LHRH-R: abarelix (trade name Plenaxis™ (e.g., 100 mg i.m. on days 1, 15 and 29, then every 4 weeks thereafter), Praecis Pharmaceuticals, Inc., Cambridge, Mass.) and cetrorelix (e.g., cetrorelix acetate, trade name Cetrotide™ (e.g., 0.25 or 3 mg s.c.), Zentaris, Frankfurt, Germany). Additional sex steroid analogs include Eulexin® (e.g., flutamide (e.g., 2 capsules 2×/day, total 750 mg/day), Schering-Plough Corp., also described in FR 7923545, WO 86/01105 and PT 100899), and dioxane derivatives (e.g., those described in EP 413209), and other LHRH analogs such as are described in EP 181236, and in U.S. Pat. Nos. 4,608,251, 4,656,247, 4,642,332, 4,010,149, 3,992,365, and 4,010,149. Combinations of agonists, combinations of antagonists, and combinations of agonists and antagonists are also included. One non-limiting analog of the invention is deslorelin (described in U.S. Pat. No. 4,218,439). For a more extensive list, of analogs, see Vickery et al. (1984) LHRH AND ITS ANALOGS: CONTRACEPTIVE & THERAPEUTIC APPLICATIONS (Vickery et al., eds.) MTP Press Ltd., Lancaster, Pa. Each analog may also be used in modified form, such as acetates, citrates and other salts thereof, which are well known to those in the art.

One non-limiting example of administration of a sex steroid ablating agent is a subcutaneous/intradermal injection of a “slow-release” depot of GnRH agonist (e.g., one, three, or four month Lupron® injections) or a subcutaneous/intradermal injection of a “slow-release” GnRH-containing implant (e.g., one or three month Zoladex®, e.g., 3.6 mg or 10.8 mg implant). These could also be given intramuscular (i.m.), intravenously (i.v.) or orally, depending on the appropriate formulation. Another example is by subcutaneous injection of a “depot” or “impregnated implant” containing, for example, about 30 mg of Lupron® (e.g., Lupron Depot®, (leuprolide acetate for depot suspension) TAP Pharmaceuticals Products, Inc., Lake Forest, Ill.). A 30 mg Lupron® injection is sufficient for four months of sex steroid ablation to allow the thymus to rejuvenate and export new naïve T cells into the blood stream.

Many of the mechanisms of inhibiting sex steroid signaling described herein are well known and some of these drugs, in particular the GnRH angonists, have been used for many years in the treatment of disorders of the reproductive organs, such as some hormone sensitive cancers including, breast and prostate cancer, endometriosis, reproductive disorders, hirsuitism, precocuis puberty, sexual deviancy and in the control of fertility.

In certain examples, the thymus of the patient is ultimately reactivated by sex steroid ablation and/or interruption or disruption of sex steroid-mediated signalling. In some cases, disruption reverses the hormonal status of the patient. According to the methods of the invention, the hormonal status of the recipient is reversed such that the hormones of the recipient approach pre-pubertal levels. By lowering the level of sex steroid hormones in the recipient, the signalling of these hormones to the thymus is lowered, thereby allowing the thymus to be reactivated. The patient may be pubertal or post-pubertal, or the patient has (or has had) a disease that at least in part atrophied the thymus. Alternatively, the patient has (or has had) a treatment of a disease, wherein the treatment of the disease at least in part atrophied the thymus of the patient. Such treatment may be anti-viral, immunosuppression, chemotherapy, and/or radiation treatment. In other embodiments, the patient is menopausal or has had sex steroid (or other hormonal levels) decreased by another means, e.g., trauma, drugs, etc.

Sex steroid ablation or interruption of sex steroid mediated signaling has one or more direct effect on the BM and/or cells of the immune system, wherein functionality is improved. The effects may occur prior to, or concurrently with, thymic reactivation.

In some embodiments, sex steroid ablation or inhibition of sex steroid signaling is accomplished by administering an anti-androgen such as an androgen blocker (e.g., bicalutamide, trade names Cosudex® or Casodex®, 5-500 mg, e.g., 50 mg po QID, AstraZeneca, Aukland, NZ), either alone or in combination with an LHRH analog or any other method of castration. Sex steroid ablation or interruption of sex steroid signaling may also be accomplished by administering cyproterone acetate (trade name, Androcor®, Shering AG, Germany; e.g., 10-1000 mg, 100 mg bd or tds, or 300 mg IM weekly, a 17-hydroxyprogesterone acetate, which acts as a progestin, either alone or in combination with an LHRH analog or any other method of castration. Other anti-androgens may be used (e.g., antifungal agents of the imidazole class, such as liarozole (Liazol® e.g., 150 mg/day, an aromatase inhibitor) and ketoconazole, flutamide (trade names Euflex® and Eulexin®, Shering Plough Corp, N.J.; 50-500 mg e.g., 250 or 750 mg po QID), megestrol acetate (Megace® e.g., 480-840 mg/day or nilutamide (trade names Anandron®, and Nilandron®, Roussel, France e.g., orally, 150-300 mg/day)). Antiandrogens are often important in therapy, since they are commonly utilized to address flare by GnRH analogs. Some antiandrogens act by inhibiting androgen receptor translocation, which interrupts negative feedback resulting in increased testosterone levels and minimal loss of libido/potency. Another class of anti-androgens useful in the present invention are the selective androgen receptor modulators (SARMS) (e.g., quinoline derivatives, bicalutamide (trade name Cosudex® or Casodex®, as above), and flutamide (trade name Eulexin®, e.g., orally, 250 mg/day)). Other well known anti-androgens include 5 alpha reductase inhibitors (e.g., dutasteride, (e.g., po 0.5 mg/day)) which inhibits both 5 alpha reductase isoenzymes and results in greater and more rapid DHT suppression; finasteride (trade name Proscar®; 0.5-500 mg, e.g., 5 mg po daily), which inhibits 5 alpha reductase 2 and consequent DHT production, but has little or no effect on testosterone or LH levels);

In other embodiments, sex steroid ablation or inhibition of sex steroid signaling is accomplished by administering anti-estrogens either alone or in combination with an LHRH analog or any other method of castration. Some anti-estrogens (e.g., anastrozole (trade name Arimidex®), and fulvestrant (trade name Faslodex®, 10-1000 mg, e.g., 250 mg IM monthly) act by binding the estrogen receptor (ER) with high affinity similar to estradiol and consequently inhibiting estrogen from binding. Faslodex® binding also triggers conformational change to the receptor and down-regulation of estrogen receptors, without significant change in FSH or LH levels. Other non-limiting examples of anti-estrogens are tamoxifen (trade name Nolvadex®); Clomiphene (trade name Clomid®) e.g., 50-250 mg/day, a non-steroidal ER ligand with mixed agonist/antagonist properties, which stimulates release of gonadotrophins; diethylstilbestrol ((DES), trade name Stilphostrol®) e.g., 1-3 mg/day, which shows estrogenic activity similar to, but greater than, that of estrone, and is therefore considered an estrogen agonist, but binds both androgen and estrogen receptors to induce feedback inhibition on FSH and LH production by the pituitary, diethylstilbestrol diphosphate e.g., 50 to 200 mg/day; as well as danazol, droloxifene, and iodoxyfene, which each act as antagonists. Another class of anti-estrogens which may be used either alone or in combination with other methods of castration, are the selective estrogen receptor modulators (SERMS) (e.g., toremifene (trade name Fareston®, 5-1000 mg, e.g., 60 mg po QID), raloxofene (trade name Evista®), and tamoxifen (trade name Nolvadex®, 1-1000 mg, e.g., 20 mg po bd), which behaves as an agonist at estrogen receptors in bone and the cardiovascular system, and as an antagonist at estrogen receptors in the mammary gland). Estrogen receptor downregulators (ERDs) (e.g., tamoxifen (trade name, Nolvadex®)) may also be used in the present invention.

Other non-limiting examples of methods of inhibiting sex steroid signalling which may be used either alone or in combination with other methods of castration, include aromatase inhibitors and other adrenal gland blockers (e.g., Aminoglutethimide, formestane, vorazole, exemestane, anastrozole (trade name Arimidex®, 0.1-100 mg, e.g., 1 mg po QID), which lowers estradiol and increases LH and testosterone), letrozole (trade name Femara®, 0.2-500 mg, e.g., 2.5 mg po QID), and exemestane (trade name Aromasin®) 1-2000 mg, e.g., 25 mg/day); aldosterone antagonists (e.g., spironolactone (trade name, Aldactone®) e.g., 100 to 400 mg/day), which blocks the androgen cytochrome P-450 receptor;) and eplerenone, a selective aldosterone-receptor antagonist) antiprogestogens (e.g., medroxypregesterone acetate, e.g. 5 mg/day, which inhibits testosterone syntheses and LH synthesis); and progestins and anti-progestins such as the selective progesterone response modulators (SPRM) (e.g., megestrol acetate e.g., 160 mg/day, mifepristone (RU 486, Mifeprex®, e.g. 200 mg/day); and other compounds with estrogen/antiestrogenic activity, (e.g., phytoestrogens, flavones, isoflavones and coumestan derivatives, lignans, and industrial compounds with phenolic ring (e.g., DDT)). Also, anti-GnRH vaccines (see, e.g., Hsu et al., (2000) Cancer Res. 60:3701; Talwar, (1999) Immunol. Rev. 171:173-92), or any other pharmaceutical which mimics the effects produced by the aforementioned drugs, may also be used. In addition, steroid receptor based modulators, which may be targeted to be thymic and/or BM specific, may also be developed and used. Many of these mechanisms of inhibiting sex steroid signaling are well known. Each drugs may also be used in modified form, such as acetates, citrates and other salts thereof, which are well known to those in the art.

Because of the complex and interwoven feedback mechanisms of the hormonal system, administration of sex steroids may result in inhibition of sex steroid signalling. For example, estradiol decreases gonadotropin production and sensitivity to GnRH action. However, higher levels of estradiol result in gonadotropin surge. Likewise, progesterone influences frequency and amount of LH release. In men, testosterone inhibits gonadotropin production. Estrogen administered to men decreases LH and testosterone, and anti-estrogen increases LH.

In other embodiments, prolactin is inhibited in the patient. Another means of inhibiting sex steroid mediated signaling may be by means of direct or indirect modulation of prolactin levels. Prolactin is a single-chain protein hormone synthesized as a prohormone. The normal values for prolactin are males and nonpregnant females typically range from about 0 to 20 ng/ml, but in pregnancy the range is typically about 10 to 300 ng/ml. Overall, several hundred different actions have been reported for prolactin. Prolactin stimulates breast development and milk production in females. Abnormal prolactin is known to be involved in pituitary tumors, menstrual irregularities, infertility, impotence, and galactorrhea (breast milk production). A considerable amount of research is in progress to delineate the role of prolactin in normal and pathologic immune responses. It appears that prolactin has a modulatory role in several aspects of immune function, yet there is evidence to suggest that hyperprolactinemia is immunosuppressive (Matera L, 1997 Neuroimmunomodulation, July-August ; 4(4): 171-80). Administration of prolactin in pharmacological doses is associated with a decreased survival and an inhibition of cellular immune functions in septic mice. (Oberbeck R, 2003 J Surg Res. August; 113(2):248-56). There are also a large number of drugs which impair dopaminergic inhibition of prolactin and give rise to hyperprolactinemia. Antidopaminergic agents include haloperidol, fluphenazine, sulpiride, metoclopramide and gastrointestinal prokinetics (e.g., bromopride, clebopride, domperidone, and levosulpiride) which have been exploited clinically for the management of motor disorders of the upper gastrointestinal tract.

Inhibin A and B peptides made in the gonads in response to gonadotropins, down regulates the pituitary and suppress FSH. Activin normally up regulates GnRH receptors and stimulate FSH synthesis, however over production may shut down sex steroid production. Thus, these hormones may also be the target of inhibition of sex steroid-mediated signalling.

In certain embodiments, an LHRH-R antagonist is delivered to the patient, followed by an LHRH-R agonist. For example, the antagonist can be administered as a single injection of sufficient dose to cause castration within 5-8 days (this is normal for, e.g., Abarelix). When the sex steroids have reached this castrate level, the agonist is given. This protocol abolishes or limits any spike of sex steroid production, before the decrease in sex steroid production, that might be produced by the administration of the agonist. In an alternate embodiment, an LHRH-R agonist that creates little or no sex steroid production spike is used, with or without the prior administration of an LHRH-R antagonist.

Inhibition of Sex Steroid Signalling

Sex steroids comprise a large number of the androgen, estrogen and progestin family of hormone molecules. Non-limiting members of the progestin family of C21 steroids include progesterone, 17α-hydroxy progesterone, 20α-hydroxy progesterone, pregnanedione, pregnanediol and pregnenolone. Non-limiting members of the androgen family of C19 steroids include testosterone, androstenedione, dihydrotesterone (DHT), androstanedione, androstandiol, dehydroepiandrosterone and 17α-hydroxy androstenedione. Non-limiting members of the estrogen family of C17 steroids include estrone, estradiol-17α, and estradiol-17β.

Signalling by sex steroids is the net result of complex outcomes of the components of the pathway that includes biosynthesis, secretion, metabolism, compartmentalization and action. Parts of this pathway are not fully understood; nevertheless, there are numerous existing and potential mechanisms for achieving inhibition of sex steroid signalling. In one embodiment of the present invention, inhibition of sex steroid signalling is achieved by modifying the bioavailable sex steroid hormone levels at the cellular level, the so called ‘free’ levels, by altering biosynthesis or metabolism, the binding to sex steroid receptors on or in target cells, and/or intracellular signalling of sex steroids.

It is possible to influence the signalling pathways either directly or indirectly. The direct methods include methods of influencing sex steroid biosynthesis and metabolism, binding to the respective receptor and intracellular modification of the signal. The indirect methods include those methods known to influence sex steroid hormone production and action such as the peptide hormone and growth factors present in the pituitary gland and the gonad. The latter include but not be limited to FSH, LH and activin made by the pituitary gland, and inhibin, activin and insulin-like growth factor-1 (IGF-1) made by the gonad.

The person skilled in the art will appreciate that inhibition of sex steroid signaling may take place by making the aforementioned modifications at the level of the relevant hormone, enzyme, receptor, binding molecule and/or ligand, either by direct action upon that molecule or by action upon a precursor of that molecule, including a nucleic acid that encodes or regulates it, or a molecule that can modify the action of sex steroid.

Direct Methods of Inhibiting Signaling

Biosynthesis

The rate of biosynthesis is the major rate determining step in the production of steroid hormones and hence the bioavailability of ‘free’ hormone in serum. Inhibition of a key enzyme such as P450 cholesterol side chain cleavage (P450scc), early in the pathway, will reduce production of all the major sex steroids. On the other hand, inhibition of enzymes later in the pathway, such as P450 aromatase (P450arom) that converts androgens to estrogens, or 5α-reductase that converts testosterone to DHT, will only effect the production of estrogens or DHT, respectively. Another important facet of sex steroid hormone biosynthesis is the family of oxidoreductase enzymes that catalyze the interconversion of inactive to bioactive steroids, for example, androstenedione to testosterone or estrone to estradiol-17□ by 17-hydroxysteroid dehydrogenase (17-HSD). These enzymes are tissue and cell specific and generally catalyze either the reduction or oxidation reaction e.g., 17β HSD type 3 is found exclusively in the Leydig cells of the testes, whereas 17β HSD type 1 is found in the ovary. They therefore offer the possibility of specifically reducing production of the active forms of androgens or estrogens.

There are many known inhibitors of the enzymes in the steroid biosynthesis pathway that are either already in clinical use or are under development. Some examples of these together with their treatment modalities are listed above. It is important that the action of these enzyme inhibitors does not unduly influence production of other steroids such as glucocorticoids and mineralocorticoids from the adrenal gland that are essential for metabolic stability. When using such inhibitors, it may be necessary to provide the patient with replacement glucocorticoids and sometimes mineralocorticoids.

Sex steroid biosynthesis occurs in varied sites and utilizing multiple pathways, predominantly produced the ovaries and testes, but there is some production in the adrenals, as well as synthesis of derivatives in other tissues, such as fat. Thus, multiple mechanisms of inhibiting sex steroid signaling may be required to ensure adequate inhibition to achieve the present invention.

Metabolism and Compartmentalization

Sex steroid hormones have a short half-life in blood, generally only several minutes, due to the rapid metabolism, particularly by the liver, and clearance by the kidney and fat. Metabolism includes conjugation by glycosylation and sulphation, as well as reduction. Some of these metabolites retain biological activity either as prohormones, for example estrone sulphate, or through intrinsic bioactivity such as the reduced androgens. Any interference in the rate of metabolism can influence the ‘free’ levels of sex steroid hormones, however methods of achieving this are not currently available as are methods of influencing biosynthesis.

Another method of reducing the level of ‘free’ sex steroid hormone is by compartmentalization by binding of the sex steroid hormone to proteins present in the serum such as sex hormone binding globulin, corticosteroid-binding globulin, albumin and testosterone-estradiol binding globulin. Binding to sex steroid ligands, such as carrier molecules may make sex steroids unavailable for receptor binding. Increased binding may result from increased levels of carriers, such as SHBG or introduction of other ligands which bind the sex steroids, such as soluble receptors. Alternatively decreased levels of carrier molecules may make sex steroids more susceptible to degradation.

Active or passive immunization against a particular sex steroid hormone is a form of compartmentalization. There are examples in the literature of this approach successfully increasing ovulation rates in animals after immunization against estrogen or androgen. Sex steroids are secreted from cells in secretory vesicles. Inhibition or modification of the secretory mechanism is another method of inhibiting sex steroid signaling

Receptors and Intracellular Signalling

The sex steroids act on cells via specific receptors that can be either intracellular, or, as shown more recently, on the target cell membrane.

The intracellular receptors are members of the nuclear receptor superfamily. They are located in the cytoplasm of the cell and are transported to the nucleus after binding with the sex steroid hormone where they alter the transcription of specific genes. Receptors for the sex steroid hormones exist in several forms. Well known in the literature are two forms of the progesterone receptor, PRA and PRB, and three forms of the estrogen receptor, ERα, ERβ1 and ERβ2. Transcription of genes in response to the binding of the sex steroid hormone receptor to the steroid response element in the promoter region of the gene can be modified in a number of ways. Co-activators and co-repressors exist within the nucleus of the target cell that can modify binding of the steroid-receptor complex to the DNA and thereby effect transcription. The identity of many of these co-activators and co-repressors are known and methods of modifying their actions on steroid receptors are the topic of current research. Representative examples of the transcription factors involved in sex steroid hormone action are NF-1, SP1, Oct-1 and TFIID. These co-regulators are required for the full action of the steroids. Methods of modifying the actions of these nuclear regulators may involve the balance between activator and repressor by the use of antagonists or through control of expression of the genes encoding the regulators.

Specific receptors for estrogens and progesterone have been identified on the membranes of cells whose structures are different from the intracellular PR. Unlike the classical steroid receptors that act on the genome, these receptors deliver a rapid, non-genomic action via intracellular pathways that are not yet fully understood. Estrogens interacting with membrane receptors may activate the sphingosine pathway that is related to cell proliferation.

There are methods available or in development to alter the action of steroids via their cytoplasmic receptors. In this case, antiandrogens, antiestrogens, and antiprogestins that interact with the specific steroid receptors, are well known in the literature and are in clinical use, as described above. Their action may be to compete for, or block the receptor, to modify receptor levels, sensitivity, conformation, associations or signaling. These drugs come in a variety of forms, steroidal and non-steroidal, competitive and noncompetitive. Of particular interest are the selective receptor modulators, SARMS, SERMS and SPRM, which are targeted to particular tissues and are exemplified above.

Down-regulation of receptors can be achieved in two ways; first, by excess agonist (steroid ligand), and second, by inhibiting transcription of the respective gene that encodes the receptor. The first method can be achieved through the use of selective agonists such as tamoxifen.

Indirect Methods of Inhibiting Signaling

Biosynthesis

One of the indirect methods of inhibiting sex steroid signalling involves down-regulation of the biosynthesis of the respective steroid by a modification to the availability or action of the pituitary gonadotrophins, FSH and LH, that are responsible for driving the biosynthesis of the sex steroid hormones in the gonad. One established inhibitor of FSH secretion is inhibin, a hormone produced by the gonads in response to FSH. Administration of inhibin to animals has been shown to reduce FSH levels in serum due to a decrease in the pituitary secretion of FSH. One known way of accomplishing a reduction in both gonadotrophins is via the hypothalamic hormone, GnRH/LHRH, which drives the pituitary synthesis and secretion of FSH and LH. Agonists and antagonists of GnRH that reduce the secretion of FSH and LH, and hence gonadal sex steroid production, are now available for clinical use, as described herein.

Another indirect method of reducing the biosynthesis of sex steroid hormones is to modify the action of FSH and LH at the level of the gonad. This can be achieved by using antibodies directed against FSH and LH, or molecules designed to compete with FSH and LH for their respective receptors on gonadal cells that produce the sex steroid hormones. Another method of modifying the action of FSH and LH on gonadal cells is by using a co-regulator of gonadotrophin action. For example, activin can reduce the capacity of the theca cells of the ovary and the Leydig cells of the testis to produce androgen in response to LH.

Modification may take place at the level of hormone precursors such as inhibition of cleavage of a signal peptide, for example the signal peptide of GnRH.

Receptors and Intracellular Signalling

Indirect methods of altering the signalling action of the sex steroid hormones include down-regulation of the receptor pathways leading to the genomic or non-genomic actions of the steroids. An example of this is the capacity of progesterone to down regulate the level of ER in target tissues. Future methods include treatment with molecules known to influence the co-regulators of the receptors in the cell nucleus leading to a decrease in the capacity of the cell to respond to the steroid.

Additional Factors

While the stimulus for the direct and indirect effects on BM functionality, BM lymphopoiesis, and immune cell functionality is fundamentally based on the inhibition of the effects of sex steroids and/or the direct effects of the LHRH analogs, it may be useful to include additional substances which can act in concert to enhance or increase (additive, synergistic, or complementary) the thymic, BM, and/or immune cell effects and functionality. Additional substances may or may not be used. Such compounds include, but are not limited to, cytokines and growth factors, such as interleukin-2 (IL-2; 100,000 to 1,000,000 IU, e.g., 600,000 IU/Kg every 8 hours by IV repeat doses), interleukin-7 (IL-7; 10 ng/kg/day to 100 mcg/kg/day subject to therapeutic discretion), interleukin-15 (IL-15; 0.1-20 mug/kg IL-15 per day), interleukin 11 (IL-11; 1-1000 μg/kg) members of the epithelial and fibroblast growth factor families, stem cell factor (SCF; also known as steel factor or c-kit ligand; 0.25-12.5 mg/ml), granulocyte colony stimulating factor (G-CSF; 1 and 15 μg/kg/day IV or SC), granulocyte macrophage stimulating factor (GM-CSF; 50-1000 μg/sq meter/day SC or IV), insulin dependent growth factor (IGF-1), and keratinocyte growth factor (KGF; 1 μg/kg to 100 mg/kg/day) (see, e.g., Sempowski et al., (2000) J. Immunol. 164:2180; Andrew and Aspinall, (2001) J. Immunol. 166:1524-1530; Rossi et al., (2002) Blood 100:682); erythropoietin (EPO; 10-500 units/kg IV or SC). A non-exclusive list of other appropriate hematopoietins, CSFs, cytokines, lymphokines, hematopoietic growth factors and interleukins for simultaneous or serial co-administration with the present invention includes, Meg-CSF (Megakaryocyte-Colony Stimulating Factor, more recently referred to as c-mpl ligand), MIF (Macrophage Inhibitory Factor), LIF (Leukemia Inhibitory Factor), TNF (Tumor Necrosis Factor), IGF, platelet derived growth factor (PDGF), M-CSF, IL-1, IL-4, IL-5, IL-6, IL-8, IL-9, IL-10, IL-12, IL-13, LIF, flt3/flk2, human growth hormone, B-cell growth factor, B-cell differentiation factor and eosinophil differentiation factor, or combinations thereof.

One or more of these additional compound(s) may be given once at the initial LHRH analog (or other castration method) application. Each treatment may be given in combination with the agonist, antagonist or any other form of sex steroid disruption. Since the growth factors have a relatively rapid half-life (e.g., in the hours) they may need to be given each day (e.g., every day for 7 days or longer). The growth factors/cytokines may be given in the optimal form to preserve their biological activities, as prescribed by the manufacturer, e.g., in the form of purified proteins. However, additional doses of any one or combination of these substances may be given at any time to further stimulate the functionality of the BM and other immune cells. In certain cases, sex steroid ablation or interruption of sex steroid signalling is done concurrently with the administration of additional cytokines, growth factors, or combinations thereof. In other cases, sex steroid ablation or interruption of sex steroid signalling is done sequentially with the administration of additional cytokines, growth factors, or combinations thereof.

As used herein “mobilizing agent” refers to agents such as SDF-1 (e.g., AMD3100), Growth Hormone, GM-CSF, G-CSF and chemotherapeutics (e.g., cyclophosphamide) which enhance mobilisation of stem cells from the BM.

G-CSF and GM-CSF are known to mobilize the production of granulocytes (primarily neutrophils) and macrophages, respectively, and also result in increased production of DC from the BM, which help provide a non-specific immune response in the patient to antigenic challenge (Janeway et al., (2001) Immunobiology 5^{th} ed., p. 325). Clinically, G-CSF and GM-CSF are used, for example, to decrease the incidence of infection (as manifested by febrile neutropenia) in patients with non-myeloid malignancies receiving myelosuppressive anti-cancer drugs, which are typically associated with a significant incidence of severe neutropenia and fever. Additionally, both of these drugs are approved clinically to prevent infections in patients receiving HSCT. Both G-CSF and GM-CSF are currently used in patients undergoing peripheral blood progenitor cell collection or therapy. Colony stimulating factors (CSFs), which stimulate the differentiation and/or proliferation of BM stem cells, have generated much interest because of their therapeutic potential for restoring depressed levels of hematopoietic stem cell-derived cells. CSFs in both human and murine systems have been identified and distinguished according to their activities. For example, granulocyte-CSF (G-CSF) and macrophage-CSF (M-CSF) stimulate the in vitro formation of neutrophilic granulocyte and macrophage colonies, respectively while GM-CSF and interleukin-3 (IL-3) have broader activities and stimulate the formation of both macrophage, neutrophilic and eosinophilic granulocyte colonies. IL-3 also stimulates the formation of mast, megakaryocyte and pure and mixed erythroid colonies (when erythropoietin is added). GM-CSF accelerates recovery of neutrophils and maintains functional capacity, yet has little demonstrable effect on platelet recovery. In contrast, IL-3 promotes a slower increase recovery in neutrophils and monocytes while accelerating the recovery of platelets.

Thus, G-CSF and/or GM-CSF are used in some endorsements of the methods of the invention. Sex steroid ablation together (sequentially or concurrently) with G-CSF and/or GM-CSF therapy results in an increase in the output from the BM of both lymphoid and myeloid cell, which in turn significantly improves both the short and long term outcomes for patients suffering, or likely to suffer from, infections. In another method, the CSFs are administered 3-4 days after chemotherapy or radiation therapy. Clinical outcomes already associated with the use of the CSFs are also greatly enhanced by an interruption to sex steroid signaling. In particular, using the methods of the instant invention together with CSF's, allows for much greater infection control in patients receiving e.g., cancer radiation or chemotherapy. Additionally, if the immune system can be effectively and promptly “rebooted,” increased dosages and/or frequency of chemotherapy drugs or radiation therapy may be used. This may occur with or without the introduction of allogeneic or autogenic HSC's which would further enhance the timely return of immune system functionality. Castration will also result in a lower number of HSC that have to be transplanted, which will be useful when only a limited number of HSC can be obtained from a donor or when cord blood stem cells are used for transplant.

For instance, the concurrent use of two separate classes of drugs (e.g., a GnRH analog, such as Lupron®, and an androgen blocker, such as Cosudex®) may allow for the same immune system regeneration but may require a reduced dosage of the G-CSF or GM-CSF. Similarly, the concurrent use of these two separate classes of drugs may allow for a greater, or more prolonged rejuvenation of immune system cells, while utilizing the same dosage of G-CSF or GM-CSF. Additionally, the concurrent use of two separate classes of drugs may allows for the same rejuvenation of immune system cells, while utilizing a reduced dosage (i.e., a reduction compared to the “normally” used dosages used for the treatment of prostate cancer, endometriosis, or breast cancer) of the drug, or combination of drugs, used to ablate or interrupt sex steroid signaling. Further, the concurrent use of these two separate classes of drugs allows for a greater, or prolonged rejuvenation of immune system cells, while utilizing a reduced dosage of the drug, or combination of drugs, used to ablate or interrupt sex steroid signaling.

Indications

The use of drugs known to cause sex steroid ablation, or which interrupt sex steroid signaling, either alone or in combination, with or without the aforementioned growth factors and cytokines may be used for the following: as an adjunct in enabling HSC engraftment (see, e.g., Example 22); as an adjunct in the effective management of allogeneic or autologous organ or cell transplants (see, e.g., Examples 21 and 22, and co-pending, co-owned U.S. Ser. Nos. 10/419,039 and 10/749,119). The use of these drugs in these diseases will either result in more effective treatment outcomes or will result in the overall treatment protocols being more efficient. Additionally, as described in, e.g., Examples 25 and 26, the doses or administration of the various chemotherapy drugs (or doses of radiation therapy) may be altered such that they now produce less side effects and/or result in better quality of life outcomes for the patients. Moreover, the coadministration of the various cytokines and growth factors may allow for a reduced number of HSC that need to be transplanted. For example, using the method of the invention, it may now be possible to use human cord blood for adult HSCT, since a reduced number of cells is required to obtain engraftment.

Pharmaceutical Compositions

The compounds used in this invention may be supplied in any pharmaceutically acceptable carrier or may be supplied without a carrier. Formulations of pharmaceutical compositions can be prepared according to standard methods (see, e.g., Remington, 2000 The Science and Practice of Pharmacy, Gennaro A. R., ed., 20^{th} edition, Williams & Wilkins PA, USA). Non-limiting examples of pharmaceutically acceptable carriers include physiologically compatible coatings, solvents and diluents. For parenteral, subcutaneous, intravenous, and intramuscular administration, the compositions may be protected such as by encapsulation. Alternatively, the compositions may be provided with carriers that protect the active ingredient(s), while allowing a slow release of those ingredients. Numerous polymers and copolymers are known in the art for preparing time-release preparations, such as various versions of lactic acid/glycolic acid copolymers. See, for example, U.S. Pat. No. 5,410,016, which uses modified polymers of polyethylene glycol (PEG) as a biodegradable coating.

Formulations intended to be delivered orally can be prepared as liquids, capsules, tablets, and the like. These compositions can include, for example, excipients, diluents, and/or coverings that protect the active ingredient(s) from decomposition. Such formulations are well known (see, e.g., Remington, 2000).

In any of the formulations of the invention, other compounds that do not negatively affect the activity of the LHRH analogs (i.e., compounds that do not block the ability of an LHRH analog to disrupt sex steroid hormone signalling) may be included. Examples are various growth factors and other cytokines as described herein.

Dose

Doses of a sex steroid analog or inhibitor used, in according with the invention, to disrupt sex steroid hormone signaling, can be readily determined by a routinely trained physician or veterinarian, and may be also be determined by consulting medical literature (e.g., THE PHYSICIAN'S DESK REFERENCE, 52ND EDITION, Medical Economics Company, 1998).

The dosage regimen involved in a method for treating the above-described conditions will be determined by the attending physician considering various factors which modify the action of drugs, e.g., the condition, body weight, sex and diet of the patient, the severity of any illness, time of administration and other clinical factors. Progress of the treated patient can be monitored by periodic assessment of the hematological profile, e.g., differential cell count and the like.

The dosing recited above would be adjusted to compensate for additional components in the therapeutic composition. These include co-administration with other CSF, cytokine, lymphokine, interleukin, hematopoietic growth factor; co-administration with chemotherapeutic drugs and/or radiation; and various patient-related issues as identified by the attending physician such as factors which modify the action of drugs, e.g., the condition, body weight, sex and diet of the patient, the severity of any illness, time of administration and other clinical factors.

In addition to dosing described above, for example, LHRH analogs and other sex steroid analogs can be administered in a one-time dose that will last for a period of time (e.g., 3 to 6 months). In certain cases, the formulation will be effective for one to two months. The standard dose varies with type of analog used, but is readily determinable by those skilled in the art without undue experimentation. In general, the dose is between about 0.01 mg/kg and about 10 mg/kg, or between about 0.01 mg/kg and about 5 mg/kg.

The length of time of sex steroid inhibition or LHRH/GnRH analog treatment varies with the degree of thymic atrophy and damage, and is readily determinably by those skilled in the art without undue experimentation. For example, the older the patient, or the more the patient has been exposed to T cell depleting reagents such as chemotherapy or radiotherapy, the longer it is likely that they will require treatment, for example with GnRH. Four months is generally considered long enough to detect new T cells in the blood. Methods of detecting new T cells in the blood are known in the art. For instance, one method of T cell detection is by determining the existence of T cell receptor excision circles (TREC's), which are formed when the TCR is being formed and are lost in the cell after it divides. Hence, TREC's are only found in new (naïve) T cells. TREC levels are an indicator of thymic function in humans. These and other methods are described in detail in WO/00 230,256.

Dose varies with the LHRH analog used. In certain embodiments, a dose is prepared to last as long as needed. For example, a formulation of an LHRH analog can be made and delivered as described herein for a period of two or more months, with additional doses delivered every two or more months as needed.

The formulation can be made to enhance the immune system. Alternatively, GM cells can be administered with the LHRH analog formulation or separately, both spatially and/or in time. As with the non-GM cells, multiple doses over time can be administered to a patient s needed to create tolerance to a given exogenous antigen.

As will be understood by persons skilled in the art, at least some of the means for disrupting sex steroid signalling will only be effective as long as the appropriate compound is administered. As a result, an advantage of certain embodiments of the present invention is that once the desired immunological affects of the present invention have been achieved, (2-3 months) the treatment can be stopped and thee subjects reproductive system will return to normal.

Delivery of Agents for Chemical Castration

Administration of sex steroid ablating agents may be by any method which delivers the agent into the body. Thus, the sex steroid ablating agent maybe be administered, in accordance with the invention, by any route including, without limitation, intravenous, subdermal, subcutaneous, intramuscular, topical, and oral routes of administration.

In addition to the methods described above, delivery of the compounds for use in the methods of this invention may be accomplished via a number of methods known to persons skilled in the art. One standard procedure for administering chemical inhibitors to inhibit sex steroid mediated signalling utilizes a single dose of an LHRH agonist that is effective for three months. For this a simple one-time i.v. or i.m. injection would not be sufficient as the agonist would be cleared from the patient's body well before the three months are over. Instead, a depot injection or an implant may be used, or any other means of delivery of the inhibitor that will allow slow release of the inhibitor. Likewise, a method for increasing the half-life of the inhibitor within the body, such as by modification of the chemical, while retaining the function required herein, may be used.

Useful delivery mechanisms include, but are not limited to, laser irradiation of the skin. This embodiment is described in more detail in co-owned, co-pending U.S. Ser. No. 10/418,727 and also in U.S. Pat. Nos. 4,775,361, 5,643,252, 5,839,446, 6,056,738, 6,315,772, and 6,251,099. Another useful delivery mechanism includes the creation of high pressure impulse transients (also called stress waves or impulse transients) on the skin. This embodiment is described in more detail in co-owned, co-pending U.S. Ser. No. 10/418,727 and also U.S. Pat. Nos. 5,614,502 and 5,658,822. Each method may be accompanied or followed by placement of the compound(s) with or without carrier at the same locus. One method of this placement is in a patch placed and maintained on the skin for the duration of the treatment.

Timing

In one case, the administration of agents (or other methods of castration) that ablate sex steroids or interrupt to sex steroid signaling occurs prior to a, e.g., a chemotherapy or radiation regimen that is likely to cause some BM marrow cell ablation and/or damage to circulating immune cells.

Cells

Injection of hematopoietic progenitor cells, e.g., broadly defined as CD34⁺ hematopoietic cells (ideally autologous) can enhance the degree and kinetics of thymic regrowth and/or increases in immune cell and BM functionality and engraftment without, prior to or concurrently with thymic regeneration. HSC may also be further defined as Thy-1 low and CD38−; CD34+CD38−; Thy-1 low cells also lack markers of other cell lineages (lin-ve) are the more primitive HSC being longer lasting or having longer-term repopulating capacity.

The methods of the various inventions described herein can be supplemented by the addition of, e.g., CD34⁺ HSC and/or epithelial stem cells. In one instance, these cells are autologous or syngeneic and have been obtained from the patient or twin prior to thymus reactivation. The HSC can be obtained by sorting CD34⁺ or CD34^{lo} cells from the patient's blood and/or BM. The number of HSC can be enhanced in several ways, including (but not limited to) by administering G-CSF (Neupogen, Amgen) to the patient prior to collecting cells, culturing the collected cells in SCGF, and/or administering G-CSF to the patient after CD34⁺ cell supplementation. Alternatively, the CD34⁺ cells need not be sorted from the blood or BM if their population is enhanced by prior injection of G-CSF into the patient

HSC may be used for genetic modification. These may be derived from BM, peripheral blood, or umbilical cord, or any other source of HSC, and may be either autologous or nonautologous. Also useful are lymphoid and myeloid progenitor cells, mesenchymal stem cells also found in the bone marrow and epithelial stem cells, also either autologous or nonautologous. The stem cells may also include umbilical cord blood. They may also include stem cells which have the potential to form into many different cell types e.g. embryonic stem cells and adult stem cells now found in may tissues, e.g., BM, pancreas, brain, and the olfactory system.

In the event that nonautologous (donor) cells are used, tolerance to these cells is created during or after thymus reactivation. During or after the initiation of blockage of sex steroid mediated signaling, the relevant (genetically modified (GM) or non-genetically modified) donor cells are transplanted into the recipient. These cells, ideally stem or progenitor cells, are incorporated into and accepted by the thymus wherein they create tolerance to the donor by eliminating any newly produced T cells which by chance could be reactive against them. They are then “belonging to the recipient” and may become part of the production of new T cells and DC by the thymus. The resulting population of T cells recognize both the recipient and donor as self, thereby creating tolerance for a graft from the donor (see co-owned, co-pending U.S. Ser. No. 10/419,039 and PCT/IB01/02740).

In another embodiment the administration of stem or precursor donor cells (genetically modified or not genetically modified) comprises cells from more than one individual, so that the recipient develops tolerance to a range of MHC types, enabling the recipient to be considered a suitable candidate for a cell, tissue or organs transplant more easily or quickly, since they are an MHC match to a wider range of donors.

The present invention also provides methods for incorporation of foreign DC into a patient's thymus. This may be accomplished by the administration of donor cells to a recipient to create tolerance in the recipient. The donor cells may be HSC, epithelial stem cells, adult or embryonic stem cells, or hematopoietic progenitor cells. The donor cells may be CD34⁺ HSC, lymphoid progenitor cells, or myeloid progenitor cells. In some cases, the donor cells are CD34+ or CD34lo HSC. The donor HSC may develop into DC in the recipient. The donor cells may be administered to the recipient and migrate through the peripheral blood system to the reactivating thymus either directly or via the BM. To enhance thymic incorporation for tolerance induction, the stem cells may also be injected intrathymically in combination with activation of thymic regrowth through use of sex steroid inhibitors, e.g., LHRH/GnRH analogues. Even non-HSC are likely to be induced to form into DC within the thymic microenvironment and its content of appropriate growth factors for such cells.

The uptake into the thymus of the hematopoietic precursor cells is substantially increased in the inhibition or absence of sex steroids. These cells become integrated into the thymus and produce DC, NK, NKT, and T cells in the same manner as do the recipient's cells. The result is a chimera of T cells, DC and the other cells. The incorporation of donor DC in the recipient's thymus means that T cells produced by this thymus will be selected such that they are tolerant to donor cells. Such tolerance allows for a further transplant from the donor (or closely matched to the donor) of a graft, such as donor cells, tissues and organs, with a reduced need for immonusuppressive drugs since the transplanted material will be recognized by the recipient's immune system as self.

Optional Genetic Modification of Stem or Progenitor Cells

The present disclosure also comprises methods for optionally altering the immune system of an individual and methods of gene therapy using genetically modified hematopoietic stem cells, lymphoid progenitor cells, myeloid progenitor cells, epithelial stem cells, or combinations thereof (GM cells). Previous attempts by others to deliver such cells have been unsuccessful, resulting in negligible levels of the modified cells. The present disclosure provides a new method for delivery of these cells which promotes uptake and differentiation of the cells into the desired T cells. The modified cells are injected into a patient. The modified stem and progenitor cells are taken up by the thymus and converted into T cells, dendritic cells, and other cells produced in the thymus. Each of these new cells contains the genetic modification of the parent stem/progenitor cell.

During or after the castration step, hematopoietic stem or progenitor cells, or epithelial stem cells, from the donor may be transplanted into the recipient patient. These cells are accepted by the thymus as belonging to the recipient and become part of the production of new T cells and DC by the thymus. Within three to four weeks of the start of blockage of sex steroid mediated signaling (approximately two to three weeks after the initiation of LHRH treatment), the first new T cells are preset in the blood stream. Full development of the T cell pool may take three to four months. The resulting population of T cells recognize both the recipient (and donor, in the case of nonautologous transplants) as self. Tolerance for a graft from the donor may also be created in the recipient.

An appropriate gene or polynucleotide (i.e., the nucleic acid sequence defining a specific protein) that will create tolerance of the patient to a donor graft is engineered into the stem and/or progenitor cells. By introducing the specific gene into the HSC, the cell differentiates into, e.g., an APC and expresses the protein as a peptide expressed in the context of MHC class I or II.

The person may be given a sex steroid analog to activate their thymus, and/or to improve their bone marrow function, which includes the increased ability to take up and produce HSC. In one case, hematopoietic cells are supplied to the patient before or concurrently with thymic reactivation, which increases the immune capabilities of the patient's body. In one embodiment, a patient receives a HSCT during or after castration. The person may be injected with their own HSC, or may be injected with HSC from an appropriate donor, which has, e.g., treatment with G-CSF for 3 days (2 injections, subcutaneously per day) followed by collection of HSC from the blood on days 4 and 5. The HSC may be transfected or transduced with a gene (e.g., encoding the protein, peptide, or antigen from the agent) to produce to the required protein or antigen. Following injection into the patient, the HSC enter the bone and bone marrow from the blood and then some exit back to the blood to be eventually converted into T cells, DC, APC throughout the body. The antigen is expressed in the context of MHC class I and/or MHC class II molecules on the surface of these APC.

Methods for isolating and transducing stems cells and progenitor cells are well known to those skilled in the art. Examples of these types of processes are described, for example, in PCT Publication Nos. WO 95/08105, WO 96/33282, WO 96/33281, U.S. Pat. Nos. 5,559,703, 5,061,620, 5,681,559 and 5,199,942.

Antisense Polynucleotides

The term “antisense” is herein defined as a polynucleotide sequence which is complementary to a polynucleotide of the present invention. The polynucleotide may be DNA or RNA. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

Catalytic Nucleic Acids

The term “catalytic nucleic acid” is herein defined as a DNA molecule or DNA containing molecule (also known in the art as a “deoxyribozyme” or “DNAzyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity. The catalytic strand cleaves a specific site in a target nucleic acid. The types of ribozymes that are particularly useful in this invention are the hammerhead ribozyme (Haseloff and Gerlach (1988) Nature 334:585), Perriman et al., (1992) Gene 113:157) and the hairpin ribozyme (Shippy et al., (1999) Mol. Biotechnol. 12:117).

dsRNA

Double stranded RNA (dsRNA) is particularly useful for specifically inhibiting the production of a particular protein. Although not wishing to be limited by theory, one group has provided a model for the mechanism by which dsRNA can be used to reduce protein production (Dougherty and Parks, (1995), Curr. Opin. Cell Biol. 7:399). This model has more recently been modified and expanded (Waterhouse et al., (1998) Proc. Natl. Acad. Sci. USA 95:13959). This technology relies on the presence of dsRNA molecules that contain a sequence that is essentially identical to the mRNA of the gene of interest, in this case an mRNA encoding a polypeptide according to the first aspect of the invention. Conveniently, the dsRNA can be produced in a single open reading frame in a recombinant vector or host cell, where the sense and antisense sequences are flanked by an unrelated sequence which enables the sense and anti-sense sequences to hybridize to form the dsRNA molecule with the unrelated sequence forming a loop structure. The design and production of suitable dsRNA molecules for the present invention are well within the capacity of a person skilled in the art, particularly considering Dougherty and Parks, (1995), Curr. Opin. Cell Biol. 7:399; Waterhouse et al., (1998) Proc. Natl. Acad. Sci. USA 95:13959; and PCT Publication Nos. WO 99/32619, WO 99/53050, WO 99/49029, and WO 01/34815.

Genes

Useful genes and gene fragments (polynucleotides) for this invention include those that code for resistance to a particular exogenous antigen, such as donor antigens or even allergens. In one non-limiting example of the invention, where the donor is related to the recipient but expresses an additional MHC molecule or a molecule expressed by the Y chromosome (e.g., where the recipient is female and the donor is male), the genes encoding that molecule could be transfected and expressed in either the donor's HSC before reconstitution of the recipient with the donor's HSC, or could transfected and expressed in the recipient's own HSC (e.g., collected from the recipient prior to or concurrent with sex steroid ablation).

These genes or gene fragments are used in a stably expressible form. These genes or gene fragments may be used in a stably expressible form. The term “stably expressible” is herein defined to mean that the product (RNA and/or protein) of the gene or gene fragment (“functional fragment”) is capable of being expressed on at least a semi-permanent basis in a host cell after transfer of the gene or gene fragment to that cell, as well as in that cell's progeny after division and/or differentiation. This requires that the gene or gene fragment, whether or not contained in a vector, has appropriate signaling sequences for transcription of the DNA to RNA. Additionally, when a protein coded for by the gene or gene fragment is the active molecule that affects the patient's condition, the DNA will also code for translation signals.

In most cases the genes or gene fragments are contained in vectors. Those of ordinary skill in the art are aware of expression vectors that may be used to express the desired RNA or protein. Expression vectors are vectors that are capable of directing transcription of DNA sequences contained therein and translation of the resulting RNA. Expression vectors are capable of replication in the cells to be genetically modified, and include plasmids, bacteriophage, viruses, and minichromosomes. Alternatively the gene or gene fragment may become an integral part of the cell's chromosomal DNA. Recombinant vectors and methodology are in general well-known.

Expression vectors useful for expressing the proteins of the present disclosure may comprise an origin of replication. Suitably constructed expression vectors comprise an origin of replication for autonomous replication in the cells, or are capable of integrating into the host cell chromosomes. Such vectors may also contain selective markers, a limited number of useful restriction enzyme sites, a high copy number, and strong promoters. Promoters are DNA sequences that direct RNA polymerase to bind to DNA and initiate RNA synthesis; strong promoters cause such initiation at high frequency.

In one embodiment, the DNA vector construct comprises a promoter, enhancer, and a polyadenylation signal. The promoter may be selected from the group consisting of HIV, such as the Long Terminal Repeat (LTR), Simian Virus 40 (SV40), Epstein Barr virus, cytomegalovirus (CMV), Rous sarcoma virus (RSV), Moloney virus, mouse mammary tumor virus (MMTV), human actin, human myosin, human hemoglobin, human muscle creatine, human metalothionein. In one embodiment, an inducible promoter is used so that the amount and timing of expression of the inserted gene or polynucleotide can be controlled.

The enhancer may be selected from the group including, but not limited to, human actin, human myosin, human hemoglobin, human muscle creatine and viral enhancers such as those from CMV, RSV and EBV. The promoter and enhancer may be from the same or different gene.

The polyadenylation signal may be selected from the group consisting of: LTR polyadenylation signal and SV40 polyadenylation signal, particularly the SV40 minor polyadenylation signal among others.

The expression vectors of the present disclosure may be operably linked to DNA coding for an RNA or protein to be used in this invention, i.e., the vectors are capable of directing both replication of the attached DNA molecule and expression of the RNA or protein encoded by the DNA molecule. Thus, for proteins, the expression vector must have an appropriate transcription start signal upstream of the attached DNA molecule, maintaining the correct reading frame to permit expression of the DNA molecule under the control of the control sequences and production of the desired protein encoded by the DNA molecule. Expression vectors may include, but are not limited to, cloning vectors, modified cloning vectors and specifically designed plasmids or viruses. An inducible promoter may be used so that the amount and timing of expression of the inserted gene or polynucleotide can be controlled.

One having ordinary skill in the art can produce DNA constructs which are functional in cells. In order to test expression, genetic constructs can be tested for expression levels in vitro using tissue culture of cells of the same type of those to be genetically modified.

Methods of Genetic Modification

Standard recombinant methods can be used to introduce genetic modifications into the cells being used for gene therapy. For example, retroviral vector transduction of cultured HSC is one successful method known in the art (Belmont and Jurecic (1997) “Methods for Efficient Retrovirus-Mediated Gene Transfer to Mouse Hematopoietic Stem Cells,” in Gene Therapy Protocols (P. D. Robbins, ed.), Humana Press, pp. 223-240; Bahnson et al., (1997) “Method for Retrovirus-Mediated Gene Transfer to CD34⁺-Enriched Cells,” in Gene Therapy Protocols (P. D. Robbins, ed.), Humana Press, pp. 249-263). Additional vectors include, but are not limited to, those that are adenovirus derived or lentivirus derived, and Moloney murine leukemia virus-derived vectors.

Also useful for genetic modification of HSC are the following methods: particle-mediated gene transfer such as with the gene gun (Yang and Ziegelhoffer, (1994) “The Particle Bombardment System for Mammalian Gene Transfer,” In Particle Bombardment Technology Gene Transfer (Yang, N.-S. and Christou, P., eds.), Oxford University Press, New York, pp. 117-141), liposome-mediated gene transfer (Nabel et al., (1992) Hum. Gene Ther. 3:649), coprecipitation of genetically modified vectors with calcium phosphate (Graham and Van Der Eb, (1973) Virol. 52:456), electroporation (Potter et al., (1984) Proc. Natl. Acad. Sci. USA 81:7161), and microinjection (Capecchi, (1980) Cell 22:479), as well as any other method that can stably transfer a gene or oligonucleotide, which may be in a vector, into the HSC and other cells to be genetically modified such that the gene will be expressed at least part of the time.

Gene Therapy

The present disclosure also provides methods for gene therapy through reactivation of a patient's thymus. This is accomplished by the administration of GM cells to a recipient and through disruption of sex steroid mediated signaling. By the methods described herein, the sex steroid-induced atrophic thymus is ultimately restored structurally and functionally to approximately its optimal pre-pubertal capacity in all currently definable terms. This includes the number, type and proportion of all T cell subsets. Also included are the complex stromal cells and their three dimensional architecture which constitute the thymic microenvironment required for producing T cells. The newly generated T cells emigrate from the thymus and restore peripheral T cell levels and function.

The ultimate reactivation of the thymus can be supplemented by the addition of CD34⁺ hematopoietic stem cells (HSC) and/or epithelial stem cells slightly before or at the time the thymus begins to regenerate. These cells may be autologous or syngeneic and have been obtained from the patient or twin prior to thymus reactivation. The HSC can be obtained by sorting CD34⁺ cells from the patient's blood and/or bone marrow. The number of HSC can be enhanced in several ways, including (but not limited to) by administering G-CSF (Neupogen, Amgen) to the patient prior to collecting cells, culturing the collected cells in Stem Cell Growth Factor, and/or administering G-CSF to the patient after CD34⁺ cell supplementation. Alternatively, the CD34⁺ cells need not be sorted from the blood or BM if their population is enhanced by prior injection of G-CSF into the patient.

In one embodiment, hematopoietic cells are supplied to the patient before or concurrent with thymic reactivation, which increases the immune capabilities of the patient's body. The hematopoietic cells may or may not be genetically modified.

The genetically modified cells may be HSC, epithelial stem cells, embryonic or adult stem cells, or myeloid or lymphoid progenitor cells. In one embodiment, the genetically modified cells are CD34+ or CD34lo HSC, lymphoid progenitor cells, or myeloid progenitor cells. In another embodiment, the genetically modified cells are CD34⁺ HSC. The genetically modified cells are administered to the patient and migrate through the peripheral blood system to the thymus. The uptake into the thymus of these hematopoietic precursor cells is substantially increased in the absence of sex steroids. These cells become integrated into the thymus and produce dendritic cells and T cells carrying the genetic modification from the altered cells. The results are a population of T cells with the desired genetic change that circulate in the peripheral blood of the recipient, and the accompanying increase in the population of cells, tissues and organs caused by the reactivating patient's thymus.

Within 3-4 weeks of the start of blockage of sex steroid mediated signalling (approximately 2-3 weeks after the initiation of LHRH treatment), the first new T cells are present in the blood stream. Full development of the T cell pool, however, may take 3-4 (or more) months.

Induction of Tolerance

The T cell population of an individual can be altered through the methods of this invention. In particular, modifications can be induced that will create tolerance of non-identical (i.e., allogenic or xenogenic) grafts. The establishment of tolerance to exogenous antigens, particularly non-self donor antigens in clinical graft situations, can be best achieved if dendritic cells of donor origin are incorporated into the recipient's thymus. This form of tolerance may also be made more effective through the use of inhibitory immunoregulatory cells (e.g., CD25+CD4+ T cells, NKT cells, γδT cells). The mechanisms underlying the development of the latter, however, are poorly understood, but again could involve dendritic cells.

Given that a major mechanism underlying the prevention of T cells reacting against self antigens is due to the negative selection (by clonal deletion) of such cells by thymic dendritic cells, the ability to create a thymus which has dendritic cells from a potential organ or tissue donor has major importance in the prevention of graft rejection. This is because the T cells which could potentially reject the graft will have encountered the donor dendritic cells in the thymus and be deleted before they have the opportunity to enter the blood stream. The blood precursor cells which give rise to the dendritic cells are the same as those which give rise to T cells themselves.

In some embodiments, the transplanted HSC may follow full myeloablation or myelodepletion, and thus result in a full HSC transplant (e.g., 5×10⁶ cells/kg body weight per transplant). In some embodiments, only minor myeloablation need be achieved, for example, 2-3 Gy irradiation (or 300 rads) followed by administration of about 3-4×10⁵ cells/kg body weight. In some embodiments, T cell depletion (TCD), and/or another method of immune cell depletion, is used (see, e.g., Example 2). It may be that as little as 10% chimerism may be sufficient to establish tolerance to a donor's graft. In some embodiments, the donor HSC are from umbilical cord blood (e.g., 1.5×10⁷ cells/kg for recipient engraftment).

The ability of the HSC to first colonise the BM and convert to blood cells (engraftment) is directly linked to the absolute number and quality of the HSC injected, and the functional capacity of the recipient bone marrow microenvironment and the HSC niches. The methods of the present invention either alone or in combination (concurrently or sequentially) with the administration of HSC mobilizing agents, such as cytokines (e.g., G-CSF or GM-CSF), or drugs (e.g., cyclophosphamide), allow faster and/or better engraftment and may also allow chemotherapy and radiation therapy to be given at higher doses and/or more frequently.

In other embodiments, patients begin to receive Lupron up to 45 days before myeloablative chemotherapy and continue on the Lupron concurrently with the BMT such that the total length of exposure to the drug is around 9 months (equivalent to 3 injections as each Lupron injection delivers drug over a 3 month period). At various intervals over the course of study, blood samples are collected for analysis of T cell numbers (particularly of new thymic emigrants) and functions (specifically, response to T cell stimuli in vitro). This embodiment is also generally applicable to HSCT for other purposes described herein.

In other embodiments, the transplanted HSC may follow lymphoablation. In some embodiments, T cells and/or B cells may be selectively ablated, to remove cells, as needed (e.g., those cells involved in autoimmunity or allergy). The selection can involve deletion of cells that are activated, or of a cell type involved in the autoimmune or allergic response. The cells may be selected based upon cell surface markers, such as CD4, CD8, B220, thy1, TCR, CD3, CD5, CD7, CD25, CD26, CD23, CD30, CD38, CD49b, CD69, CD70, CD71, CD95, CD96, antibody specificity or Ig chain, or upregulated cytokine receptors e.g., IL2-R B chain, TGFβ. One well known method for depletion is the use of antilymphocyte globulin. Other methods of selecting and sorting cells are well known and include magnetic and fluorescent cell separation, centrifugation, and more specifically, hemapheresis, leukopheresis, and lymphopheresis.

In some embodiments, HSCT is performed without myeloablation, myelodepletion, lymphodepletion, T cell ablation, and/or other selective immune cell ablation.

In other embodiments, the methods of the invention further comprise immunosuppressing the patient by e.g., administration of an immunosuppressing agent (e.g., cyclosporine, prednisone, ozothioprine, FK506, Imunran, and/or methotrexate) (see, e.g., U.S. Pat. No. 5,876,708). In an embodiment, immunosuppression is performed in the absence of HSCT. In one embodiment, immunosuppression is performed in conjunction with (e.g., prior to, concurrently with, or after) HSCT. In another embodiment, immunosuppression is performed in the absence of myeloablation, lymphoablation, T cell ablation and/or other selective immune cell ablation, deletion, or depletion. In yet another embodiment, immunosuppression is performed in conjunction with (e.g., prior to, concurrently with, or after) myeloablation, lymphoablation, T cell ablation, and/or other selective immune cell ablation, deletion, or depletion.

As described above, myeloablation, myelodepletion, lymphoablation, immunosuppression, T cell ablation, and/or other selective immune cell ablation, are nonlimiting exemplary types of immune cell ablation, which are used throughout this application. The general term “immune cell depletion” is defined herein as encompassing each of these methods, i.e., myeloablation, myelodepletion, lymphoablation, T cell ablation, and/or other selective immune cell ablation (e.g., B cell or NK cell depletion). As will be readily understood by one skilled in the art, in practicing the inventions provided herein, any one of these “depletion” methods may be replaced with any one (or more) of the other “depletion” methods.

In one embodiment, NK cells are depleted. NK antibodies useful for depleting the NK populations are known in the art. For example, one source of anti-NK antibody is anti-human thymocyte polyclonal anti-serum. U.S. Pat. No. 6,296,846 describes NK and T cell depletion methods, as well as non-myeloablative therapy and formation of a chimeric lymphohematopoietic population, all of which may be used in the methods of the invention.

In some embodiments, the methods of the invention further comprise, e.g., prior to HSCT, absorbing natural antibodies from the blood of the recipient by hemoperfusing an organ (e.g., the liver or kidney) obtained from the donor.

In another embodiment the present invention further includes a T cell help-reducing treatment, such as increasing the level of the activity of a cytokine which directly or indirectly (e.g., by the stimulation or inhibition of the level of activity of a second cytokine) promotes tolerance to a graft (e.g., IL-10, IL-4, or TGF-.beta.), or which decreased the level of the activity of a cytokine which promotes rejection of a graft (i.e., a cytokine which is antagonistic to or inhibits tolerance (e.g., IFN.beta., IL-1, IL-2, or IL-12)). In some embodiments, a cytokine is administered to promote tolerance. The cytokine may be derived from the donor species or from the recipient species (see, e.g., U.S. Pat. No. 5,624,823, which describes DNA encoding porcine interleukin-10 for such use). The duration of the help-reducing treatment may be approximately equal to, or is less than, the period required for mature T cells of the recipient species to initiate rejection of an antigen after first being stimulated by the antigen (in humans this is usually 8-12 days). In other embodiments, the duration is approximately equal to or is less than two-, three-, four-, five-, or ten times the period required for mature T cells of the recipient to initiate rejection of an antigen after first being stimulated by the antigen. The short course of help-reducing treatment may be administered in the presence or absence of a treatment which may stimulate the release of a cytokine by mature T cells in the recipient, e.g., in the absence of Prednisone (17,21-dihydroxypregna-1,4diene-3,11,20-trione). The help-reducing treatment may be begun before or about the time the graft is introduced. The short course of help-reducing treatment may be pre-operative or post-operative. In some embodiments, the donor and recipient are class I matched.

Moreover, the ability to enhance the uptake into the thymus of hematopoietic stem cells means that the nature and type of dendritic cells can be manipulated. For example, the stem cells could be transfected with specific gene(s) which eventually become expressed in the dendritic cells in the thymus (and elsewhere in the body). In one non-limiting example of the invention, where the donor is related to the recipient but expresses an additional MHC molecule or a molecule expressed by the Y chromosome (e.g., where the recipient is female and the donor is male), the genes encoding that molecule could be transfected and expressed in either the donor's HSC before reconstitution of the recipient with the donor's HSC, or could transfected and expressed in the recipient's own HSC (e.g., collected from the recipient prior to or concurrent with sex steroid ablation). Methods of genetically modifying cells to, e.g., insert MHC (HLA or SLA) genes are know in the art (see, e.g., U.S. Pat. Nos. 5,614,187, 6,030,833, 6,306,651 and 6,558,663). Some of the HSC, whether donor or recipient, develop into dendritic cells, and so educate the newly formed T cells that the additional molecule is “self”. T cells thus educated, when encountering such a molecule expressed by the donor graft tissue, will recognize the tissue as self and not attempt to reject it. Indeed, positive selection can involve multiple cell types: the cortical epithelium provides the specific differentiation molecules and third party cells provide the MHC/peptide ligands.

Effects on BM and HSC

The present invention provides methods for increasing the function of BM in a patient, including increasing production of HSC and enhancing haemopoiesis. These methods are useful in a number of applications. For example, one of the difficult side effects of chemotherapy or radiotherapy, whether given for cancer or for another purpose, can be its negative impact on the patient's BM. Depending on the dose of chemotherapy, the BM may be damaged or ablated and production of blood cells may be impeded. Administration of a dose of a sex steroid analog (such as an LHRH analog) according to this invention after chemotherapy treatment aids in recovery from the damage done by the chemotherapy to the BM and blood cells. Alternatively, administration of the LHRH analog in the weeks prior to delivery of chemotherapy increases the population of HSC and other blood cells so that some of the deleterious effects of chemotherapy will be decreased.

The improvement in BM function may be applicable to, for example, patients with blood disorders. As used herein, “blood disorders” is meant any disorder or malady that involves the cells of the blood system in a patient, either directly or indirectly, including, but not limited to, disorders associated with hematopoiesis, e.g., leukemia. Thus, for example, the methods of the present invention are useful to replace the cancerous blood system cells with new cells from a donor (matched or unmatched) in an allogenic HSCT, or following autologous HSCT with the patient's own cells.

Increased HSC production by the BM causes consequential increase in red blood cells, which are, in turn, useful for management of RBC production. This can be easily determined by looking for, e.g., increased hematocrit.

In some examples, the increased HSC are CD34⁺ HSC. Mobilized HSCs (e.g., using G-CSF) can assist in the “repair” or rejuvenation of tissues, such as with heart tissue and lung tissue. HSC have the potential to generate non-hematopoietic tissue. While much of the work has been carried out in vitro, a study at the Mayo Clinic, Rochester has shown that after BMT a small number of cadiomyocytes are donor derived. Similarly, the Beuamont Hospital, Michigan have used HSC to repair damaged heart muscle, although it is unclear whether the HSC become myocytes or vasculature. Mice experiments have also shown the potential of HSC to become insulin producing β-cells. Other work, has shown HSC are capable of becoming skeletal muscle (myocytes), liver (hepatocytes), bone, connective tissue, epithelial tissue (e.g. of lung, gut and skin), vasculature, neurons, and islet □ cells.

The methods described herein are useful to repair damage to the BM and/or assist in the replacement of blood cells that may have been injured or destroyed by various therapies (e.g., cancer chemotherapy drugs, radiation therapy ) or diseases (e.g., HIV, chronic renal failure).

In some chemotherapy regimens, such as high dose chemotherapy to treat any of the blood cancers, ablation of the BM is a desired effect. The methods of the invention may be used immediately after ablation occurs to stimulate the BM and increase the production of HSC and their progeny blood cells, so as to decrease the patient's recovery time. Following administration of the chemotherapy, usually allowing one or more days for the chemotherapy to clear from the patient's body, a dose of LHRH analog according to the methods described herein is administered to the patient. This can be in conjunction with the administration of autologous or heterologous BM or hematopoietic stem or progenitor cells, as well as other factors such as colony stimulating factors (CSFs) and stem cell factor (SCF).

Alternatively, a patient may have suboptimal (or “tired”) BM function and may not be producing sufficient or normal numbers of HSC and other blood cells. This can be caused by a variety of conditions, including normal ageing, prolonged infection, post-chemotherapy, post-radiation therapy, chronic disease states including cancer, genetic abnormalities, and immunosuppression induced in transplantation. Further, radiation, such as whole-body radiation, can have a major impact on the BM productivity. These conditions can also be either pre-treated to minimize the negative effects (such as for chemotherapy and/or radiation therapy, or treated after occurrence to reverse the effects.

Effects on T Cells

Sex steroids in males and females can be inhibited temporarily by taking disruptors of sex steroid mediated signaling (e.g., GnRH agonists). It has been shown that loss of steroids causes a reactivation of thymus function and enhanced production of naïve T cells. Furthermore, even before those new T cells have had a chance to leave the thymus, the preexisting T cells are much more sensitive to stimulation, which results in a much more effective immune response. This increased responsiveness is evident within days of the loss of sex steroids (see, e.g., Example 23). This may be because there are no inhibitory effects of sex steroids. This may be because of the production of “helper” or “adjuvant” factors by the reactivating or reactivated thymus, which are able to costimulate the T cells in conjunction with the foreign stimulus. Also, since many cells of the immune system have surface receptors for GnRH, the GnRH, itself, may provide an additional stimulation for the T cells. Since the effect of the loss of sex steroids on peripheral T cells is so rapid, GnRH can be given as a single treatment simultaneously with the delivery of, for example, a vaccine. A one month formulation is useful which has the beneficial effects of stimulating immune responses but without the side effects of longer loss of sex steroids. A subsequent “booster” injection of the antigen can also be administered.

Sex steroid inhibitors (e.g., GnRH analogs) are useful to boost all forms of immunotherapy in cancer patient, particularly for the removal of cancer cells with have escaped chemotherapy or surgery, but also for the defense against opportunistic infections. These analogs may also be used prophylacticly to improve immune response to vaccination programs designed to prevent, e.g., infections or cancer.

The methods of the invention utilize inhibition of sex steroid signaling. Sex steroids suppress the function of the thymus, BM, and also T and B lymphocytes throughout the body, which are concentrated in the major lymphoid areas of the body including, but not limited to, the blood, lymph nodes, mucosal tissue (e.g., respiratory, gastrointestinal, genital). It has surprisingly been discovered that ablation of sex steroids and/or interruption of sex steroid-mediated signaling may be used not only to regenerate the thymus (and thus the number and ‘quality’ of T cells), but also to improve the functionality of preexisting and newly produce T cells (and other cells of the immune system) either without, prior to, or concurrently with, thymus regeneration.

A poor immune response can have immediate and clinically important consequences. It can mean an increased susceptibility to common infections (e.g., influenza), increased susceptibility to cancers and tumors, and/or poor responsiveness to vaccinations.

An increase in the number and/or proportion of naïve T cells in the total T cell pool has a positive immediate therapeutic effect on a number of clinical (or potentially clinical) conditions and diseases, including, but not limited to, cancer, immunodeficiency (particularly viral infections, e.g., Acquired Immune Deficiency Syndrome (ADS) and Severe Acute Respiratory Syndrome (SARS), or influenza), autoimmunity, transplantation, allergies, as well as improving the general efficacy of vaccination programs. Each of these applications is described in detail in co-owned and co-pending U.S. application Ser. Nos. 10/418,747, 10/419,039, 10/418,953, 10/418,727, 10/419,066, and 10/419,068.

The inhibition of sex steroid signaling (e.g., using LHRH/GnRH analogs) may also be given on multiple occasions. The immediate effects are due to enhancing the functionality of preexisting lymphoid (and non-lymphoid) cells. With time, the reduction of sex steroids increases the production and functionality of T cells, B cells and APC, which additively, synergistically, or complementarily continue to enhance the response. Increases of new APC, as well as T cells, B cells, and other immune cells, together with increased sensitivity of preexisting T cells, B cells, and APC to stimulation, may then be used to enhance a patient's tolerance to a donor graft.

Thus, disruptors of sex steroid signaling are used according to the methods of the instant invention, to cause a clinically positive effect by initiating an increased activation or functionality of these immune cells even before these drugs have been able to cause significant thymic regrowth.

Effects on DC

The present invention also provides methods for increasing DC functionality and/or DC number. Following sex steroid ablation (e.g., following delivery of an LHRH analog) DC are increased in the thymus, and in the periphery, which may also assist the T cell stimulation.

### EXAMPLES

The following Examples provide specific examples of methods of the invention, and are not to be construed as limiting the invention to their content.

### Example 1

### Reversal of Aged-Induced Thymic Atrophy

Materials and Methods

Animals.

CBA/CAH and C57B16/J male mice were obtained from Central Animal Services, Monash University and were housed under conventional conditions. C57B16/J Ly5.1⁺ were obtained from the Central Animal Services Monash University, the Walterand Eliza Hall Institute for Medical Research (Parkville, Vicotoria) and the A.R.C. (Perth, Western Australia) and were housed under conventional conditions. Ages ranged from 4-6 weeks to 26 months of age and are indicated where relevant.

Surgical Castration.

Animals were anesthetized by intraperitoneal injection of 0.3 ml of 0.3 mg xylazine (Rompun®; Bayer Australia Ltd., Botany NSW, Australia) and 1.5 mg ketamine hydrochloride (Ketalar®; Parke-Davis, Caringbah, NSW, Australia) in saline. Surgical castration was performed by a scrotal incision, revealing the testes, which were tied with suture and then removed along with surrounding fatty tissue. The wound was closed using surgical staples. Sham-castration followed the above procedure without removal of the testes and was used as controls for all studies.

Bromodeoxyuridine (BrdU) Incorporation.

Mice received two intraperitoneal injections of BrdU (Sigma Chemical Co., St. Louis, Mo.) at a dose of 100 mg/kg body weight in 100 μl of PBS, 4-hours apart (i.e., at 4 hour intervals). Control mice received vehicle alone injections. One hour after the second injection, thymuses were dissected and either a cell suspension made for FACS analysis, or immediately embedded in Tissue Tek (O.C.T. compound, Miles Inc., Indiana), snap frozen in liquid nitrogen, and stored at −70° C. until use.

Flow Cytometric Analysis.

Mice were killed by CO₂ asphyxiation and thymus, spleen, and mesenteric lymph nodes were removed. Organs were pushed gently through a 200 μm sieve in cold PBS/1% FCS/0.02% Azide, centrifuged (650 g, 5 min, 4° C.), and resuspended in either PBS/FCS/Az. Spleen cells were incubated in red cell lysis buffer (8.9 g/liter ammonium chloride) for 10 min at 4° C., washed and resuspended in PBS/FCS/Az. Cell concentration and viability were determined in duplicate using a hemocytometer and ethidium bromide/acridine orange and viewed under a fluorescence microscope (Axioskop; Carl Zeiss, Oberkochen, Germany).

For 3-color immunofluorescence, cells were labeled with anti-αβTCR-FITC, anti-CD4-PE and anti-CD8-APC (all obtained from Pharmingen, San Diego, Calif.) followed by flow cytometry analysis. Spleen and lymph node suspensions were labeled with either αβTCR-FITC/CD4-PE/CD8-APC or B220-B (Sigma) with CD4-PE and CD8-APC. B220-B was revealed with streptavidin-Tri-color conjugate purchased from Caltag Laboratories, Inc., Burlingame, Calif.

For BrdU detection of cells, cells were surface labeled with CD4-PE and CD8-APC, followed by fixation and permeabilization as previously described (Carayon and Bord, (1989) J. Imm. Meth. 147:225). Briefly, stained cells were fixed overnight at 4° C. in 1% paraformaldehyde (PFA)/0.01% Tween-20. Washed cells were incubated in 500 μl DNase (100 Kunitz units, Roche, USA) for 30 mins at 37° C. in order to denature the DNA. Finally, cells were incubated with anti-BrdU-FITC (Becton-Dickinson) for 30 min at room temperature, washed and resuspended for FACS analysis.

For BrdU analysis of TN subsets, cells were collectively gated out on Lin-cells in APC, followed by detection for CD44-biotin and CD25-PE prior to BrdU detection. All antibodies were obtained from Pharmingen (San Diego, Calif.).

For 4-color Immunofluorescence, thymocytes were labeled for CD3, CD4, CD8, B220 and Mac-1, collectively detected by anti-rat Ig-Cy5 (Amersham, U.K.), and the negative cells (TN) gated for analysis. They were further stained for CD25-PE (Pharmingen, San Diego, Calif.) and CD44-B (Pharmingen, San Diego, Calif.) followed by Streptavidin-Tri-color (Caltag, Calif.) as previously described (Godfrey and Zlotnik, (1993) Immunol. Today 14:547). BrdU detection was then performed as described above.

Samples were analyzed on a FACSCalibur™ (Becton-Dickinson). Viable lymphocytes were gated according to 0° and 90° light scatter profiles and data was analyzed using CellQuest™ software (Becton-Dickinson).

Immunohistology.

Frozen thymus sections (4 μm) were cut using a cryostat (Leica) and immediately fixed in 100% acetone.

For two-color immunofluorescence, sections were double-labeled with a panel of monoclonal antibodies: MTS6, 10, 12, 15, 16, 20, 24, 32, 33, 35 and 44 (Godfrey et al., (1990) Immunol. 70:66; Table 1) produced in this laboratory and the co-expression of epithelial cell determinants was assessed with a polyvalent rabbit anti-cytokeratin Ab (Dako, Carpinteria, Calif.). Bound mAb was revealed with FITC-conjugated sheep anti-rat Ig (Silenus Laboratories, Victoria, Australia) and anti-cytokeratin was revealed with TRITC-conjugated goat anti-rabbit Ig (Silenus Laboratories, Victoria, Australia).

For BrdU detection of sections, sections were stained with either anti-cytokeratin followed by anti-rabbit-TRITC or a specific mAb, which was then revealed with anti-rat Ig-Cγ3 (Amersham, Uppsala, Sweden). BrdU detection was then performed as previously described (Penit et al., (1996) Proc. Natl. Acad. Sci, USA 86:5547). Briefly, sections were fixed in 70% Ethanol for 30 mins. Semi-dried sections were incubated in 4M HCl, neutralized by washing in Borate Buffer (Sigma), followed by two washes in PBS. BrdU was detected using anti-BrdU-FITC (Becton-Dickinson).

For three-color immunofluorescence, sections were labeled for a specific MTS mAb together with anti-cytokeratin. BrdU detection was then performed as described above.

Sections were analyzed using a Leica fluorescent and Nikon confocal microscopes.

Migration Studies (i.e., Analysis of Recent Thymic Emigrants (RTE)).

Animals were anesthetized by intraperitoneal injection of 0.3 ml of 0.3 mg xylazine (Rompun®; Bayer Australia Ltd., Botany NSW, Australia) and 1.5 mg ketamine hydrochloride (Ketalar®; Parke-Davis, Caringbah, NSW, Australia) in saline.

Details of the FITC labeling of thymocytes technique are similar to those described elsewhere (Scollay et al., (1980) Proc. Natl. Acad. Sci, USA 86:5547; Berzins et al., (1998) J. Exp. Med. 187:1839). Briefly, thymic lobes were exposed and each lobe was injected with approximately 10 μm of 350 μg/ml FITC (in PBS). The wound was closed with a surgical staple, and the mouse was warmed until fully recovered from anesthesia. Mice were killed by CO₂ asphyxiation approximately 24 hours after injection and lymphoid organs were removed for analysis.

After cell counts, samples were stained with anti-CD4-PE and anti-CD8-APC, then analyzed by flow cytometry. Migrant cells were identified as live-gated FITC⁺ cells expressing either CD4 or CD8 (to omit autofluorescing cells and doublets). The percentages of FITC⁺ CD4 and CD8 cells were added to provide the total migrant percentage for lymph nodes and spleen, respectively. Calculation of daily export rates was performed as described by Berzins et al., ((1998) J. Exp. Med. 187:1839).

Data analyzed using the unpaired student ‘t’ test or nonparametrical Mann-Whitney U-test was used to determine the statistical significance between control and test results for experiments performed at least in triplicate. Experimental values significantly differing from control values are indicated as follows: *p≦0.05, **p≦0.01 and ***p≦0.001.

Results

I. The Effect of Age on Thymocyte Populations.

(i) Thymic Weight and Thymocyte Number

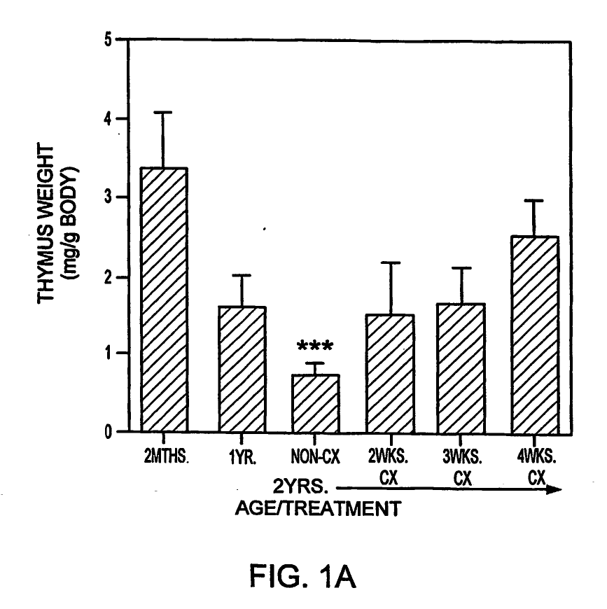

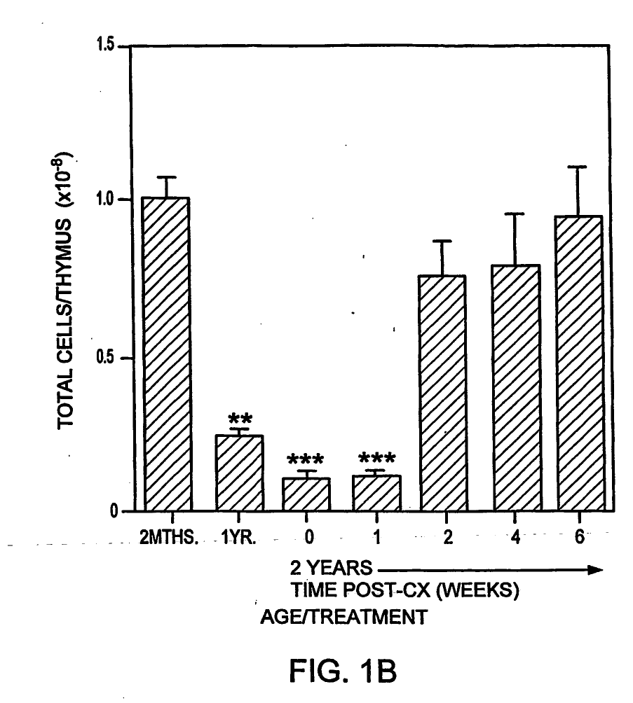

With increasing age there is a highly significant (p≦0.0001) decrease in both thymic weight (FIG. 1A) and total thymocyte number (FIG. 1B) in mice. Relative thymic weight (mg thymus/g body) in the young adult has a mean value of 3.34 which decreases to 0.66 at 18-24 months of age (adipose deposition limits accurate calculation). The decrease in thymic weight can be attributed to a decrease in total thymocyte numbers: the 1-2 month (i.e., young adult) thymus contains about 6.7×10⁷ thymocytes, decreasing to about 4.5×10⁶ cells by 24 months. By removing the effects of sex steroids on the thymus by castration, thymocyte cell numbers are regenerated and by 4 weeks post-castration, the thymus is equivalent to that of the young adult in both weight (FIG. 1A) and cellularity (FIG. 1B). Interestingly, there was a significant (p≦0.001) increase in thymocyte numbers at 2 weeks post-castration (1.2×10⁸), which is restored to normal young levels by 4 weeks post-castration.

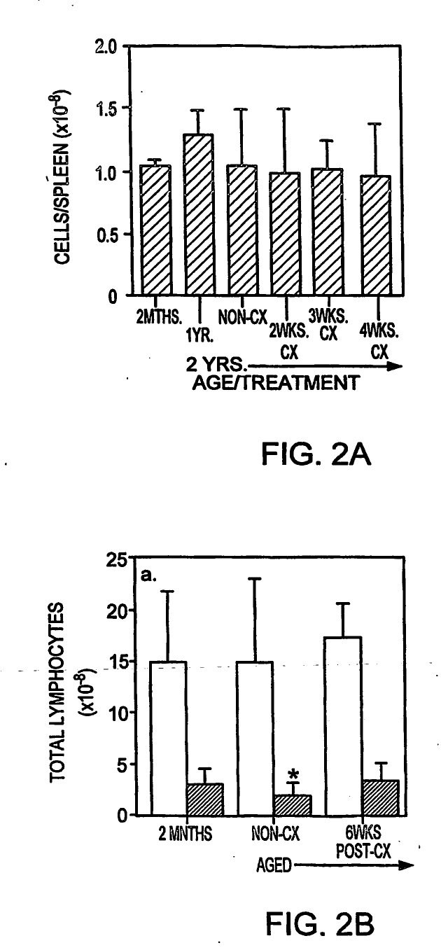

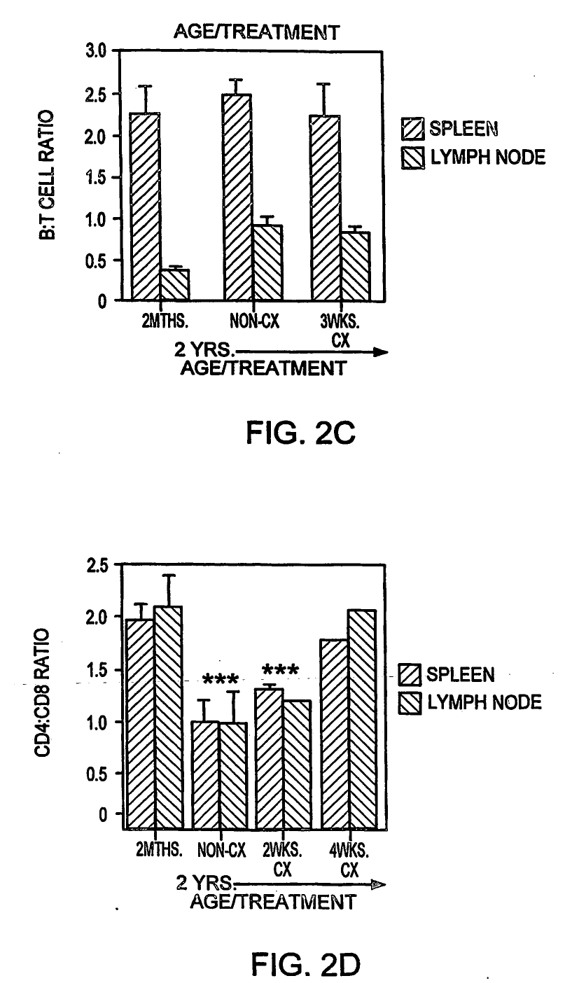

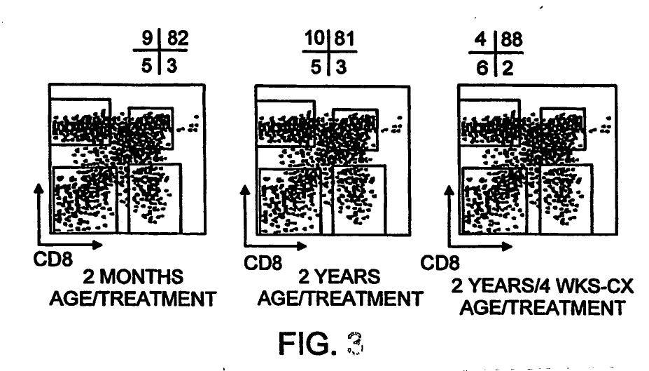

The decrease in T cell numbers produced by the thymus is not reflected in the periphery, with spleen cell numbers remaining constant with age (FIGS. 2A and 2B). Homeostatic mechanisms in the periphery were evident since the B cell to T cell ratio in spleen and lymph nodes was not affected with age and the subsequent decrease in T cell numbers reaching the periphery (FIG. 2C). However, the ratio of CD4⁺ to CD8⁺ T cell significantly decreased (p<0.001) with age from 2:1 at 2 months of age, to a ratio of 1:1 at 2 years of age (FIG. 2D). Following castration and the subsequent rise in T cell numbers reaching the periphery, no change in peripheral T cell numbers was observed: splenic T cell numbers and the ratio of B:T cells in both spleen and lymph nodes was not altered following castration (FIGS. 2A-C). The reduced CD4:CD8 ratio in the periphery with age was still evident at 2 weeks post-castration but was completely reversed by 4 weeks post-castration (FIG. 2D)

(ii) Thymocyte Subpopulations with Age and Post-Castration

To determine if the decrease in thymocyte numbers seen with age was the result of the depletion of specific cell populations, thymocytes were labeled with defining markers in order to analyze the separate subpopulations. In addition, this allowed analysis of the kinetics of thymus repopulation post-castration. The proportion of the main thymocyte subpopulations was compared with those of the young adult (2-4 months) thymus (FIG. 3) and found to remain uniform with age. In addition, further subdivision of thymocytes by the expression of αβTCR revealed no change in the proportions of these populations with age. At 2 and 4 weeks post-castration, thymocyte subpopulations remained in the same proportions and, since thymocyte numbers increase by up to 100-fold post-castration, this indicates a synchronous expansion of all thymocyte subsets rather than a developmental progression of expansion.

The decrease in cell numbers seen in the thymus of aged (2 year old) animals thus appears to be the result of a balanced reduction in all cell phenotypes, with no significant changes in T cell populations being detected. Thymus regeneration occurs in a synchronous fashion, replenishing all T cell subpopulations simultaneously rather than sequentially.

II. Proliferation of Thymocytes

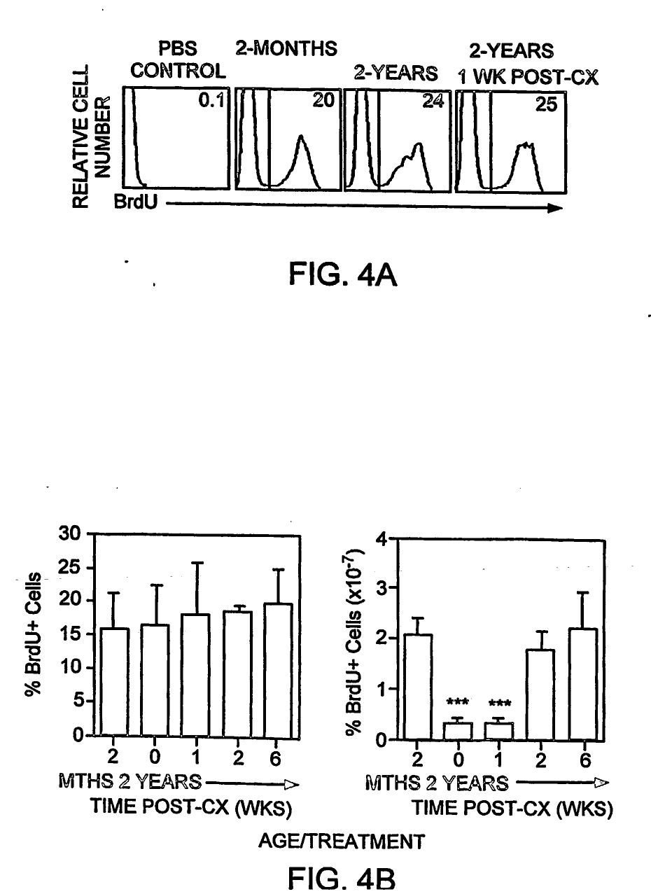

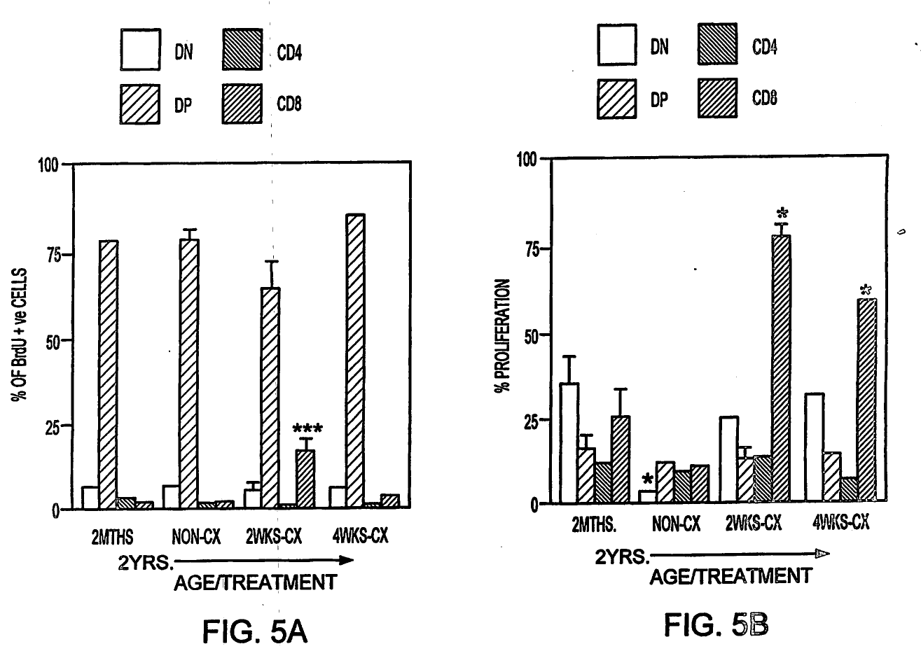

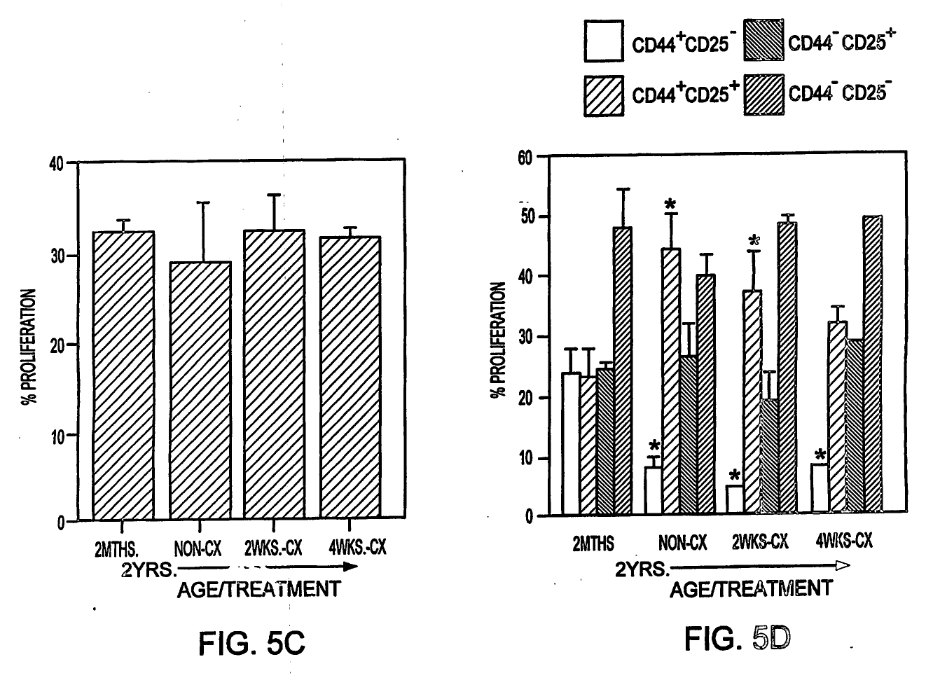

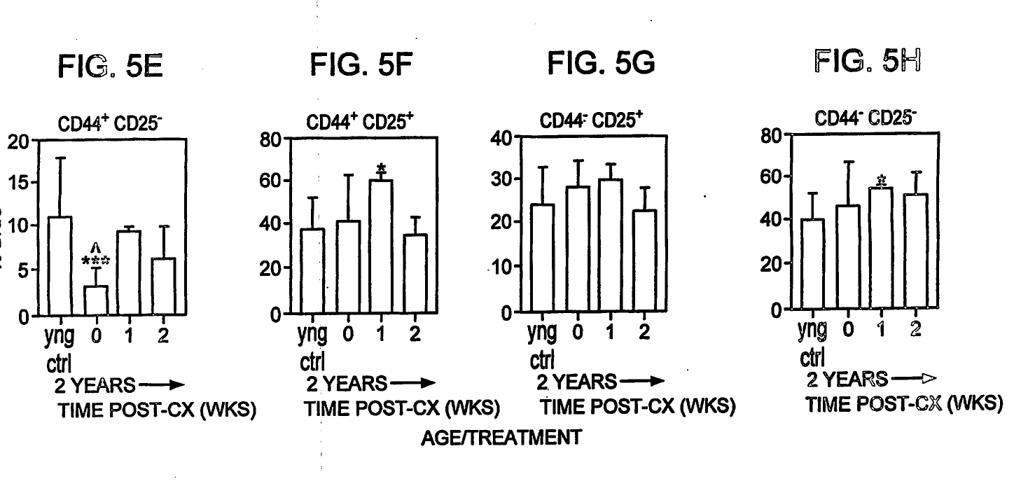

As shown in FIGS. 4A-4B, 15-20% of thymocytes were proliferating at 2-4 months of age. The majority (about 80%) of these are double positive (DP). i.e., CD4+, CD8+) with the triple negative (TN) (i.e., CD3⁻CD4⁻CD8⁻) subset making up the second largest population at about 6% (FIG. 5A). These TN cells are the most immature cells in the thymus and encompass the intrathymic precursor cells. Accordingly, most division is seen in the subcapsule and cortex by immunohistology. Some division is seen in the medullary regions aligning with FACS analysis which revealed a proportion of single positive (i.e., CD4+CD8− or CD4−CD8+) cells (9% of CD4+ T cells and 25% of CD8+ T cells) in the young (2 months) thymus, dividing (FIG. 5B).

Although cell numbers were significantly decreased in the aged mouse thymus (2 years old), the total proportion of proliferating thymocytes remained constant (FIGS. 4B and 5C), but there was a decrease in the proportion of dividing cells in the CD4−CD8− and proliferation of CD4−CD8+ T cells was also significantly (p≦0.001) decreased (data not shown). Immunohistology revealed the distribution of dividing cells at 1 year of age to reflect that seen in the young adult (2-4 months); however, at 2 years, proliferation is mainly seen in the outer cortex and surrounding the vasculature with very little division in the medulla.

As early as one week post-castration there was a marked increase in the proportion of proliferating CD4−CD8− cells and the CD4−CD8+ cells (data not shown). Castration clearly overcomes the block in proliferation of these cells with age. There was a corresponding proportional decrease in proliferating CD4+CD8− cells post-castration (data not shown). At 2 weeks post-castration, although thymocyte numbers significantly increase, there was no change in the overall proportion of thymocytes that were proliferating, again indicating a synchronous expansion of cells (FIGS. 4A, 4B, and 5C). Immunohistology revealed the localization of thymocyte proliferation and the extent of dividing cells to resemble the situation in the 2-month-old thymus by 2 weeks post-castration.

The DN subpopulation, in addition to the thymocyte precursors, contains □□TCR+CD4−CD8− thymocytes, which are thought to have down-regulated both co-receptors at the transition to SP cells (Godfrey and Zlotnik, (1993) Immunol. Today 14:547). By gating on these mature cells, it was possible to analyze the true TN compartment (CD3⁻CD4⁻CD8⁻) and their subpopulations expressing CD44 and CD25. FIGS. 5E-H illustrate the extent of proliferation within each subset of TN cells in young, old and castrated mice. This showed a significant (p<0.001) decrease in proliferation of the TN1 subset (CD44⁺CD25⁻CD3⁻CD4^{-CD}8⁻), from about 10% in the normal young to around 2% at 18 months of age (FIG. 5E) which was restored by 1 week post-castration.

III. Thymocyte Emigration

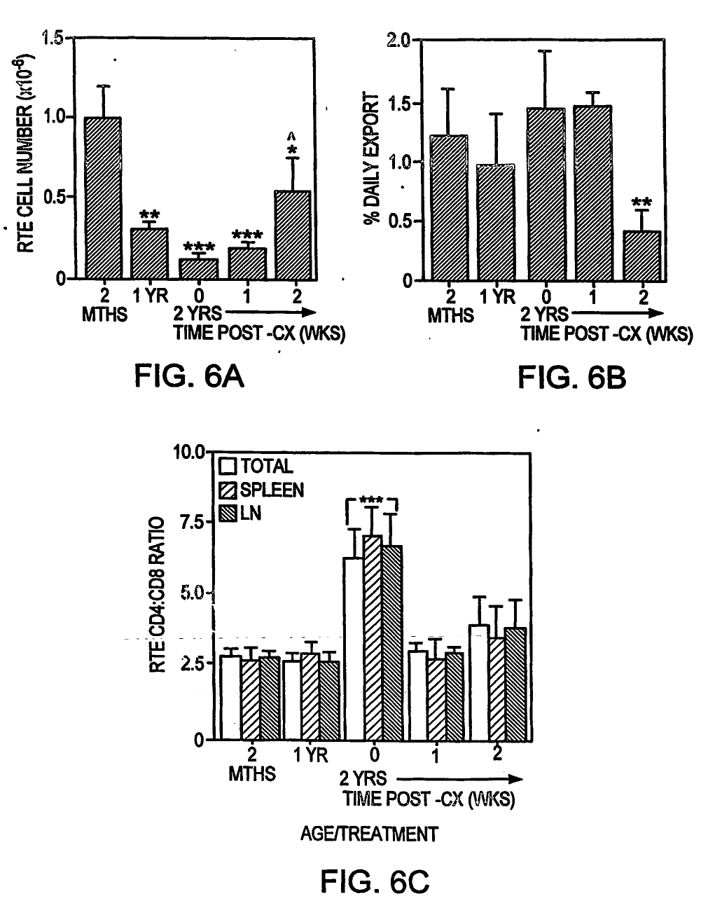

Approximately 1% of T cells migrate from the thymus daily in the young mouse (Scollay et al., (1980) Proc. Natl. Acad. Sci, USA 86:5547). Migration in castrated mice was found to occur at a proportional rate equivalent to the normal young mouse at 14 months and even 2 years of age, although significantly (p≦0.0001) reduced in number (FIGS. 6A and 6B). There was an increase in the CD4:CD8 ratio of the recent thymic emigrants from about 3:1 at 2 months to about 7:1 at 26 months (FIG. 6C). By 1 week post-castration, this ratio had normalized (FIG. 6C). By 2-weeks post-castration, cell number migrating to the periphery had substantially increased with the overall rate of migration reduced to 0.4% reflecting the expansion of the thymus (FIG. 6B).

Discussion

It has been shown that aged thymus, although severely atrophic, maintains its functional capacity with age, with T cell proliferation, differentiation and migration occurring at levels equivalent to the young adult mouse. Although thymic function is regulated by several complex interactions between the neuro-endocrine-immune axes, the atrophy induced by sex steroid production exerts the most significant and prolonged effects illustrated by the extent of thymus regeneration post-castration.

Thymus weight is significantly reduced with age as shown previously (Hirokawa and Makinodan, (1975) J. Immunol. 114:1659, Aspinall, (1997) J. Immunol. 158:3037) and correlates with a significant decrease in thymocyte numbers. The stress induced by the castration technique, which may result in further thymus atrophy due to the actions of corticosteroids, is overridden by the removal of sex steroid influences with the 2-week castrate thymus increasing in cellularity by 20-30 fold from the pre-castrate thymus. By 3 weeks post-castration, the aged thymus shows a significant increase in both thymic size and cell number, surpassing that of the young adult thymus presumably due to the actions of sex steroids already exerting themselves in the 2 month old mouse.

The data confirms previous findings that emphasise the continued ability of thymocytes to differentiate and maintain constant subset proportions with age (Aspinall, (1997) J. Immunol. 158:3037). In addition, thymocyte differentiation has been shown to occur simultaneously post-castration indicative of a synchronous expansion in thymocyte subsets. Since thymocyte numbers are decreased significantly with age, proliferation of thymocytes was analysed to determine if this was a contributing factor in thymus atrophy.

Proliferation of thymocytes was not affected by age-induced thymic atrophy or by removal of sex-steroid influences post-castration with about 14% of all thymocytes proliferating. However, the localisation of this division differed with age: the 2 month mouse thymus shows abundant division throughout the subcapsular and cortical areas (TN and DP T cells) with some division also occurring in the medulla. Due to thymic epithelial disorganisation with age, localisation of proliferation was difficult to distinguish but appeared to be less uniform in pattern than the young and relegated to the outer cortex. By 2 weeks post-castration, dividing thymocytes were detected throughout the cortex and were evident in the medulla with similar distribution to the 2 month thymus.

The phenotype of the proliferating population as determined by CD4 and CD8 analysis, was not altered with age or following castration. However, analysis of proliferation within thymocyte subpopulations, revealed a significant decrease in proliferation of both the TN and CD8⁺ cells with age. Further analysis within the TN subset on the basis of the markers CD44 and CD25, revealed a significant decrease in proliferation of the TN1 (CD44⁺CD25⁻) population which was compensated for by an increase in the TN2 (CD44⁻CD25⁺) population. These abnormalities within the TN population, reflect the findings by Aspinall ((1997) J. Immunol. 158:3037). Surprisingly, the TN subset was proliferating at normal levels by 2 weeks post-castration indicative of the immediate response of this population to the inhibition of sex-steroid action. Additionally, at both 2 weeks and 4 weeks post-castration, the proportion of CD8⁺ T cells that were proliferating was markedly increased from the control thymus, possibly indicating a role in the re-establishment of the peripheral T cell pool.

Thymocyte migration was shown to occur at a constant proportion of thymocytes with age conflicting with previous data by Scollay et al., ((1980) Proc. Natl. Acad. Sci, USA 86:5547) who showed a ten-fold reduction in the rate of thymocyte migration to the periphery. The difference in these results may be due to the difficulties in intrathymic FITC labelling of 2 year old thymuses or the effects of adipose deposition on FITC uptake. However, the absolute numbers of T cells migrating was decreased significantly as found by Scollay resulting in a significant reduction in ratio of RTEs to the peripheral T cell pool. This will result in changes in the periphery predominantly affecting the T cell repertoire (Mackall et al., (1995) N. Eng. J. Med. 332:143). Previous papers (e.g., Mackall et al., (1995) N. Eng. J. Med. 332:143) have shown a skewing of the T cell repertoire to a memory rather than naïve T cell phenotype with age. The diminished T cell repertoire however, may not cope if the individual encounters new pathogens, possibly accounting for the rise in immunodeficiency in the aged. Obviously, there is a need to re-establish the T cell pool in immunocompromised individuals. Castration allows the thymus to repopulate the periphery through significantly increasing the production of naïve T cells.

In the periphery, T cell numbers remained at a constant level as evidenced in the B:T cell ratios of spleen and lymph nodes, presumably due to peripheral homeostasis (Mackall et al., (1995) N. Eng. J. Med. 332:143; Berzins et al., (1998) J. Exp. Med. 187:1839). However, disruption of cellular composition in the periphery was evident with the aged thymus showing a significant decrease in CD4:CD8 ratios from 2:1 in the young adult to 1:1 in the 2 year mouse, possibly indicative of the more susceptible nature of CD4⁺ T cells to age or an increase in production of CD8⁺ T cells from extrathymic sources. By 2 weeks post-castration, this ratio has been normalised, again reflecting the immediate response of the immune system to surgical castration.

The above findings have shown firstly that the aged thymus is capable of functioning in a nature equivalent to the pre-pubertal thymus. In this respect, T cell numbers are significantly decreased but the ability of thymocytes to differentiate is not disturbed. Their overall ability to proliferate and eventually migrate to the periphery is again not influenced by the age-associated atrophy of the thymus. However, two important findings were noted. Firstly, there appears to be an adverse affect on the TN cells in their ability to proliferate, correlating with findings by Aspinall ((1997) J. Immunol. 158:3037). This defect could be attributed to an inherent defect in the thymocytes themselves. Yet the data shown here, and previous work has shown thymocyte differentiation, although diminished, still occurs and stem cell entry from the BM is also not affected with age (Hirokawa, (1998), “Immunity and Ageing,” in PRINCIPLES AND PRACTICE OF GERIATRIC MEDICINE, (M. Pathy, ed.) John Wiley and Sons Ltd; Mackall and Gress, (1997) Immunol. Rev. 160:91). Secondly, the CD8⁺ T cells were significantly diminished in their proliferative capacity with age and, following castration, a significantly increased proportion of CD8⁺ T cells proliferated as compared to the 2 month mouse. The proliferation of mature T cells is thought to be a final step before migration (Suda and Zlotnik, (1991) J. Immunol. 146:3068), such that a significant decrease in CD8⁺ proliferation would indicate a decrease in their migrational potential. This hypothesis is supported by the finding that the ratio of CD4:CD8 T cells in RTEs increased with age, indicative of a decrease in CD8 T cells migrating. Alternatively, if the thymic epithelium is providing the key factor for the CD8 T cell maintenance, whether a lymphostromal molecule or cytokine influence, this factor may be disturbed with increased sex-steroid production. By removing the influence of sex-steroids, the CD8 T cell population can again proliferate optimally.

The defect in proliferation of the TN1 subset which was observed indicates that loss of cortical epithelium affects thymocyte development at the crucial stage of TCR gene rearrangement whereby the cortical epithelium provides factors such as IL-7 and SCF necessary for thymopoiesis (Godfrey and Zlotnik, (1993) Immunol. Today 14:547; Aspinall, (1997) J. Immunol. 158:3037). Indeed, IL-7^{-/-} and IL-7R^{-/-} mice show similar thymic morphology to that seen in aged mice (Wiles et al., (1992) Eur. J. Immunol. 22:1037; Zlotnik and Moore, (1995) Curr. Opin. Immunol. 7:206); von Freeden-Jeffry, (1995) J. Exp. Med. 181:1519).

In conclusion, the aged thymus still maintains its functional capacity, however, the thymocytes that develop in the aged mouse are not under the stringent control by thymic epithelial cells as seen in the normal young mouse due to the lack of structural integrity of the thymic microenvironment. Thus the proliferation, differentiation and migration of these cells will not be under optimal regulation and may result in the increased release of autoreactive/immunodysfunctional T cells in the periphery. The defects within both the TN and particularly, CD8⁺ populations, may result in the changes seen within the peripheral T cell pool with age. Restoration of thymus function by castration will provide an essential means for regenerating the peripheral T cell pool and thus in re-establishing immunity in immunosuppressed, immunodeficient, or immunocompromised individuals.

### Example 2

### Reversal of Chemotherapy- or Radiation-Induced Thymic Atrophy

Materials and Methods

Materials and methods were as described in Example 1. In addition, the following methods were used.

BM Reconstitution.

Recipient mice (3-4 month-old C57BL6/J) were subjected to 5.5 Gy irradiation twice over a 3-hour interval. One hour following the second irradiation dose, mice were injected intravenously with 5×10⁶ donor BM cells. BM cells were obtained by passing RPMI-1640 media through the tibias and femurs of donor (2-month old congenic C57BL6/J Ly5.1⁺) mice, and then harvesting the cells collected in the media.

Irradiation.

3-4 month old mice were subjected to 625 Rads of whole body □-irradiation.

T Cell Depletion Using Cyclophosphamide.

Old mice (e.g., 2 years old) were injected with cyclophosphamide (200 mg/kg body wt over two days) and castrated.

Results

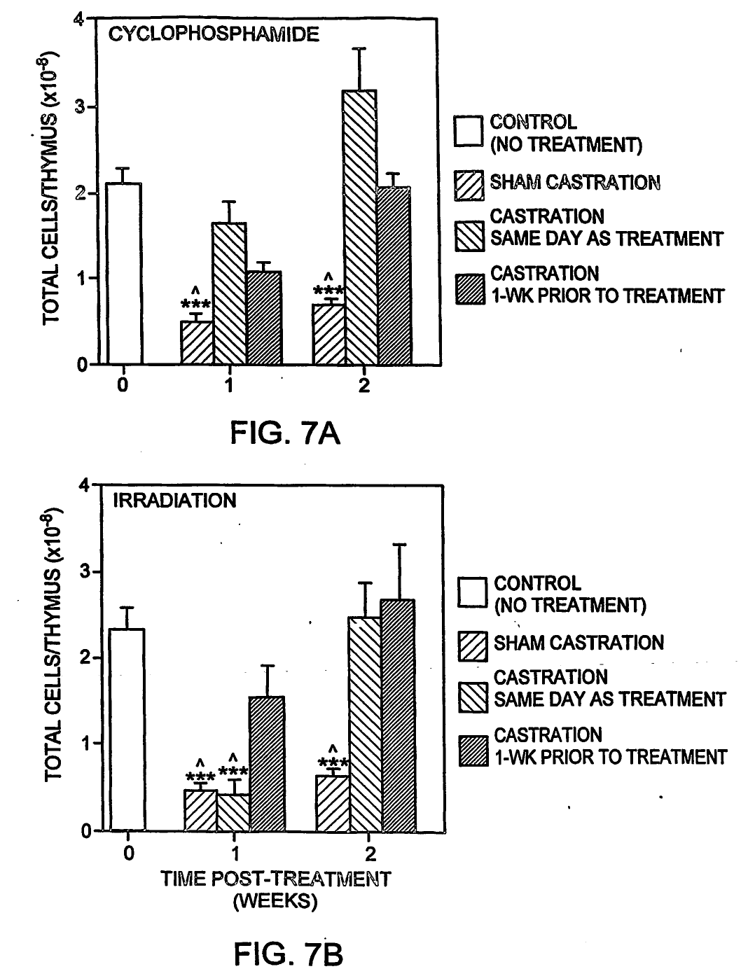

Castration enhanced regeneration following severe T cell depletion (TCD). For both models of T cell depletion studied (chemotherapy using cyclophosphamide or sublethal irradiation using 625 Rads), castrated (Cx) mice showed a significant increase in the rate of thymus regeneration compared to their sham-castrated (ShCx) counterparts (FIGS. 7A and 7B). By 1 week post-treatment castrated mice showed significant thymic regeneration even at this early stage (FIGS. 7 and 9-11). In comparison, non-castrated animals, showed severe loss of DN and DP thymocytes (rapidly-dividing cells) and subsequent increase in proportion of CD4 and CD8 cells (radio-resistant). This is best illustrated by the differences in thymocyte numbers with castrated animals showing at least a 4-fold increase in thymus size even at 1 week post-treatment. By 2 weeks, the non-castrated animals showed relative thymocyte normality with regeneration of both DN and DP thymocytes. However, proportions of thymocytes are not yet equivalent to the young adult control thymus. Indeed, at 2 weeks, the vast difference in regulation rates between castrated and non-castrated mice was maximal (by 4 weeks thymocyte numbers were equivalent between treatment groups).

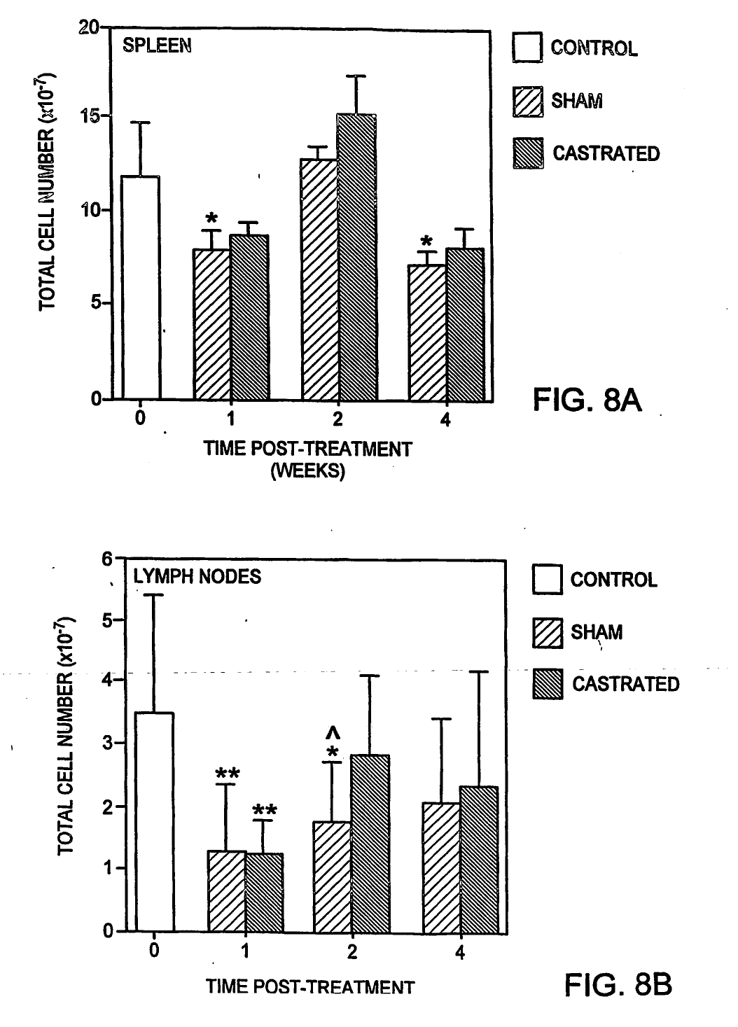

Thymus cellularity was significantly reduced in ShCx mice 1-week post-cyclophosphamide treatment compared to both control (untreated, aged-matched; p≦0.001) and Cx mice (p≦0.05) (FIG. 7A). No difference in thymus regeneration rates was observed at this time-point between mice castrated 1-week earlier or on the same day as treatment, with both groups displaying at least a doubling in the numbers of cells compared to ShCx mice (FIGS. 7A and 8A). Similarly, at 2-weeks post-cyclophosphamide treatment, both groups of Cx mice had significantly (5-6 fold) greater thymocyte numbers (p≦0.001) than the ShCx mice (FIG. 7A). In control mice there was a gradual recovery of thymocyte number over 4 weeks but this was markedly enhanced by castration, even within one week (data not shown). Similarly spleen and lymph node numbers were increased in the castrate mice after one week (data not shown).

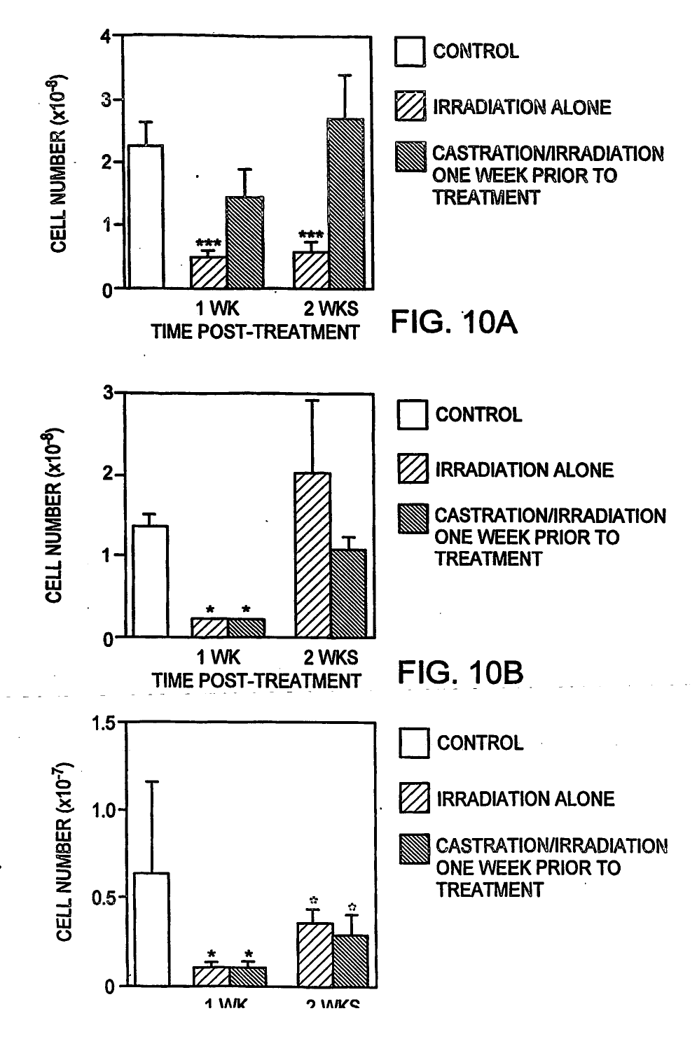

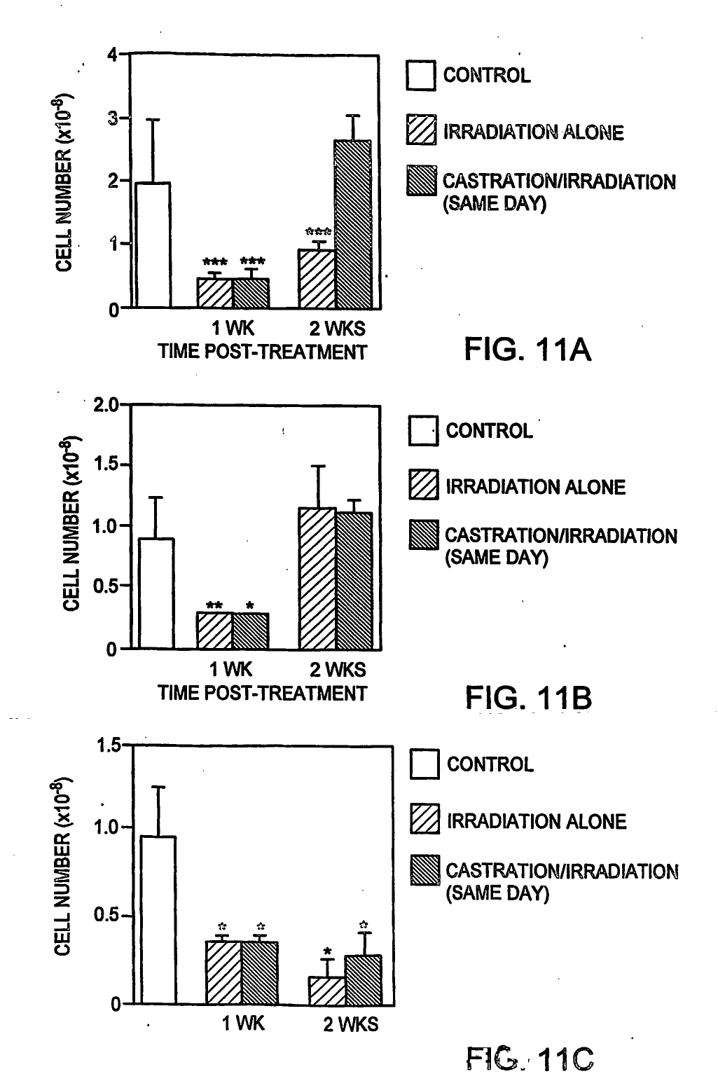

The effect of the timing of castration on thymic recovery was examined by castration one week prior to either irradiation (FIG. 10) or on the same day as irradiation (FIG. 11). When performed one week prior, castration had a more rapid impact on thymic recovery (FIG. 10A compared to FIG. 11A). By two weeks the same day castration had “caught up” with the thymic regeneration in mice castrated one week prior to treatment. In both cases there were no major effects on spleen or lymph nodes (FIGS. 10B and 10C, and FIGS. 11B and 11C) respectively.

Following irradiation treatment, both ShCx and mice castrated on the same day as treatment (SDCx) showed a significant reduction in thymus cellularity compared to control mice (p≦0.001) (FIGS. 7B and 11A) and mice castrated 1-week prior to treatment (p≦0.01) (FIG. 7B). At 2 weeks post-treatment, the castration regime played no part in the restoration of thymus cell numbers with both groups of castrated mice displaying a significant enhancement of thymus cellularity post-irradiation (PIrr) compared to ShCx mice (p≦0.001) (FIGS. 7B, 10A, and 11A). Therefore, castration significantly enhances thymus regeneration post-severe T cell depletion, and it can be performed at least 1-week prior to immune system insult.

Interestingly, thymus size appears to ‘overshoot’ the baseline of the control thymus. Indicative of rapid expansion within the thymus, the migration of these newly derived thymocytes does not yet (it takes about 3-4 weeks for thymocytes to migrate through and out into the periphery). Therefore, although proportions within each subpopulation are equal, numbers of thymocytes are building before being released into the periphery.

Following cyclophosphamide treatment of young mice (about 2-3 months), total lymphocyte numbers within the spleen of Cx mice, although reduced, were not significantly different from control mice throughout the time-course of analysis (data not shown). However, ShCx mice showed a significant decrease in total splenocyte numbers at 1- and 4-weeks post-treatment (p≦0.05) (FIG. 8A). Within the lymph nodes, a significant decrease in cellularity was observed at 1-week post-treatment for both sham-castrated and castrated mice (p≦0.01) (FIG. 8B), possibly reflecting the influence of stress steroids. By 2-weeks post-treatment, lymph node cellularity of castrated mice was comparable to control mice however sham-castrated mice did not restore their lymph node cell numbers until 4-weeks post-treatment, with a significant (p≦0.05) reduction in cellularity compared to both control and Cx mice at 2-weeks post-treatment (FIG. 8B). These results indicate that castration may enhance the rate of recovery of total lymphocyte numbers following cyclophosphamide treatment.

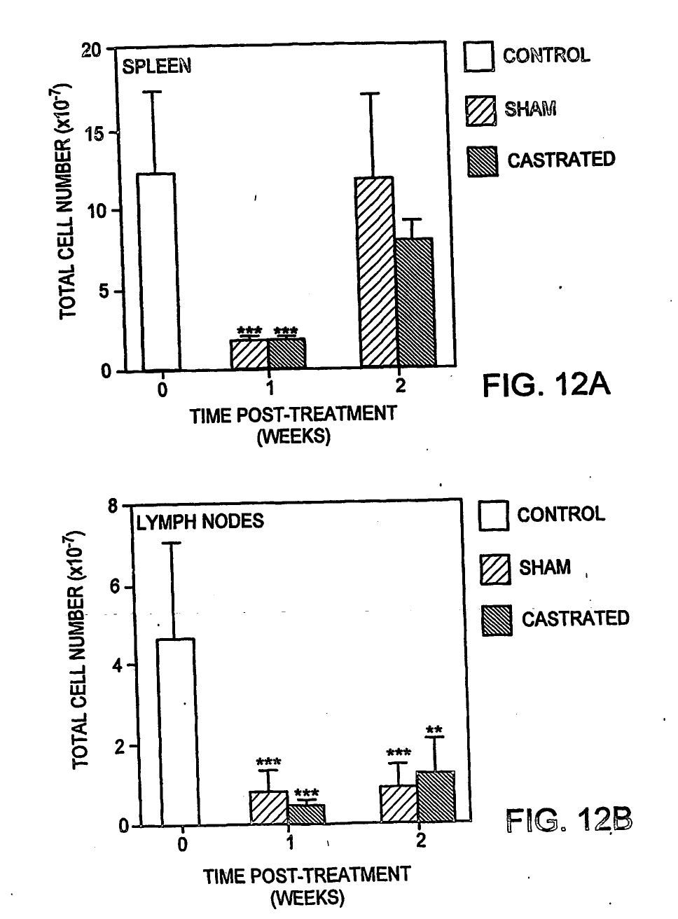

Sublethal irradiation (625 Rads) induced a profound lymphopenia such that at 1-week post-treatment, both treatment groups (Cx and ShCx), showed a significant reduction in the cellularity of both spleen and lymph nodes (p≦0.001) compared to control mice (FIGS. 12A and 12B). By 2 weeks post-irradiation, spleen cell numbers were similar to control values for both castrated and sham-castrated mice (FIG. 12A), whilst lymph node cell numbers were still significantly lower than control values (p≦0.001 for sham-castrated mice; p≦0.01 for castrated mice) (FIG. 12B). No significant difference was observed between the Cx and ShCx mice.

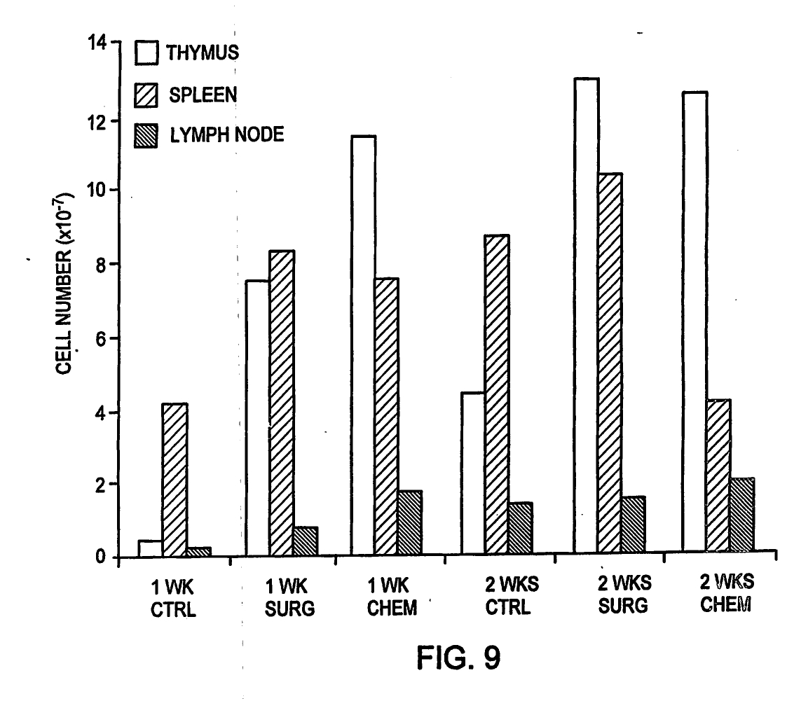

FIG. 9 illustrates the use of chemical castration compared to surgical castration in enhancement of T cell regeneration. The chemical used in this example, Deslorelin (an LHRH-A), was injected for four weeks, and showed a comparable rate of regeneration post-cyclophosphamide treatment compared to surgical castration. The enhancing effects were equivalent on thymic expansion and also the recovery of spleen and lymph node. The kinetics of chemical castration are slower than surgical, that is, mice take about 3 weeks longer to decrease their circulating sex steroid levels. However, chemical castration is still as effective as surgical castration and can be considered to have an equivalent effect.

Discussion

The impact of castration on thymic structure and T cell production was investigated in animal models of immunodepletion. Specifically, Example 2 examined the effect of castration on the recovery of the immune system after sublethal irradiation and cyclophosphamide treatment. These forms of immunodepletion act to inhibit DNA synthesis and therefore target rapidly dividing cells. In the thymus these cells are predominantly immature cortical thymocytes, but all subsets are effected (Fredrickson and Basch, (1994) Dev. Comp. Immunol. 18:251). In normal healthy aged animals, the qualitative and quantitative deviations in peripheral T cells seldom lead to pathological states. However, major problems arise following severe depletion of T cells because of the reduced capacity of the thymus for T cell regeneration. Such insults occur in HIV/AIDS, and particularly following chemotherapy and radiotherapy in cancer treatment (Mackall et al., (1995) N. Eng. J. Med. 332:143).

In both sublethally irradiated and cyclophosphamide treated mice, castration markedly enhanced thymic regeneration. Castration was carried out on the same day as and seven days prior to immunodepletion in order to appraise the effect of the predominantly corticosteroid induced, stress response to surgical castration on thymic regeneration. Although increases in thymus cellularity and architecture were seen as early as one week after immunodepletion, the major differences were observed two weeks after castration. This was the case whether castration was performed on the same day or one week prior to immunodepletion.

Immunohistology demonstrated that in all instances, two weeks after castration the thymic architecture appeared phenotypically normal, while that of noncastrated mice was disorganised. Pan epithelial markers demonstrated that immunodepletion caused a collapse in cortical epithelium and a general disruption of thymic architecture in the thymii of noncastrated mice. Medullary markers supported this finding. Interestingly, one of the first features of castration-induced thymic regeneration was a marked upregulation in the extracellular matrix, identified by MTS 16.

Flow cytometry analysis data illustrated a significant increase in the number of cells in all thymocyte subsets in castrated mice. At each time point, there was a synchronous increase in all CD4, CD8 and αβ-TCR-defined subsets following immunodepletion and castration. This is an unusual but consistent result, since T cell development is a progressive process it was expected that there would be an initial increase in precursor cells (contained within the CD4⁻CD8⁻ gate) and this may have occurred before the first time point. Moreover, since precursors represent a very small proportion of total thymocytes, a shift in their number may not have been detectable. The effects of castration on other cells, including macrophages and granulocytes were also analysed. In general there was little alteration in macrophage and granulocyte numbers within the thymus.

In both irradiation and cyclophosphamide models of immunodepletion thymocyte numbers peaked at every two weeks and decreased four weeks after treatment. Almost immediately after irradiation or chemotherapy, thymus weight and cellularity decreased dramatically and approximately 5 days later the first phase of thymic regeneration begun. The first wave of reconstitution (days 5-14) was brought about by the proliferation of radioresistant thymocytes (predominantly double negatives) which gave rise to all thymocyte subsets (Penit and Ezine, (1989) Proc. Natl. Acad. Sci, USA 86:5547). The second decrease, observed between days 16 and 22 was due to the limited proliferative ability of the radioresistant cells coupled with a decreased production of thymic precursors by the BM (also effected by irradiation). The second regenerative phase was due to the replenishment of the thymus with BM derived precursors (Huiskamp et al., (1983) Radiat. Res. 95:370).

In adult mice the development from a HSC to a mature T cell takes approximately 28 days (Shortman et al., (1990) Sem. Immunol. 2:3). Therefore, it is not surprising that little change was seen in peripheral T cells up to four weeks after treatment. The periphery would be supported by some thymic export, but the majority of the T cells found in the periphery up to four weeks after treatment would be expected to be proliferating cyclophosphamide or irradiation resistant clones expanding in the absence of depleted cells. Several long term changes in the periphery would be expected post-castration including, most importantly, a diversification of the TCR repertoire due to an increase in thymic export.

### Example 3

### Thymic Regeneration Following Inhibition of Sex Steroids Results in Restoration of Deficient Peripheral T Cell Function

Materials and Methods

Materials and methods were as described in Examples 1 and 2. In addition, the following methods were used.

HSV-1 Immunization.

Aged (≧18 months) mice were surgically castrated. 6 weeks after castration (following thymus reactivation). Following anesthetic, mice were injected in the hind leg (foot-hock) with 4×10⁵ plaque forming units (pfu) of HSV-1 (KOS strain) in sterile PBS using a 20-gauge needle. Infected mice were housed in isolated cages and humanely killed on D5 post-immunization at which time the popliteal (draining) lymph nodes were removed for analysis.

Virus was obtained from Assoc. Prof. Frank Carbone (Melbourne University). Virus stocks were grown and titrated on VERO cell monolayers in MEM supplemented with 5% FCS (Gibco-BRL, Australia).

Analysis of the draining (popliteal) lymph nodes was performed on D5 post-infection. For HSV-1 studies, popliteal lymph node cells were stained for anti-CD25-PE, anti-CD8-APC and anti-Vβ10-biotin. For detection of DC, an FcR block was used prior to staining for CD45.1-FITC, I-A^{b}-PE and CD11c-biotin. All biotinylated antibodies were detected with streptavidin-PerCP. For detection of HSC, BM cells were gated on Lin⁻ cells by collectively staining with anti-CD3, CD4, CD8, Gr-1, B220 and Mac-1 (all conjugated to FITC). HSC were detected by staining with CD117-APC and Sca-1-PE. For TN thymocyte analysis, cells were gated on the Lin⁻ population and detected by staining with CD44-biotin, CD25-PE and c-kit-APC.

Cytotoxicity Assay of Lymph Node Cells.

Lymph node cells were incubated for three days at 37° C., 6.5% CO₂. Specificity was determined using a non-transfected cell line (EL4) pulsed with gB₄₉₈₋₅₀₅ peptide (gBp) and EL4 cells alone as a control. A starting effector:target ratio of 30:1 was used. The plates were incubated at 37° C., 6.5% CO₂ for four hours and then centrifuged 650_{gmax} for 5 minutes. Supernatant (100 μl) was harvested from each well and transferred into glass fermentation tubes for measurement by a Packard Cobra auto-gamma counter.

Results

To determine the functional consequences of thymus regeneration (e.g., whether castration can enhance the immune response, herpes simplex virus (HSV) immunization was examined as it allows the study of disease progression and role of CTL. Castrated mice were found to have a qualitatively and quantitatively improved responsiveness to the virus.

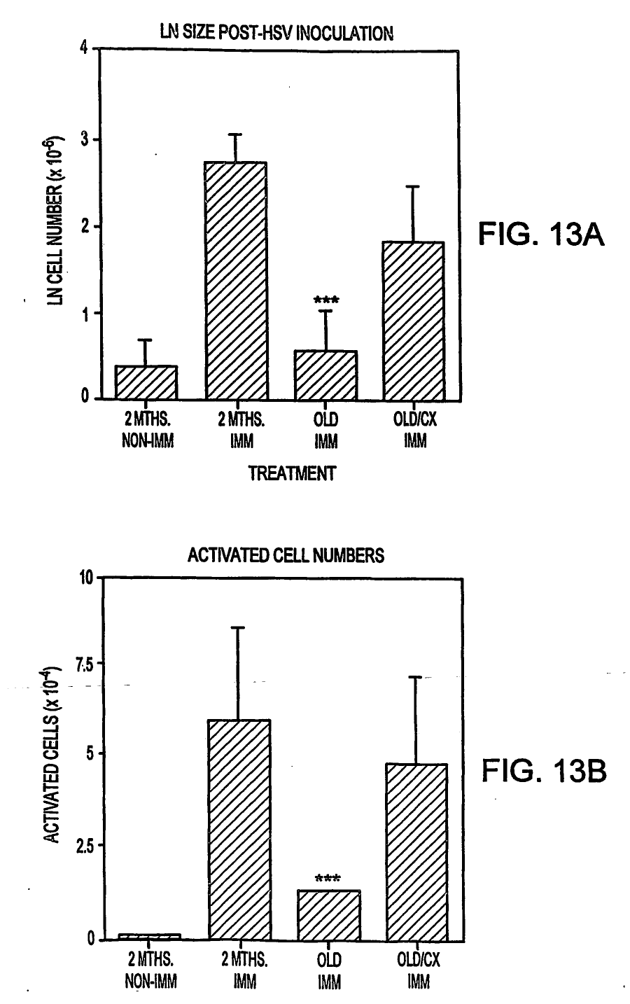

Mice were immunized in the footpad and the popliteal (draining) lymph node analyzed at D5 post-immunization. In addition, the footpad was removed and homogenized to determine the virus titer at particular time-points throughout the experiment. The regional (popliteal) lymph node response to HSV-1 infection (FIGS. 13-17) was examined.

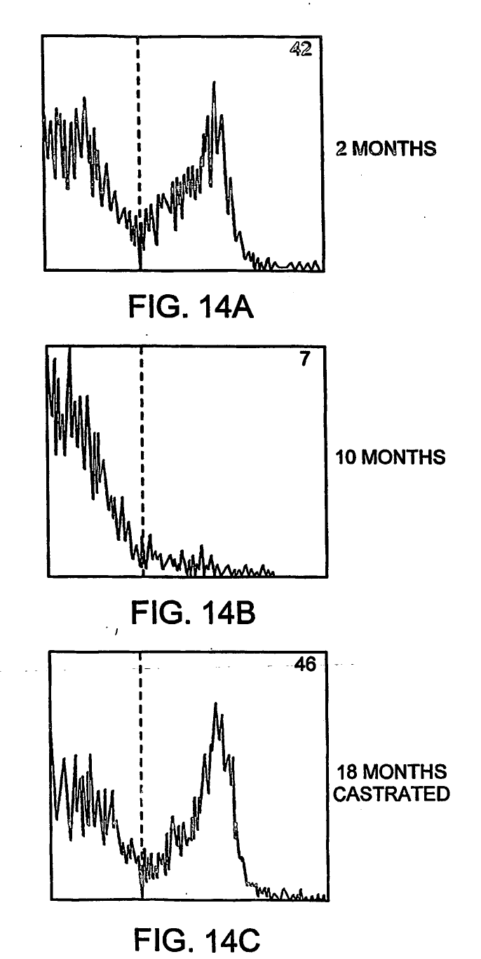

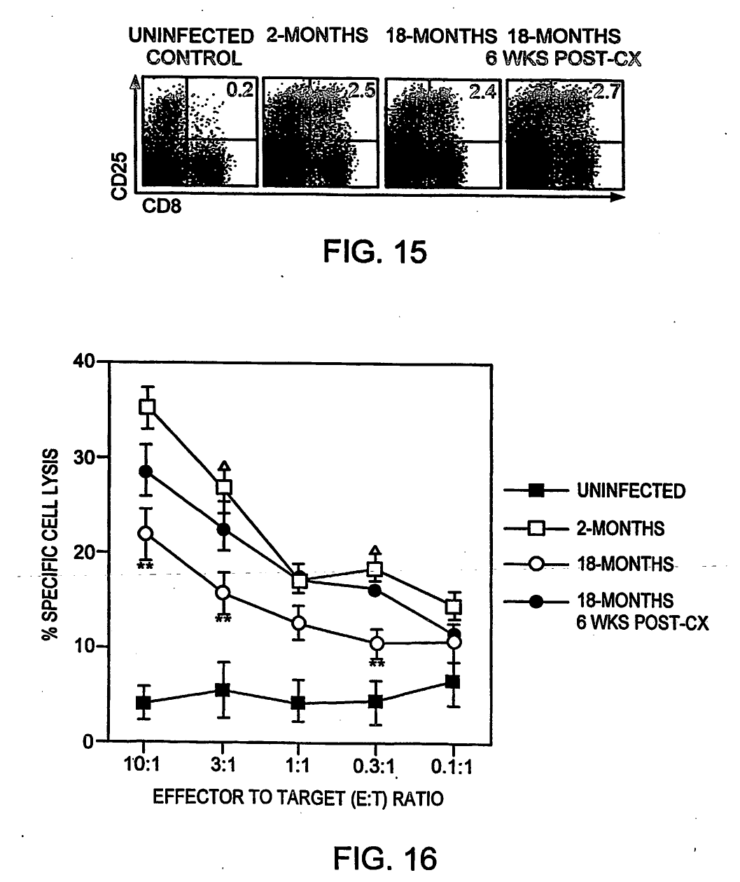

A significant decrease in lymph node cellularity was observed with age (FIGS. 13A, 13B, and 14). At D5 (i.e., 5 days) post-immunization, the castrated mice have a significantly larger lymph node cellularity than the aged mice (data not shown). Although no difference in the proportion of activated (CD8⁺CD25⁺) cells was seen with age or post-castration (FIG. 15), activated cell numbers within the lymph nodes were significantly increased with castration when compared to the aged controls (data not shown). Further, activated cell numbers correlated with that found for the young adult (data not shown), indicating that CTLs were being activated to a greater extent in the castrated mice, but the young adult may have an enlarged lymph node due to B cell activation. This was confirmed with a CTL assay detecting the proportion of specific lysis occurring with age and post-castration (FIG. 16). Aged mice showed a significantly reduced target cell lysis at effector:target ratios of 10:1 and 3:1 compared to young adult (2-month) mice (FIG. 16). Castration restored the ability of mice to generate specific CTL responses post-HSV infection (FIG. 16).

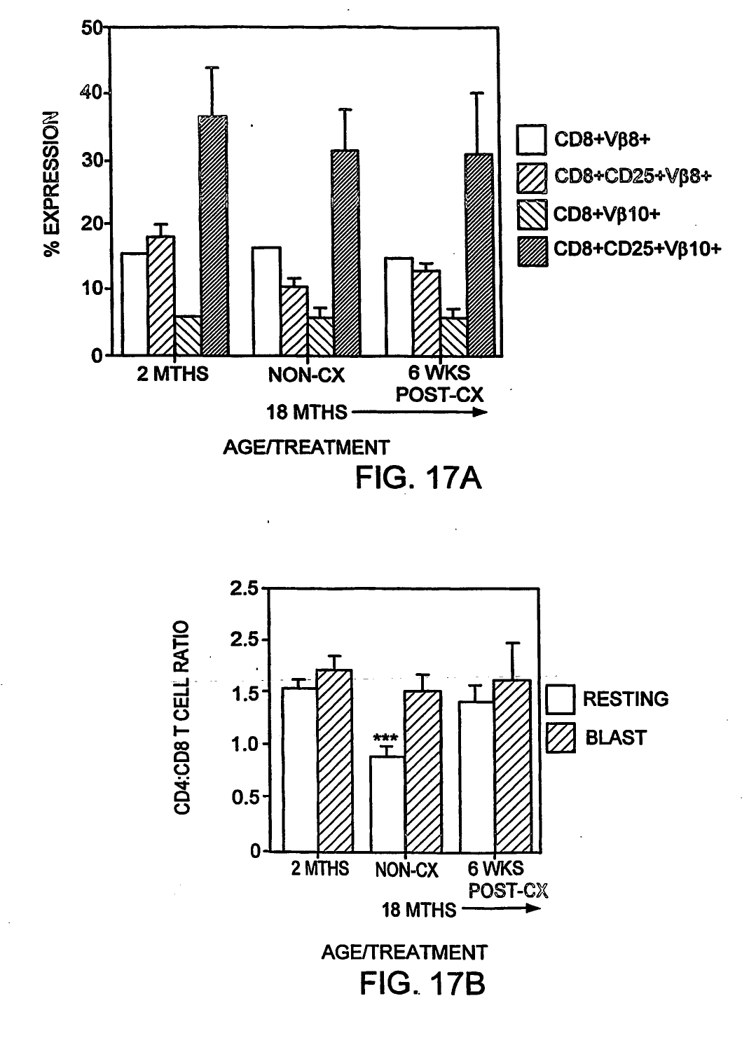

In addition, while overall expression of Vβ10 by the activated cells remained constant with age (data not shown), a subgroup of aged (18-month) mice showed a diminution of this clonal response (FIGS. 14A-C). By six weeks post-castration, the total number of infiltrating lymph node cells and the number of activated CD25⁺CD8⁺ cells had increased to young adult levels (data not shown). More importantly however, castration significantly enhanced the CTL responsiveness to HSV-infected target cells, which was greatly reduced in the aged mice (FIG. 16) and restored the CD4:CD8 ratio in the lymph nodes (FIG. 17B). Indeed, a decrease in CD4+ T cells in the draining lymph nodes was seen with age compared to both young adult and castrated mice (FIG. 17B), thus illustrating the vital need for increased production of T cells from the thymus throughout life, in order to get maximal immune responsiveness.

### Example 4

### Inhibition of Sex Steroids Enhances Uptake of New Hematopoietic Precursor Cells into the Thymus which Enables Chimeric Mixtures of Host and Donor Lymphoid Cells (T, B, and DC)

Materials and methods were as described in Examples 1-3. In addition, the following techniques were used:

Previous experiments have shown that microchimera formation plays an important role in organ transplant acceptance. DC have also been shown to play an integral role in tolerance to graft antigens. Therefore, the effects of castration on thymic chimera formation and dendritic cell number was studied.

In order to assess the role of stem cell uptake in thymus regeneration, BM reconstitution was performed as described in Example 2

For the syngeneic experiments, three month old mice (n=4) were used per treatment group. All controls were age matched and untreated.

Results

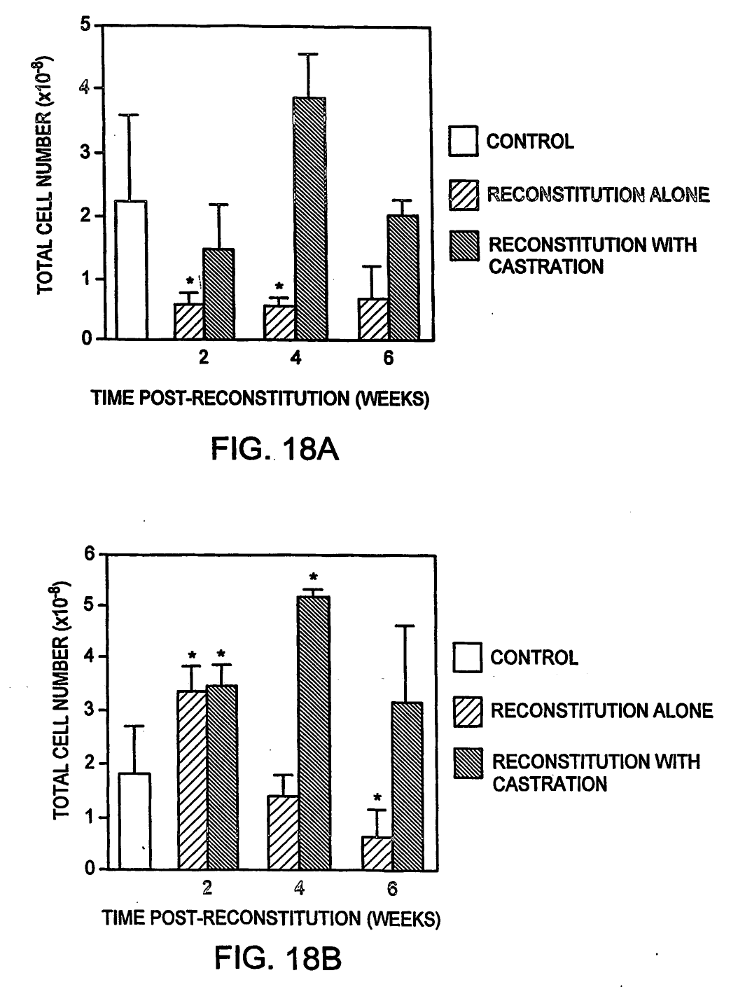

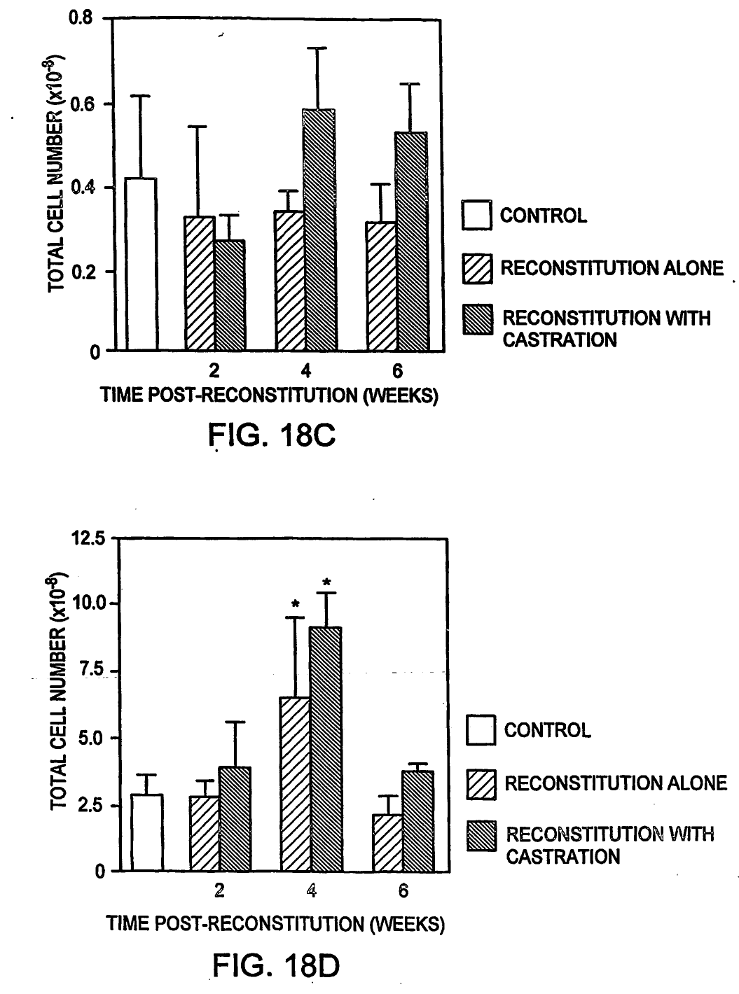

The total thymus cell numbers of castrated and noncastrated reconstituted mice were compared to untreated-age matched-controls and are summarized in FIG. 18A. In mice castrated 1 day prior to reconstitution, there was a significant increase (p≦0.01) in the rate of thymus regeneration compared to sham-castrated (ShCx) control mice. Thymus cellularity in the sham-castrated mice was below untreated control levels (7.6×10⁷±5.2×10⁶) 2 and 4 weeks after congenic BMT, while thymus cellularity of castrated mice had increased above control levels at 4-weeks post-BMT (FIG. 18A). At 6 weeks, cell numbers remained below control levels. However, those of castrated mice were three fold higher than the noncastrated mice (p≦0.05) (FIG. 18A).

There were also significantly more cells (p≦0.05) in the BM of castrated mice 4 weeks after BMT (FIG. 18D). BM cellularity reached untreated control levels (1.5×10⁷±1.5×10⁶) in the sham-castrates by 2 weeks, whereas BM cellularity was increased above control levels in castrated mice at both 2 and 4 weeks after congenic BMT (FIG. 18D). Mesenteric lymph node cell numbers were decreased 2-weeks after irradiation and reconstitution, in both castrated and noncastrated mice; however, by the 4 week time point cell numbers had reached control levels. There was no statistically significant difference in lymph node cell number between castrated and noncastrated treatment groups (FIG. 18C). Spleen cellularity reached untreated control levels (1.5×10⁷±1.5×10⁶) in the sham-castrates and castrates by 2 weeks, but dropped off in the sham group over 4-6 weeks, whereas the castrated mice still had high levels of spleen cells (FIG. 18B). Again, castrated mice showed increased lymphocyte numbers at these time points (i.e., 4 and 6 weeks post-reconstitution) compared to non-castrated mice (p≦0.05) although no difference in total spleen cell number between castrated and noncastrated treatment groups was seen at 2-weeks (FIG. 18B).

Thus, in mice castrated 1 day prior to reconstitution, there was a significant increase (p≦0.01) in the rate of thymus regeneration compared to sham-castrated (ShCx) control mice (FIG. 18A). Thymus cellularity in the sham-castrated mice was below untreated control levels (7.6×10⁷±5.2×10⁶) 2 and 4 weeks after congenic BMT, while thymus cellularity of castrated mice had increased above control levels at 4-weeks post-BMT (FIG. 18A). Castrated mice had significantly increased congenic (Ly5.2) cells compared to non-castrated animals.

In noncastrated mice, there was a profound decrease in thymocyte number over the 4 week time period, with little or no evidence of regeneration (data not shown). In the castrated group, however, by two weeks there was already extensive thymopoiesis which by four weeks had returned to control levels, being 10 fold higher than in noncastrated mice. Flow cytometric analysis of the thymuses with respect to CD45.2 (donor-derived antigen) demonstrated that no donor derived cells were detectable in the noncastrated group at 4 weeks, but remarkably, virtually all the thymocytes in the castrated mice were donor-derived at this time point (data not shown). Given this extensive enhancement of thymopoiesis from donor-derived hemopoietic precursors, it was important to determine whether T cell differentiation had proceeded normally. CD4, CD8 and TCR defined subsets were analyzed by flow cytometry. There were no proportional differences in thymocytes subset proportions 2 weeks after reconstitution (data not shown). This observation was not possible at 4 weeks, because the noncastrated mice were not reconstituted with donor-derived cells. However, at this time point the thymocyte proportions in castrated mice appear normal.

In a parallel set of experiments, 3 month old, young adults, C57/BL6 mice were castrated or sham-castrated 1 day prior to BMT. For congenic BMT, the mice were subjected to 800 RADS TBI and IV injected with 5×10⁶ Ly5.1⁺ BM cells. Mice were killed 2 and 4 weeks later and the BM, thymus and spleen were analyzed for immune reconstitution. Donor/Host origin was determined with anti-CD45.1 antibody, which only reacts with leukocytes of donor origin.

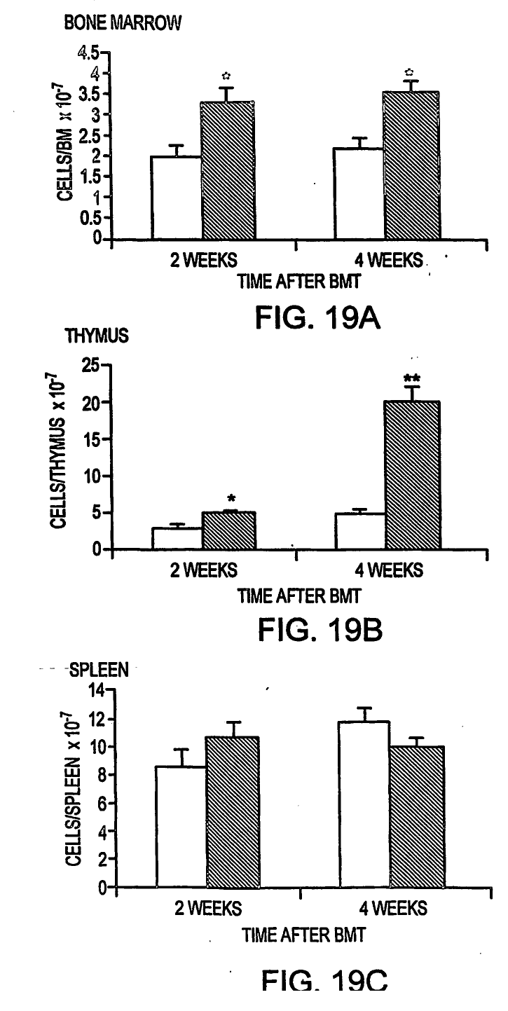

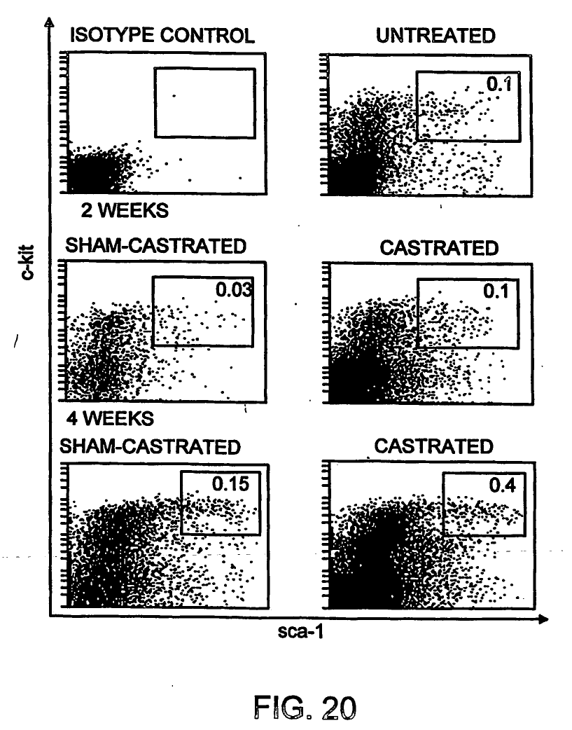

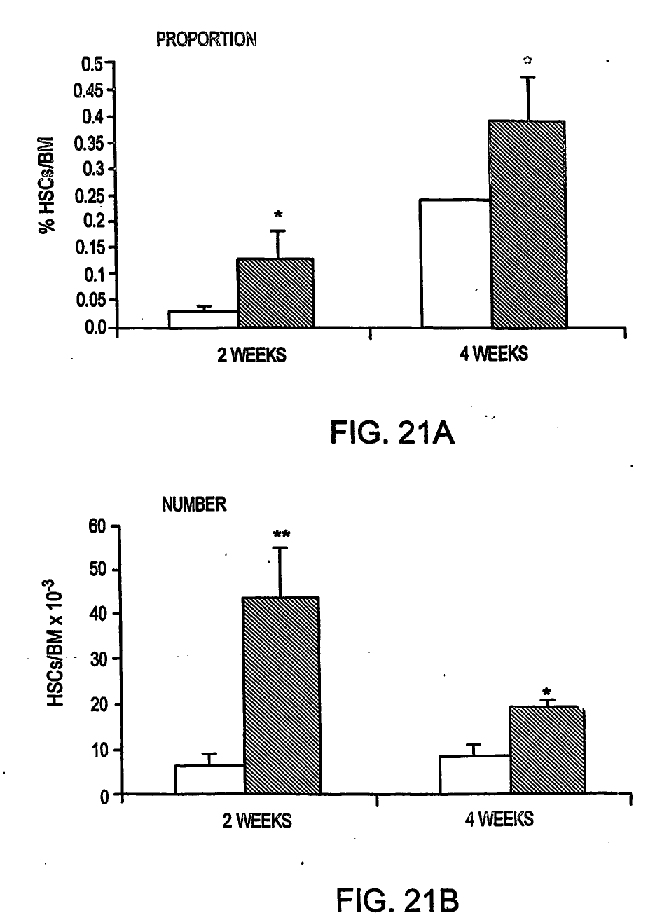

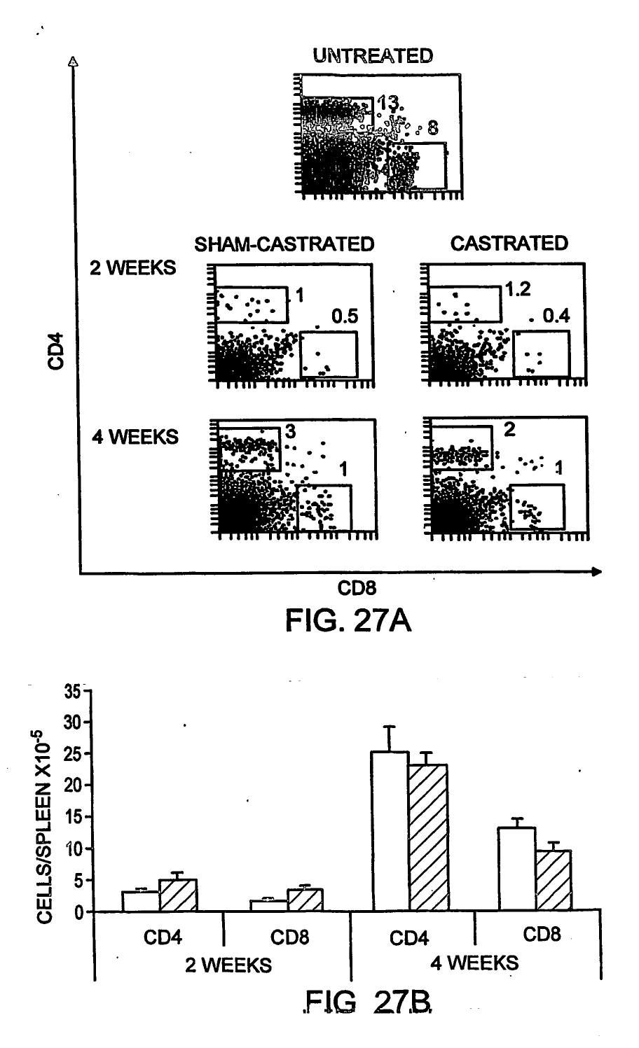

The results from this parallel set of experiments are shown in FIGS. 19-28. FIGS. 20 and 21 show an increase in the number and proportion of donor derived HSC in the BM of castrated animals. This indicates improved engraftment and suggests faster recovery from BMT.

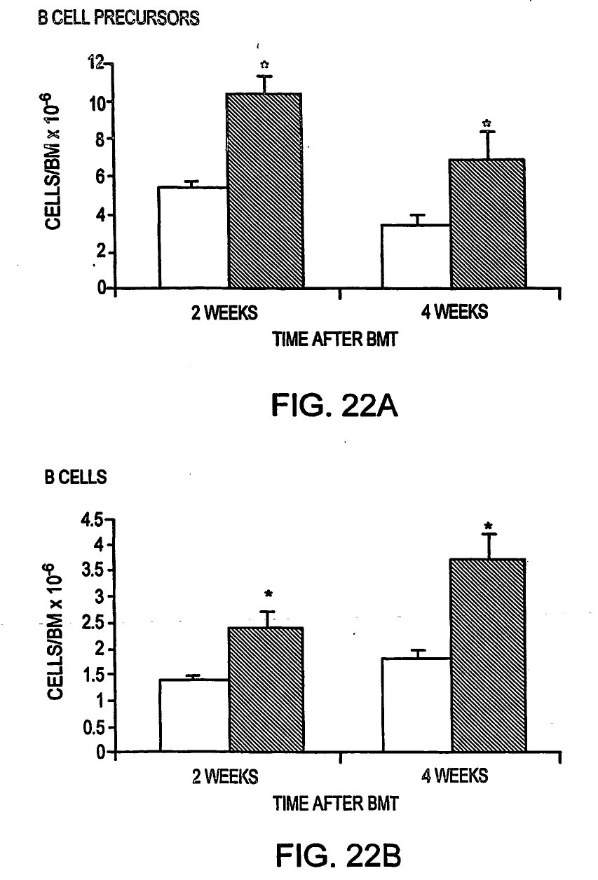

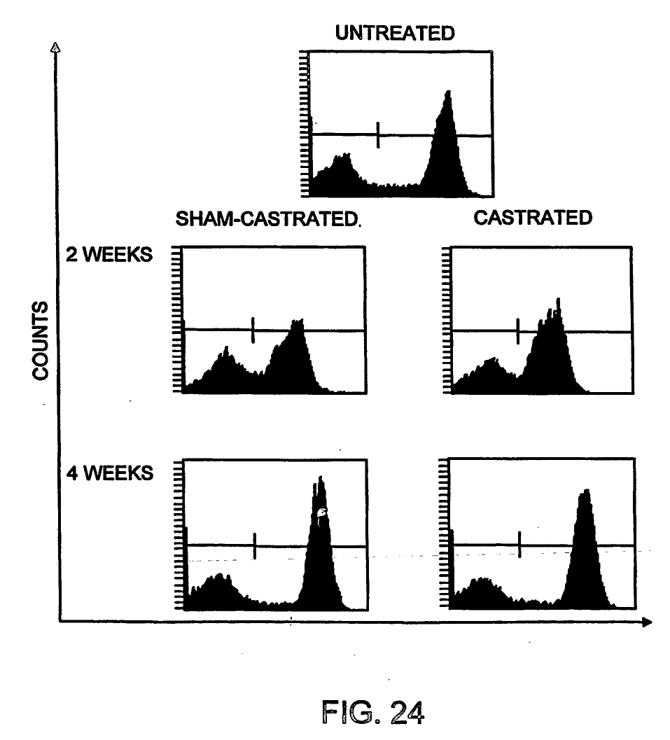

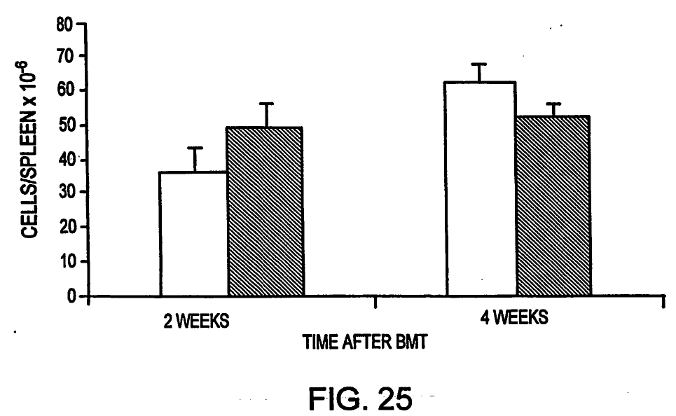

FIG. 22 shows an increase in donor derived B cell precursors and B cells in the BM of castrated mice. However, FIGS. 24 and 25 show castration does not alter the number or proportion of B cells in the periphery at 2 and 4 weeks post castration.

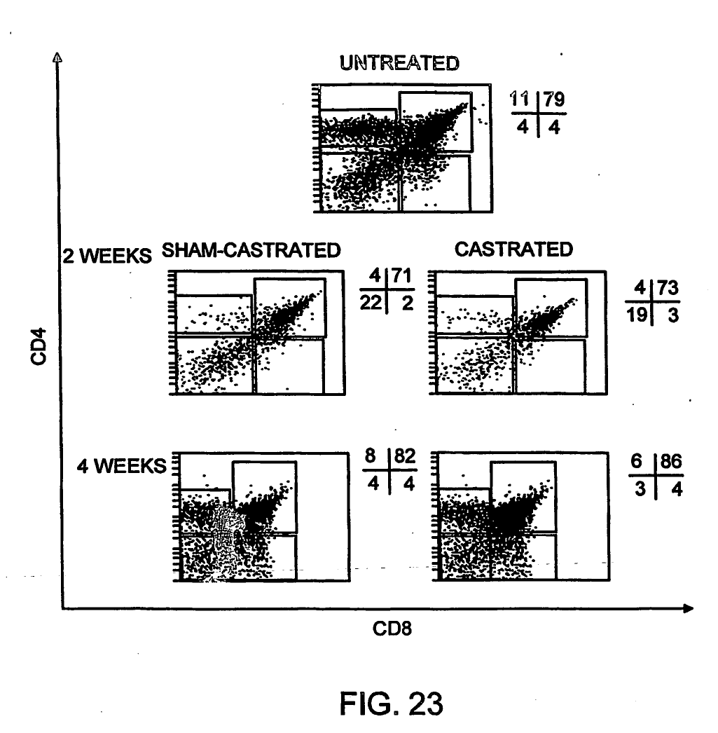

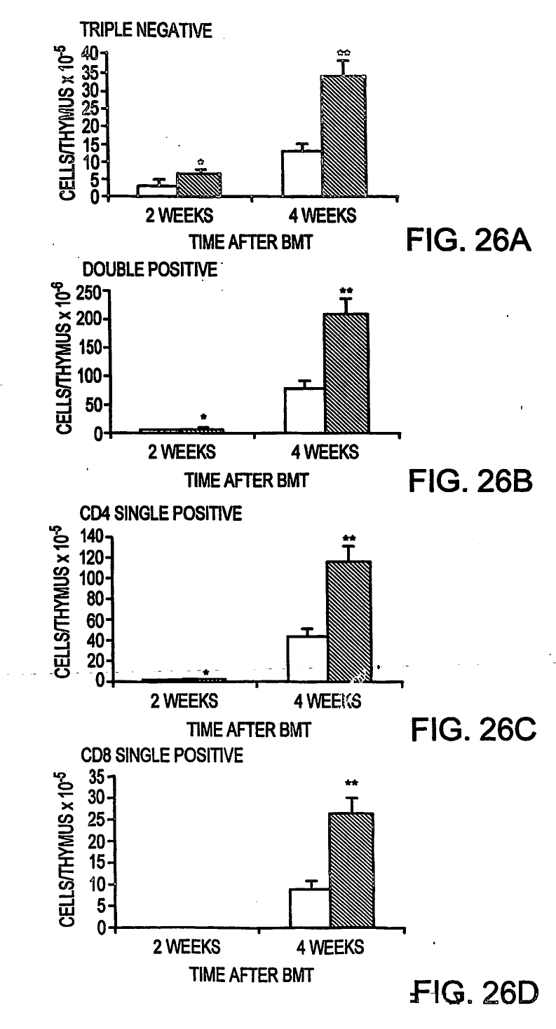

FIG. 26 shows castration increased numbers of donor derived TN, DP, CD4 and CD8 cells in the thymus. However FIG. 23 shows castration does not alter the donor thymocyte proportions of CD4 and CD8 cells. In the periphery, there are very few CD4 or CD8 cells and at the time points considered, there was no increase in these cells with castration.

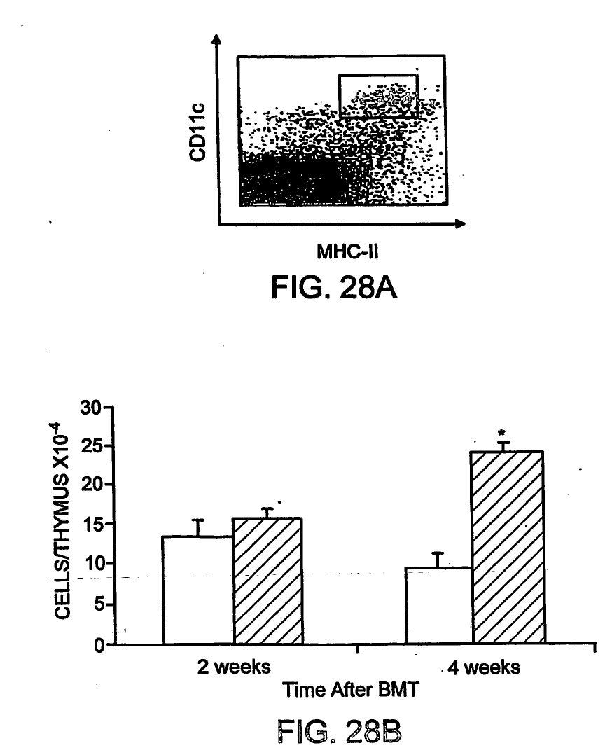

Importantly, FIG. 28 shows and increased number of donor DC in the thymus by 4 weeks post castration.

Discussion

Example 4 shows the influence of castration on syngeneic and congenic BM transplantation. Starzl et al., (1992) Lancet 339:1579 reported that microchimeras evident in lymphoid and nonlymphoid tissue were a good prognostic indicator for allograft transplantation. That is it was postulated that they were necessary for the induction of tolerance to the graft (Starzl et al., (1992) Lancet 339:1579). Donor-derived DC were present in these chimeras and were thought to play an integral role in the avoidance of graft rejection (Thomson and Lu, (1999) Immunol. Today 20:20). DC are known to be key players in the negative selection processes of thymus and if donor-derived DC were present in the recipient thymus, graft reactive T cells may be deleted.

In order to determine if castration would enable increased chimera formation, a study was performed using syngeneic foetal liver transplantation. The results showed an enhanced regeneration of thymii of castrated mice. These trends were again seen when the experiments were repeated using congenic (Ly5) mice. Due to the presence of congenic markers, it was possible to assess the chimeric status of the mice. As early as two weeks after foetal liver reconstitution there were donor-derived dendritic cells detectable in the thymus, the number in castrated mice being four-fold higher than that in noncastrated mice. Four weeks after reconstitution the noncastrated mice did not appear to be reconstituted with donor derived cells, suggesting that castration may in fact increase the probability of chimera formation. Given that castration not only increases thymic regeneration after lethal irradiation and fetal liver reconstitution and that it also increases the number of donor-derived dendritic cells in the thymus, along-side stem cell transplantation this approach increases the probability of graft acceptance.

### Example 5

### Immune Cell Depletion

In order to prevent interference with the graft by the existing T cells in the potential graft recipient patient, the patient underwent T cell depletion (ablation). One standard procedure for this step is as follows. The human patient received anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg as needed. This treatment did not affect early T cell development in the patient's thymus, as the amount of antibody necessary to have such an affect cannot be delivered due to the size and configuration of the human thymus. The treatment was maintained for approximately 4-6 weeks to allow the loss of sex steroids followed by the reconstitution of the thymus.

The prevention of T cell reactivity may also be combined with inhibitors of second level signals such as interleukins, accessory molecules (e.g., antibodies blocking, e.g., CD28), signal transduction molecules or cell adhesion molecules to enhance the T cell ablation and/or other immune cell depletion. The thymic reconstitution phase would be linked to injection of donor HSC (obtained at the same time as the organ or tissue in question either from blood, pre-mobilized from the blood with G-CSF (2 intradermal injections/day for 3 days) or collected directly from the BM of the donor. The enhanced levels of circulating HSC would promote uptake by the thymus (activated by the absence of sex steroids and/or the elevated levels of GnRH). These donor HSC would develop into intrathymic DC and cause deletion of any newly formed T cells which by chance would be “donor-reactive”. This would establish central tolerance to the donor cells and tissues and thereby prevent or greatly minimize any rejection by the host. The development of a new repertoire of T cells would also overcome the immunodeficiency caused by the T cell-depletion regime.

The depletion of peripheral T cells minimizes the risk of graft rejection because it depletes non-specifically all T cells including those potentially reactive against a foreign donor. Simultaneously, however, because of the lack of T cells the procedure induces a state of generalized immunodeficiency which means that the patient is highly susceptible to infection, particularly viral infection.

### Example 6

### Sex Steroid Ablation Therapy

The patient was given sex steroid ablation therapy in the form of delivery of an LHRH agonist. This was given in the form of either Leucrin (depot injection; 22.5 mg) or Zoladex® (implant; 10.8 mg), either one as a single dose effective for 3 months. This was effective in reducing sex steroid levels sufficiently to reactivate the thymus. In some cases it is also necessary to deliver a suppresser of adrenal gland production of sex steroids. Cosudex® (5 mg/day or 50 mg/day) may also be given as one tablet per day for the duration of the sex steroid ablation therapy. Alternatively, the patient is given a GnRH antagonist, e.g., Cetrorelix or Abarelix as a subcutaneous injection

Reduction of sex steroids in the blood to minimal values takes about 1-3 weeks post surgical castration, and about 3-4 weeks following chemical castration. In some cases it is necessary to extend the treatment to a second 3 month injection/implant. The thymic expansion may be increased by simultaneous enhancement of blood HSC either as an allogeneic donor (in the case of grafts of foreign tissue) or autologous HSC (by injecting the host with G-CSF to mobilize these HSC from the BM to the thymus.

### Example 7

### Alternative Delivery Method

In place of the 3 month depot or implant administration of the LHRH agonist, alternative methods can be used. In one example the patient's skin may be irradiated by a laser such as an Er:YAG laser, to ablate or alter the skin so as to reduce the impeding effect of the stratum corneum.

Laser ablation or alteration is described in U.S. Pat. Nos. 6,251,100, 6,419,642 and 4,775,361.

In another example, delivery is by means of laser generated pressure waves. A dose of LHRH agonist is placed on the skin in a suitable container, such as a plastic flexible washer (about 1 inch in diameter and about 1/16 inch thick), at the site where the pressure wave is to be created. The site is then covered with target material such as a black polystyrene sheet about 1 mm thick. A Q-switched solid state ruby laser (20 ns pulse duration, capable of generating up to 2 joules per pulse) is used to generate a single impulse transient, which hits the target material. The black polystyrene target completely absorbs the laser radiation so that the skin is exposed only to the impulse transient, and not laser radiation. The procedure can be repeated daily, or as often as required, to maintain the circulating blood levels of the agonist.

### Example 8

### Administration of Donor Cells

Where practical, the level of hematopoietic stem cells (HSC) in the donor blood is enhanced by injecting into the donor granulocyte-colony stimulating factor (G-CSF) at 10 μg/kg for 2-5 days prior to cell collection (e.g., one or two injections of 10 μg/kg per day for each of 2-5 days). The donor may also be injected with LHRH agonist and/or a cytokine, such as G-CSF or GM-CSF, prior to (e.g., 7-14 days before) collection to enhance the level or quality of stem cells in the blood. CD34⁺ donor cells are purified from the donor blood or BM, such as by using a flow cytometer or immunomagnetic beading. Antibodies that specifically bind to human CD34 are commercially available (from, e.g., Research Diagnostics Inc., Flanders, N.J.; Miltenyi-Biotec, Germany). Donor-derived HSC are identified by flow cytometry as being CD34⁺. These CD34+ HSC may also be expanded by in vitro culture using feeder cells (e.g., fibroblasts), growth factors such as stem cell factor (SCF), and LIF to prevent differentiation into specific cell types. At approximately 3-4 weeks post LHRH agonist delivery (i.e., just before or at the time the thymus begins to regenerate) the patient is injected with the donor HSC, optimally at a dose of about 2-4×10⁶ cells/kg. G-CSF may also be injected into the recipient to assist in expansion of the donor HSC. If this timing schedule is not possible because of the critical nature of clinical condition, the HSC could be administered at the same time as the GnRH. It may be necessary to give a second dose of HSC approximately 2-3 weeks later to assist in the thymic regrowth and the development of donor DC (particularly in the thymus). Once the HSC have engrafted (i.e., incorporated into) and/or migrated to the BM and thymus, the effects should be permanent since HSC are self-renewing.

The reactivating or reactivated thymus takes up the donor HSC and converts them into donor-type T cells and DC, while converting the recipient's HSC into recipient-type T cells and DC. By inducing deletion by cell death, or by inducing tolerance through immunoregulatory cells, the donor and host DC tolerize any new T or NK cells that are potentially reactive with donor or recipient cells.

### Example 9

### Transplantation of Graft

In one embodiment of the invention, while the recipient is still undergoing continuous T cell depletion and/or other immune cell depletion and/or immunosuppressive therapy, an organ, tissue, or group of cells that has been at least partly depleted of donor T cells is transplanted from the donor to the recipient patient. The recipient thymus has been activated by GnRH treatment and infiltrated by exogenous HSC.

Within about 3-4 weeks of LHRH therapy the first new T cells will be present in the blood stream of the recipient. However, in order to allow production of a stable chimera of host and donor hematopoietic cells, immunosuppressive therapy may be maintained for about 3-4 months. The new T cells will be purged of potentially donor reactive and host reactive cells, due to the presence of both donor and host DC in the reactivating thymus. Having been positively selected by the host thymic epithelium, the T cells will retain the ability to respond to normal infections by recognizing peptides presented by host APC in the peripheral blood of the recipient. The incorporation of donor dendritic cells into the recipient's lymphoid organs establishes an immune system situation virtually identical to that of the host alone, other than the tolerance of donor cells, tissue and organs. Hence, normal immunoregulatory mechanisms are present. These may also include the development of regulatory T cells which switch on or off immune responses using cytokines such as IL4, 5, 10, TGF-beta, TNF-alpha.

### Example 11

### Alternative Protocols

In the event of a shortened time available for transplantation of donor cells, tissue or organs, the timeline as used in Examples 1-10 is modified. T cell ablation and/or other immune cell depletion and sex steroid ablation are begun at the same time. T cell ablation and/or other immune cell depletion is maintained for about 10 days, while sex steroid ablation is maintained for around 3 months. In one embodiment, HSC transplantation is performed when the thymus starts to reactivate, at around 10-12 days after start of the combined treatment.

In an even more shortened time table, the two types of ablation and the HSC transplant are started at the same time. In this event, T cell ablation and/or other immune cell depletion is maintained 3-12 months, for example, for 3-4 months.

### Example 12

### Termination of Immunosuppression

When the thymic chimera is established and the new cohort of mature T cells have begun exiting the thymus, blood is taken from the patient and the T cells examined in vitro for their lack of responsiveness to donor cells in a standard mixed lymphocyte reaction (see, e.g., Current Protocols In Immunology, John E. Coligan et al., (eds), Wiley and Sons, New York, N.Y. (1994), and yearly updates including 2002). If there is no response, the immunosuppressive therapy is gradually reduced to allow defense against infection. If there is no sign of rejection, as indicated in part by the presence of activated T cells in the blood, the immunosuppressive therapy is eventually stopped completely. Because the HSC have a strong self-renewal capacity, the hematopoietic chimera so formed will be stable theoretically for the life of the patient (that is a normal, non-tolerized and non-grafted person).

### Example 13

### Use of LHRH Agonist to Reactivate the Thymus in Humans

Materials and Methods:

In order to show that a human thymus can be reactivated by the methods of this invention, these methods were used on patients who had been treated with chemotherapy for prostate cancer.

Patients.

Sixteen patients with Stage I-III prostate cancer (assessed by their prostate specific antigen (PSA) score) were chosen for analysis. All subjects were males aged between 60 and 77 who underwent standard combined androgen blockade (CAB) based on monthly injections of GnRH agonist 3.6 mg goserelin acetate (Zoladex®) or 7.5 mg leuprolide (Lupron®) treatment per month for 4-6 months prior to localized radiation therapy for prostate cancer as necessary.

FACS Analysis.

The appropriate antibody cocktail (20 □1) was added to 200 □1 whole blood and incubated in the dark at room temperature (RT) for 30 min. RBC, were lysed and remaining cells washed and resuspended in 1% PFA for FACS analysis. Samples were stained with antibodies to CD19-FITC, CD4-FITC, CD8-APC, CD27-FITC, CD45RA-PE, CD45RO-CyChrome, CD62L-FITC and CD56-PE (all from Pharmingen, San Diego, Calif.).

Statistical Analysis.

Each patient acted as an internal control by comparing pre- and post-treatment results and were analyzed using paired student t-tests or Wilcoxon signed rank tests.

Results:

Prostate cancer patients were evaluated before and 4 months after sex steroid ablation therapy. The results are summarized in FIGS. 19-23. Collectively the data demonstrate qualitative and quantitative improvement of the status of T cells in many patients.

I. The Effect of LHRH Therapy on Total Numbers of Lymphocytes and T cells Subsets Thereof:

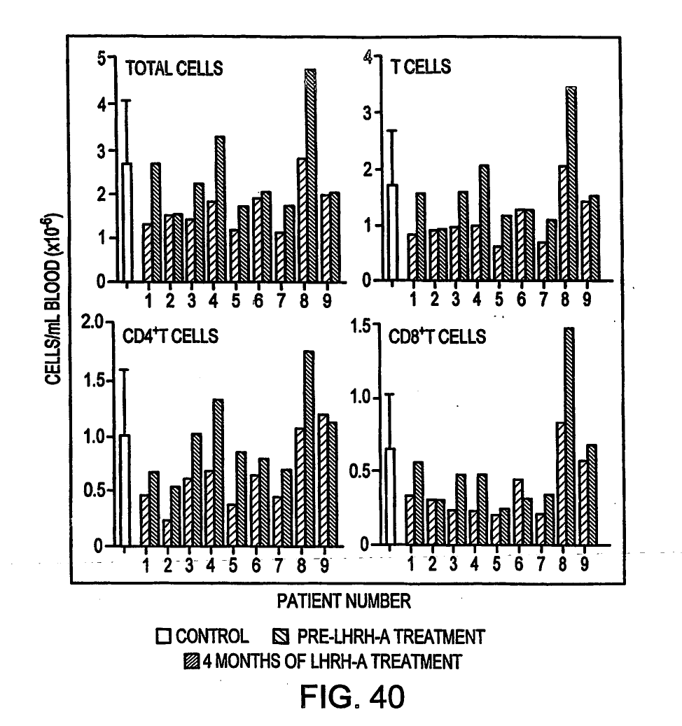

The phenotypic composition of peripheral blood lymphocytes was analyzed in patients (all >60 years) undergoing LHRH agonist treatment for prostate cancer (FIG. 40). Patient samples were analyzed before treatment and 4 months after beginning LHRH agonist treatment. Total lymphocyte cell numbers per ml of blood were at the lower end of control values before treatment in all patients.

Following treatment, six out of nine patients showed substantial increases in total lymphocyte counts (in some cases a doubling of total cells was observed). Correlating with this was an increase in total T cell numbers in six out of nine patients. Within the CD4⁺ subset, this increase was even more pronounced with eight out of nine patients demonstrating increased levels of CD4⁺ T cells. A less distinctive trend was seen within the CD8⁺ subset with four out of nine patients showing increased levels albeit generally to a smaller extent than CD4⁺ T cells.

II. The Effect of LHRH Therapy on the Proportion of T Cells Subsets:

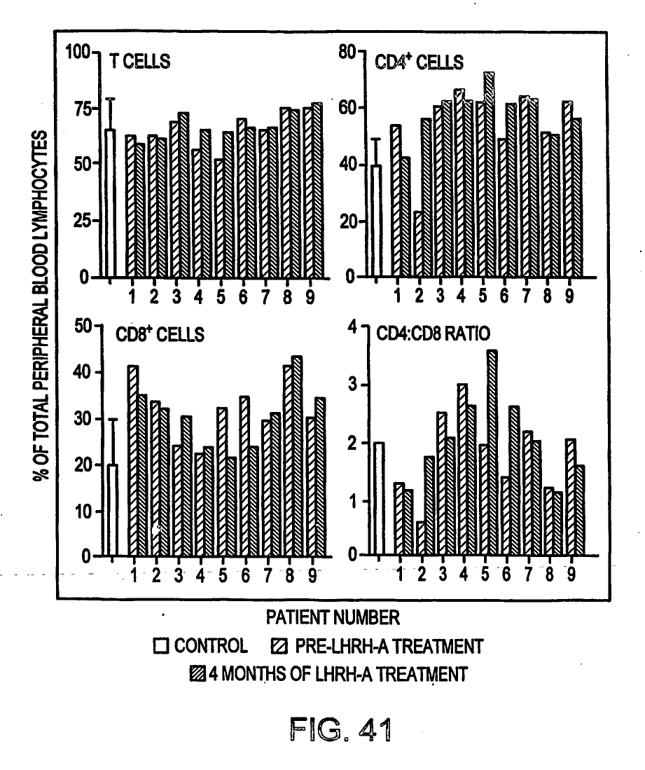

Analysis of patient blood before and after LHRH agonist treatment demonstrated no substantial changes in the overall proportion of T cells, CD4⁺ or CD8⁺ T cells and a variable change in the CD4⁺:CD8⁺ ratio following treatment (FIG. 41). This indicates that there was little effect of treatment on the homeostatic maintenance of T cell subsets despite the substantial increase in overall T cell numbers following treatment. All values were comparative to control values.

III. The Effect of LHRH Therapy on the Proportion of B Cells and Myeloid Cells:

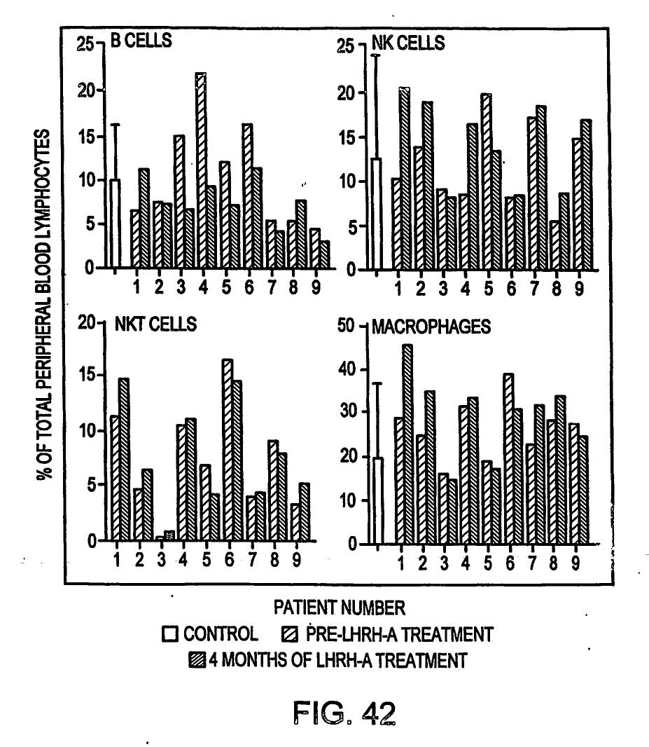

Analysis of the proportions of B cells and myeloid cells (NK, NKT and macrophages) within the peripheral blood of patients undergoing LHRH agonist treatment demonstrated a varying degree of change within subsets (FIG. 42). While NK, NKT and macrophage proportions remained relatively constant following treatment, the proportion of B cells was decreased in four out of nine.

IV. The Effect of LHRH Agonist Therapy on the Total Number of B Cells and Myeloid Cells:

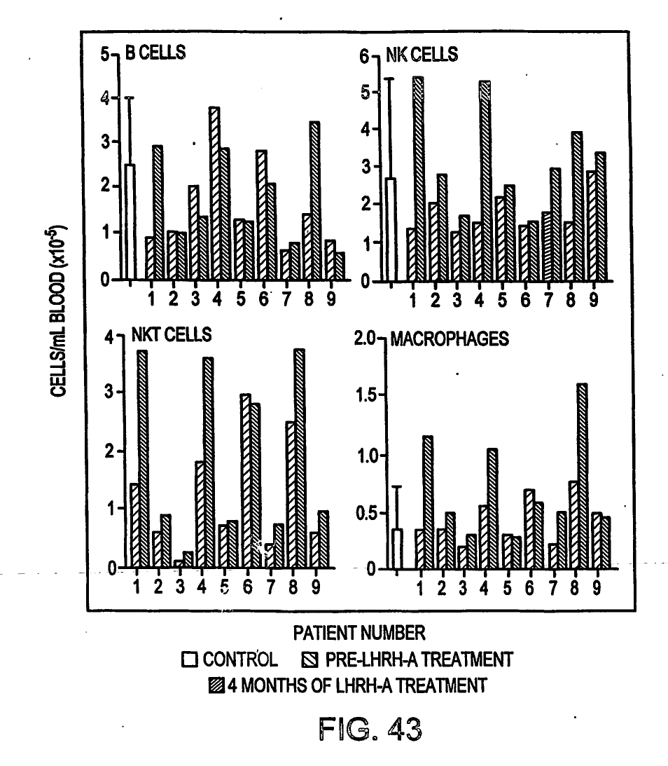

Analysis of the total cell numbers of B and myeloid cells within the peripheral blood post-treatment showed clearly increased levels of NK (five out of nine patients), NKT (four out of nine patients) and macrophage (three out of nine patients) cell numbers post-treatment (FIG. 43). B cell numbers showed no distinct trend with two out of nine patients showing increased levels; four out of nine patients showing no change and three out of nine patients showing decreased levels.

V. The Effect of LHRH Therapy on the Level of Naïve Cells Relative to Memory Cells:

The major changes seen post-LHRH agonist treatment were within the T cell population of the peripheral blood. In particular there was a selective increase in the proportion of naïve (CD45RA⁺) CD4⁺ cells, with the ratio of naïve (CD45RA⁺) to memory (CD45RO⁺) in the CD4⁺ T cell subset increasing in six out of nine patients (data not shown).

VI. Conclusion

Thus it can be concluded that LHRH agonist treatment of an animal such as a human having an atrophied thymus can induce regeneration of the thymus. A general improvement has been shown in the status of blood T lymphocytes in these prostate cancer patients who have received sex-steroid ablation therapy. It is likely that such cells are derived from the thymus as no other source of mainstream (TCRαβ+CD8 αβ chain) T cells has been described. Gastrointestinal tract T cells are predominantly TCR γδ or CD8 αα chain,

### Example 14

### Induction of Tolerance in Humans

A male human patient requiring a skin or organ transplant is administered a standard combined androgen blockade (CAB) based on GnRH agonist (Lucrin, 3.6 mg) treatment, as described above in Example 10, for 0-6 months. While the androgen-blocking treatment is ongoing, the patient is given an intravenous injection of CD34+ cells collected from the peripheral blood of an allogeneic donor. To collect the CD34+ cells, peripheral blood of the donor (i.e., the person who will be donating his/her organ or skin to the recipient) is collected, and CD34+ cells isolated-from the peripheral blood according to standard methods. One non-limiting method is to incubate the peripheral blood with an antibody that specifically binds to human CD34 (e.g., a murine monoclonal anti-human CD34+ antibody commercially available from Abcam Ltd., Cambridge, UK), secondarily stain the cells with a detectably labeled anti-murine antibody (e.g., a FITC-labeled goat anti-mouse antibody), and isolate the FITC-labeled CD34+ cells through fluorescent activated cell sorting (FACS). Because of the low number of CD34+ cells found in circulating peripheral blood, multiple collection and cell sorting may be required from the donor. The CD34+ may be cryopreserved until used to reconstitute the recipient patient. In one example, at least 5×10⁵ HSC per kg body weight are administered to the recipient patient.

The recipient patient will be monitored to detect the presence of donor blood and dendritic cells in his/her peripheral blood. When such donor cells are detected, the transplantation of the donor tissue (i.e., skin and/or organ) is made. The donor tissue is accepted by the recipient to a greater degree (i.e., survives longer in the recipient) than in a recipient who had not had his thymus reactivated and had not been reconstituted with donor CD34+ cells.

### Example 15

### Graft Acceptance Facilitated by Genetically Modified HSC Cells

MHC matched male and female mice are used to assess if genetic modification of HSC can facilitate graft acceptance.

To do this, aged (i.e., 2 year old) female Balb/cJ (H-2d) mice are either surgically castrated (e.g., by removing the ovaries according to standard methods), or are chemically castrated. For chemical castration, mice are injected subcutaneously with 10 mg/kg Lupron (a GnRH agonist) as a 1 month slow release formulation. Alternatively mice are injected with a GnRH antagonist (e.g., Cetrorelix or Abarelix). Confirmation of loss of sex steroids is performed by standard radioimmunoassay of plasma samples following manufacturer's instructions, and may be monitored throughout the procedure. Castrate levels (<0.5 ng estrogen/ml) should normally be achieved by 3-4 weeks post injection.

Bone marrow cells from female Balb/cJ are transfected, under conditions for expression, with a gene encoding the H-Y protein, which expressed on the cells of male, but not female, Balb/cJ mice. In other words, the H-Y protein-encoding gene (or cDNA) is inserted into an expression vector (e.g., a plasmid or a viral vector, such as a retroviral vector), and then transfected into female Balb/cJ bone marrow cells (see, e.g., Bonyhadi et al., (1997) J. Virol. 71:4707). Expression of the H-Y antigen on the transfected cells is determined by standard methods (e.g., Western blotting, Northern blotting, cell surface staining).

The transfected bone marrow cells are then administered to the myeloablated or immunosupressed, castrated (chemically or surgically) female mice to reconstitute their thymus, as described above. Concurrently, or a week to a month following reconstitution, a skin graft from a male Balb/cJ mouse is transplanted onto the reconstituted, castrated female mouse.

The results will show that the skin graft from the male Balb/cJ mouse “takes” better on a castrated female mouse reconstituted with the H-Y protein-encoding gene transfected female Balb/cJ mouse bone marrow cells than on a castrated female mouse who has not been reconstituted with the H-Y protein-encoding gene transfected female Balb/cJ mouse bone marrow cells.

Once the recipient female Balb/cJ mouse fully accepts the graft from the male Balb/cJ mouse, if she is chemically castrated, the administration of the chemical can be stopped, allowing her thymus to atrophy and her fertility to be restored.

### Example 16

### Regeneration of the Peripheral Immune Cell Pool Following Hematopoietic Stem Cell Transplantation in Humans

I. Allogeneic and Autologous HSCT

This example relates to clinical trials undertaken with HSCT patients. To assess the clinical potential for restoring thymus and bone marrow function in humans, prostate cancer patients (>60 years) who routinely undergo sex-steroid ablation therapy based on LHRH-agonist (chemical castration) treatment have been analyzed. Patients were examined at the time of presentation and after 4-months of treatment by which time serum testosterone concentration was at castrate levels for all patients.

Materials and Methods:

Patients.

Eighty-two Patients were all due to undergo high-dose therapy (HDT) with PBSCT for malignant disease or bone marrow failure (n=22 for allogeneic control patients, n=20 for allo LHRH-A treated patients, n=20 for autologous controls and n=20 for autologous LHRH-A treated patients). Test patients were given 3.6 mg (effective for 4 weeks) Zoladex (LHRH-A) 3-weeks prior to autologous or allogeneic stem cell transplantation and then monthly injections for 4-months. All patients were analyzed pre-treatment, weekly for 5-weeks after transplantation and then monthly up to 12 months. Ethics approval was obtained from The Alfred Committee for Ethical Research on Humans (Trial Number 01/006).

FACS Analysis of Whole Peripheral Blood.

The appropriate antibody cocktail (20 □l) was added to 200 □l whole blood and incubated in the dark at room temperature (RT) for 30 min. RBC were lysed and remaining cells washed and resuspended in 1% PFA for FACS analysis. Samples were stained with antibodies to CD19-FITC, CD4-FITC, CD8-APC, CD27-FITC, CD45RA-PE, CD45RO-CyChrome, CD62L-FITC and CD56-PE (all from Pharmingen, San Diego, Calif.).

Ki67 Analysis.

For detection of proliferating cells, samples were surface stained with CD27-FITC, CD45RO-CyChrome, and CD4- or CD8-APC (Pharmingen, San Diego, Calif.). Following red cell lysis, samples were incubated for 20 min, RT, in the dark in 500 μl of 1×FACS permeabilizing solution (Becton-Dickinson, USA; 1× solution was made from 10× stock in R.O.H₂O). Washed samples (2 ml FACS buffer, 5 min., 600_{gmax}, RT) were incubated with either anti-Ki67-PE or anti-Ki67-FITC (or the appropriate isotype controls) for 30 min. at RT, in the dark. Samples were then washed and resuspended in 1% PFA for analysis.

Preparation of PBMC.

Purified lymphocytes were prepared for T-cell stimulation assays and TREC analysis, by ficoll-hypaque separation and following centrifugation, the plasma layer was removed and stored at −20° C. prior to analysis of sex steroid levels. Cells not used for T lymphocyte stimulation assays were resuspended in freezing media and stored in liquid nitrogen prior to TREC analysis.

T Lymphocyte Stimulation Assay.

For mitogen stimulation, purified lymphocytes were plated out in 96-well round-bottom plates at a concentration of 1×10⁵ cells/well in 100 μl of RPMI-FCS. Cells were incubated at 37° C., 5% CO₂ with PHA in doses from 1-10 μg/ml. For TCR-specific stimulation, cells were incubated for 48 hours on plates previously coated with purified anti-CD3 (1-10 μg/ml) and anti-CD28 (10 μg/ml). Following plaque formation (48-72 hours), 1 μCi of ³H-thymidine was added to each well and plates incubated for a further 16-24 hours. Plates were harvested onto filter mats and incorporation of ³H-Thymidine was determined using liquid scintillation on a β-counter (Packard-Coulter, USA).

TREC Analysis:

Cell Sorting.

Frozen samples were rapidly thawed and stained with anti-CD4-FITC and anti-CD8-APC for 30 min on ice, washed (2 ml FACS buffer) and fixed with 3% formalin in PBS (with agitation). Samples were incubated for a further 30 min., washed and resuspended in 500 μl FACS buffer for sorting. CD4⁺ and CD8⁺ cell populations were sorted on a MoFlo® cell sorter (Cytomation Inc.).

DNA Isolation.

Cells were sorted and resuspended in Proteinase K (PK) digestion buffer (2×10⁵ cells/20 μl of a 0.8 mg/mL solution). Samples were incubated for 1 hour at 56° C. followed by 10 min at 95° C. to inactivate the proteinase.

Real-Time PCR using Molecular Beacons.

Real-time PCR for analysis of TREC content in sorted cells was performed as described previously (Zhang et al., (1999) J. Exp. Med. 190:725). The primers were sense, 5′-GGATGGAAAACACAGTGTGACATGG-3′ (SEQ ID NO:4) and antisense, 5′-CTGTCAACAAAGGTGATGCCACATCC-3′ (SEQ ID NO:5). One cycle of denaturation (95° C. for 10 min) was performed, followed by 45 cycles of amplification (94° C. for 30 s, 60° C. for 30 s, and 72° C. for 30s). To normalize for cell equivalents in the input DNA, a separate real-time PCR assay was used to quantify the CCR5 coding sequence, which contains no pseudogenes.

Statistical Analysis.

Statistical analysis was performed using Instat II software. For prostate cancer HSCT studies, a Mann-Whitney U-test was performed. For human studies, each patient acted as an internal control by comparing pre- and post-treatment results and were analyzed using paired student t-tests or wilcoxon signed rank tests.

Results:

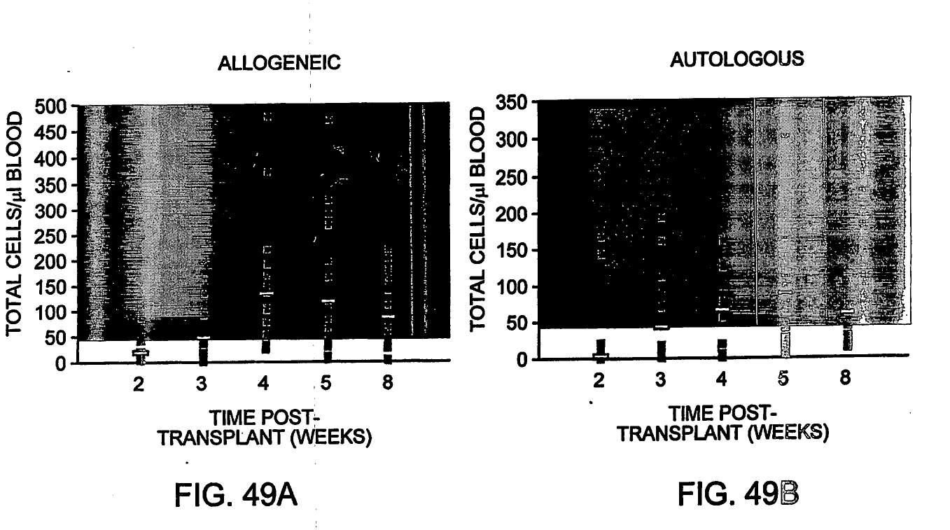

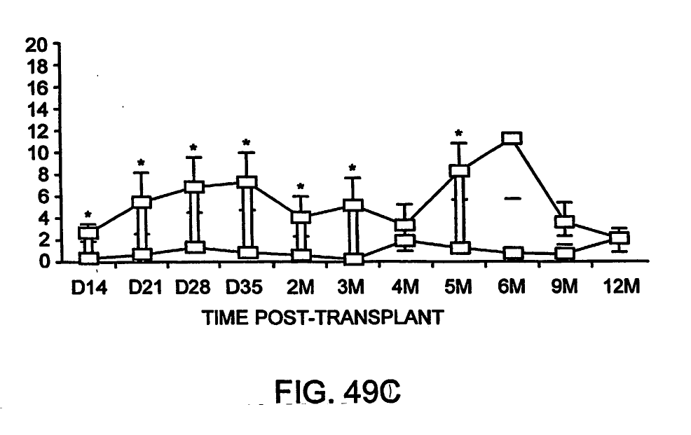

FIG. 49 depicts analysis of natural killer (NK) cell recovery at various time points (2-8 weeks) following HSCT in control patients. As shown in FIGS. 49A-B, respectively, a similar trend was observed for both control allogeneic and autologous transplant recipients. In contrast, allogeneic patients who were given LHRH-A treatment 3 weeks prior to HSCT showed a significantly higher number of NKT (V□24+V□11+) cells from D14-5M post-transplant (FIG. 49C; data is expressed as mean±1 SEM of 6-20 patients; *=p≦0.05). NKT cells were analyzed based on their V□24⁺V□11⁺ phenotype.

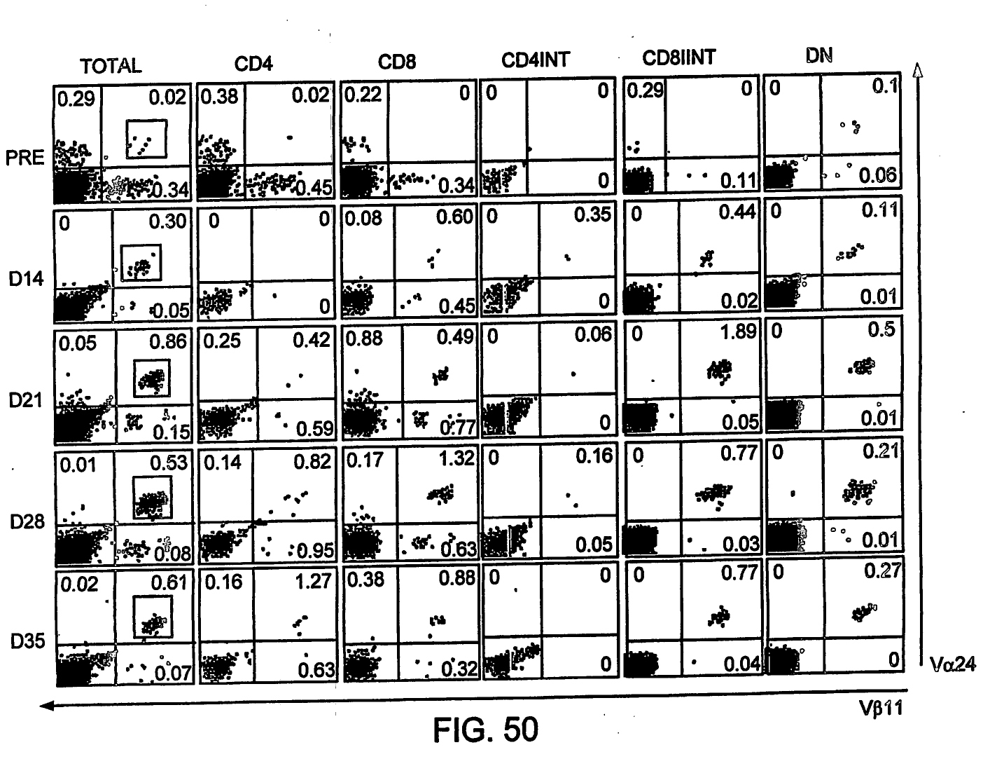

FIG. 50 depicts FACS analysis of NKT cell reconstitution at various time points (day 14, 21, 28 and 35) following HSCT in control patients. An early recovery was observed in allogeneic patients, and was seen predominantly within the CD8+ population early post-transplant, which indicated extrathymic routes of regeneration. Also, CD4+NKT cells were evident from 1 month post-transplant.

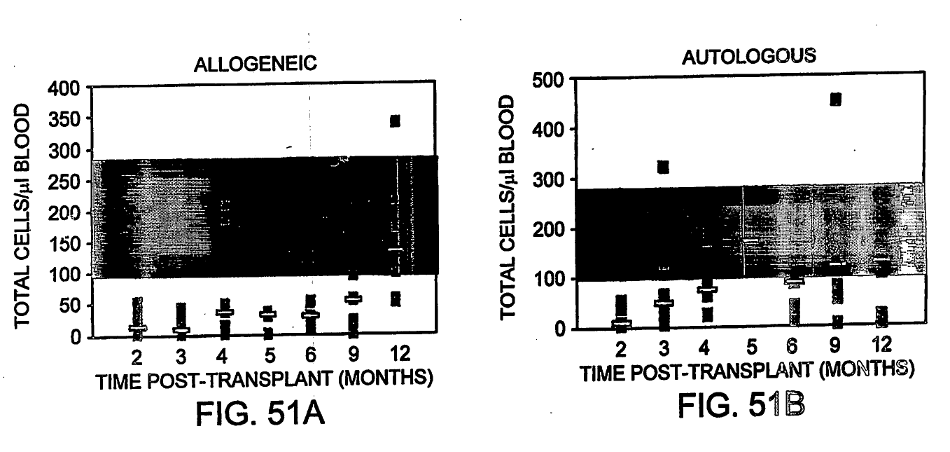

FIG. 51 depicts B cell reconstitution following HSCT at various time points (2-12 months) following HSCT in control patients. As shown in FIG. 51B, B cell regeneration occurs relatively faster in autologous transplant patients as compared to that of allogeneic patients (FIG. 51A). However, a return to control values (shaded) was not evident until at least 6 months post-transplant in both groups.

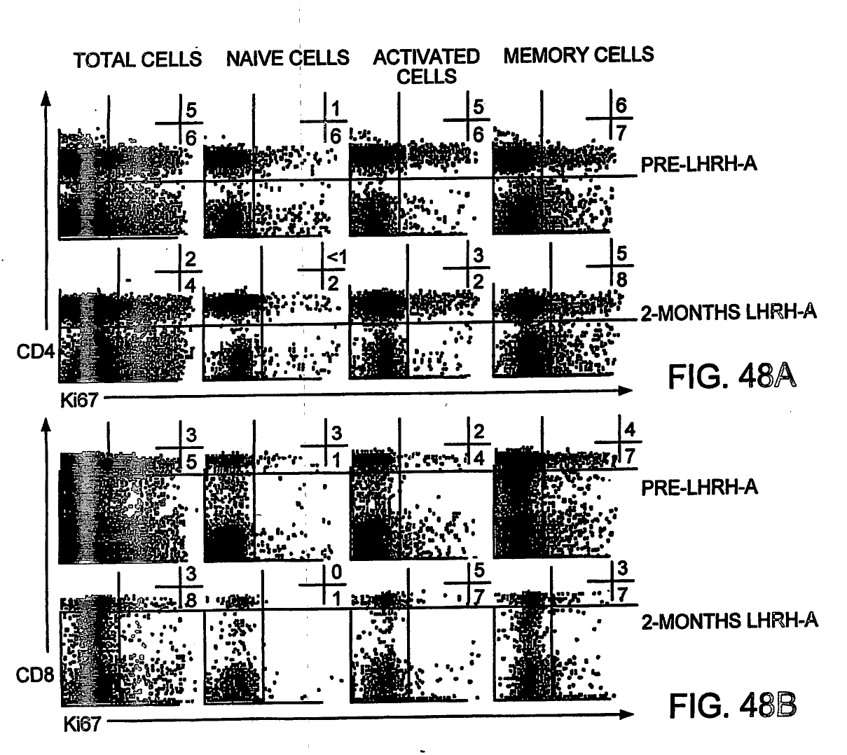

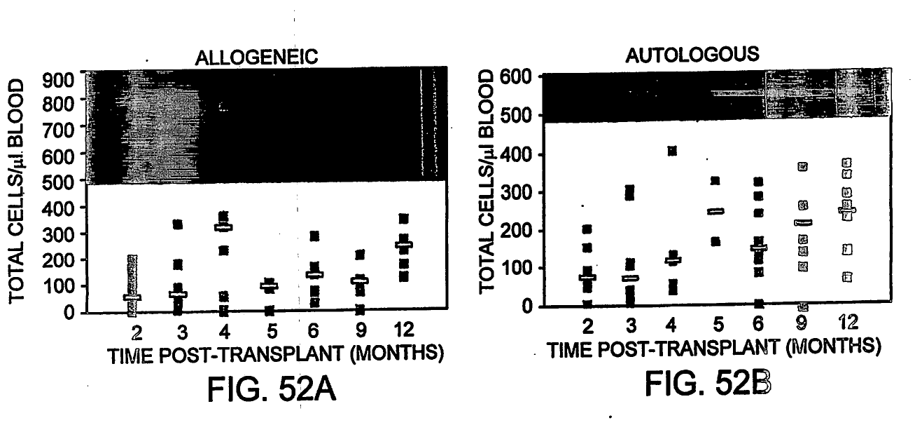

FIG. 52 depicts CD4⁺ reconstitution following HSCT at various time points (2-12 months) following HSCT in control patients. While B cell numbers were returning to control values by 6 months post-transplant (see FIGS. 48A-B), CD4⁺ T cell numbers were severely reduced, even at 12 months post-transplant, in both autologous (FIG. 52B) and allogeneic (FIG. 52A) recipients.

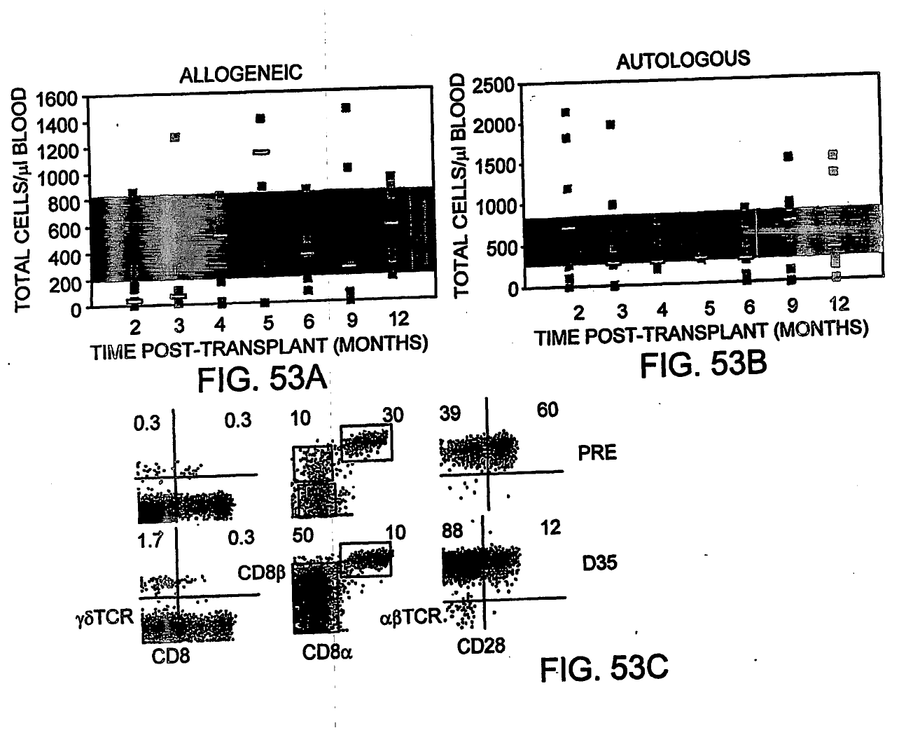

FIG. 53 depicts CD8⁺ regeneration following HSCT at various time points (2-12 months) following HSCT in control patients. As shown in FIG. 53A-B, CD8⁺ T cell numbers regenerated quite rapidly post-transplant in both allogeneic and autologous recipients, respectively. However, as shown in FIG. 53C, the CD8⁺ T cells are mainly of extrathymic origin as indicated by the increase in TCRγδ⁺ T CD8⁺ T cells, CD8αα T cells, and CD28⁻CD8⁺ T cells.

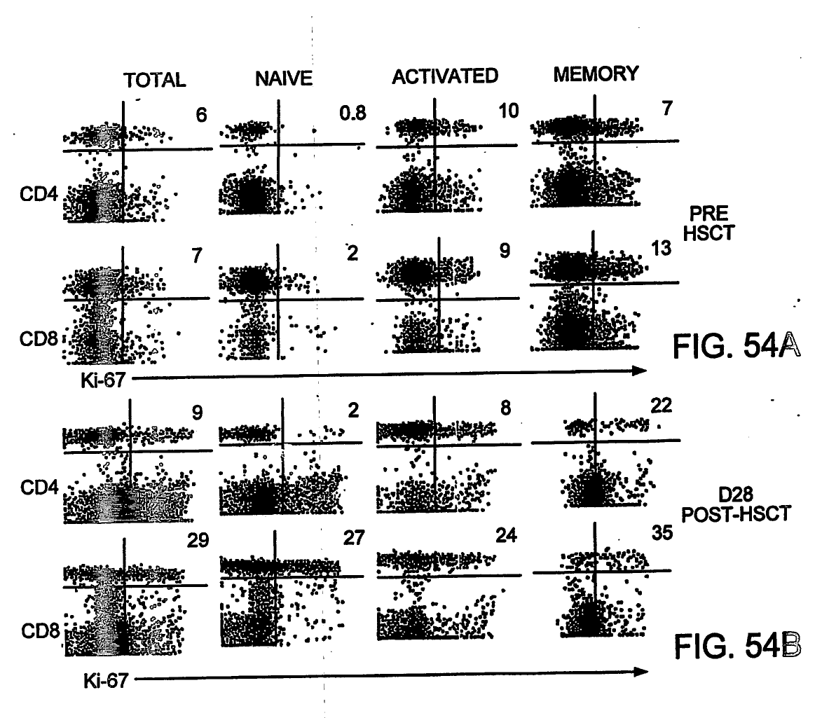

FIG. 54 depicts FACS analysis of proliferation in various populations of CD4⁺ and CD8⁺ T cells before (FIG. 54A) and 28 days after (FIG. 54B) HSCT in control patients using the marker Ki-67. Cells were analyzed on the basis of naïve, memory and activated phenotypes using the markers CD45RO and CD27. The majority of proliferation occurred in CD8⁺ T cell subset, which further indicated that these cells were extrathymically derived and that the predominance of proliferation occurred within peripheral T cell subsets.

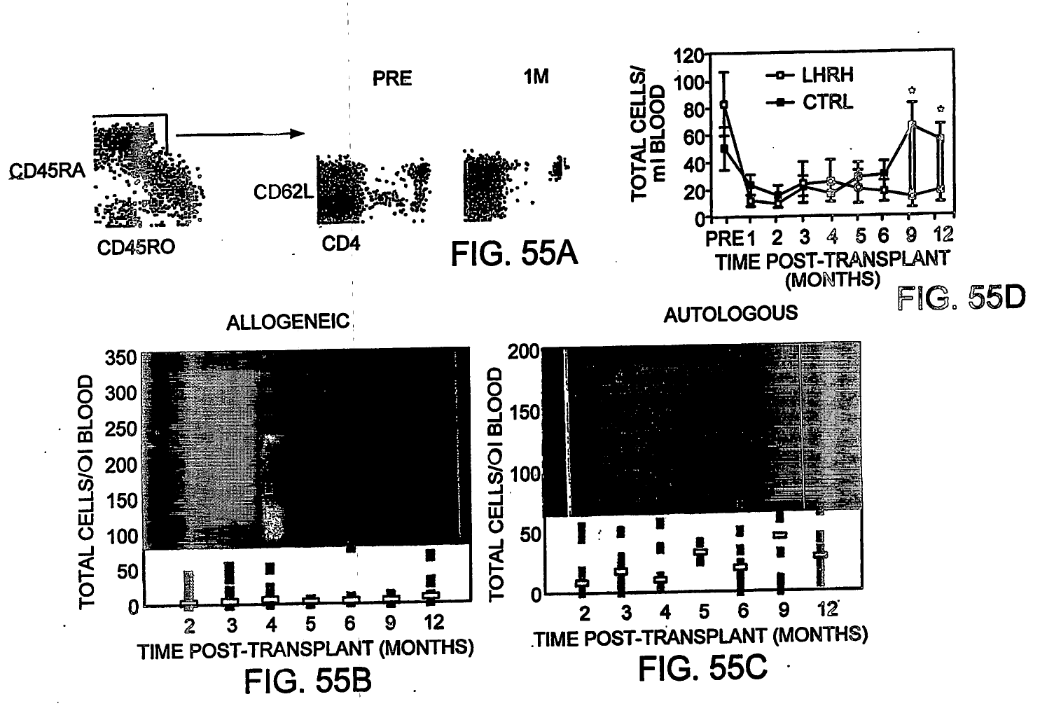

FIG. 55 depicts naïve CD4+ T cell regeneration at various time points (2-12 months) following HSCT in control patients and LHRH-A treated patients. FIG. 55A depicts FACS analysis of naïve CD4⁺ T cells (CD45RA+CD45RO−CD62L⁺), and shows a severe loss of these cells throughout the study. As shown in FIGS. 55B-C, naïve CD4⁺ T cell began to regenerate by 12 months post-HSCT in autologous transplant patients (FIG. 55C) but were still considerably lower than the control values in allogeneic patients (FIG. 55B). These results indicated that the thymus was unable to restore adequate numbers of naïve T cells post-transplant due to the age of the patients. In contrast, in patients that were given LHRH-A 3-weeks prior to allogeneic HSCT showed a significantly higher number of naïve CD4⁺ T cells at both 9 & 12 months post-transplant compared to controls (p≦0.05 both 9 & 12 months post-transplant compared to control (non-LHRH-A treated) (FIG. 55D). This indicates enhanced regeneration of the thymic-dependent T cell pathway with sex steroid ablation therapy.

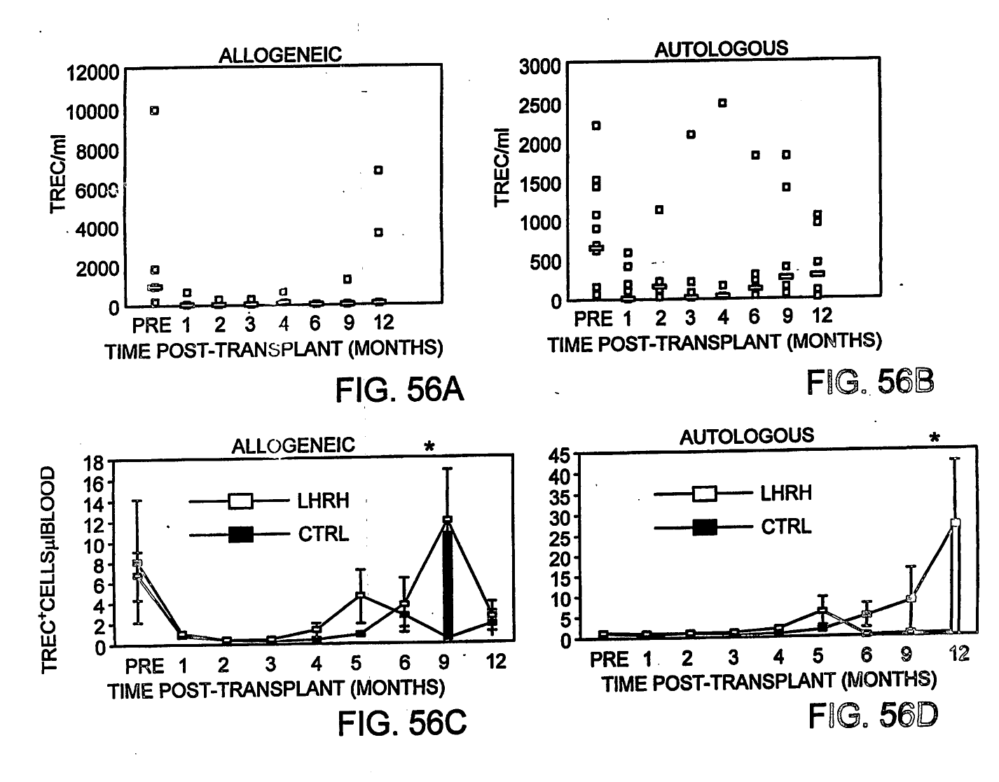

FIG. 56 depicts TREC levels at various time points (1-12 months) following HSCT in control patients. Analysis of TREC levels, which are only seen in recent thymic emigrants (RTE), emphasized the inability of the thymus to restore levels following transplant in both allogeneic (FIG. 52A) and autologous (FIG. 52B) patients. Again, this was due to the age of the patients, as well as the lack of thymic function due to thymic atrophy, which has considerable implications in the morbidity and mortality of these patients. In contrast, patients undergoing allogeneic peripheral blood stem cell transplantation demonstrated a significant increase in CD4+TREC+ cells/ml blood when treated with an LHRH-A prior to allogeneic transplantation (p≦0.01 at 9 months post-transplant compared to control (non-LHRH-A treated). Allogeneic patients who were given LHRH-A treatment showed a significantly higher number of CD4⁺TREC⁺ cells/ml blood at 9 months post-transplant (FIG. 56C) compared to controls. Autologous LHRH-A treated patients also showed significantly higher levels at 12 months post-.transplant (FIG. 56D). This indicates enhanced regeneration of the thymus with sex steroid ablation therapy. Data is expressed as mean±1 SEM of 5-18 patients. *=p≦0.01.

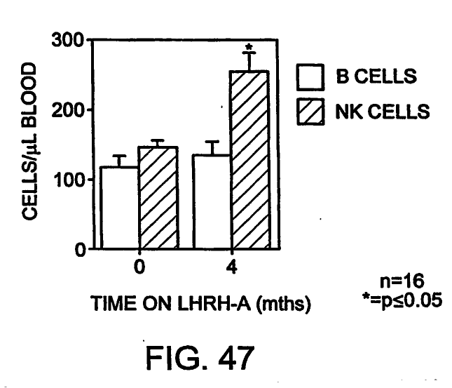

LHRH-A administration significantly increases NK but not B cell numbers in the peripheral blood. Overall, no significant change in B cell numbers was observed with LHRH-A treatment (FIG. 47). However, a significant increase in NK cell numbers was observed with treatment (p≦0.01) (FIG. 47). Therefore, removal of sex steroid results in significantly increased numbers of T cells and NK cells.

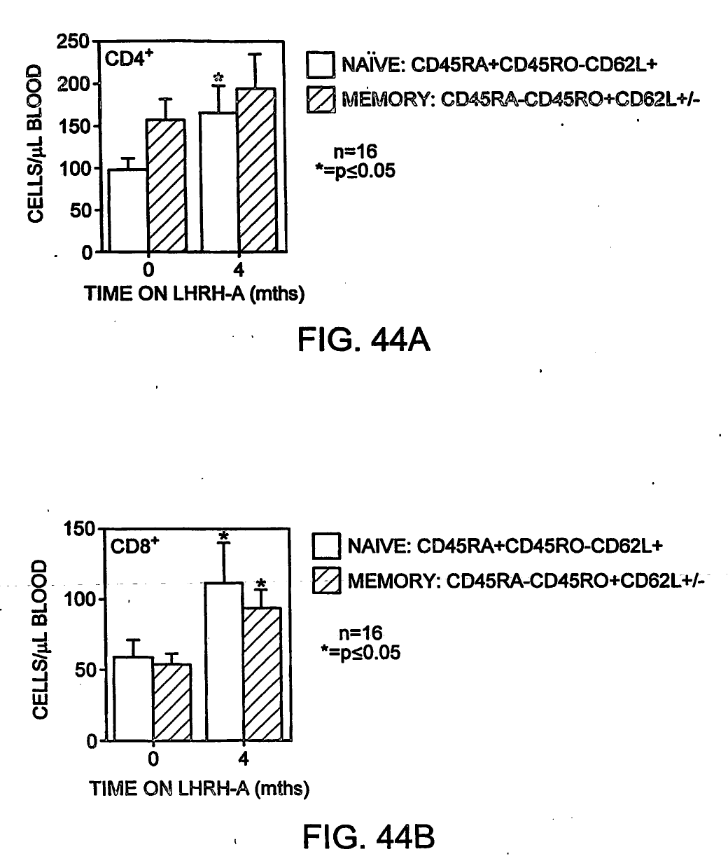

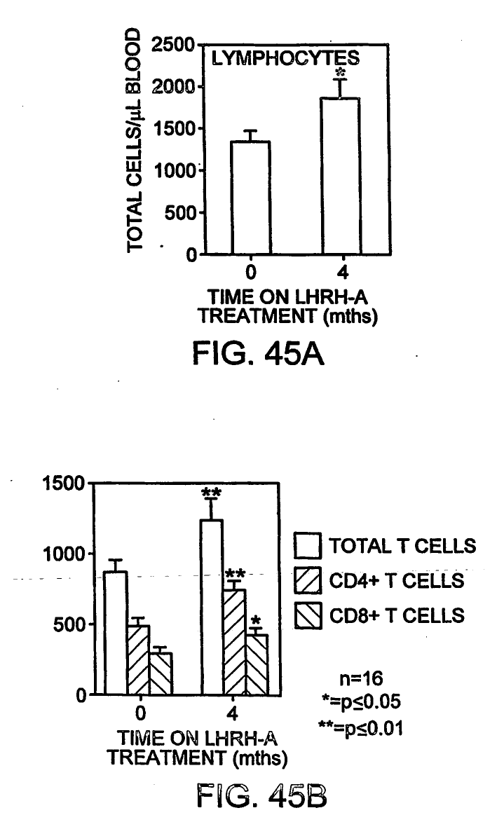

A significant increase in the total lymphocyte, T cell (predominantly CD4⁺) and NK cells was observed (FIGS. 45 and 47) consistent with previous studies of patients treated with LHRH-agonists (Garzetti et al., (1996) Obstet. Gynecol. 88: 23440; Oliver et al., (1995) Urol. Int., 54:226-229; Umesaki et al., (1999) Gynecol. Obstet. Invest. 48:66-8). More detailed analysis of the T cell compartment revealed a significant increase in the numbers of naïve CD4⁺ T cells and both naïve and memory CD8⁺ T cells following LHRH-A treatment (FIG. 44).

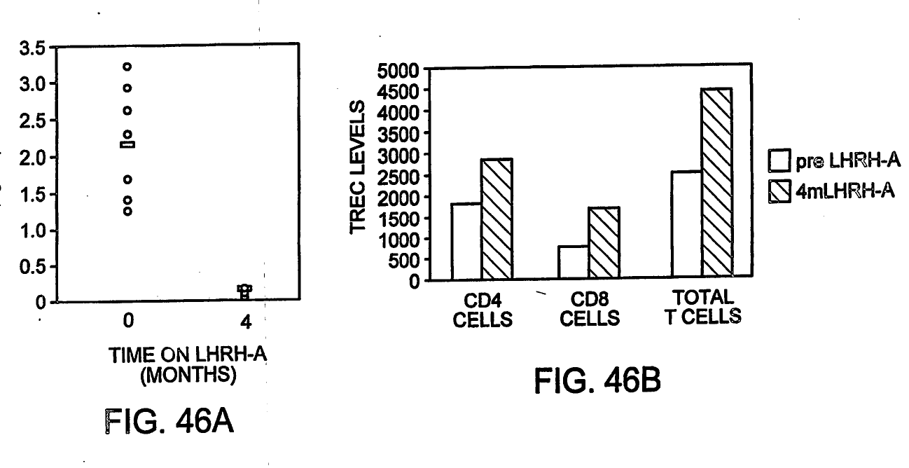

To determine if the increase in naïve T cells was through peripheral expansion (as seen for example with IL-7 administration (Soares et al., 1998 J. Immunol. 161:5909-5917) or as a direct result of thymic reactivation, analysis of cellular proliferation (Ki-67 antigen⁺), together with TREC levels was performed (Hazenberg et al., (2001) J. Mol. Med. 79:631-40). No change in the level of proliferation was seen with agonist treatment in naïve, activated or memory populations of both CD4⁺ (FIG. 48A) and CD8⁺ T cells (FIG. 48B) (remaining at a low 2-4%). This indicates that the treatment does not directly induce proliferation of T cells, and that the levels of TRECs would not be influenced by excessive proliferation in the periphery. This does not rule out the possibility of peripheral expansion at earlier time-points. However this would presumably only account for increased activated/memory cell levels. Direct evidence for an increase in thymic function and T cell export was found following analysis of TREC levels in 10 patients (FIG. 46B). Within both the CD4⁺ and CD8⁺ T cell population, five out of ten patients showed an increase (>25% above initial presentation values) in absolute TREC levels (per ml of blood) by 4 months of LHRH-A treatment. This was also reflected in a proportional increase (per 1×10⁵ cells). This correlated with six out of ten patients showing an overall increase in total TREC levels. Only 1 patient showed a decrease in total TRECs (about 30% decrease). Since TRECs are diluted out with mitosis (Zhang et al., (1999) J. Exp. Med. 190:725-732), which could occur intrathymically as part of normal T cell development or following export (Hazenberg et al., (2001) J. Mol. Med. 79:631-40), the absolute TREC levels would represent very much an underestimate of T cell export. The marked increase in total TREC⁺ cells in the periphery following treatment with the agonist is thus fully consistent with regeneration of the thymus-dependent T cell pathway (Douek et al., (1998) Nature 396:690-695 (1998); Douek et al., (2001) J. Immunol. 167:6663-8; Hochberg et al., (2001) Blood 98:1116-21). Together, these data demonstrate the ability of sex steroid inhibition to improve thymic output in adult humans and provides a basis for restoring naïve T cell numbers following severe T-cell depletion in many clinical conditions.

### Example 17

### Sex Steroid Ablation Enhances Immune Reconstitution Following Hematopoietic Stem Cell Transplantation in Mice

This experiment was done to test the hypothesis that sex steroid inhibition in recipients of an allogeneic HSCT can improve their post-transplant immune reconstitution. Thus, these experiments aimed to establish whether sex steroid ablation influenced hematopoietic recovery following allogeneic HSCT. Fourteen days after HSCT, BM and thymic cell numbers were significantly increased in the castrated mice compared to sham controls. These remained elevated at day 28 at which time splenic cellularity was also increased in the castrates. In the thymus, T cell precursors and DC were significantly after HSCT and castration. BM precursors and developing B cells were also significantly increased after HSCT and castration. These central increases translated to a significant increase in donor-derived peripheral T and B cells after allogeneic HSCT. Every immune-enhancing strategy carries the risk of exacerbating the development of graft-versus-host disease (GVHD). Mice were castrated at the same time as GVHD induction in an allogeneic setting. There was no significant difference in GVHD incidence or severity when comparing castrated and sham-castrated mice. Furthermore, GVT activity was not diminished in the absence of sex steroid. It has been previously shown that lymphoid recovery is enhanced in allo-HSCT recipients after IL-7 treatment. The combination of IL-7 treatment and castration appeared to have an additive effect in the thymus following HSCT. These results indicate that castration and the resulting ablation of sex steroids enhance hematopoietic recovery following allogeneic HSCT without increasing GVHD and maintaining GVT.

Materials and Methods:

Reagents.

Antimurine CD16/CD32 FcR block (2.4G2) and all following fluorochrome-labeled antibodies against murine antigens were obtained from Pharmingen (San Diego, Calif.): Ly-9.1(30C7), CD3(145-2C11), CD4 (RM4-5), CD8β.2(53-5.8), T-cell receptor-β (TCR-β; H57-597), CD45R/B220 (RA3-6B2), CD43 (S7), IgM-FITC (R6-60.2), CD11b (M1/70), Ly-6G(Gr-1) (RB6-8C5), c-kit (2B8), Sca-1 (D7), CD11c (HL3) I-A^{k} (11-5.2), isotypic controls: rat IgG2a-k (R35-95), rat IgG2a-1 (B39-4), rat IgG2b-(A95-1), rat IgG1-k (R3-34), hamster IgG-group1-k (A19-3), hamster IgG-group 2-1 (Ha4/8), and 2.4G2 and Fcr (FcR blocking). Streptavidin-FITC, PercP-phycoerythrin (PE) also were obtained from Pharmingen (San Diego, Calif.). Recombinant human IL-7 was provided by Dr Michel Morre (Cytheris, Vanves, France).

To confirm that the human recombinant IL-7 could stimulate murine cells, thymidine incorporation proliferation assays were performed with an IL-7-dependent murine pre-B cell line 2E8 and found that the human IL-7 used in the studies had a proliferative effect on murine cells that was equal to murine IL-7. Tissue culture medium consisted of RPMI 1640 supplemented with 10% heat inactivated fetal calf serum, 100 U/mL penicillin, 100 mg/nL streptomycin, and 2 mM L-glutamine (as well as 50 mM 2-mercaptoethanol for the culture of 32Dp210 cells and proliferation assays).

Mice and HSCT.

Male C57BL/6J (B6, H-2b), C3FeB6F1/J([B6 3 C3H]F1; H-2b/k), B10.BR (H-2k), B6D2F1/J (H-2b/d), CBA/J (H-2k), Balb/c (H2-d), IL7−/− and KGF−/− mice were obtained from the Jackson Laboratory (Bar Harbor, Maine) and used in experiments when they were between 8 and 12 weeks of age. KGF−/− and IL7−/− were used between 4 and 7 months of age. HSCT protocols were approved by the Memorial Sloan-Kettering Cancer Center Institutional Animal Care and Use Committee. The BM cells were removed aseptically from femurs and tibias. Donor BM was depleted of T cells by incubation with anti-Thy-1.2 for 30 minutes at 4° C. followed by incubation with Low-TOX-M rabbit complement (Cedarlane Laboratories, Hornby, ON, Canada) for 1 hour at 37° C. Splenic T cells (for GVHD analysis) were obtained by purification over a nylon wool column followed by red cell removal with ammonium chloride red cell lysis buffer. Cells (5×10⁶ BM cells with or without splenic T cells and leukemia cells) were resuspended in Dulbecco modified essential medium (Life Technologies, Grand Island, N.Y.) and transplanted by tail vein infusion (0.25 mL total volume) into lethally irradiated recipients on day 0. Prior to transplantation, on day 0, recipients received 1300 cGy total body irradiation (¹³⁷Cs source) as split dose with 3 hours between doses (to reduce gastrointestinal toxicity). Mice were housed in sterilized microisolator cages and received normal chow and autoclaved hyperchlorinated drinking water (pH 3.0).

Surgical Castration.

Mice were anaesthetized and a small scrotal incision was made to reveal the testes. These were sutured and removed along with surrounding fatty tissue. The wound was closed using surgical staples. Sham-castration required the same surgical procedure, except for the removal of the testes. Castration was performed one day prior to BM transplant for both immune reconstitution and GVHD studies.

Administration of IL-7.

IL-7 were either given from days 0 to 13 or 21 to 27 intraperitoneally at 10 μg/day for immune reconstitution studies. PBS was injected into control mice at the same time points.

Flow Cytometric Analysis.

BM cells, splenocytes or thymocytes were washed in FACS buffer (phosphate buffered saline (PBS)/2% bovine serum albumin (BSA)/0.1% azide) and 1-3×10⁶ cells were incubated for 30 minutes at 4° C. with CD16/CD32 FcR block. Cells were then incubated for 30 minutes at 4° C. with primary antibodies and washed twice with FACS buffer. Where necessary, cells were incubated with conjugated Streptavidin for a further 30 minutes at 4° C. The stained cells were resuspended in FACS buffer and analyzed on a FACSCalibur™ flow cytometer (Becton Dickinson, San Jose, Calif.) with CellQuest™ software.

Proliferation Assays.

Splenocytes (4×10⁵ cells/well) were incubated for 5 days with irradiated (2000 cGy) BALB/C splenocytes as stimulators (2×10⁵ cells/well) in 96-well plates and splenocytes (4×10⁵ cells/well) were stimulated with αCD3 (145-2c11) and αCD28 (37.51) (2.5 mg/mL as a final concentration) for 4 days. Cultures were pulsed during the final 18 hours with 1 mCi/well [3H]-thymidine and DNA was harvested on a Harvester 96 (Packard). Stimulation indices (SI) were calculated as the ratio of stimulated cells (cpm) over unstimulated cells (cpm).

51Cr Release Assays.

Target cells were labelled with 100 mCi 51Cr at 2×10⁶ cells/mL for 2 hours at 37° C. and 5% CO₂. After 3 washes, labelled targets were plated at 2.5×10³cells/well in U-bottomed plates (Costar, Cambridge, Mass.). Splenocytes cultured with irradiated BALB/C splenocytes (1:2 ratio) for 5 days were added at various effector-to-target ratios in a final volume of 200 mL to 4 to 6 wells and incubated for 4 to 6 hours at 37° C. and 5% CO₂. Subsequently, 35 mL supernatant was removed from each well and counted in a gamma counter (Packard, Meriden, Conn.) to determine experimental release. Spontaneous release was obtained from wells receiving target cells and medium only, and total release was obtained from wells receiving 5% Triton X-100. Percent cytotoxicity was calculated by the following formula: percent toxicity=100×[(experimental release−spontaneous release)/(total release−spontaneous release)].

Detection of Alloreactive T-Cell Clones with Intracellular IN-γ Staining

Briefly, cells were incubated for 12 to 15 hours (for secondary allogeneic stimulation with T cell-depleted [TCD], irradiated stimulator cells) with Brefeldin A (10 mg/mL), harvested, washed, stained with primary (surface) fluorochrome (FITC, PerCP, and APC)-conjugated antibodies, fixed, and permeabilized with the Cytofix/Cytoperm kit (Pharmingen), and subsequently stained with αIFNγ-PE. FACS analysis was conducted by gating for the designated populations. Flow cytometer and software were used as mentioned below.

Delayed Type Hypersensitivity Assay.

Sham-castrated and castrated mice were sensitized day 42 after allo-BMT by tail vein injection with 200 μl of 0.01% sheep red blood cells (Colorado Serum, Denver, Colo.) in PBS. Sensitized animals were challenged at day 46 in the right hind footpad with 50 μl of 20% sheep RBC suspension while the left hind footpad received the same volume of 50 μl of PBS solution as a control. 24 and 48 hr later footpad swelling was measured with a dial-thickness gauge (Mitutoyo, Kanagawa, Japan). The magnitude of the response was determined by subtracting measurements of PBS-injected left footpads from the experimental right ones.

Assessment of GVHD.

The severity of GVHD was assessed with a clinical GVHD scoring system as first described by Cooke et al. ((1996) Blood 88:3230-9). Briefly, ear-tagged animals in coded cages were individually scored every week for 5 clinical parameters (weight loss, posture, activity, fur, and skin) on a scale from 0 to 2. A clinical GVHD index was generated by summation of the 5 criteria scores (0-10). Survival was monitored daily. Animals with scores of 5 or more were considered moribund and were humanely killed.

Assessment of GVT-P815 (H1-2d) Mastocytoma Induction and Assessment of Mastocytomic Death Versus Death from GVHD.

B6D2F1/J recipients received 1×10³ P815 (H-2d) cells intravenously on day 0 of allogeneic HSCT (5×10⁶ T cell depleted (TCD) BM cells and 5×10⁵ T cells of C57/BL6 origin). Survival was monitored daily and the cause of death after HSCT was determined by necropsy as previously described. Briefly, death from leukemia was characterized by hepatosplenomegaly and the presence of mastocytoma cells in liver and spleen on microscopic examination, whereas death from GVHD was defmed as the absence of hepatosplenomegaly and leukemic cells in liver and spleen, and the presence of clinical symptoms of GVHD as assessed by the clinical GVHD scoring system at the time of death.

Semi-Quantitative RT-PCR.

Total cellular RNA from whole BM was reverse-transcribed using Superscript II reverse transcriptase (Life Technologies, Rockville, USA). cDNA was PCR-amplified for 35 cycles (94° C. for 30 secs; 56° C. for 30 secs; 72° C. for 60 secs) with PCR Master Mix (Promega, Madison, USA. HPRT: 5′ CACAggACTAgAACACCT gC 3′ and 5′ gCTggTgAAAAggACCTCT 3′ TGFβ₁: 5′ CTACTgCTTCAgCTC CACAg 3′ and 5′ TgCACTTgCAggAgCgCAC 3′ and KGF: 5′ gCCTTgTCACg ACCTgTTTC 3′ and 5′ AgTTCACACTCgTAgCCgTTTg 3′.

Enzymic Digestion of IL7−/− Thymii.

IL7−/− mice contain a large proportion of CD45-thymic stromal cells and each thymus was subjected to enzymic digestion in 0.125% (w/v) collagenase/dispase (Roche Applied Sciences, Indianapolis, USA) with 0.1% (w/v) DNase, releasing most of the stromal and haematopoietic cells from the thymi allowing for the accurate calculation of thymic cellularity. Anti-CD45 was used to identify CD45-stromal cells.

Statistics.

All values are expressed as mean±SEM. The Mantel-Cox log-rank test was used for survival data and all other statistical analysis was performed with the nonparametric, unpaired Mann-Whitney U test. A P value of less than 0.05 was considered statistically significant.

Results:

I. Castration Increases BM, Thymic and Splenic Cellularity Following Allogeneic HSCT.

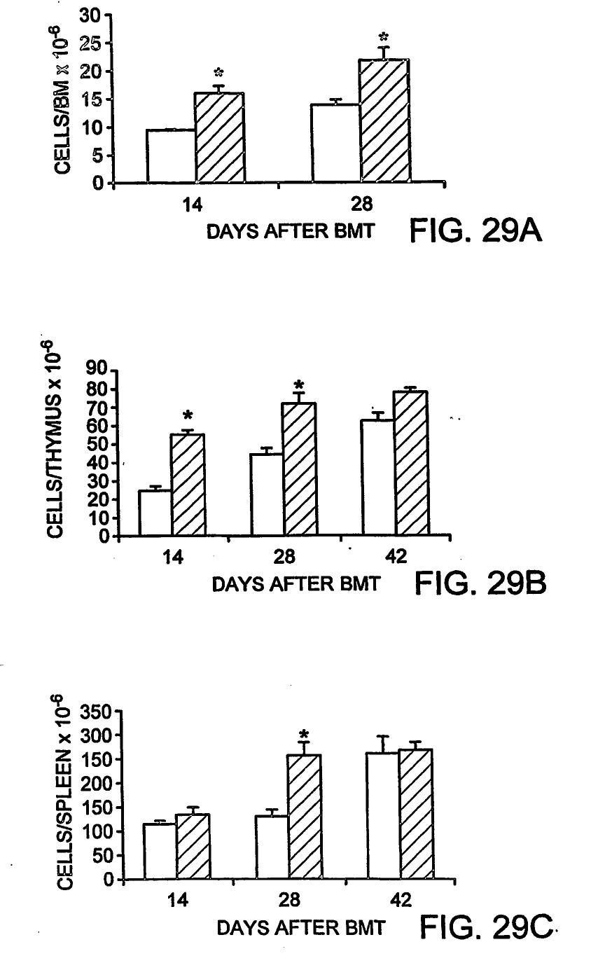

Male CBA mice were castrated one day prior to allogeneic HSCT. Mice were subjected to 1300 cGy total body irradiation followed by 5×10⁶ B10.BR TCD BM cells. There were significantly more cells in the BM (16×10⁶±1.4×10⁶) and thymus (55.4×10⁶±1.8×10⁶) of castrated mice, compared to the sham castrated controls (9.5×10⁶±3.0×10⁵ and 25×10⁶±2.6×10⁶, respectively), as early as 14 days after HSCT FIGS. 29A-B). These numbers remained significantly elevated in castrated mice 28 days after HSCT (BM: 22×10⁶±4.0×10⁶ vs. 14×10⁶±2.2×10⁶; thymus: 72×10⁶±5.9×10⁶ vs. 45×10⁶±2.9×10⁶). Splenic cellularity in the castrated mice was also significantly elevated above sham-castrated spleen cell numbers at day 28 (253×10⁶±28.4×10⁶ vs. 126×10⁶±13.9×10⁶) (FIG. 29C). The castrated mice had begun to approach pre-transplant cellularities by day 28. By 42 days after HSCT there was no longer a significant difference between castrated and sham-castrated mice with respect to thymic and splenic cellularity. Since the sham-recipients were young mice they had active post-transplant lymphopoiesis but the time required to generate normal cellularity in the primary and secondary lymphoid tissues was markedly delayed compared to castrated recipients.

II. There are Significantly More Donor-Derived HSCs in the BM of Castrated Mice 28 Days After HSCT.

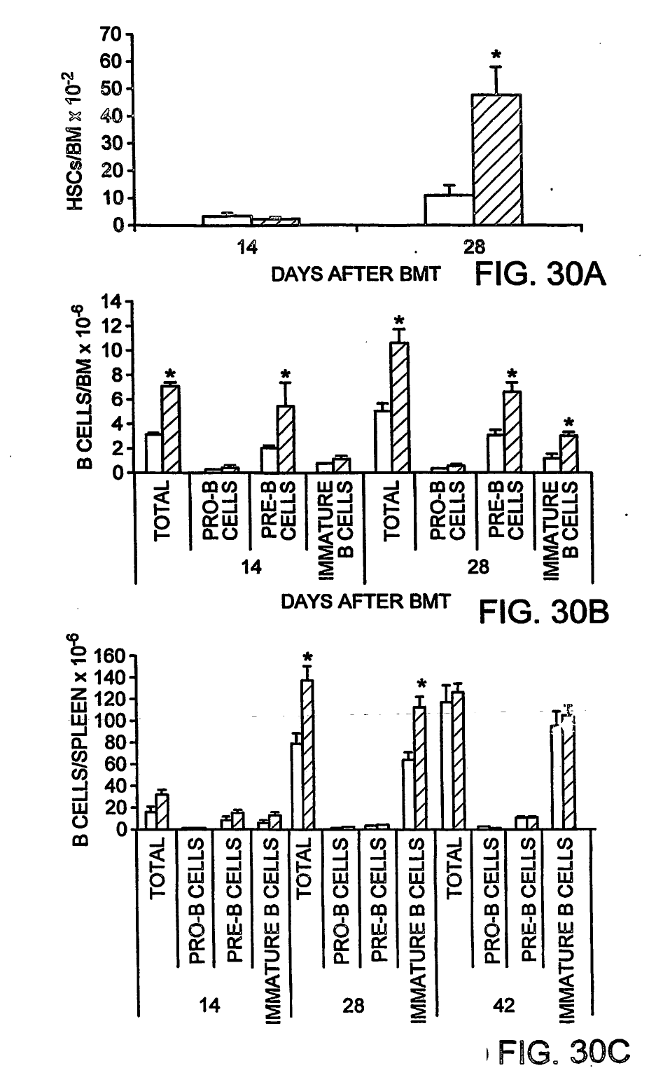

Several studies have shown that sex steroids inhibit the proliferation and/or differentiation of early hematopoietic precursors (Thurmond et al., (2000) Endocrinol. 141:2309-2318; Medina et al., (2001) Nat. Immunol. 2:718; Kouro et al., (2001) Blood 97:2708). Therefore, the impact of castration on the HSC numbers in the allogeneic transplant setting has been investigated. The number of donor-derived HSC was very low in both sham-castrated and castrated mice 14 days after allogeneic HSCT (2.98×10²±1.25×10² and 2.66×10²±8.8×10¹ respectively) (FIG. 30A). However, by day +28 there are significantly more Ly9.1⁺ Lin⁻ Sca-1⁺ c-kit⁺ donor-derived HSCs in the castrated mice (4.8×10³±1.1×10³), compared to the sham-castrated controls (1.1×10³±4.1×10²) (FIG. 30A).

III. Castration Prior to Allogeneic HSCT Enhances Donor-Derived B Cell Recovery.

In the analysis of B cell recovery three stages in B cell development was distinguished: Pro-B cells (CD45R⁺CD43⁺IgM⁻), pre-B cells (CD45R⁺CD43⁻IgM⁻) and immature B cells (CD45R⁺CD43⁻IgM⁻). Fourteen days after allogeneic HSCT, there were significantly more pre-B cells in the BM of castrated mice (5.5×10⁶±1.7×10⁶) compared to the sham-castrated controls (2.08×10⁶±5.0×10⁴) (FIG. 30B). At 28 days there were also significantly more pre-B cells (sham-cx: 3.1×10⁶±3.7×10⁵ c.f. cx: 6.6×10⁶±6.6×10⁵) and immature B cells (sham-cx: 1.3×10⁶±22.6×10⁵ c.f. cx: 3.0×10⁶±3.4×10⁵) in the BM of castrated mice (FIG. 30B). The increase in BM B cells and their precursors translated to a significant increase in the number of immature B cell in the spleens of castrated mice, 28 days after HSCT (sham-cx: 64.9×10⁶±6.4×10⁶ cf. cx: 112.0×10⁶±10.0×10⁶) (FIG. 30C). These results are in agreement with previous studies that suggest that castration enhances B cell production and export from the BM.

IV. T Cell Reconstitution Following Allogeneic HSCT is Enhanced by Castration.

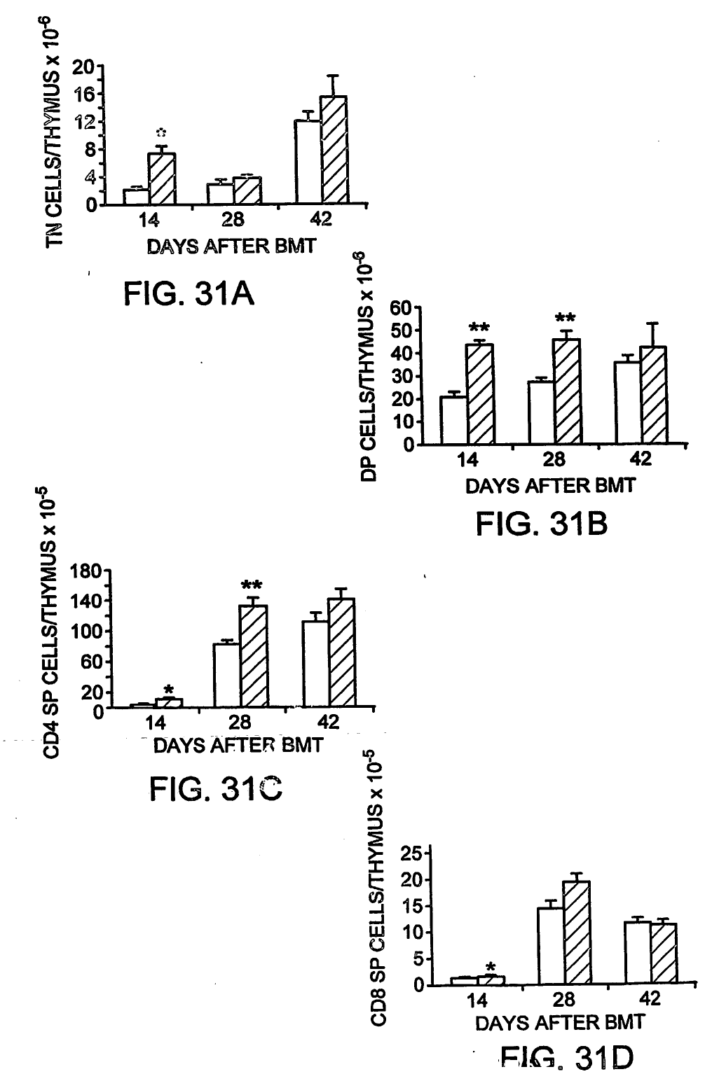

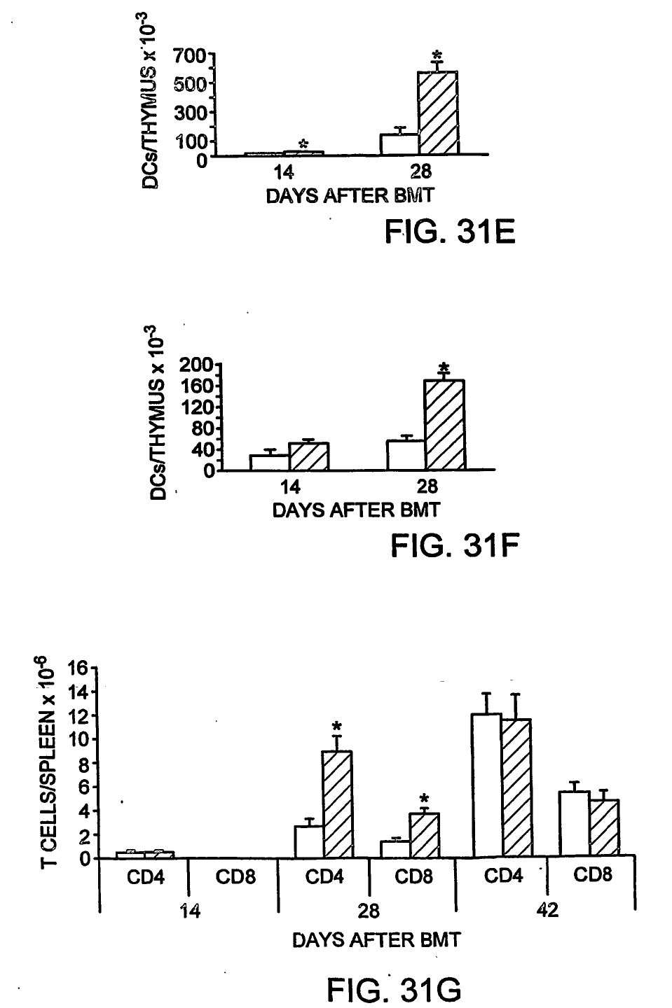

Thymocytes and peripheral cells were divided into developmental stages on the basis of expression of CD3, CD4 and CD8: Triple Negatives (TN) (CD3⁻CD4⁻CD8⁻), double positive (DP) (CD4⁺CD8⁺), single positive CD4 (SP CD4) CD3⁺CD4⁺CD8⁻ and single positive CD8 (SP CD8) CD3⁺CD4⁻CD8⁺ (FIGS. 31A-D). As early as 14 days after allogeneic HSCT there are significantly more TN, DP, SP CD4 and SP CD8 thymocytes in castrated mice compared to sham castrated controls. 28 days after HSCT, DP and CD4 SP cell numbers remain significantly elevated in the castrated group. By day 42, all thymocyte subsets are equivalent in sham-castrated and castrated mice. Both host and donor-derived DC are thought to play an integral role in the avoidance of graft rejection (Morelli et al., (2001) Semin. Immunol. 13:323-335). Fourteen days after allogeneic HSCT, there are significantly more host-derived CD11c^{hi} DC in the thymii of castrated mice. Both host and donor-derived DC in the thymus were significantly increased in castrated mice 28 days after allogeneic HSCT (FIGS. 31E-F). The increase in thymocyte numbers in castrated mice translated to a significant increase in the number of donor-derived mature CD4⁺ and CD8⁺ T cells in the spleens of castrated mice compared to the sham-castrated controls at day 28 (FIG. 31G).

V. On a Per Cell Basis, There is no Significant Difference Between T Cells from Sham-Castrated and Castrated Mice.

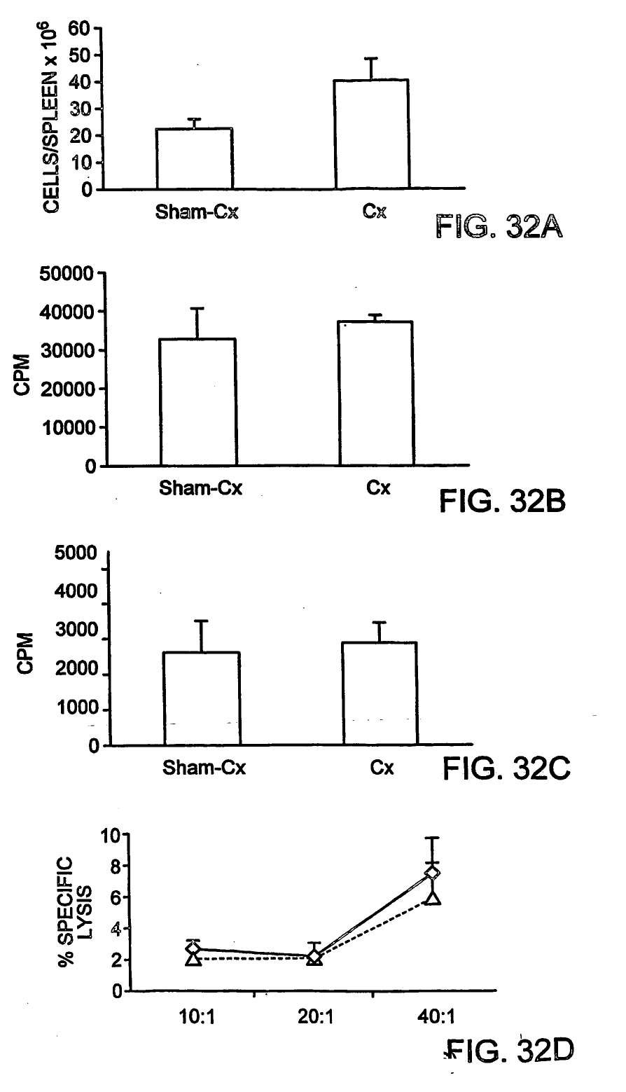

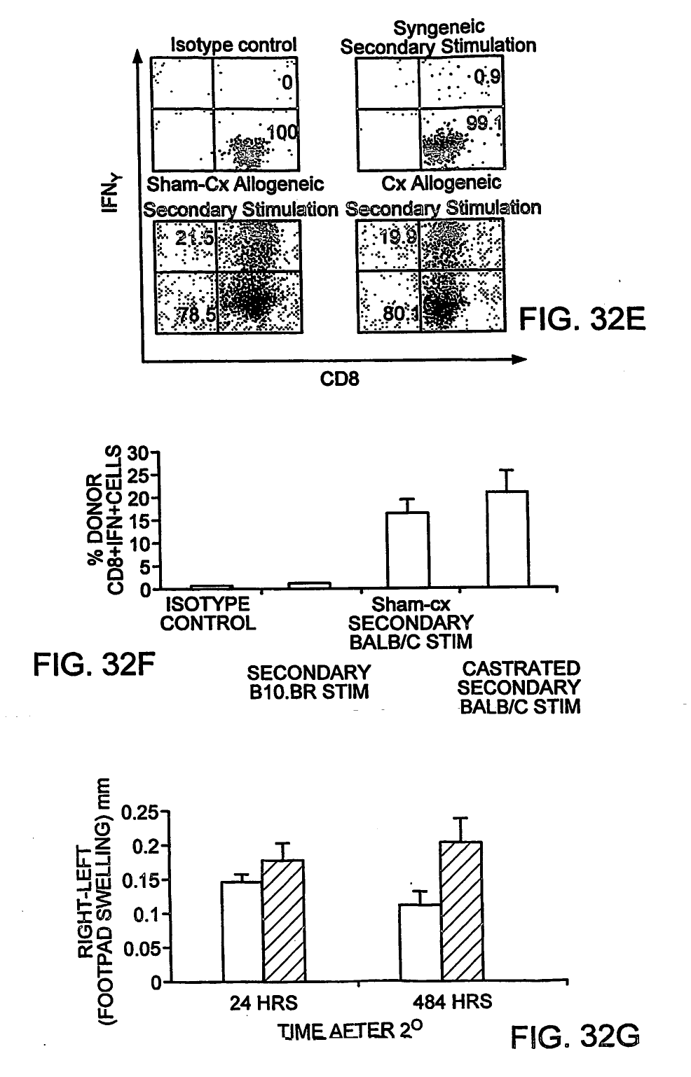

In order to determine the functional potential of peripheral T cells in castrated mice after allo-HSCT, a series of in vitro assay were performed. The number of donor-derived T cells in castrated and sham-castrated controls 6 weeks after allo-HSCT are represented in FIG. 32A. The proliferative capacity of the splenic T cells was tested in 2 ways: αCD3/αCD28 cross-linking (FIG. 32B) and in a 3^{rd} party MLR (using irradiated BALB/C splenocytes as stimulators) (FIG. 32C). There is no significant difference in the proliferative capacity of peripheral T cells when comparing sham-castrated and castrated mice in either of these settings. Six weeks after allo-HSCT splenocytes were cultured with irradiated BALB/C splenocytes (3^{rd} party) for 5 days. Following 5 days of allogeneic stimulation the vast majority of cells in culture were CD8⁺ T cells. Half these cells were used in a CTL (⁵¹Cr release) assay to determine the cytotoxicity of splenocytes from sham-castrated and castrated mice. Splenocytes were tested for their ability to kill ⁵¹Cr loaded A20 (BALB/C B cell lymphoma tumour cell line) cells at different effector:_target ratios (FIG. 32D). There was no significant difference between sham-castrated and castrated mice with respect to cytotoxicity. The other half of the cells cultured for 5 days were restimulated overnight with either 3^{rd} party (BALB/C) or syngeneic (B10.BR) irradiated splenocytes and Brefeldin A to determine IFN-γ production. FIG. 32E shows IFNγ production by donor-derived CD8⁺ splenic T cells following BALB/C primary stimulation and either BALB/C or B10.BR secondary stimulation (control). This is represented graphically in FIG. 32F. There is no significant difference in the proportion IFN-γ producing donor-derived CD8⁺ when comparing sham-castrated and castrated mice.

In order to assess immune function in vivo a DTH assay was used whereby, 42 days after castration and allo-BMT mice were sensitised with sRBCs. On day 46 days they were challenged and 24 and 48 hr later footpad swelling was determined. The DTH response is significantly enhanced 48 hrs after challenge when mice are castrated at the time of allo-HSCT compared to sham-castrated controls (FIG. 32G).

These functional assays demonstrate that the T cells in castrated recipients are comparable on a per cell basis with T cells from sham-castrated recipients and are capable to respond to novel antigens with intact proliferation, cytotoxicity and cytokine production. However, the significantly more rapid T cell reconstitution in castrated recipients translates in an enhanced DTM response even at 6 weeks after transplant.

VI. Castration Prior to Allogeneic-HSCT does not Exacerbate GVHD and Maintains GVT Activity.

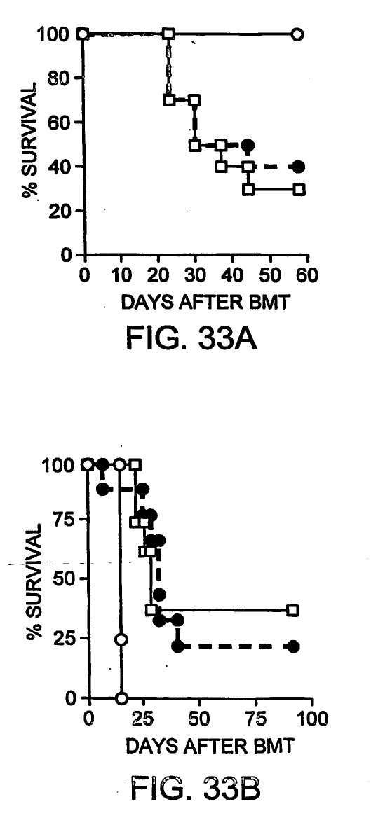

Both GVHD and GVT are mediated, primarily, by alloreactive donor-derived T cells, which are transferred with the allograft. Any treatment used to enhance immune reconstitution has the potential to exacerbate GVHD or, conversely, decrease GVT activity. To establish that castration does not have a stimulatory effect on alloreactive T cells of donor origin, GVHD was induced by the addition of allogeneic donor T cells to the allograft. There was no significant difference in morbidity or mortality due to GVHD when comparing castrated and sham-castrated mice (FIG. 33A). To assess the effects of castration on GVT activity the mastocytoma cell line P815 (H-2d) was injected into B6D2F1/J recipients at the time of transplant. Animals that died during the experiment were autopsied and the cause of mortality (tumor vs. GVHD) was determined. Mortality due to mastocytoma remained unchanged following castration (six out of nine mice) when compared to sham-castrated controls (five out of eight mice). This suggests that castration does not diminish GVT response following HSCT (FIG. 33B).

VII. IL-7 and Castration Have and Additive Effect Following Allogeneic HSCT.

It has previously been shown that IL-7 treatment can increase the number of T and B cells in otherwise untreated animals and can also enhance lymphoid recovery following cyclophosphamide treatment, irradiation, syngeneic and allogeneic HSCT (Alpdogan et al., (2001) Blood 98:2256; Bolotin et al., (1996) Blood 88:1887; Faltynek et al., (1992) J. Immunol. 149:1276; Morrissey et al., (1991) J. Immunol. 146:1547). IL-7 is known to increase T cell numbers through increased thymic activity as well as peripheral expansion.

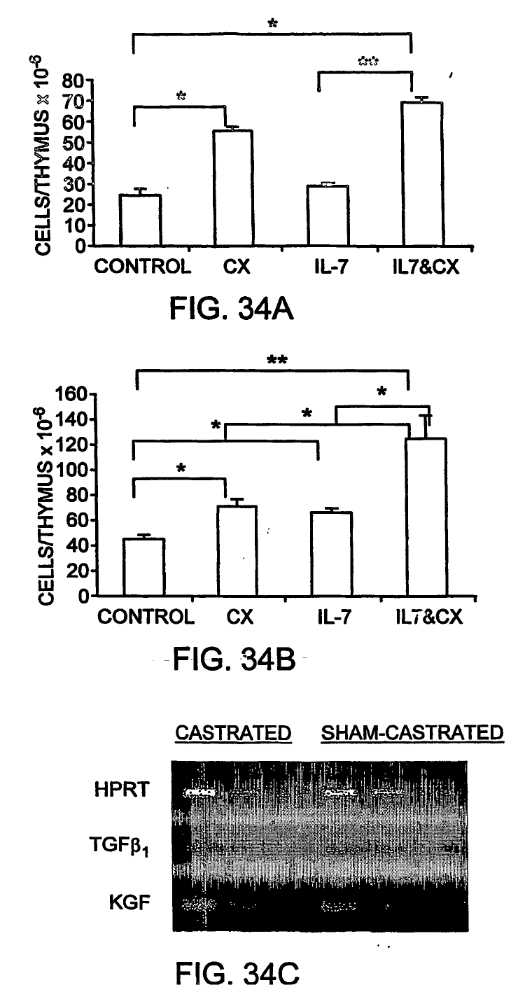

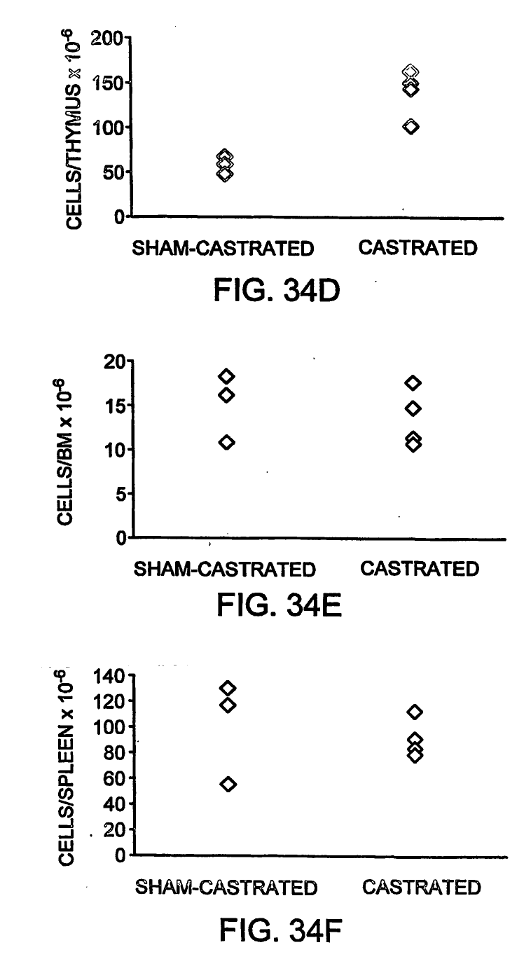

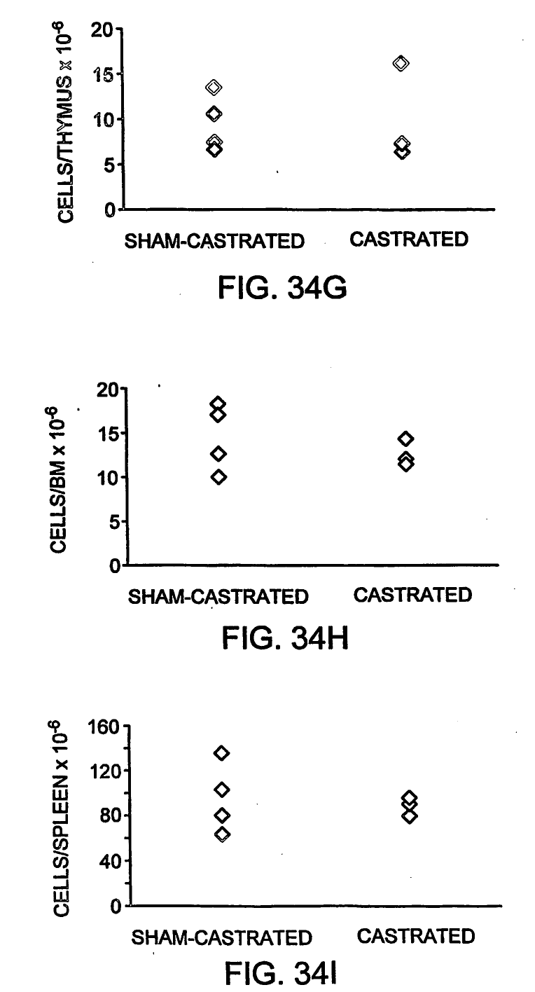

It was, therefore, decided to combine IL-7 administration with castration in recipients of allogeneic HSCT. 14 days after treatment there are significantly more cells in the thymi of castrated mice and those given the combined treatment (castration and IL-7 administration). At this early time point there is no difference seen between the PBS treated, sham-castrated controls and the IL-7 treated, sham-castrated mice. There is also no significant difference seen between the castrated group and those receiving the combined treatment suggesting that it is only the effects of castration acting 14 days after allo-HSCT, IL-7 treatment and castration (FIG. 34A). At a later time point, 28 days after allo-HSCT, the cellularity of the thymi in both the castration alone group and the IL-7 alone group is significantly higher that the control group. The combination of IL-7 treatment and castration had an additive effect on thymic cellularity when analyzed 28 days after allogeneic HSCT (FIGS. 34B).

VIII. Semi-Quantitative RT-PCR for IL-7, TGF-β₁ and KGF Reveals an Increase in KGF and a Decrease in TGF-β₁ Following Allo-HSCT and Castration.

RT-PCR analysis of whole bone marrow cells revealed undetectable levels of IL-7 transcript in both sham-castrated and castrated mice as late as 6 weeks after allo-HSCT. When template from control, untransplanted mice was used IL-7 was detected (data not shown). TGFβ₁ and KGF are known to be key mediators of hematopoiesis. Using HPRT equilibrated template there appears to be a decrease in TGFβ₁and an increase in KGF 2 weeks after castration and allo-BMT (FIG. 34C).

IX. Changes that Occur Following Castration were Seen in KGF^{-/-} Mice but not IL7^{-/—} Mice.

In order to further study the possible mechanisms behind the enhanced immune reconstitution following castration KGF^{-/-} and IL7^{-/-} mice (4-6 months old) were castrated and 2 weeks later thymus, spleen and BM were analysed (TGFβ₁^{-/-} mice die prepubertally, Shull et al. (1992) Nature 359:693). Thymic cellularity is significantly increased when comparing sham-castrated and castrated KGF^{-/-} mice. Although no differences were seen in the total cellularity of the BM and spleen at this early time point, changes were seen in the B cell compartment of the BM, as seen previously in wild-type mice (Ellis et al. (2001) Int. Immunol. 13:553; data not shown). Due to the fact that a large proportion of cells in the thymi of IL7^{-/-} mice are CD45⁻ stromal cells, enzymic digestion was used to obtain a single cell suspension when using these mice. By doing this many more cell are released into suspension which accounts for the slightly larger thymic cellularity seen in this experiment compared to previous literature (von Freeden-Jeffry et al. (1995) J. Exp. Med. 181:1519). No differences were seen in the thymii (FIG. 34G), spleen (FIG. 34H) or BM (FIG. 34I) of IL7^{-/-} mice when comparing castrated mice and sham-castrated controls. These finding suggest an important role for IL-7 in the enhanced immune reconstitution seen following castration and allo-HSCT.

Discussion:

Recipients of an allogeneic HSCT experience a prolonged period of immune deficiency, which is associated with life-threatening infections. With increasing age of the recipient, this infection risk increases, as does the time it takes for full immunological reconstitution. The period of immunodeficiency following HSCT can be greater than one year, and recent long-term studies demonstrated a decrease in TREC+CD4+ T cells in older HSCT patients compared to their donors (Storek et al., (2001) Blood 98:3505); Lewin et al., (2002) Blood 100:2235). This suggests that thymic damage and the subsequent decline in T cell production may be more prolonged than once thought. The majority of post-HSCT infections are associated with a lack of CD4+ peripheral T cells (Storek et al., (1997) Am. J. Hematol. 54:131). Therefore, the increase in peripheral T cell number that occurs following castration may decrease the incidence of these infections leading to enhanced overall survival of transplant patients.

Many groups have focused their research on hematopoietic reconstitution following HSCT, and the most promising results have come with the use of haematopoietic growth factors and cytokines. G-CSF, for example, is used to mobilise donor stem cells (Dreger et al., (1993) Blood 81:1404). Noach et al., showed that pre-treatment with SCF and IL-11 or SCF and Flt-3 ligand resulted in enhanced donor cell engraftment ((2002) Blood 100:312). KGF appears to enhance engraftment and reconstitution in both syngeneic and allogeneic settings as well as ameliorating GVHD (Panoskaltsis-Mortari et al., (2000) Blood 96:4350; Krijanovski, et al., (1999) Blood 94:825). IL-7 enhances immune reconstitution following syngeneic HSCT (Bolotin et al., (1996) Blood 88:1887) and can enhance immune reconstitution and maintain GVT activity without exacerbation of GVHD after allogenic HSCT (Alpdogan et al., (2001) Blood 98:2256).

Several studies have shown that sex steroid ablation, be it by surgical or chemical castration, of male mice increases both BM and splenic B cell numbers (Ellis et al., (2001) Int. Immunol. 13:553; Erben et al., (2001) Horm. Metab. Res. (2001) 33:491; Wilson et al., (1995) Blood 85:1535; Masuzawa et al., (1994) J. Clin. Invest. 94:1090). The increase in peripheral B cell number is predominantly due to an increase in B220^{lo}CD24^{hi} recent BM emigrants Ellis et al., (2001) Int. Immunol. 13:553). Olsen et al., have demonstrated that androgens enhance the production of TGF-β by stromal cells within the BM, which in turn suppresses B cell development (J. Clin. Invest. (2001) 108:697). In addition, neutralization of TGF-β in vitro reverses B cell suppression by dihydrotestosterone. TGF-β has also been shown to down-regulate stromal IL-7 production and subsequently inhibit the proliferation of B cell progenitors (Tang et al., (1997) J. Immunol. 159:117). Therefore one possible explanation for the effects of castration/androgen ablation, in this instance, following allogeneic HSCT, suppresses the production of TGF-β, in turn enhancing B cell development, explaining the increased B cell numbers in the BM and spleen of castrated mice compared to the sham-castrated controls.

The proliferation of hematopoietic stem cells is also regulated by TGF-β. Batard et al., have demonstrated that physiological concentrations of TGF-β₁ inhibit the proliferation and differentiation of HSCs in vitro ((2000) J. Cell. Sci. 113:383-90). Furthermore, disruption of TGF-β signaling in HSCs (via the transient expression of a mutant type II receptor) enhances survival and proliferation of these cells (Fan et al., (2002) J. Immunol. 168:755-62). It is therefore possible that the increased number of HSCs seen 28 days after allogeneic HSCT and castration may be due do a decrease in the production of TGF-β by BM stromal cells.

Both estrogen and androgen can effect the differentiation and proliferation of HSCs (Thurmond et al., Endocrinol., (2000) 141:2309-18; Medina et al., (2001) Nat. Immunol. 2:718-24; Kouro et al., (2001) Blood 97:2708-15). Estrogen directly inhibits the proliferation and differentiation of HSC, as well as some lymphoid precursor subsets (Medina et al., (2001) Nat. Immunol. 2:718; Kouro et al. (2001) Blood 97:2708). HSCs express functional estrogen receptors (ERs) and estrogen administration decreases the number of Lin⁻c-kit⁺Sca-1⁺ HSCs (Thurmond et al., (2000) Endocrinol. 141:2309; Kouro et al., Blood (2001) 97:2708). The study conducted by Thurmond et al., suggests that the transition between c-kit⁺Sca-1⁺ precursors and the more mature subsets (c-kit⁺Sca-1⁻ and c-kit⁻Sca-1⁻ is blocked when ERα is present in the hematopoietic cells of the BM (Endocrinol. (2000) 141:2309). ERs are also present on BM stromal cells (Girasole et al., (1992) J. Clin. Invest. 89:883; Smithson et al., (1995) J. Immunol. 155:3409) suggesting that estrogen may also have an effect on the production of growth factors by the stroma, which in turn affects HSC proliferation and/or differentiation. Although most evidence suggests an indirect effect of androgens on HSCs via the BM stroma, the presence of functional androgen receptors on lymphoid components of the BM does not exclude a direct effect.

Olsen et al. have shown that it is the presence of a functional androgen receptor on the thymic epithelium but not the thymocytes that is essential for age-related thymic involution and the subsequent regeneration via sex steroid ablation Olsen et al., (2001) Endocrinol. 142:1278).

Although the molecular mechanisms for thymic involution and/or regeneration remain unknown there are several potential candidates. Thymic IL-7 levels decline with age (Aspinall, et al., (2000) Vaccine 18:1629; Andrew et al., (2002) Exp. Gerontol. 37:455; Ortman et al., (2002) Int. Immunol. 14:813). It remains unclear as to whether this is due to a decrease in the number of cells that produce IL-7 or a decrease in the ability of the existing cells to produce the cytokine. However, IL-7 treatment of old mice can reverse age-related increases in thymic apoptosis and enhance thymopoiesis (Andrew et al., (2001) J. Immunol. 166:1524). Stem cell factor (SCF) and M-CSF mRNA expression is also decreased in the mouse thymus with age (Andrew et al., (2002) Exp. Gerontol. 37:455). At the intracellular signaling level, E2A a transcription factor essential for the development of DN thymocytes is decreased, as is Foxn1 (whn) a transcription factor present in and involved in the proliferation and differentiation of thymic epithelial cells (Ortman et al., (2002) Int. Immunol. 14:813). Sempowski et al., have monitored mRNA steady-state levels in ageing humans and shown a significant increase in Leukemia Inhibitory Factor (LIF), Oncostatin M, IL-6 and SCF-mRNA (J. Immunol. (2000) 164: 2180).

The above studies suggest that the response to castration is multifactorial. The experiments with castration of IL7^{-/-} mice suggest that increased production of IL-7 is an important component of the castration effect. However, an additive effect on thymic cellularity was observed when recipients were treated with both high dose IL-7 and castration, which would suggest that castration provides more thymopoietic effects than increased IL-7 levels alone.

DC are the key mediators of negative selection in the thymus (Jenkinson et al., (1985) Transplantat. 39:331; Matzinger et al., (1989) Nature 338:74-60) and in a transient setting have been implicated in inducing graft acceptance by presenting alloantigens in the thymus after transplantation, removing donor-specific T cells. Tomita et al. have demonstrated that donor-derived cells in the thymii of MHC class I mismatched recipients mediate deletion of donor reactive cells (Tomita et al., (1994) J. Immunol. 153:1087-1098). They have also shown that thymus-derived DC injected intravenously traffic to the host thymus. Furthermore, it has been shown that intrathymic injection of both host cells pulsed with allo-antigen, donor cells, or donor soluble peptides increases graft acceptance (Garrovillo et al., (1999) Transplantation 68:1827; Ali et al., (2000) Transplantation 69:221; Garrovillo et al., (2001) Am. J. Transplant. 1:129; Oluwole et al., (1993) Transplantation 56:1523-1527). These studies show that castration significantly increased the number of host and donor-derived DC in the thymus following allogeneic HSCT. That is, sex steroid ablation enhances the differentiation and/or proliferation of thymic DC. Thus, castration, used in conjunction with hematopoietic stem cells and solid organ transplantation, increases graft acceptance.

Conclusion:

The current study has revealed that blocking sex steroids has a profound positive effect on immune reconstitution following myeloablation and HSCT. HSCs, and B and T progenitors are markedly enhanced. This provides an important platform for increasing the efficiency of engraftment and post-transplant strategies that depend on an intact hematopoietic system, such as vaccination against tumor or microbial antigens or gene therapy targeting donor HSCT. The increase in DC in the thymus have importance in inducing and sustaining tolerance to allogeneic grafts. In addition, GVHD is not exacerbated and GVT activity is not diminished in castrated recipients. These results demonstrate that transient sex steroid ablation (using, e.g., LHRH analogs) are useful as a prophylactic therapy to enhance immune reconstitution.

### Example 18

### Sex Steroid Ablation Enhances TCR-Specific Stimulation Following Hematopoietic Stem Cell Transplantation

In order to assess the functional nature of regenerated T cells, patients were analyzed for responsiveness to TCR specific stimulation.

Materials and Methods:

Patients.

Test patients were given Zoladex (LHRH-A) 3-weeks prior to stem cell transplantation and then monthly injections for 4-months. All patients were analysed pre-treatment, weekly for 5-weeks after transplantation and monthly up to 12 months. Ethics approval was obtained from The Alfred Committee for Ethical Research on Humans (Trial Number 01/006).

Preparation of PBMC.

Purified lymphocytes were used for T-cell stimulation assays and TREC analysis and were prepared as above.

T Lymphocyte Stimulation Assay.

Analysis of TCR specific stimulation was performed using anti-CD3 and anti-CD28 cross-linking from 1-12 months post-transplant, unless otherwise indicated. For TCR-specific stimulation, cells were incubated for 48 hours on plates previously coated with purified anti-CD3 (1-10 μg/ml) and anti-CD28 (10 μg/ml). Following plaque formation (48-72 hours), 1 μCi of ³H-Thymidine was added to each well and plates incubated for a further 16-24 hours. Plates were harvested onto filter mats and incorporation of ³H-thymidine was determined using liquid scintillation on a β-counter (Packard-Coulter, USA).

I. LHRH-A Administration Enhances Responsiveness to TCR Specific stimulation Following Allogeneic stem Cell Transplantation.

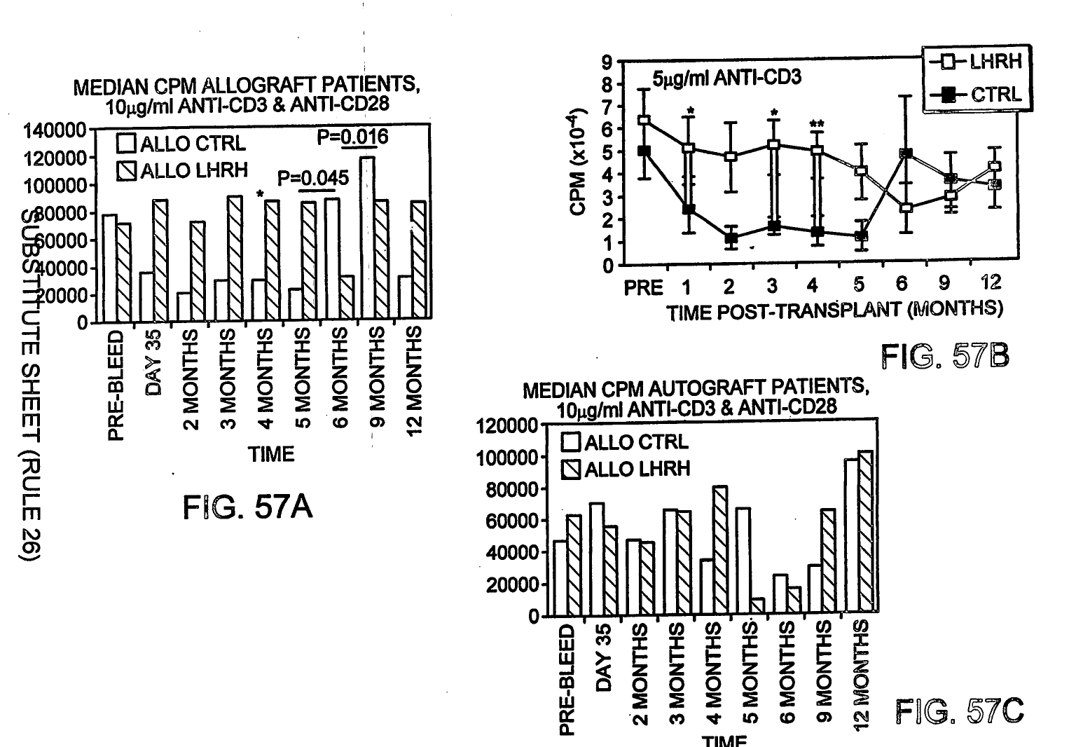

LHRH-A treated patients showed enhanced proliferative responses (assessed by ³H-thymidine incorporation) compared to control patients at all time-points except 6 and 9 months due to low patient numbers analyzed at this time; (FIGS. 57A-B). In allogeneic transplant patients treated with a LHRH-A, a significant increase in responsiveness to anti-CD3/CD28 stimulation was observed at 4 and 5-months post-transplant compared to control patients. While control patients showed an enhanced response at both 6 and 9 months post-transplant, LHRH-A treated patients showed a greater responsiveness at 12-months post-transplant. At 6 and 9 months post-transplant control patients had similar responsiveness to pre-treatment values. However at all other time-points, they were considerably lower. In contrast, LHRH-A treated patients had equivalent responsiveness at all time-points except 6 months compared to pre-treatment. LHRH-A treated patients showed enhanced proliferative responses (assessed by ³H-Thymidine incorporation) compared to control patients at 1, 3 and 4 months post-transplant. This indicates a contribution of direct peripheral T cell effects, as new CD4⁺ T cells are not evident until at least 1-2 months post-transplant (FIG. 57A-B).

II. LHRH-A Administration Enhances Responsiveness to TCR Specific Stimulation Following Autologous Stem Cell Transplantation.

A similar response as that seen in allograft recipients was also observed with autograft recipients (FIG. 57C). Those patients treated with a LHRH-A demonstrating an enhanced proliferative response to TCR stimulation at both 4 and 9 months post-transplant. LHRH-A treated patients showed enhanced proliferative responses (assessed by ³H-thymidine incorporation) compared to control patients at all time-points except 5 months. Restoration to pre-treatment values was observed by 12 months post-transplant in both control and LHRH-A treated patients.

III. LHRH-A Administration Enhances Responsiveness to TCR Specific Stimulation Following Treatment for Chronic Cancer Sufferers.

Patients with chronic haematological malignancies who were Immunosuppressed patients as determined by documented serious infection associated with: CD4<0.4×109/L; or lymphoproliferative disorder (e.g., CLL, myeloma, lymphoma) and receiving regular prophylactic intravenous gammaglobulin (documented hypogammaglobulinemia); or previous treatment with fludarabine, deoxycorfomycin and 2-CdA within 4 years; or prior allogeneic or autologous stem cell transplant within 2 years were enrolled in the study: Ethics approval was obtained from The Alfred Committee for Ethical Research on Humans (Trial number 01/006). Patents were given LHRH-A as follows and results are presented up to 6-weeks post-LHRH-A administration.

| | |
|---|---|
| d −1 (or before): | consent signed and pre-treatment investigations |
| | performed |
| d0: | Zoladex administered (males 10.8 mg; females 3.6 mg) |
| d +28: | females-Zoladex 3.6 mg administered |
| d +56: | females-Zoladex 3.6 mg administered |
| d +84: | females and males-Zoladex 3.6 mg administered |

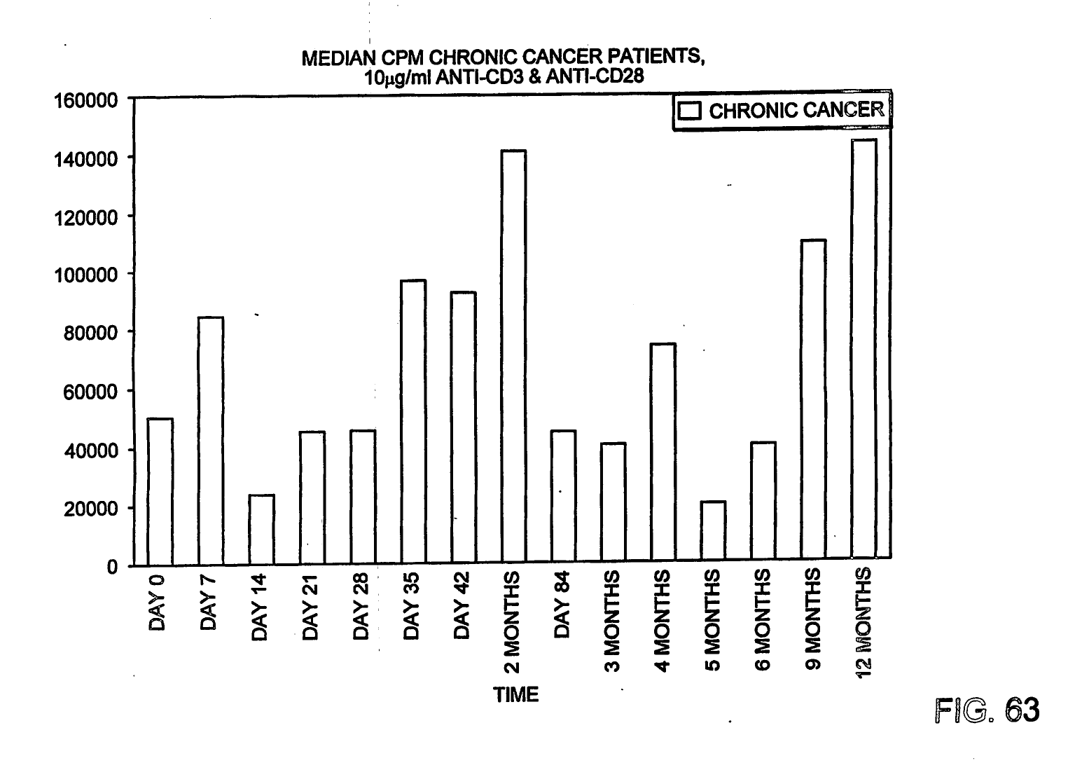

Analysis of TCR specific stimulation was performed using anti-CD3 and anti-CD28 cross-linking from D7 post-administration. LHRH-A treated patients showed enhanced proliferative responses (assessed by ³H-Thymidine incorporation) compared to pre-treatment levels in a “cyclical” fashion (FIG. 63). That is, on the day of injection, an increase in T cell proliferation was observed and this appeared to decrease slightly prior to the subsequent injection. This also indicates the probability of a direct influence of LHRH-A on existing peripheral T cells. This reflected the administration of the agonist with monthly depot injections. These results indicate an influence directly on peripheral T cells. However, the enhanced response seen at 12-months post-treatment reflect changes in thymic-derived T cells as well, since agonist administration was ceased from 4-months for all patients.

Conclusion

Since an increased responsiveness to TCR-specific stimulation was observed as early as D35 post-transplantation, this is due predominantly to pre-existing T cells, since newly-derived T cells only just begin to exit the thymus at this stage. The influence of newly-derived thymic-generated T cells is only observed after this time-point, so it can be concluded that the increased T cell function is due to an improvement in the pre-existing T cells and not T cells derived from the regenerated thymus.

### Example 19

### Sex Steroid Ablation Enhances Mitogenic Stimulation Following Hematopoeetic Stem Cell Transplantation

In order to assess the functional nature of regenerated T cells, patients were analyzed for responsiveness to mitogenic stimulation.

Materials and Methods:

Patients.

Patients were those enrolled in Clinical Trial protocol No. 01/006 as above. Prior to stem cell transplant, patients were given LHRH-A (3-weeks prior). Patients who did not receive the agonist were used as control patients.

Preparation of PBMC.

Purified lymphocytes were used for T-cell stimulation assays and TREC analysis and were prepared as above.

Mitogen Stimulation Assay.

Analysis of mitogenic responsiveness was performed using pokeweed mitogen (PWM) and tetanus toxoid (TT) from 1-12 months post-transplant. For mitogen stimulation, PBMC were plated out in 96-well round-bottom plates at a concentration of 1×10⁵/well in 100 μl of RPMI-FCS. Cells were incubated at 37° C., 5% CO₂ with TT (2LFAU/ml) or PWM (10 ug/ml). Following plaque formation (48-72 hours), 1 μCi of ³H-thymidine was added to each well and plates incubated for a further 16-24 hours. Plates were harvested onto filter mats and incorporation of ³H-thymidine was determined using liquid scintillation on a P-counter (Packard-Coulter, USA).

I. LHRH-A Administration Enhances Responsiveness to Mitogenic Stimulation Following Allogeneic Stem Cell Transplantation.

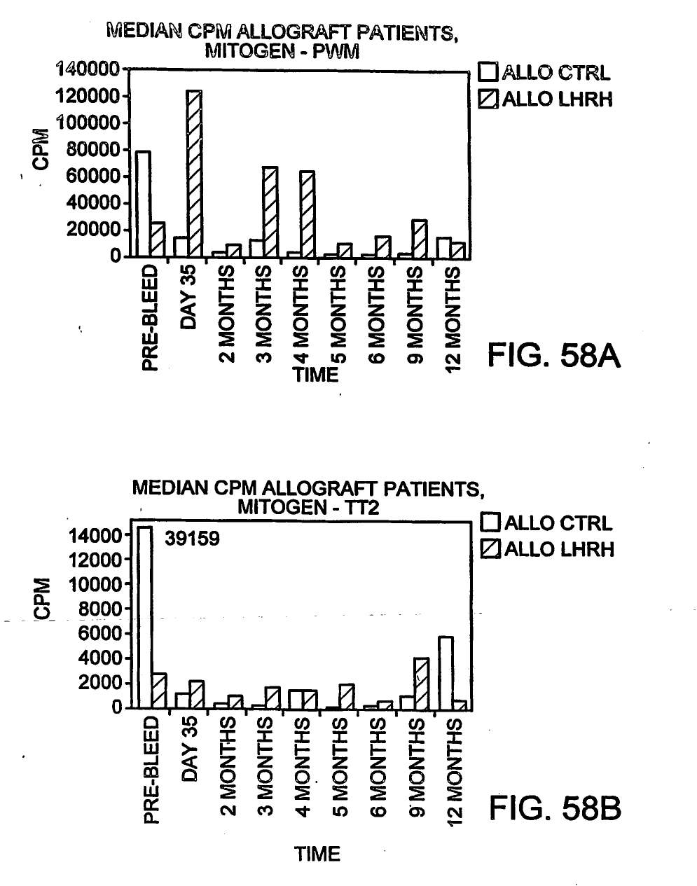

Analysis of mitogen responsiveness showed that allogeneic patients undergoing LHRH-A treatment had an increased responsiveness to PWM at all time-points post-transplant compared to controls (FIG. 58A). That is, patients treated with LHRH-A prior to stem cell transplantation showed an enhanced responsiveness to PWM stimulation at all time-points studied compared to control patients.

Similar results were evident following analysis of response to TT (FIG. 58B). LHRH-A treated patients had enhanced responses at all time-points compared to control patients except at 12 months post-transplantation.

II. LHRH-A Administration Enhances Responsiveness to Mitogenic Stimulation Following Autologous Stem Cell Transplantation.

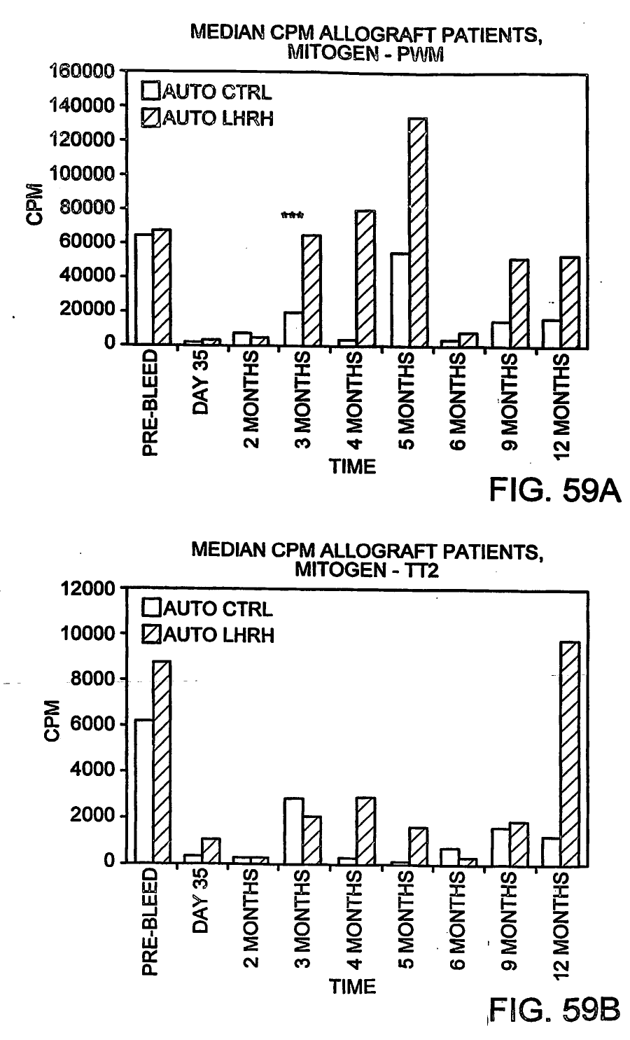

Patients treated with LHRH-A prior to stem cell transplantation showed an enhanced responsiveness to PWM stimulation at the majority of time-points studied compared to control patients (p≦0.001 at 3 months) (FIG. 59A). By 12-months post-transplantation, LHRH-A treated patients had restored responsiveness to pre-treatment levels while control patients were still considerably reduced.

Also, similar to allograft patients above (FIG. 58B), autograft patients also showed an increased response to TT when given LHRH-A prior to treatment Patients treated with LHRH-A prior to stem cell transplantation showed an enhanced responsiveness to TT stimulation at the majority of time-points studied compared to control patients (FIG. 59B). By 12-months post-transplantation, LHRH-A treated patients had restored responsiveness to pre-treatment levels while control patients were still considerably reduced.

Since an increased responsiveness to mitogenic stimulation was observed as early as D35 post-transplantation, this is due predominantly to pre-existing T cells since newly-derived T cells are only just beginning to exit the thymus at this stage. The influence of newly-delivered thymic-generated T cells is observed only after this time-point.

### Example 20

### Sex Steroid Ablation Enhances Rate of Engraftment in Hematopoeetic Stem Cell Transplant Patients

Materials and Methods:

Materials and methods used for these experiments are described above. Additional materials and methods are as follows:

Allogeneic and autologous patients (control or LHRH-A treated) were analyzed for total WBC and total granulocyte or neutrophil numbers following HSCT. Three weeks prior to HSCT, patients were treated with LHRH-A. Patients who did not receive the agonist were used as control patients. Total white blood cell (WBC) counts, granulocyte (G) and neutrophil counts per μl of blood were determined up to 35 days post transplant. A sample of whole peripheral blood was analyzed either using a Cell-Dyn 1200 automated cell counter (Abbott) or hemocytometer counts done in duplicate. This allows calculation of total white blood cells, lymphocytes and granulocyte numbers following transplant. Analysis of engraftment was performed from D14-D35 post-transplant.

Results:

I. Autologous Stem Cell Transplant Patients Undergoing LHRH-A Treatment Prior to Transplant Enhance Rate of Engraftment.

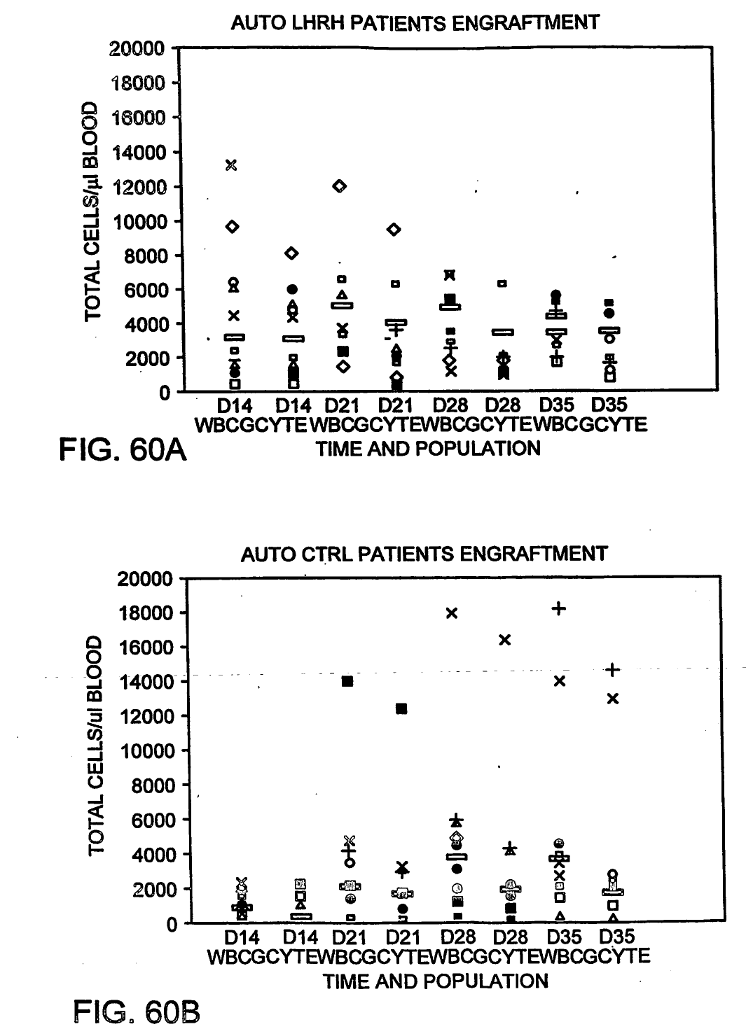

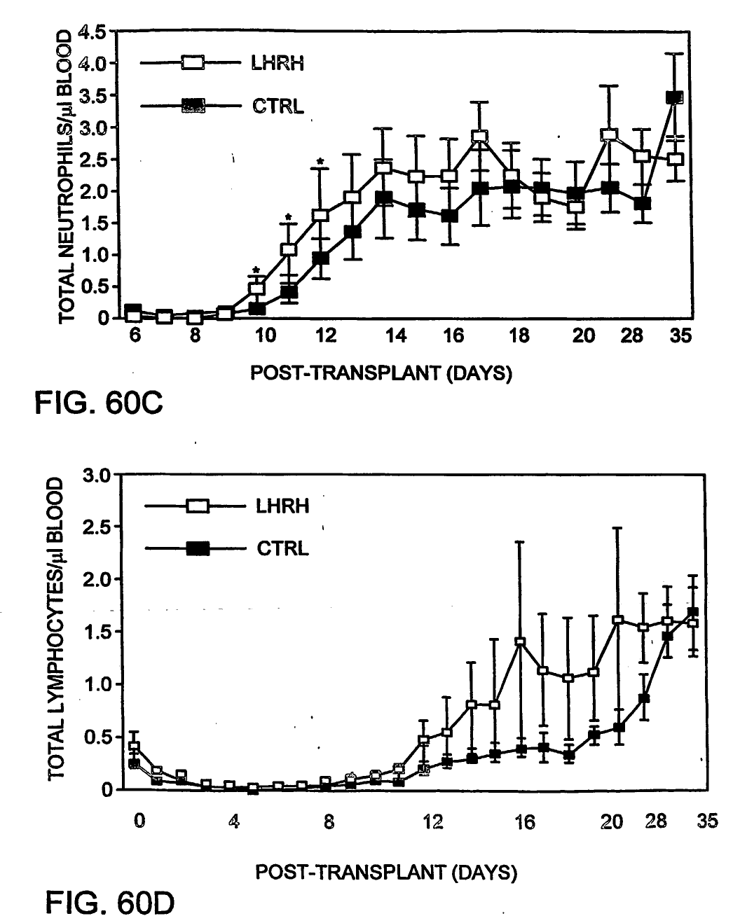

Total white blood cell (WBC) counts and granulocyte (G) counts per μl of blood were determined at days 14, 28, and 35 post transplant. As shown in FIGS. 60A-D, autologous patients who were given LHRH-A treatment showed a significantly higher number of WBC at D14 post-transplant compared to controls (FIGS. 60B) (p≦0.05), with 87% showing granulocyte engraftment (≧500 cells/μl blood) compared to 45% of controls (p≦0.05) at this time point. Autologous patients who were given LHRH-A treatment also showed a significantly higher number of neutrophils at D10-12 post-transplant compared to controls (FIG. 60C; data is expressed as mean±1SEM of 8-20 patients. *=p≦0.05). In addition, although not significant, autologous patients had higher lymphocyte counts throughout the time-points analyzed in LHRH-A treated compared to control group (FIG. 60D). This indicates that LHRH-A therapy significantly increases lymphocyte levels following stem cell transplantation.

II. Allogeneic Stem Cell Transplant Patients Undergoing LHRH-A Treatment Prior to Transplant Enhance Rate of Engraftment.

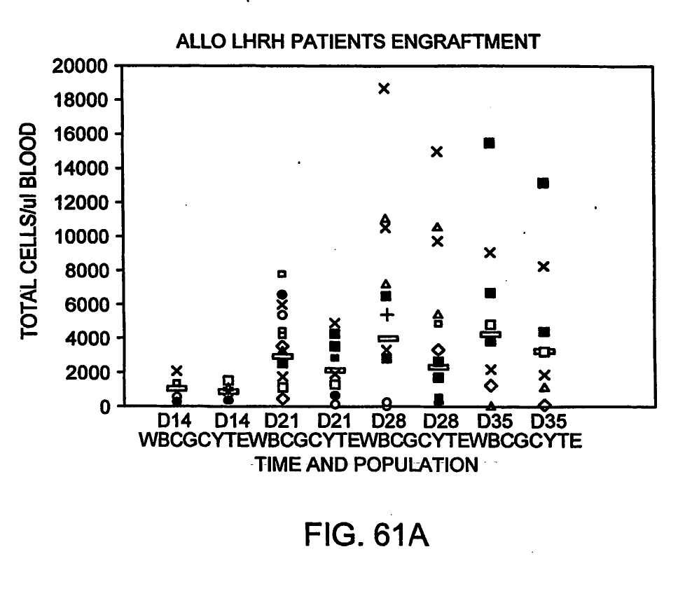

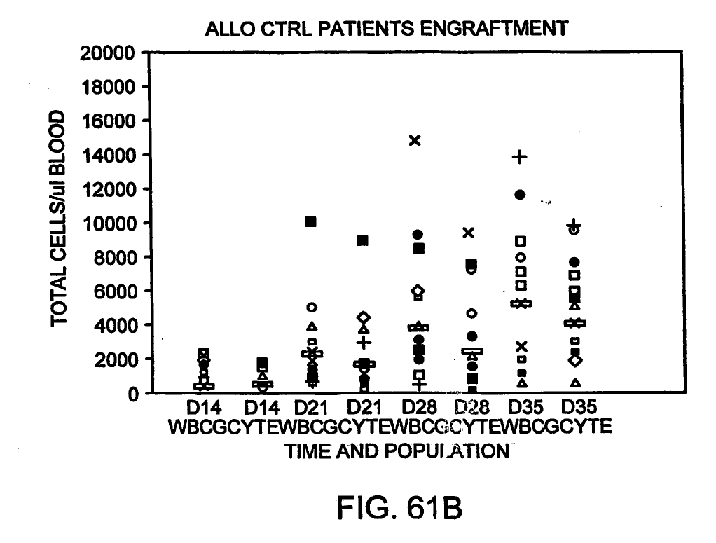

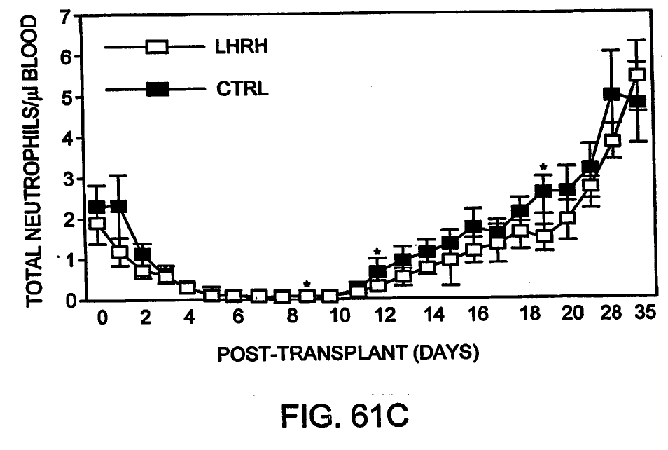

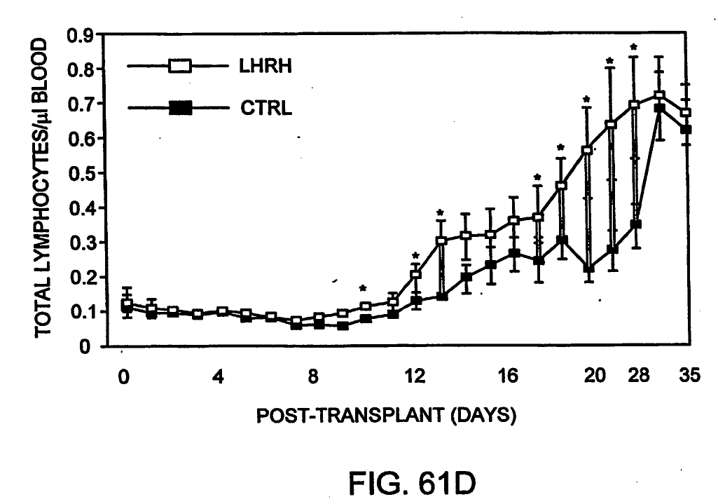

As shown in FIGS. 61A, C and D, allogeneic patients who were given LHRH-A treatment showed a significantly higher number of WBC at D14 post-transplant compared to controls (FIG. 61A) (p≦0.05) with 64% showing granulocyte engraftment (≧500 cells/μl blood) compared to 44% of controls at this time point. In addition, allogeneic patients who were given LHRH-A treatment showed a significantly higher number of neutrophils at D9, 12 and 19 post-transplantation compared to controls (FIG. 61C; data is expressed as mean±1SEM of 8-20 patients. *=p≦0.05). Additionally, analysis of patients undergoing peripheral blood stem cell transplantation demonstrated a significant increase in lymphocyte counts when treated with an LHRH-A prior to allogeneic transplantation (p≦0.05 at days 10, 12, 13 and 17-21 post-transplantation) (FIG. 61D).

In both the allogeneic and autologous transplant models, a significant increase in WBC and granulocyte numbers at D14 post-transplant was observed with LHRH-A treated patients compared to controls (FIGS. 60 and 61). This enhanced rate of engraftment is crucial for the overall patient morbidity with neutropenia (≦200 neutrophils/ml blood) indicative of increased infection rates. As such, an early recovery of WBC and granulocyte numbers demonstrates a better survival rate for LHRH-A treated patients. Inhibition of sex steroids enhances engraftment and reconstitution prior to full thymic regeneration or the release of new T cells as a result of full thymic regeneration.

### Example 21

### Sex Steroid Ablation Increases T Cell Proliferative Responses Within One Week

These studies were conducted to determine if sex steroid ablation was capable of enhancing proliferative responses as early as 3 to 7 days following castration in mice.

Materials and Methods:

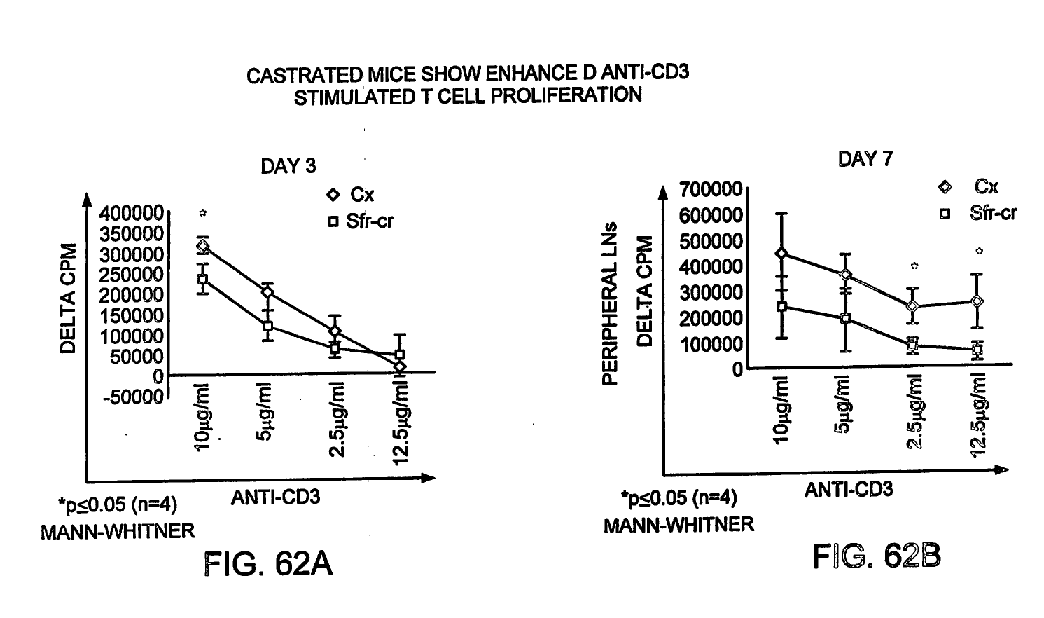

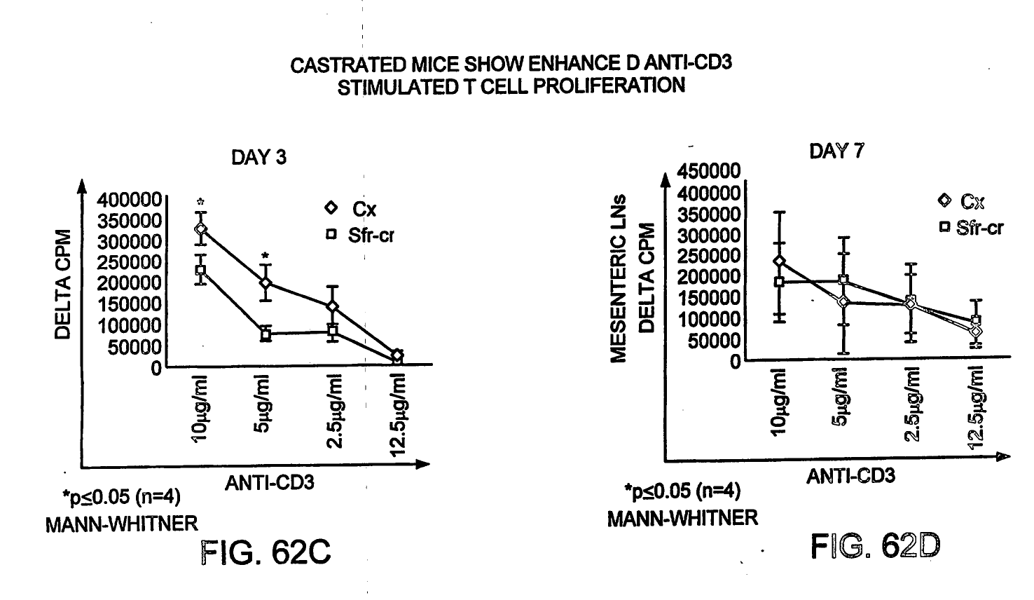

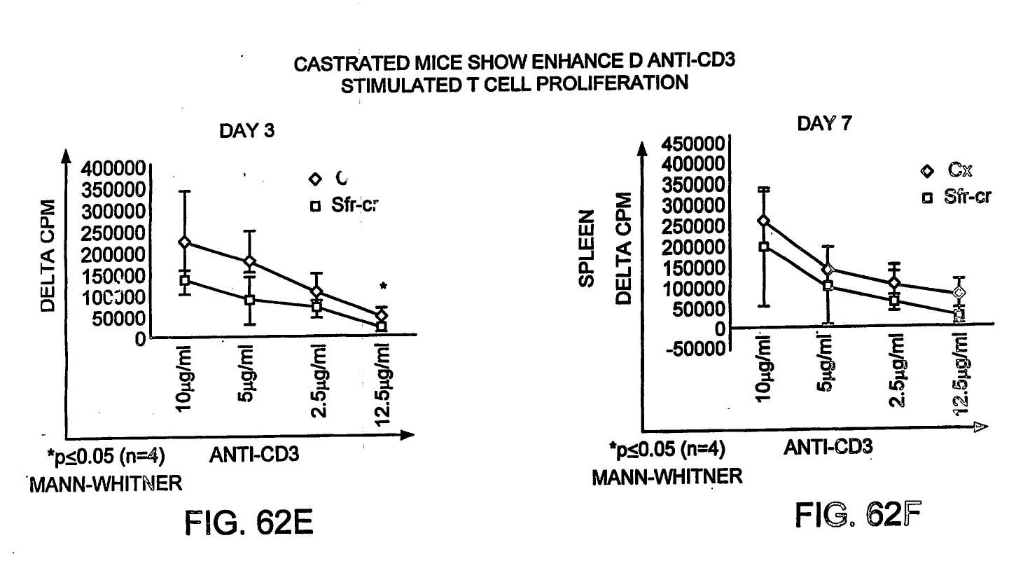

Eight week-old mice were castrated and analyzed for anti-CD3/anti-CD28 stimulated T cell proliferative response 3 days (FIGS. 62A, C, and E) and 7 days (FIGS. 62B, D, and F) after surgery. Peripheral (cervical, ancillary, brachial and inguinal) lymph node (FIGS. 62A and B), mesenteric lymph node (FIGS. 62C and D), and spleen cells (FIGS. 62E and F) were stimulated with varying concentrations of anti-CD3 and co-stimulated with anti-CD28 at a constant concentration of 10 μg/ml for 48 hours. Cells were then pulsed with tritiated thymidine for 18 hours and proliferation was measured as ³H-T incorporation. Control mice were sham-castrated, n=4, *p≦0.05 (non-parametric, unpaired, Mann-Whitney statistical test).

Results:

Sex steroid ablated mice show enhanced CD28/CD3-stimulated T cell proliferation at 3 days (FIGS. 62A, C, and E) and 7 days (FIGS. 62 B, D, and F) post-castration. T cells isolated from the peripheral LN showed a significant increase in proliferative responses at 3 days (10 μg/ml anti CD-3) and 7 days (2.5 μg/ml and 1.25 μg/ml anti-CD3) post-castration.

Additionally, T cells isolated from the mesenteric lymph nodes (FIGS. 62C and D) and spleen (FIGS. 62E and F) also showed a significant increase in anti-CD3-stimulated proliferation over sham-castrated mice at 3 days post-castration.

As early as 3-7 days post castration (prior to new T cells migrating from the thymus), there is an increase in responsiveness of T cells to stimulation with anti-CD3 and CD28 cross-linking. These data indicate that sex steroid ablation has direct effects on the functionality of the peripheral T cell pool, prior to thymic reactivation.

To determine the extent of direct effects of LHRH-A on peripheral T cells in human patients, studies are conducted. Control patient T cells are incubated with various doses of LHRH-A and are analyzed at varying time-points (D3, D7 and D14) for the level of proliferation compared to control (media alone) samples. This allows the determination of whether the LHRH-A acts directly on the existing T cells by causing their activation, as was observed in the mouse model.

### Example 22

### Sex Steroid Ablation Enhances Haemopoiesis Following Congenic HSCT

I. Castration Enhances Engraftment in the BM, Thymus, and Spleen Following HSCT.

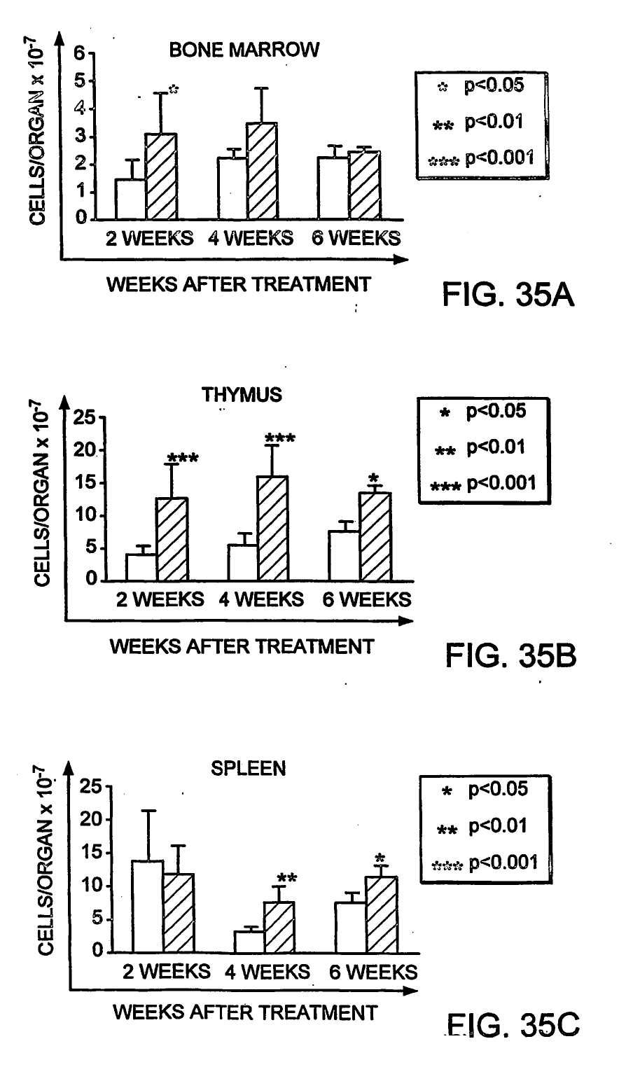

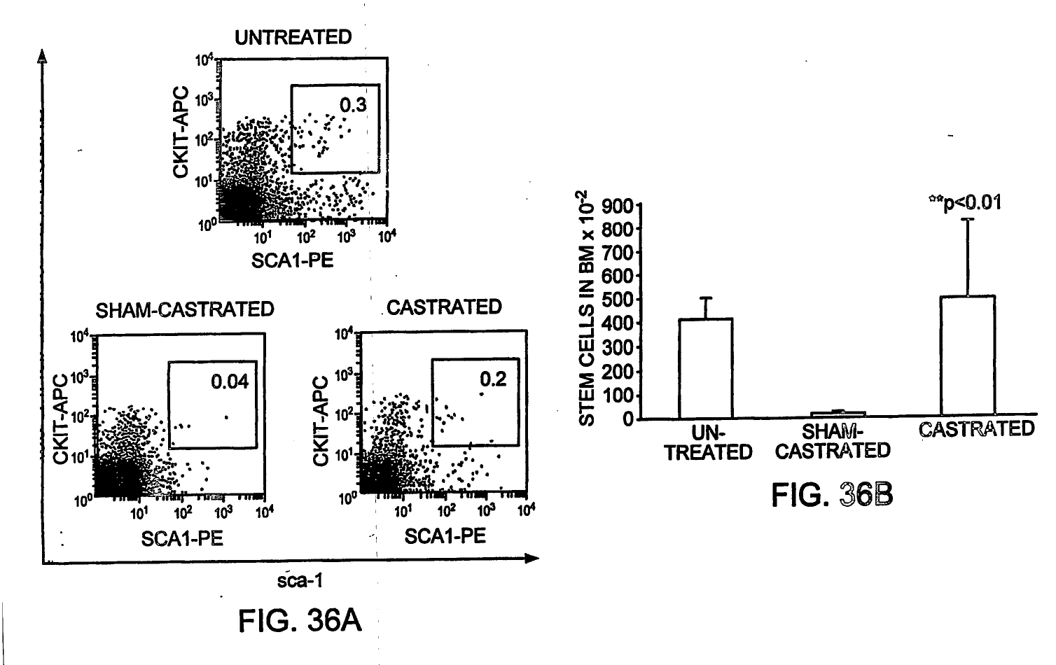

Mice were castrated 1 day before congenic HSCT. 5×10⁶ Ly5.1⁺ BM cells were injected intravenously into irradiated (800 rads) C57/BL6 mice. The BM, spleen and thymus were analyzed by-flow cytometry at various time points (2-6 weeks) post-transplant (FIG. 35). As shown in FIG. 35B, two weeks after castration and HSCT, there are significantly more cells in the BM of castrated mice as compared to sham-castrated controls. Similarly, as shown in FIG. 35C, there is a significant increase in thymic cell number 2, 4 and 6 weeks post-transplant as compared to sham castrated controls. As shown in FIG. 35C, in the periphery, splenic cell numbers are also significantly higher than controls 4 and 6 weeks post-transplant in the castrated recipients.

II. Castration Enhances Engraftment of HSC in the BM Following Congenic HISCT.

Mice were castrated 1 day before congenic HSCT. 5×10⁶ Ly5.1⁺ BM cells were injected intravenously into irradiated (800 rads) C57/BL6 mice. The BM was analyzed for lin-c-kit+sca-1+ HSC by flow cytometry at two weeks post-transplant (FIG. 36). Two weeks after BMT transplantation and castration there are significantly more donor-derived HSCs in the BM of castrated mice compared to sham castrated controls.

III. Castration Enhances Engraftment of HSC in the BM Following Congenic HSCT (2.5×10⁶ Cells).

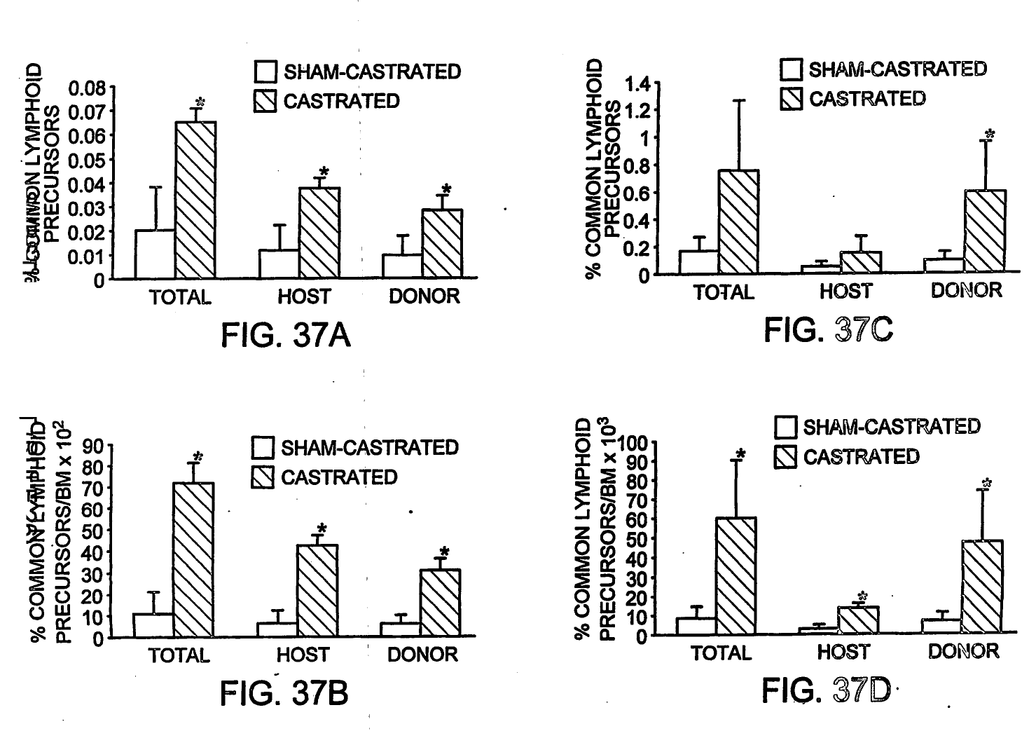

Mice were castrated 1 day before congenic HSCT. 2.5×10⁶ Ly5.1⁺ BM cells were injected intravenously into irradiated (800 rads) C57/BL6 mice. The BM was analyzed for lin-c-kit+sca-1+ HSC by flow cytometry at two weeks post-transplant (FIG. 37A-B). FIG. 37A depicts percent of common lymphoid precursors in the BM. FIG. 37B depicts the number of common lymphoid precursors in the BM. Two weeks after BMT transplantation and castration there is a significantly increased proportion of donor-derived HSCs in the BM of castrated mice compared to sham castrated controls.

IV. Castration Enhances Engraftment of HSC in the BM Following Congenic HSCT (5×10⁶ Cells).

5×10⁶ Ly5.1⁺ BM cells were injected intravenously into irradiated (800 rads) C57/BL6 mice. The BM was analyzed for lin-c-kit+sca-1+ HSC by flow cytometry at two weeks post-transplant (FIG. 37C-D). FIG. 37C depicts percent of common lymphoid precursors in the BM. FIG. 37D depicts the number of common lymphoid precursors in the BM. Two weeks after BMT transplantation and castration there is a significantly increased proportion of donor-derived HSCs in the BM of castrated mice compared to sham castrated controls.

V. Castration Enhances the Rate of Engraftment of Donor-Derived DC in the Thymus Following Congenic HSCT.

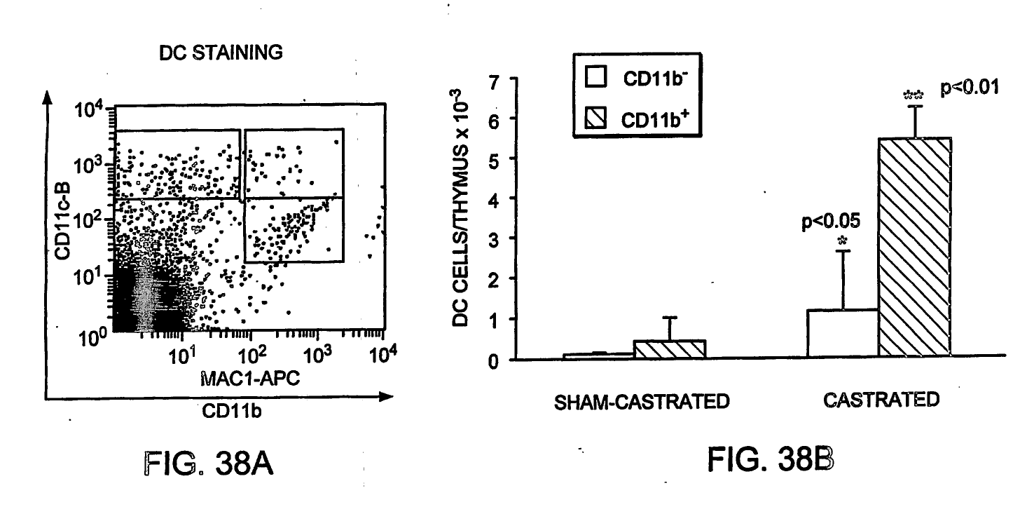

5×10⁶ Ly5.1⁺ BM cells were injected intravenously into irradiated (800 rads) C57/BL6 mice. Thymocytes were analyzed by flow cytometry at two weeks post-transplant. Donor-derived DC were defmed as CD45.1⁺CD11c⁺MHC class II⁺CD11b^{+ or -}. Donor-derived CD11b⁺ and CD11b⁻ DC are significantly increased in the thymii of castrated mice compared to sham-castrated controls 2 weeks after BMT (FIG. 38).

VI. Castration Enhances the Rate of Engraftment of Donor-Derived B Cells in the Spleen Following Congenic HSCT.

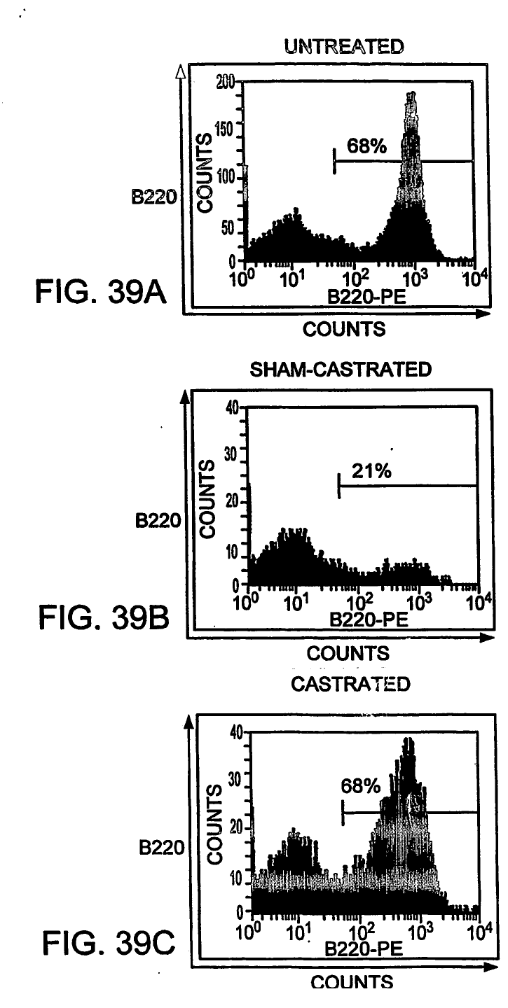

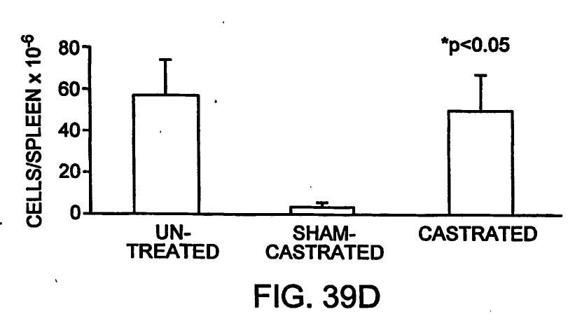

5×10⁶ Ly5.1⁺ BM cells were injected intravenously into irradiated (800 rads) C57/BL6 mice. Splenocytes were analyzed by flow cytometry at two weeks post-transplant. There are significantly more B220⁺ B cells in the spleens of castrated mice, as compared the sham-castrated controls, 2 weeks after congenics BMT (FIG. 39).

### Example 23

### LHRH-A Treatment Effectively Depletes Serum Testosterone

Materials and Methods:

Detection of sex steroid levels in patient sera was performed using a ¹²⁵I-Testosterone radioimmunoassay (RIA). Prior to the assay, all reagents, samples and controls were brought to room temperature. Control tubes had either buffer alone—non-specific binding (NSB) tube or 0 ng/ml testosterone standard (B₀). Buffer alone, standards (0-10 ng/ml testosterone) or test samples were added to each tube, followed by sex binding globulin inhibitor (SBGI) to limit non-specific binding of the radio-labeled testosterone. The ¹²⁵I-testosterone was added to each tube followed by an anti-testosterone antibody (except for the NSB tubes). Tubes were then incubated at 37° C. for 2 hours. Following this, a secondary antibody was added to all tubes which were vortexed and incubated for a further 60 mins. Tubes were centrifuged (1000_{gmax}) for 15 mins, supernatant removed and the precipitate counted on a Packard Cobra auto-beta counter. Triplicate cpm results were averaged and a standard curve constructed using the formula for percent bound testosterone (B/B₀):

$$\% B / {B}_{0} = \frac{Sample - NSB}{{B}_{0} - NSB}$$

Sample=average cpm of particular test sample
NSB=average cpm of non-specific binding tube
B₀=average cpm of 0 ng/ml standard (total binding tube)

The level of testosterone in each test sample was determined from the standard curve.

Results:

LHRH-A Administration to Prostate Cancer Patients Results in Castrate Levels of Serum Testosterone.

In order to determine the efficacy of LHRH-A treatment, serum testosterone levels were analyzed for all patients before treatment and at 4 months of treatment with LHRH-A. Analysis was performed using a radioimmunoassay (RIA) with ¹²⁵I-Testosterone. The concentration of serum testosterone was within the range of 1-3 ng/ml testosterone (mean=2.3 ng/ml) prior to hormonal treatment (FIG. 46A). At 4-months of treatment, patients had essentially no detectable serum testosterone indicating successful abrogation of sex steroid release.

LHRH-A Administration does not Affect the percent of Lymphocyte Subsets Within the Peripheral Blood.

Following 4-months of treatment with LHRH-A, no changes in the proportion of any lymphocyte subset was observed compared to pre-treatment values. These values are all within normal ranges. Peripheral blood lymphocytes were analyzed by FACS for proportions and cell numbers of T, B, and myeloid-derived (NK and macrophages) cells. No change in proportion of any cell subset was observed following LHRH-A administration. Furthermore, the proportions of all lymphocyte subsets were within normal ranges for this age group (FIG. 84C); Hannet et al., 1992; Xu et al., 1993).

### Example 24

### Thymic Reactivation Following Transient Ablation of Sex Steroids with Goserelin Acetate (Zoladex®) in Patients Undergoing Autologous or Allogeneic Stem Cell Transplantation

In this example, goserelin acetate (Zoladex®) is administered prior to autologous or allogeneic peripheral blood stem cell transplantation (PBSCT). The primary endpoint is thymic re-activation as measured by in vitro assays. Patients will be followed for six months post-transplant. Twenty (10 allografts and 10 autografts) patients will be entered into the study. This example investigates the effect of inhibiting sex steroid production at the level of LHRH, using agonists thereof to desensitize the pituitary and hence prevent release of LH and FSH. In turn this causes a block in the gonadal production of androgens and estrogen which removes the inhibitory effects on thymic function. The groups examined in this trial are patients undergoing high-dose chemoradiotherapy (HDT) and PBSCT.

Goserelin acetate (Zoladex®) is a potent synthetic decapeptide analogue of LHRH. When given acutely, goserelin acetate will release LH from the pituitary gland. However, following chronic administration, goserelin acetate is a potent inhibitor of gonadotrophin production resulting in gonadal suppression and, consequently, sex organ regression. In animals and humans, following an initial stimulation of pituitary, LH secretion and a transient elevation in serum testosterone, chronic administration results in inhibition of gonadotrophin secretion. The result is a sustained suppression of pituitary LH occurring within approximately three weeks of initiation of therapy and a reduction in serum testosterone levels in males to a range normally seen in surgically castrated men. This suppression is then maintained as long as therapy is continued.

Patients are male or female, aged 18 yrs. or older that are due to undergo high-dose therapy (HDT) with PBSCT for malignant disease or BM failure. The 10.8 mg implant formulation (for men) of the Zoladex® is dispersed in a cylindrical rod of biodegradable and biocompatible polyglactins and is released continuously over 12 weeks following subcutaneous injection. The 3.6 mg implant (for women) is dispersed in a cylindrical rod of a biodegradable and biocompatible polyglactin and is released continuously over 28 days following subcutaneous injection. The implants are commercially supplied in a purpose-designed applicator with 14-16 gauge needles.

Reduction of sex steroids in the blood to minimal values may take several weeks. Consequently, 21 days prior to PBSC infusion (day 0), patients are injected with the sex steroid-ablation therapy in the form of LHRH agonist Zoladex® (implant). For males, 10.8 mg goserelin as a single dose (effective for 3 months) are administered on day −21 with a further injection of 3.6 mg on day 63 (effective for 28 days). For females 3.6 mg (effective for 28 days) are administered on day −21 and days 7, 35 and 63. This should be effective in reducing sex steroid levels sufficiently to reactivate the thymus (predicted 4 months post PBSCT). Thus, in this example, only 4-6 months of treatment are administered. Other doses may be deemed acceptable as determined readily by those in the art.

PBPCs are infused on day 0. The reactivated thymus takes up the infused precursor cells and convert them into new T lymphocytes and epithelial thymic cells. Maximum sex steroid ‘ablation’ is at the time of PBSC infusion, and hence infused PBSC is able to assist in thymic reconstitution. Within 3-4 weeks after PBSCT the first new T cells are present in the blood stream but the therapy will be maintained for 3 months post-PBSCT to allow complete normalization of the immune system.

Thymic function is determined by assessment of T cell subsets by flow cytometry, T cell responses in vitro, and production of TRECs.

Prior to the start of the study, routine pre-HDT investigations are performed, and baseline FBE, electrolytes, LFTs documented. Other pre-treatment analyses include serum HCG (women), thymus CT, bone density studies, protein electrophoresis and immunoelectrophoresis, hormone studies: TFTs, FSH, LH, estrogen, progesterone, testosterone. Additionally, various baseline T cell assays are performed. Leukocytes are purified from 50 ml of blood and examined as follows:

(a) Flow Cytometry

Naïve vs. Memory T Cells
CD27-FITC, CD45RA-PE, CD45RO-PerCP, CD4- or CD8-APC
CD27-FITC, CD45RO-PerCP, CD4/CD8-APC, Ki-67-PE
CD62L, CD45RO-PerCP, CD103, CD4/CD8-APC

T Cell subsets
CD4-FITC, CD8-APC, αβTCR-PE, γδTCR-B/S-PerCP
CD25-PE, CD69-CyChrome, CD4-FITC, CD8-APC
CD28-CyChrome, αβTCR-PE, CD4-FITC, CD8-APC

B Cells/Myeloid Cells
CD19-FITC, CD3-PerCP, CD56-PE, CD34-APC
CD11b-CyChrome, CD11c-PE, CD4-FITC, CD8-FITC

Cytokines
IL-4-PE, IFNg-APC, CD4-FITC, CD8

Other Markers
CD11a, CD95, HLA-DR, CD2, CD5

All patients serve as internal controls because they are examined pre- and post-treatment. Staining specificity controls include isotype controls with FITC/PE/APC and blocking of FcR prior to staining.

(b) T Cell Function

Blood lymphocytes examined for their ability to respond to CD3 cross-linking in vitro.

(c) TREC Analysis

Naïve T cells will be isolated and probed for the presence of T cell receptor excision circles which are formed as a result of rearrangement of the TCR genes as described above. Their presence is a very strong indication of export from the thymus (being the only source of mainstream T cell production). Because cell division is associated with thymic development post-rearrangement of the TCR genes, TREC levels may be an underestimate of thymic migrants (about 10% of actual levels).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in biology or related fields are intended to be within the scope of the following claims.

### Example 25

### Castration Induces Tolerance to Allogeneic Graft

The-following mice are purchased from the Jackson Laboratory (Bar Harbor, Maine), and are housed under conventional conditions: C57BL/6J (black; H-2b); DBA/1J (dilute brown; H-2q); DBA/2J (dilute brown; H-2d); and Balb/cJ (albino; H-2d). Ages range from 4-6 weeks to 26 months of age and are indicated where relevant.

C57BL/6J mice are used as recipients for donor BM reconstitution. As described above, the recipient mice (C57BL/6/J older than 9 months of age, because this is the age at which the thymus has begun to markedly atrophy) are subjected to 5.5 Gy irradiation twice over a 3-hour interval. One hour following the second irradiation dose, the recipient mice are injected intravenously with 5×10⁶ donor BM cells from DBA/1J, DBA/2J, or Balb/cJ mice. BM cells are obtained by passing RPMI-1640 media through the tibias and femurs of donor (2-month old DBA/1J, DBA/2J, or Balb/cJ) mice, and then harvesting the cells collected in the media .

As described above, in recipient mice castrated either at the same time as the reconstitution or up to one week prior to reconstitution, there is an significant increase in the rate of thymus regeneration compared to sham-castrated (ShCx) control mice. In addition, as compared to the sham-castrated mice, castrated mice are found to have increased thymus cellularity, have more cells in their BM, and have enhanced generation of B cell precursors and B cells in their BM following BMT. Since the MHC (i.e., the H-2 locus in mice) of the recipient mice is different from that of the donor mice, detecting an increased number of donor-derived blood cells in castrated mice as compared to sham-castrated mice is straightforward. There is also the normal level and distribution of host and donor-derived DC in the chimeric thymus which are exerting negative selection (tolerance induction) to the host and donor.

Four to six weeks after reconstitution of the recipient mice with donor BM cells, skin grafts are taken from the donor mice and placed onto the recipient mice, according to standard methods (see, e.g., Unit 4.4 in Current Protocols In Immunology, John E. Coligan et al., (eds), Wiley and Sons, New York, N.Y. 1994, and yearly updates including 2002). Briefly, the dermis and epidermis of an anesthetized recipient mouse (e.g., a C57BL/6J mouse reconstituted with Balb/cJ BM) are removed and replaced with the dermis and epidermis from a Balb/cJ. Because the hair of the donor skin is white, it is easily distinguished from the native black hair of the recipient C57BL/6J mouse. The health of the transplanted donor skin is assessed daily after surgery.

The results will show that donor Balb/cJ skin transplanted onto a donor-reconstituted C57BL/6J mouse who has been castrated “takes” (i.e., is accepted) better than the donor skin transplanted onto a donor-reconstituted C57BL/6J mouse who is sham-castrated, e.g., because the sham-castrated mouse does not have adequate uptake of donor HSC into the host thymus to produce DC. A donor skin graft is found not to take on a recipient, sham-castrated, C57BL/6J mouse who has not been reconstituted with Balb/cJ BM.

An experiment is also performed to determine if a recipient mouse transplanted with donor BM can induce tolerance of a MHC matched, but otherwise different, skin graft. Briefly, male C57BL/6J mice (H-2b) are either castrated or sham-castrated. The next day, the mice are reconstituted with Balb/cJ BM (H-2d) as described above. Four weeks after reconstitution, two skin grafts (i.e., including the dermis and epidermis) are placed onto the recipient C57BL/6J mice. The first skin graft is from a DBA/2J (dilute brown; H-2d) mouse. The second skin graft is from a Balb/cJ mouse (albino; H-2d). Because the coat colors of C57BL/6J mice, Balb/cJ mice, and DBA/2J mice all differ, the skin grafts are easily distinguishable from one another and from the recipient mouse.

As described above, the skin graft from the Balb/cJ mouse is found to “take” onto the Balb/cJ-BM reconstituted castrated recipient mouse better than a Balb/cJ-BM reconstituted sham-castrated recipient mouse or a recipient mouse who has been sham-castrated and has not been reconstituted with donor BM. In addition, the skin graft from the DBA/2J mouse is found to “take” onto the Balb/cJ-BM reconstituted castrated recipient mouse better than a Balb/cJ-BM reconstituted sham-castrated recipient mouse or a recipient mouse who has been sham-castrated and has not been reconstituted with donor BM.

### Example 26

### Castration Effect on BM and Spleen in Thymectomized Mice

These preliminary experiments were performed to determine if castration effects were apparent in thymectomized patients (mice). The results indicated that disruption of sex steroid signaling has direct or indirect effects on the immune system (e.g., immune cells in the BM and thymus), irrespective of the presence of a regenerated thymus.

Materials and Methods

Mice were castrated and thymectomized using routine methods known in the art. Mice were divided into the following groups: untreated (i.e., naïve, “untreated”), sham castrated (“sham-cx”), and castrated (“cx”), and each of those three groups was then thymectomized (“tx”) or sham thymectomized (“shtx”) for a total of six groups analysed. Each of the six groups was analysed as 2 weeks and 4 weeks following myeloablation and BMT (see methods above).

Results

I. Thymectomy does not Impact the Effect of Sex Steroid Inhibition on the BM.

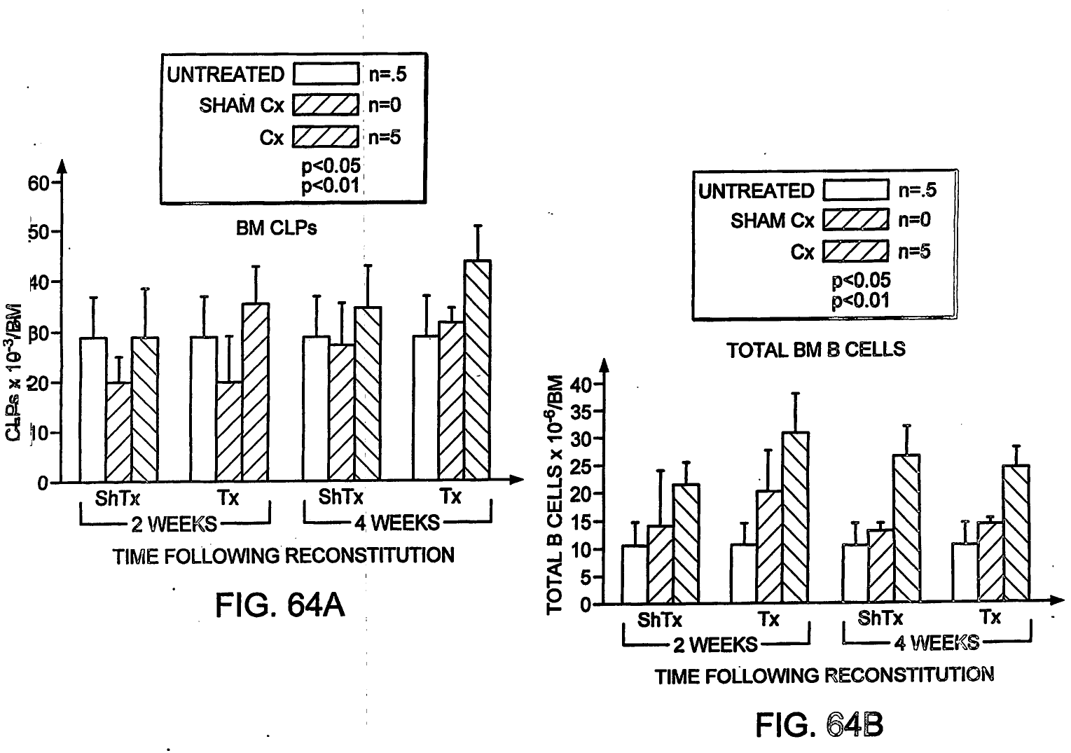

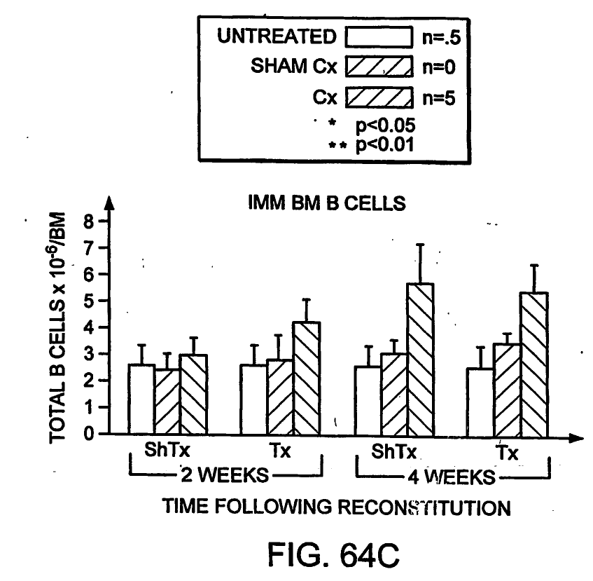

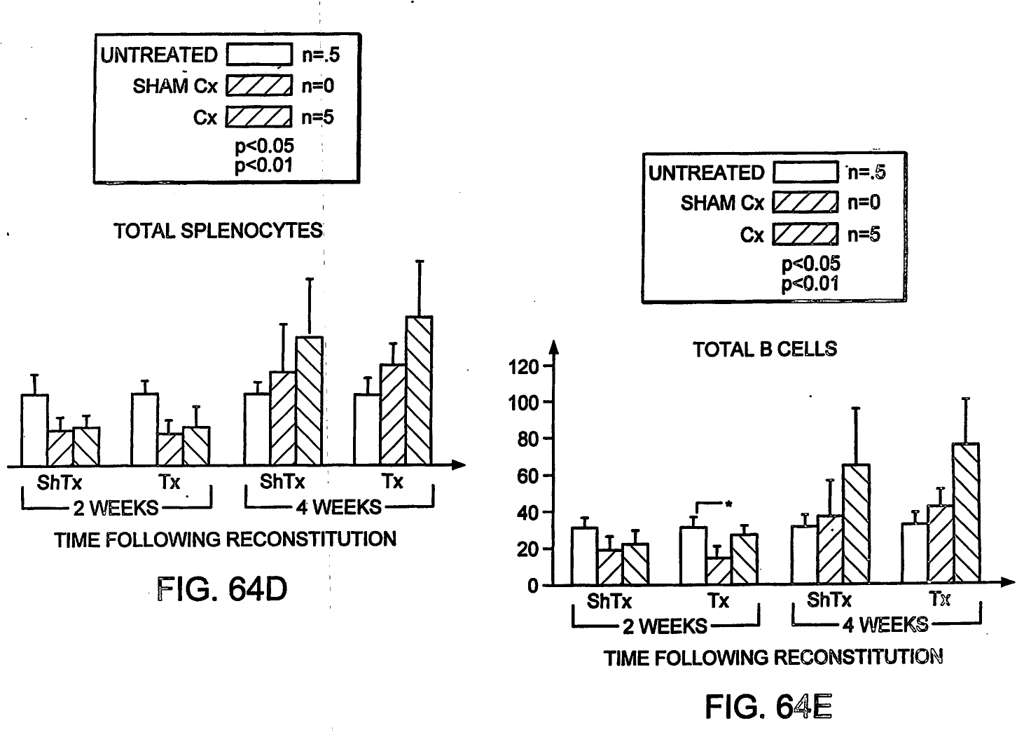

As shown in FIG. 64A, at 4 weeks post-BMT, Tx/Cx mice had an increase in the number of BM common lymphoid progenitors (CLPs), which is comparable to the ShTx/Cx mice.

As shown in FIG. 64B, at 4 weeks post-BMT, Tx/Cx mice have an increase in the total number of B cells in the BM, which is comparable to the ShTx/Cx mice. The Tx/Cx mice and the ShTx/Cx mice also have increased numbers of B cells in the BM, as compared to the either the ShamCx/Tx or ShamCx/ShTx controls.

As shown in FIG. 64C, at 4 weeks post-BMT, Tx/Cx mice also have an increase in the total number of immature B cells in the BM, which is comparable to the ShTx/Cx mice. The Tx/Cx mice and the ShTx/Cx mice also have increased numbers of immature B cells in the BM, as compared to the either the ShamCx/Tx or ShamCx/ShTx controls.

Thus, the results in FIGS. 64A-C support the conclusion that the effect of castration on increasing the number and functionality of cells in the BM, including increasing engraftment, does not require a reactivated thymus, and is instead due to direct effects on the BM and other cells of the immune system.

II. Thymectomy does not Impact the Effect of Sex Steroid Inhibition on the Spleen.

As shown in FIG. 64D, at 4 weeks post-BMT, Tx/Cx mice also appear to have an increase in the total number of cells in the spleen, which is comparable to the ShTx/Cx mice. The Tx/Cx mice and the ShTx/Cx mice also have increased total numbers of splenocytes, as compared to the either the ShamCx/Tx or ShamCx/ShTx controls.

As shown in FIG. 64E, at 4 weeks post-BMT, Tx/Cx mice also appear to have an increase in the total number of B cells in the spleen, which is comparable to the ShTx/Cx mice. The Tx/Cx mice and the ShTx/Cx mice also have increased numbers of B cells in the spleen, as compared to the either the ShamCx/Tx or ShamCx/ShTx controls.

Thus, the results in FIGS. 64D-E support the conclusion that the effect of castration on increasing the number and functionality of immune cells in the spleen, including enhanced reconstitution, does not require a reactivated thymus, and is instead due to direct effects on the BM and other cells of the immune system.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in biology or related fields are intended to be within the scope of the following claims.

Those skilled in the art will recognize, or be able to ascertain, using no more than routine experimentation, numerous equivalents to the specific embodiments described specifically herein. Such equivalents are intended to be encompassed in the scope of the following claims.

1-32. (canceled)

33. A method for improving tolerance in a patient to a graft from a mismatched donor without thymus reactivation, comprising:
depleting immune cells of the patient;
disrupting sex steroid-mediated signaling in the patient; and
administering cells from the donor to the patient, the cells being selected from the group consisting of stem cells, progenitor cells, and combinations thereof,
wherein the patient has increased tolerance to the donor graft without thymus reactivation compared to an untreated patient.

34. The method of claim 33, wherein the thymus of the patient is at least in part atrophied.

35. The method of claim 34, wherein the patient has a disease that at least in part atrophied the thymus of the patient.

36. The method of claim 34, wherein the patient has had a treatment of a disease that at least in part atrophied the thymus of the patient.

37. The method of claim 36, wherein the treatment of the disease is immunosuppression, chemotherapy, or radiation treatment.

38. The method of claim 33, wherein the stem cells are selected from the group consisting of hematopoietic stem cells, epithelial stem cells, and combinations thereof.

39. The method of claim 33, wherein the progenitor cells are selected from the group consisting of lymphoid progenitor cells, myeloid progenitor cells, and combinations thereof.

40. The method of claim 33, wherein the cells are hematopoietic stem cells.

41. The method of claim 38, wherein the hematopoietic stem cells are CD34⁺.

42. The method of claim 33, wherein the cells are administered at the time disruption of sex steroid-mediated signaling is begun.

43. The method of claim 33, further comprising administering to the patient a substance selected from the group consisting of a cytokine, a hematopoietin, a lymphokine, a interleukin, a CSF, a growth factor, and a combination thereof.

44. The method of claim 43, wherein the cytokine is selected from the group consisting of Interleukin 1 (IL-1), Interleukin 2 (IL-2), Interleukin 3 (IL-3), Interleukin 4 (IL-4), Interleukin 5 (IL-5), Interleukin 6 (IL-6), Interleukin 7 (IL-7), Interleukin 8 (IL-8), Interleukin 9 (IL-9), Interleukin 10 (IL-10), Interleukin 11 (IL-11), Interleukin 12 (IL-12), Interleukin 13 (IL-13), Interleukin 15 (IL-15), Interferon gamma (IFN-γ), and combinations thereof.

45. The method of claim 43, wherein the growth factor is selected from the group consisting of members of the epithelial growth factor family, members of the fibroblast growth factor family, stem cell factor, granulocyte colony stimulating factor (G-CSF), keratinocyte growth factor (KGF), granulocyte-macrophage colony stimulating factor (GM-CSF), insulin-like growth factor-1 (IGF-1), a growth hormone, a thyroid hormone, M-CSF, Meg-CSF, MIF, LIF, TNF, PDGF, human growth hormone, B cell growth factor, B cell differentiation factor, eosinophil differentiation factor, and combinations thereof.

46. The method of claim 33, wherein the sex steroid-mediated signaling is disrupted by surgical castration or chemical castration.

47. The method of claim 33, wherein the sex steroid-mediated signaling is disrupted by administration of a pharmaceutical.

48. The method of claim 47, wherein the pharmaceutical is selected from the group consisting of an LHRH agonist, an LHRH antagonist, an anti-LHRH vaccine, an anti-androgen, an anti-estrogen, a SERM, a SARM, a SPRM, an ERD, an aromatase inhibitor, an adrenal gland blocker, an aldosterone antagonist, an antiprogestogen, a progestin, an antiprogestin, a dioxalan derivative, and combinations thereof.

49. The method of claim 48, wherein the LHRH agonist is selected from the group consisting of goserelin, leuprolide, lupron, triptorelin, meterelin, buserelin, histrelin, nafarelin, lutrelin, leuprorelin, deslorelin, cystorelin, decapeptyl, gonadorelin, and acetates, citrates and other salts thereof, and combinations thereof.

50. The method of claim 48, wherein the LHRH antagonist is selected from the group consisting of abarelix, cetrorelix, acetates, citrates, and other salts thereof, and combinations thereof.

51. The method of claim 48, wherein the anti-androgen is selected from the group consisting of Cosudex®, bicalutamide, cyproterone acetate, liarozole, ketoconazole, flutamide, megestrol acetate, dutasteride, finasteride, eulexin, and combinations thereof.

52. The method of claim 48, wherein the anti-estrogen is selected from the group consisting of anastrozole, fulvestrant, tamoxifen, clomiphene, diethylstilbestrol, diethylstilbestrol diphosphate, danazol, droloxifene, iodoxyfene, toremifene, raloxofene, and combinations thereof.

53. The method of claim 48, wherein the adrenal gland blocker is selected from the group consisting of aminoglutethimide, formestane, vorazole, exemestane, anastrozole, letrozole, exemestane, and combinations thereof.

54. The method of claim 33, wherein the donor graft is selected from the group consisting of a cell of the donor, a tissue of the donor, an organ of the donor, and combinations thereof.

55. The method of claim 33, wherein the sex steroid-mediated signaling to the thymus is disrupted by reducing the level of a sex steroid hormone.

56. The method of claim 33, where the cells from the mismatched donor are genetically modified.

57. The method of claim 33, wherein the method results in the generation of a chimera selected from the group consisting of a chimeric thymus, a chimeric hemopoietic cell population, a chimeric lymphoid cell population, a chimeric T cell population, a chimeric B cell population, a chimeric dendritic cell-population, a chimeric lymphoid organ, and any combination thereof.

58. The method of claim 33, further comprising an allograft transplant having the same histocompatibility as that of the mismatched donor to the patient.

59. The method of claim 33, wherein tolerance is induced by at least one of: enhancing bone marrow hemopoiesis; enhancing bone marrow functionality; enhancing engraftment of donor cells in bone marrow; and increasing functionality of the patient's pre-existing immune cells.

60. A method for increasing the number of, or enhancing the mobilization of, hemopoietic stem cells in a donor, comprising disrupting sex steroid-mediated signaling in the donor prior to isolating hemopoietic stem cells, blood cells, and/or bone marrow from the donor.

61. The method of claim 60, further comprising administering to the donor an HSC mobilizing agent selected from the group consisting of cytokines, GM-CSF, G-CSF, CSF, chemotherapeutics, cyclophosphamide, flt-3 ligand, KGF/FGF7, other members of the FGF family, and IL-7.

62. The method of claim 60, wherein the sex steroid-mediated signaling is disrupted by surgical castration, or chemical castration.

63. The method of claim 60, wherein the sex steroid-mediated signaling is disrupted by administration of a pharmaceutical.

64. The method of claim 60, wherein the pharmaceutical is selected from the group consisting of an LHRH agonist, an LHRH antagonist, an anti-LHRH vaccine, an anti-androgen, an anti-estrogen, a SERM, a SARM, a SPRM, an ERD, an aromatase inhibitor, an adrenal gland blocker, an aldosterone antagonist, an antiprogestogen, a progestin, an antiprogestin, a dioxalan derivative, and combinations thereof.

65. The method of claim 64, wherein the LHRH agonist is selected from the group consisting of goserelin, leuprolide, lupron, triptorelin, meterelin, buserelin, histrelin, nafarelin, lutrelin, leuprorelin, deslorelin, cystorelin, decapeptyl, gonadorelin, and acetates, citrates and other salts thereof, and combinations thereof.

66. The method of claim 64, wherein the LHRH antagonist is selected from the group consisting of abarelix, cetrorelix, acetates, citrates, and other salts thereof, and combinations thereof.

67. The method of claim 64, wherein the anti-androgen is selected from the group consisting of Cosudex®, bicalutamide, cyproterone acetate, liarozole, ketoconazole, flutamide, megestrol acetate, dutasteride, finasteride, eulexin, and combinations thereof.

68. The method of claim 64; wherein the anti-estrogen is selected from the group consisting of anastrozole, fulvestrant, tamoxifen, clomiphene, diethylstilbestrol, diethylstilbestrol diphosphate, danazol, droloxifene, iodoxyfene, toremifene, raloxofene, and combinations thereof.

69. The method of claim 64, wherein the adrenal gland blocker is selected from the group consisting of aminoglutethimide, formestane, vorazole, exemestane, anastrozole, letrozole, exemestane, and combinations thereof.

70. The method of claim 60, wherein the sex steroid-mediated signaling to the thymus is disrupted by reducing the level of a sex steroid hormone.
